(19) United States
(12) Patent Application Publication
(10) Pub. No.: US 2012/0195831 A1
(43) Pub. Date: Aug. 2, 2012

(54) GENERATION, CHARACTERIZATION AND USES THEREOF OF ANTI-HER3 ANTIBODIES

(76) Inventors: Ningyan Zhang, Houston, TX (US); Hans Huber, Lansdale, PA (US)

(21) Appl. No.: 13/500,724

(22) PCT Filed: Oct. 7, 2010
(86) PCT No.: PCT/US10/51739

§ 371 Date: Apr. 6, 2012

Related U.S. Application Data
(60) Provisional application No. 61/250,060, filed on Oct. 9, 2009.

(51) Int. Cl. A61K 39/395; C07K 16/46; G01N 33/574; C07K 17/02; A61P 35/00; A61K 51/10; A61K 49/16; A61K 49/22; A61K 49/04; C07K 16/28; C12N 9/96

(52) U.S. Cl. 424/1.49; 530/389.1; 530/387.3; 435/7.23; 424/174.1; 530/391.7; 435/188; 530/391.3; 436/501; 424/9.341; 424/9.5; 424/9.3

(57) ABSTRACT

The present invention relates to Her3 specific antibodies, preferably fully human or humanized antibodies and antigen-binding portions thereof. Nucleic acid molecules encoding the Her3 antibodies as well as methods of use thereof are also disclosed. Also included are pharmaceutical compositions comprising these antibodies and methods of using the antibodies and compositions thereof for treatment and diagnosis of pathological hyperproliferative oncogenic disorders associated with aberrant expression of Her3 or Her2 including aberrant activation of each of these receptors.

### FIELD OF THE INVENTION

The present invention relates to a pharmaceutical composition comprising as an active agent an inhibitor of Her3 activity, particularly an anti-Her3 antibody. Further, the use of this composition for the diagnosis, prevention or treatment of hyperproliferative diseases, particularly tumor diseases is disclosed.

### BACKGROUND OF THE INVENTION

Intrinsic, cell-autonomous factors as well as non-autonomous, short-range and long-range signals guide cells through distinct developmental paths. An organism frequently uses the same signaling pathway within different cellular contexts to achieve unique developmental goals. Her3 signaling is an evolutionarily conserved mechanism used to control cell fates through local cell interactions. Signaling pathways between the extracellular environment and the nucleus of a cell involve the formation of many molecular complexes in which multiple proteins are assembled to directly or indirectly induce molecular events, such as enzyme activation or de-activation, Gomperts et al, Signal Transduction (Academic Press, N.Y., 2002). Such pathways and their components have been the subject of intense investigation because of the role aberrant pathway behavior plays in many disease conditions, especially cancer, e.g. McCormick, Trends in Cell Biology, 9: 53-56 (1999); Blume-Jensen and Hunter, Nature, 411: 355-365 (2001); Nicholson et al, Cellular Signalling, 14: 381-395 (2002). For example, investigators have observed that many cancers are associated with an accumulation of mutations or other genetic alterations that affect components of signaling pathways (e.g. by over expression), particularly those pathways involved with cell proliferation, cell motility, differentiation, and cell death, e.g. Blume-Jensen and Hunter, supra. Indeed, accumulating evidence suggests that cancer in humans is linked to the activity of non-viral, endogenous oncogenes, and that a substantial portion of these oncogenes code for protein tyrosine kinases. Many of the growth factor receptor proteins function as tyrosine kinases and it is by this process that they effect signaling. The interaction of growth factors with these receptors is a necessary event in normal regulation of cell growth. However, under certain conditions, as a result of either mutation or over expression, these receptors can become deregulated, the result of which is uncontrolled cell proliferation which can lead to tumor growth and ultimately to the disease known as cancer [Walks, A. F., Adv. Cancer Res., 60, 43 (1993) and Parsons, J. T.; Parsons, S. J., Important Advances in Oncology, DeVita, V. T. Ed., J. B. Lippincott Co., Phila, 3 (1993)]. The class of receptor tyrosine kinases is so named because when activated by dimerization, the intracellular domain of RTKs acquire tyrosine kinase activity that can, in turn, activate a variety signal transduction pathways. The predominant biological activity of some receptor tyrosine kinases is the stimulation of cell growth and proliferation, while other receptor tyrosine kinases are involved in arresting growth and promoting differentiation. In some instances, a single tyrosine kinase can inhibit, or stimulate cell proliferation depending on the cellular environment in which it is expressed.

Prominent among this class of enzymes implicated in the etiology of cancer are the receptor tyrosine kinases, which are a subclass of cell-surface growth-factor receptors with an intrinsic, ligand-controlled tyrosine-kinase activity. Ligand-mediated receptor tyrosine kinases are believed to function as “master switches” for a coordinated cellular communication network that regulates the normal proliferation of eukaryotic cells. This is generally accomplished by catalyzing the transfer of a phosphate group from ATP to a tyrosine residue located on a protein substrate. Schlessinger, Cell, 103: 211-225 (2000). As mentioned supra, cellular growth is generally instigated via ligand-stimulated tyrosine phosphorylation of intracellular substrates. By binding to specific peptide ligands these receptors are able to integrate these external stimuli with internal signal transduction pathways, and thereby instigate an intracellular web of biochemical processes with a capacity to drive dramatic cellular transitions, such as proliferation and migration. (Schlessinger, J. and Ullrich, A., Neuron, 9(3):383-391, 1992.).

A promising set of targets for therapeutic intervention in the treatment of cancer includes the members of the epidermal growth factor receptor since the reversible phosphorylation of tyrosine residues is required for activation of the EGFR pathway. In other words, EGFR-TKIs block a cell surface receptor responsible for triggering and/or maintaining the cell signaling pathway that induces tumor cell growth and division. The epidermal growth factor family can be subdivided into four groups based on their receptor-binding specificities (Her1, Her2, Her3, and Her4). These receptors are structurally related and include three functional domains: an extracellular ligand-binding domain, a transmembrane domain, and a cytoplasmic tyrosine kinase domain (Plowman, Culouscou et al. 1993). The extracellular domain can be further divided into four subdomains (I-IV), including two cysteine-rich regions (II and IV) and two flanking regions (I and III). Among the ErbB family, Her3 is inimitable because of its catalytically deficient kinase domain, its high propensity to self associate in the absence of ligand and the ability of the monomeric species of the extracellular domains of Her3 to assume a locked conformation, using an intramolecular tether.

Under normal physiological conditions, activation of the ERBB receptors is controlled by the spatial and temporal expression of their ligands, which are members of the EGF family of growth factors. Ligand binding to ERBB receptors induces the formation of receptor homo- and heterodimers. Dimerization consequently stimulates the intrinsic tyrosine kinase activity of the receptors and triggers autophosphorylation of specific tyrosine residues within the cytoplasmic tail. These phosphorylated residues serve as docking sites for a range of proteins, the recruitment of which ignites a cascade of signaling pathways that include various downstream adaptor and effector. Ultimately, downstream effects on gene expression determine the biological response to receptor activation. Nature Reviews Cancer 5, 341-354 (May 2005) Nancy E. Hynes. In essence, the secondary signal transducer molecules generated by activated receptors result in a signal cascade that regulates cell functions such as cell division or differentiation. Reviews describing intracellular signal transduction include Aaronson, S. A., Science, 254:1146-1153, 1991; Schlessinger, J. Trends Biochem. Sci., 13:443-447, 1988; and Ullrich, A., and Schlessinger, J., Cell, 61:203-212, 1990.

As one of the four members of the human epidermal growth factor receptor (EGFR) family, Her2 distinguishes itself in several ways. First, Her2 is an orphan receptor. The activation of the Her2 oncogene is believed to follow the binding of a yet unidentified growth factor ligand to the Her2 receptor complex, which leads to heterodimerization, triggering a cascade of growth signals that culminates in gene activation. increasing evidence suggests that it acts mainly as a co-receptor, increasing the affinity of ligand binding to dimeric receptor complex. Second, Her2 is a preferred partner for other EGFR family members (Her1/EGFR, Her3, and Her4) for the formation of heterodimers, which show high ligand affinity and superior signaling activity. Third, full-length Her2 undergoes proteolytic cleavage, releasing a soluble extracellular domain (ECD). Shedding of the ECD has been shown to represent an alternative activation mechanism of full-length Her2 both in vitro and in vivo, as it leaves a membrane-anchored fragment with kinase activity. Her2/neu is a very active tyrosine kinase, but cells expressing Her2/neu alone, and not other members of the EGFR family, fail to bind heregulin. The Her2 gene (c-erbB-2, neu) encodes a 185 kDa transmembrane tyrosine kinase receptor that has partial homology with other members of the epidermal growth factor receptor family [Shih, C., Padhy, L. C., Murray, M., et al. Transforming genes of carcinomas and neuroblastomas introduced into mouse fibroblasts, Nature, 290, 261-264 (1981)]. It is now known that normal human cells express a small constitutive amount of Her2 protein on the plasma membrane.

There is a growing body of evidence that members of the HER family are involved in breast cancer development and progression, but the protein expression pattern of all four HER receptors remains poorly understood [Bieche et al., Int J Cancer 106: 758-765 (2003); Bianchi et al., J Cell Physiol 206: 702-708 (2006); Auora, supra.]. The central role of Her2 in EGFR family signaling correlates with its involvement in the oncogenesis of several types of cancers, such as breast, ovarian, colon, endometrium, salivary gland, lung, kidney, colon and bladder and gastric cancers, regardless of its expression level (Slamon, D., et al., 1989, Science 244:707; Hynes, N., et al., 1994, Biochem. Biophys. Acta. 1198:165). Her2 may also render tumor cells resistant to certain chemotherapeutics (Pegram, M., et al., 1997, Oncogene 15:537). Both the erbB and erbB-2 genes have been shown to be activated as oncogenes by mechanisms involving overexpression or mutations that constitutively activate the catalytic activity of their encoded receptor proteins [Bargmann et al., Cell 45:649-657 (1986); Velu et al., Science 238:1408-1410 (1987)]. They are frequently upregulated in solid epithelial tumors of, by way of example, the prostate, lung and breast, and are also upregulated in glioblastoma tumors. Publications discussing EGFR and cancer are too numerous to disclose herein, but include Zeillinger et al., Clin. Biochem. 26:221-227, 1993; where it is asserted: Increased expression of this receptor [EGFR] has been found in various malignancies. In carcinomas of the cervix, ovaries, esophagus, and stomach, positive EGF-R status is definitely associated with the aggressiveness of the tumor. Likewise, over expression of the receptor kinase product of the erbB-2 oncogene has been associated with human breast and ovarian cancers [Slamon, D. J., et. al., Science, 244, 707 (1989) and Science, 235, 1146 (1987); David F. Stern, J. mammary Gland Biol. Neoplasm 13: 215-223 (2008).)]. Deregulation of EGF-R kinase has been associated with epidermoid tumors [Reiss, M., et al., Cancer Res., 51, 6254 (1991)], and tumors involving other major organs [Gullick, W. 3., Brit. Med. Bull., 47, 87 (1991). Some reports suggest hat Her2 protein overexpression occurs in approximately 30% of invasive human breast cancers, with Her2 gene amplification detected in 95% or more of the specimens found to over express Her2 protein, [Gebhardt et al., Biochem. Biophys. Res. Commun., 247, 319-323 (1998)]. Other reports suggest erbB-2 overexpression correlates with enhanced tumor aggressiveness and a high risk of relapse and death, and there is evidence that the overexpressed Her2 receptor leads to aggressive malignancies [Slamon et al., Science 235, 177-182 (1987); Dougall et al., DNA Cell Biol. 15, 31-40 (1996); Yarden, Y., 2001, Oncology 1:1) and altered sensitivity to hormonal and chemotherapeutic agents in transfection studies in cellular and animal models [Pegram et al., Oncogene, 15, 537-547 (1997); Witton et al., J Pathol 200: 290-297 (2003); P Arora, Oncogene 27: 4434-4445 (2008)]. As well, erbB-2 overexpression has been reported to be an important prognostic indicator of particularly aggressive tumors (Slamon et al, supra].

Furthermore, in many tumours EGF-related growth factors are produced either by the tumour cells themselves or are available from surrounding stromal cells, leading to constitutive EGFR activation [Sunpaweravong et al., 3. Cancer Res. Clin. Oncol. 131, 111-119 (2005); Salomon et al., Crit. Rev. Oncol. Hematol. 19, 183-232 (1995)].

WO 00/31048 discloses a quinazoline derivative which acts as an inhibitor of receptor tyrosine kinases such as EGFR, Her2 and Her4. An inhibition of Her3 is however not disclosed.

WO 00/78347 discloses methods for arresting or inhibiting cell growth, comprising preventing or reducing ErbB-2/ErbB-3 heterodimer formation. For example, the agent may be a combination of an anti-Her2 extracellular domain antibody and an anti-Her3 antibody, e.g. the Her3 antibody H3.105.5 purchased from Neomarkers. It is however not clear which type of anti-Her3 antibody is required to obtain desirable therapeutic effects.

U.S. Pat. No. 5,804,396 describes a method for identifying an agent for treatment of a proliferative disorder, comprising the steps of assaying a potential agent for activity in inhibition of signal transduction by a Her2/Her3 or Her2/Her4 or Her3/Her4 heterodimer. The patent is innocently silent relative to specific Her3 inhibitors.

Other references discussing cancer and EGFR include Karameris et al., Path. Res. Pract. 189:133-137, 1993; Hale et al., J. Clin. Pathol 46:149-153, 1993; Caraglia et al., Cancer Immunol Immunother 37:150-156, 1993; and Koenders et al., Breast Cancer Research and Treatment 25:21-27, 1993).

In the context of experimental cancer immunotherapy, the Human epidermal growth factor receptor 3 (also called Her3) signaling network is acquiring increasing importance for its possible roles in neoplastic cells and the immune system. Her3 is a transmembrane glycoprotein encoded by the c-erbB3 gene is a member of the epidermal growth factor receptor (EGFR) subfamily of type 1 receptor protein tyrosine kinase (RTK) family, which also includes EGFR, Her2/neu, and Her4 (see, e.g. U.S. Pat. No. 5,183,884; Ullrich et al., (1984) Nature 309, 418-425; Schechter et al., (1985) Science 229, 976-978; Plowman et al., (1993) Proc. Natl. Acad. Sci. U.S.A. 90, 1746-1750.) The ErbB3 receptor, together with ErbB2, is an important receptor involved in cellular growth and differentiation. Particular attention has focused on the role of ErbB3 as a coreceptor of ErbB2 in the area of cancer research.

Her3 distinguishes itself from other epidermal growth factor receptors in that it is a kinase defective receptor, e.g., low tyrosine kinase activity (see, e.g. Guy et al. (1994) Proc Natl Acad Sci USA 91(17), 8132-6; Carraway et al., (1994) J. Biol. Chem. 269, 14303-14306). However, it makes up for this deficiency in that it functions most effectively as a ligand binding receptor for neuregulins NRG-1 and NRG-2. As well, since ErbB-2 is devoid of an activating ligand, it can act only in the context of a heterodimer with a ligand-bound receptor. Consequently, Her2 requires Her3 in order to transform normal cells into cancer cells. 1 Her3 signaling thus relies on the formation of signaling-competent heterodimers with other ErbB members. Amongst the numerous combinations that are possible, the most mitogenic “couple” amongst the ErbB receptors is Her2/Her3. (Citri et al., 2003). Indeed, this receptor pair (Her2/Her3) forms the most potent signaling module of the ErbB-receptor family in terms of cell growth and transformation. [Karamouzis et al., Int'l J. of Biochemistry & Cell Biol., 39: 851-856 (2007); Citri et al., Experimental, Cell Research, 284: 54-65 (2003)]. Hence, both Her2 and Her3 are active only in the context of ErbB heterodimers, and ErbB-2. This dimerization enables Her2 to activate the PI3K signaling pathway. Indeed, increased expression of Her3 increases the signaling potency of Her2, whereas decreased Her3 expression results in the loss of Her2 activity. Her3 is involved in Her2-mediated tumorigenesis through dimerization with Her2. Hsieh A C, Moasser M M. Br J Cancer. 2007; 97:453-457. A characteristic feature of attendant the potency of signaling by the ligand-activated ErbB-2/ErbB-3 heterodimer lies in the fact that this dimer has the capacity to signal very potently, both through the Ras-Erk pathway for proliferation, and through the phosphatidylinositol-3′-kinase (PI3K)-Akt pathway for survival. More, this receptor dimer evades downregulation mechanisms, leading to prolonged signaling. That the most potent signaling module is formed by partners that are incapable of productively signaling in isolation suggests that evolutionary forces formed these mechanisms as a measure to tightly control the output of the network.

Several lines of evidence have emerged in recent years that provide support for the pivotal role of Her3 in human tumorigenesis. An important observation pertaining to ErbB heterodimer collaboration during tumor development is that expression of ErbB3 is seen in many of the same tumor types that overexpress ErbB2, including breast, bladder and melanomas [Rajkumar et al., Clin. Mol. Path., 49: M199-M202 (1996)]. Furthermore, many ErbB2-overexpressing breast tumors display elevated levels of phosphotyrosine on ErbB3 probably as a result of spontaneous dimerization with ErbB2. [Siegel P M, Ryan E D, Cardiff R D and Muller W S. (1999). EMBO J. 18,2141/2164.] Likewise, Mosesson et al., Seminars in Cancer Biology, 14: 262-270 (2004) report that ErbB-3, is abundantly expressed in several carcinomas (e.g., breast, colon and gastric tumors). Increased expression of EGFR and ErbB3 is also correlated with reduced breast cancer patient survival [Nicholson R I, Gee J M, Harper M E. (2001). EGFR and cancer prognosis. Eur J Cancer 37: S9-S15; Witton et al. J Pathol 200: 290-297 (2003).](Nicholson et al., 2001; Witton et al., 2003). Her2/Her3 heterodimers have been shown to be constitutively active in breast cancer cells with Her2 gene amplification. Simultaneous overexpression of Her2 and Her3 has been detected in 12-50% of invasive breast cancers, and the increased drug resistance in many Her2-overexpressing cancers depends on augmented levels of Her3 and/or EGFR [Abd El-Rehim et al. British Journal of Cancer pp. 1532-1542 (2004)]. Her3 has been found to be overexpressed in various organs including breast, lung, pancreas and stomach. Furthermore, its overexpression has been documented in 20-30% of invasive and in approximately one third of in situ breast carcinomas, and is associated with poor prognostic factors [Badra et al., Cancer Letters, 244: 34-41 (2006)].

Presently, two approaches are being utilized to target the EGFR's. One approach proposes the use of monoclonal antibodies (mAbs) to target the extracellular domain of the EGFR to block natural ligand binding. [Wheeler et al., Oncogene (2008) 27, 3944-3956; doi:10.1038/one.2008.19; published online 25 Feb. 2008]. Representative examples include Herceptin, and Cetuximab. The second approach involves the use of small molecule tyrosine kinase inhibitors (TKIs) that bind to the ATP-binding site in the tyrosine kinase domain (TKD) of the EGFR and Her2. Three anti-EGFR TKIs are representative of this group-erlotinib (OSI-774, Tarceva), gefitinib (ZD1839, Iressa) and lapatinib (GW572016, Tykerb). Each has be approved by the FDA for use in oncology.

While both approaches to EGFR inhibition show considerable clinical promise, increasing evidence suggests that patients who initially respond to EGFR inhibitors may subsequently become refractory [Wheeler et al. supra; Pao et al., PLoS Med 2: 1-11. (2005)]. With respect to the antibody approach, resistance to monoclonal antibodies targeting Her2 are well documented. [Lu Y et al., J Natl Cancer Inst 2001, 93: 1852-1857]. Additional limitations relative to the use of antibody based therapy for targeting Her2 have been noted. First, its inhibitory effect is restricted to the Her2 displayed on the cell surface; intracellular Her2 molecules are still available for mitogenic signaling. Second, Herceptin can be bound and thus “neutralized” by circulating ECDs that are released by proteolysis of membrane-bound Her2 (Brodowicz, T., et al., 1997, Int. J. Cancer 73:875). Finally, as with many other drugs, prolonged treatment with Herceptin leads to acquired resistance (Kute, T., et al., 2004, Cytometry Part A 57A:86). Another anti-Her2 antibody, pertuzurnab, has been shown in a phase II clinical trial to have activity in ovarian cancer (Gordon, M. S., et al., 2006, J. Clin. Oncol. 24:4324). However, only patients with highly amplified Her2 respond significantly to Herceptin therapy, thus limiting the number of patients suitable for therapy. In addition the development of resistance to drugs or a change in the expression or epitope sequence of Her2 on tumor cells may render even those approachable patients unreactive with the antibody and therefore abrogating its therapeutic benefits.

Along with the biological impact of Her3 signaling on ERBB2-amplified cellular proliferative disorders such as breast cancer, increasing evidence links active Her3 to resistance to breast cancer therapeutics targeted at ERBB2 and ER. Indeed, a significant limitation in using these compounds is that recipients thereof may develop a resistance to their therapeutic effects after they initially respond to therapy, or they may not respond to EGFR-TKIs to any measurable degree ab initio. In fact, only 10-15 percent of advanced non-small cell lung cancer patients respond to EGFR kinase inhibitors. Since Her3 has no catalytic activity, it appears that Her3 promotes drug resistance by enabling autocrine or paracrine ligands (NRG1 and NRG2) to activate catalytically competent RTKs, and through its capacity to channel signaling to PI3K/Akt signaling pathways. Finally, it is formally possible that Her3 affects response to ERBB inhibitors indirectly, through protection of ERBB2 kinase domain or extracellular domain in heterodimers from phosphatases or inhibitors, or by reducing formation of ERBB2 homodimers, or dimers with other receptors such as ERBB4 that may have protective value for patients. [Stern, J. mammary Gland Biol. Neoplasm 13: 215-223 (2008)]. It appears that when Her2 is targeted using a tyrosine kinase inhibitor, tumor cells can compensate by upregulating Her3 activation making it more difficult to reverse the process of Her2:Her3 phosphorylation. Thus, Her3 does not remain “turned off” after tyrosine kinase inhibitor treatment and is able to be phosphorylated, which allows for activation of the PI3K/Akt pathway. [Sergina et al., Nature. 2007; 445:437-441.] For patients that develop resistance, this potentially life-saving therapeutic mechanism fails to achieve what they had hoped for and so desperately needed—an active therapy for cancer. Thus, although the compounds may, at first, exhibit strong anti-tumor properties, they may soon become less potent or entirely ineffective in the treatment of cancer.

Recent studies further support numerous prior epidemiologic reports indicating that a considerable proportion of middle-aged women are at a high risk for cardiovascular disease (CVD) independent of prior history of breast cancer treatment. Apparently, patients with early-stage Her2/neu-positive breast cancer seem to be at an even greater risk of CVD than these women due to the direct and indirect toxic effects of adjuvant breast cancer therapy. [Cancer Epidemiology Biomarkers & Prevention 16: 1026-1029 (2007); Lee W. Jones 1 Internal Medicine News, Feb. 15, 2006 by Bruce Jancin.

Murine or chimeric Her3 antibodies have been reported, such as in U.S. Pat. No. 5,968,511, U.S. Pat. No. 5,480,968 and WO03013602. A monoclonal antibody against Her3 (Rajkumar et al., Br. J. Cancer 70 (1994), 459-456) had an agonistic effect on the anchorage-independent growth of cell lines expressing Her3. On the other hand, anti-Her3 antibodies described in U.S. Pat. No. 5,968,511 are reported to reduce Heregulin-induced formation of Her2/Her3 heterodimers. Such an activity, however, is only demonstrated for an antibody which increases Heregulin binding to Her3. Likewise, while van der Horst E H, Murgia M, Treder M, et al; Int J Cancer 115:519-527, 2005 have demonstrated the inhibitory effect of an antibody directed against Her3 (α-Her3ECD) on Her3-mediated signaling, it is unclear if the antibody had any effect in-vivo. As well, the antibody is a murine antibody liable to induce a HAMA response in humans. Towards this end, the authors speculate that it would be very interesting to see whether the α-Her3ECD in vitro data could be translated to an in vivo setting. Thus, at present it is not clear which type of anti-Her3-antibody—if any—has potential of being used for therapeutic applications.

As well, the prior art suggests that while interfering with signaling through the ERBB2-Her3 dimer might offers an alternative therapeutic strategy to targeting ERBB2 alone, such inhibition appeared to be transitory in some cases and compensatory mechanisms allowed cells to restore PI3K-Akt pathway signaling and tumorigenesis. As well, the prior art suggests that there were concerns regarding the dose required to achieve a complete blockade of the signaling pathway. “A central role for HER3 in HER2-amplified breast cancer: implications for targeted therapy. Cancer Res. 68, 5878-5887 (2008); Nature Reviews Cancer 9, 463-475 (July 2009)

While the proposed partnership between Her2/Her3 have created opportunities for improving efficacy of ERBB-targeted pharmaceuticals, by interfering with coupling of ERBB2 to ERBB3 through dimerization inhibitors, the art is completely silent as to the identification of a Her3 selective antagonist that is therapeutically effective in treating Her3 mediated cellular proliferative disorders. Indeed, despite encouraging results, the failure of Herceptin therapies for many ERBB2-amplified breast cancers, the absence of a Her3 selective antibody that is therapeutically effective together with the eventual development of therapeutic resistance in cases where robust responses occur at first, has created a need for an alternative, more potent, Her3 inhibitor. More, in spite of the discouraging landscape attendant conventional tyrosine kinase inhibitors, in particular, Her3 antibodies, the inventors endeavored to develop effective Her3 antagonists, the details of which are disclosed herein.

Disclosed herein are novel Her3 selective agents, e.g., antibodies that are unencumbered by the deficiencies attendant current Her2/Her3 moieties. Also disclosed is a pharmaceutical composition comprising as an active agent a specific type of inhibitor of Her3 activity, e.g., antibody and pharmaceutically acceptable carriers, diluents and/or adjuvants. Methods of using the Her3 antibodies are also disclosed.

### SUMMARY OF THE INVENTION

Embodiments of this invention are made available by the development of antibodies that retain favorable affinity to the Her3 receptor protein, particularly human Her3 receptor protein. The antibodies described infra, (“Invention Antibodies”) offer an important new approach to the treatment of various disorders of cell fate, in particular hyperproliferative disorders (e.g., cancer). Disorders involving aberrant Her3 or Her2 receptor activation or undesirable levels of expression or activity of Her3 protein are also included.

A broad aspect of the invention relates to at least one monoclonal antibody, or binding fragment thereof described herein that binds specifically to an antigen present in various cancers mediated by or related to Her3 activation or dysregulation, wherein the antigen is Her3.

Another broad aspect of the invention provides a plurality of anti-Her3 antibodies, preferably anti-Her3 monoclonal antibodies. The monoclonal antibodies of the invention bind to the human Her3 receptor (Her3) and can thus be useful in methods to treat or diagnose pathological hyperproliferative oncogenic disorders mediated by Her3 expression or dysplastic cells associated with increased expression or activity of the Her3 receptor protein.

An embodiment of this invention relates to the antibodies described herein, including the sequences of the VRs, FRs and CDRs polypeptides and the polynucleotides encoding them. Variant antibodies exemplified by diabody, bi-specific, trivalent & tetravalent antibodies or other antibodies derived from the herein described invention antibodies are also encompassed by the invention.

Another aspect of the invention relates to the use of these antibodies in methods or assays for detecting Her3 activation or expression in patients suspected of having a Her3 related disease or disorder. Such diseases or disorders may include, but not limited to, cerebral autosomal dominant arteriopathy with subcortical infarcts and leukoencephalopathy (CADASIL), T-cell acute lymphoblastic leukemia, lymphoma, Alagille syndrome, liver disease involving aberrant vascularization; diabetes, ovarian cancer, diseases involving vascular cell fate, rheumatoid arthritis, pancreatic cancer, plasma cell neoplasms (such as multiple myeloma, plasma cell leukemia, and extramedullary plasmacytoma), and neuroblastoma.

Another aspect of the invention relates to the screening of a patient suspected of having a Her3 related disease or condition to determine if such a patient would benefit from treatment with an anti-Her3 antibody. Such detection includes both cell surface detection as well as soluble Her3 found in the serum of said patient. See infra.

The invention also provides an isolated cell line that produces at least one anti-Her3 antibody as described herein. An embodiment of the invention thus provides an isolated cell line which produces at least one or more of the monoclonal antibodies as detailed herein that binds specifically an antigen present in one of T cell acute Lymphoblastic leukemia (T-ALL), human breast cancer, human colon cancer, melanoma, human lung cancer and human prostate cancer, the antigen being Her3 receptor protein (i) a polypeptide having a molecular weight of about 270 kDa as determined by SDS-PAGE under reducing conditions.

In certain embodiments, at least one invention described herein binds to the ligand binding domain of the Her3 receptor.

In yet another embodiment, at least one antibody of the invention binds to the negative regulator region, resident in the extracellular domain of the Her3 receptor.

It is understood that the term “antibody” includes “antibodies” such as one or more of the Her3 specific antibodies described herein including those that also bind Her3. As well, it includes monoclonal, polyclonal, multivalent, bispecific, and trivalent or optimized antibodies including fragments thereof. The invention also contemplates the use of single chains such as the variable heavy and light chains of the antibodies. Generation of any of these types of antibodies or antibody fragments is well known to those skilled in the art. In the present case, monoclonal antibodies to Her3 receptor proteins have been generated and have been isolated and shown to have high affinity to Her3.

The invention also includes modifications to the invention antibodies including variants thereof which do not significantly affect their binding properties. Such variants may have enhanced or decreased activity towards its binding partner.

Another embodiment of the invention encompasses monoclonal antibody or binding fragment thereof that may be Fab fragments, F(ab′)₂ fragments, Fab′ fragments, F(ab′)2 fragments, Fd fragments, Fd′ fragments or Fv fragments, Fv, scFv, scFv-Fc or diabodies or any functional fragment whose half-life would have been increased by a chemical modification, especially by PEGylation, or by incorporation in a liposome. It may also be an anti-idiotypic antibody. Plasma protein binding can be an effective means of improving the pharmacokinetic properties of otherwise short lived molecules.

One general strategy of reducing a therapeutic proteins intrinsic rate of clearance is via amino acid substitutions. In case of a protein, this strategy may entail amino acid substitutions that reduce receptor binding affinity in intracellular endosomal compartments, thereby leading to increased recycling in the ligand-sorting process and consequently resulting in longer half-life in extracellular medium. See Sarkar C. A., Lowenhaupt K., Horan T., Boone T. C., Tidor B., Lauffenburger D. A. Nat. Biotechnol. (2002) 20:908-913. A second approach is to express the therapeutic protein as a genetic fusion with a natural protein that has a long serum half-life; either 67 kDa serum albumin (SA)—Syed S., Schuyler P. D., Kulczycky M., Sheffield W. P. Blood (1997) 89:3243-3252) or the Fc portion of an antibody, which adds an additional 60-70 kDa in its natural dimeric form, depending on glycosylation (Mohler et al., J. Immunol., 151:1548-1561 (1993). As a consequence, an embodiment of the invention provides modifications to at least one antibody disclosed herein resulting in a fusion protein comprising an antibody of the invention fused to albumin. See Dennis et al., “Albumin binding as a general strategy for improving the pharmacokinetics of proteins.” J Biol. Chem., 277:35035-43 (2002).

Glycosylated variants (Glycoforms) of the invention antibodies are also envisioned. In one embodiment of the invention, antibodies, or fragments thereof, are modified to reduce or eliminate potential glycosylation sites. The amino acids where carbohydrate, such as oligosaccharide, is attached are typically asparagine (N-linkage), serine (O-linkage), and threonine (O-linkage) residues. In order to identify potential glycosylation sites within an antibody or antigen-binding fragment, the sequence of the antibody is examined, for example, by using publicly available databases such as the website provided by the Center for Biological Sequence Analysis (see http://www.cbs.dtu.dk/services/NetNGlyc/ for predicting N-linked glycosylation sites) and http://www.ebs.dtu.dk/services/NetOGlyc/ for predicting O-linked glycosylation sites). Additional methods for altering glycosylation sites of antibodies are described in U.S. Pat. Nos. 6,350,861 and 5,714,350, the entire content of each of which is incorporated herein in its entirety. In order to improve the binding affinity of an antibody or antigen-binding fragment thereof, glycosylation sites of the antibody can be altered, for example, by mutagenesis (e.g., site-directed mutagenesis). Such modified antibodies having reduced glycosylation sites or carbohydrates relative to the unmodified form are referred to as “aglycosylated” antibodies. An “afucosylated” anti-Her3 antibody derived from one or more antibodies described herein is representative of such an antibody that falls within the scope of the invention. See Li et al., Nat. Biotechnol., 24: 210-215 (2006); Shields, R. L. et al. Lack of fucose on human IgG1 N-linked oligosaccharide improves binding to human FcγRIII and antibody-dependent cellular toxicity. J. Biol. Chem. 277, 26733-26740 (2002). In alternative embodiment, the invention antibodies or antigen binding fragments thereof are modified to enhance glycosylation.

Fc engineered variants of the invention antibodies are also encompassed by the present invention. Such variants include antibodies or antigen binding fragments thereof which have been engineered so as to introduce mutations or substitutions in the Fc region of the antibody molecule so as to improve or modulate the effector functions of the underlying antibody molecule relative to the unmodified antibody. In general, improved effector functions is included o refer to such activities as CDC and ADCC. Further thereto, the invention provides Fc variants that have improved function and/or solution properties as compared to the aglycosylated form of the parent Fc polypeptide. Improved functionality herein includes but is not limited to binding affinity to an Fc ligand. Improved solution properties herein includes but is not limited to stability and solubility. In one embodiment, the proposed Fc variants bind to an FeγR with an affinity that is within about 0.5-fold of the glycosylated form of the parent Fc polypeptide. In an alternate embodiment, the aglycosylated Fc variants bind to an FcγR with an affinity that is comparable to the glycosylated parent Fc polypeptide. In an alternate embodiment, the Fc variants bind to an FeγR with an affinity that is greater than the glycosylated form of the parent Fc polypeptide.

Another broad aspect of the invention comprises an antibody or a binding fragment thereof that comprises a light chain comprising at least one complementarity determining region CDR having an amino acid sequence as set forth in one or more appendices detailed herein (Tables 1-4) or at least one CDR whose sequence has at least 80%, preferably 85%, 90%, 95% and 98% identity, after optimum alignment, with the sequences set forth in one or more appendices described herein or a heavy chain comprising at least one CDR comprising an amino acid sequence selected from the group set forth in one or more appendices set forth herein or at least one CDR whose sequence has at least 80%, preferably 85%, 90%, 95% and 98% identity, after optimum alignment, with said at least one CDR as set forth in one or more appendices set forth herein. Alternatively, the antibody of the invention comprises at least one heavy chain and/or a light chain comprising at least one amino acid sequence as set forth in one of Tables 5 or 6. Nucleic acid molecules comprising nucleotide sequences encoding at least one or more of the above referenced amino acid sequences are also contemplated. See Appendices I-III.

The light chain may likewise comprise the amino acid sequence as set forth in one or more table detailed herein, while the heavy chain may comprise the amino acid sequence as set forth in one or more table as set forth herein. See Tables 1-6.

Antibodies that compete with any one or more of the antibodies described herein for binding with Her3 1 are also within the scope of the invention.

In recent years, various strategies have been developed for preparing scFv as a multimeric derivative. This is intended to lead, in particular, to recombinant antibodies with improved pharmacokinetic and biodistribution properties as well as with increased binding avidity. In order to achieve multimerization of the scFv, scFv may be prepared as fusion proteins with multimerization domains. The multimerization domains may be, e.g. the CH3 region of an IgG or coiled coil structure (helix structures) such as Leucine-zipper domains. However, there are also strategies in which the interaction between the V_{H}/V_{L} regions of the scFv are used for the multimerization (e.g. di-, tri- and pentabodies).

Also considered are multivalent antibody constructs that are Her3 antagonists or agonists. In one embodiment, a multivalent antibody construct comprises at least one antigen recognition site specific for Her3 receptor protein. In certain embodiments, at least one of the antigen recognition sites is located on a scFv domain, while in other embodiments, all antigen recognition sites are located on scFv domains.

Also contemplated herein is a multivalent, multispecific antibody or fragment thereof comprising more than one antigen binding site having an affinity toward a Her3 target antigen and one or more hapten binding sites having affinity towards hapten molecules. Also preferred, the multivalent, multispecific antibody or fragment thereof further comprises a diagnostic/detection and/or therapeutic agent.

The phrase “multivalent antibody” or “multivalent antibody construct” refers to an antibody or antibody construct comprising more than one antigen recognition site. For example, a “bivalent” antibody construct has two antigen recognition sites, whereas a “tetravalent” antibody construct has four antigen recognition sites. The terms “monospecific”, “bispecific”, “trispecific”, “tetraspecific”, etc. refer to the number of different antigen recognition site specificities (as opposed to the number of antigen recognition sites) present in a multivalent antibody construct of the invention. For example, a “monospecific” antibody construct's antigen recognition sites all bind the same epitope. A “bispecific” antibody construct has at least one antigen recognition site that binds a first epitope and at least one antigen recognition site that binds a second epitope that is different from the first epitope. A “multivalent monospecific” antibody construct has multiple antigen recognition sites that all bind the same epitope. A “multivalent bispecific” antibody construct has multiple antigen recognition sites, some number of which bind a first epitope and some number of which bind a second epitope that is different from the first epitope.

In still another embodiment, the antibody construct is monospecific. In still another embodiment, the multivalent antibody is tetravalent. In one embodiment of the invention, the antibody is a monospecific tetravalent antibody, wherein the antibody comprises four Her3 antigen recognition sites. In still another embodiment, the antibody construct is specific for an epitope on Her3.

In another embodiment of the invention, the antibody construct is bispecific. In one embodiment, the antibody construct has two Her3-specific antigen recognition sites and two Her3-specific recognition sites.

In another embodiment of the invention, the antibody construct is a trivalent antibody construct specific for the Her3 receptor protein. In yet another embodiment, the invention contemplates an antibody construct having two Her3-specific antigen recognition sites and two Her3-specific recognition sites.

Other antibody constructs may be multispecific for different epitopes on human Her3 receptor proteins. In any of the multispecific antibody constructs, at least one antigen recognition site may be located on a scFv domain, and in certain embodiments, all antigen recognition sites are located on scFv domains.

In one aspect, the invention provides an antibody fragment comprising: (i) a first polypeptide comprising a light chain variable domain (and in some embodiments further comprising a light chain constant domain), (ii) a second polypeptide comprising a heavy chain variable domain, a first Fc polypeptide sequence (and in some embodiments further comprising a non-Fc heavy chain constant domain sequence), and (iii) a third polypeptide comprising a second Fc polypeptide sequence. Generally, the second polypeptide is a single polypeptide comprising a heavy chain variable domain, heavy chain constant domain (e.g., all or part of CH1) and the first Fc polypeptide. For example, the first Fc polypeptide sequence is generally linked to the heavy chain constant domain by a peptide bond [i.e., not a non-peptidyl bond]. In one embodiment, the third polypeptide comprises an N-terminally truncated heavy chain which comprises at least a portion of a hinge sequence at its N terminus. In one embodiment, the third polypeptide comprises an N-terminally truncated heavy chain which does not comprise a functional or wild type hinge sequence at its N terminus. In some embodiments, the two Fc polypeptides of an antibody of the invention or a fragment thereof are covalently linked. For example, the two Fc polypeptides may be linked through intermolecular disulfide bonds, for instance through intermolecular disulfide bonds between cysteine residues of the hinge region.

In one aspect, the invention provides a composition comprising a population of immunoglobulins wherein at least (or at least about) 50%, 75%, 85%, 90%, 95% of the immunoglobulins are antibody fragments of the invention. A composition comprising said population of immunoglobulins can be in any of a variety of forms, including but not limited to host cell lysate, cell culture medium, host cell paste, or semi-purified or purified forms thereof. Purification methods are well known in the art, some of which are described herein.

Another embodiment of the invention provides a Her3-specific diabody antibody. By diabody the skilled person means a bivalent homodimeric scFv derivative (Flu et al., 1996, PNAS 16: 5879-5883). The shortening of the Linker in a scFv molecule to 5-10 amino acids leads to the formation of homodimers in which an inter-chain V_{H}/N_{L}-superimposition takes place. Diabodies may additionally be stabilized by the incorporation of disulphide bridges. Examples of diabody-antibody proteins from the prior art can be found in Perisic et al. (1994, Structure 2: 1217-1226).

By minibody the skilled person means a bivalent, homodimeric scFv derivative. It consists of a fusion protein which contains the CH3 region of an immunoglobulin, preferably IgG, most preferably IgG1 as the dimerization region which is connected to the scFv via a Hinge region (e.g. also from IgG1) and a Linker region. The disulphide bridges in the Hinge region are mostly faulted in higher cells and not in prokaryotes. Preferably the minibody is a Her3-specific minibody antibody fragment. Examples of minibody-antibody proteins from the prior art can be found in Hu et al. (1996, Cancer Res. 56: 3055-61).

By triabody the skilled person means a: trivalent homotrimeric scFv derivative (Kortt et al. 1997 Protein Engineering 10: 423-433). ScFv derivatives wherein V_{H}-V_{L} are fused directly without a linker sequence lead to the formation of trimers.

The skilled person will also be familiar with so-called miniantibodies which have a bi-, tri- or tetravalent structure and are derived from scFv. The multimerization is carried out by di-, tri- or tetrameric coiled coil structures (Pack et al., 1993 Biotechnology II:, 1271-1277; Lovejoy et al. 1993 Science 259: 1288-1293; Pack et al., 1995 J. Mol. Biol. 246: 28-34).

Therefore, an alternative embodiment proposes a Her3-specific multimerized molecule based on the abovementioned antibody fragments and may be, for example, a triabody, a tetravalent miniantibody or a pentabody.

A related aspect of the invention provides monoclonal antibodies or functional fragments thereof that specifically binds human Her3 with specified affinities. In certain embodiments, these antibodies bind human Her3 with an ED₅₀ in the range of about 10 pM to about 500 nM. In certain embodiments, these antibodies bind human Her3 with an ED₅₀ in the range of about 500 pM to about 300 nM.

The present invention further provides an antibody-recognized surface antigen present on host cell, including but not limited to T-cell acute lymphoblastic leukemia/lymphoma, human colon cancer, melanoma, human lung cancer and human prostate cancer, the antigen being Her3 or a biologically equivalent variant or fragment thereof.

Also provided is a monoclonal antibody of the invention or a binding fragment thereof that is bound to a solid matrix.

Antibodies to Her3 as described herein may also be used in production facilities or laboratories to isolate additional quantities of the proteins, such as by affinity chromatography. For example, the antibodies of the invention may also be utilized to isolate additional amounts of Her3.

In one aspect, the invention provides isolated, purified or recombinant polypeptides having an amino acid sequence that is at least 90%, 95%, 98% or 99% identical to an amino acid sequence as set forth in one or more appendices herein described. In certain embodiments the application provides an amino acid sequence that is at least 90%, 95%, 98%, 99%, 99.3%, 99.5% or 99.7% identical to the target amino acid sequence herein described.

The present invention further relates to a polynucleotide encoding an antibody of the invention. In accordance therewith, the invention further provides: isolated nucleic acid encoding the inventive antibodies disclosed herein including the heavy and/or light chain or antigen-binding portions thereof. Thus, an aspect of the invention provides isolated nucleic acid molecules selected from the nucleotide sequences described in any one or more of the appendices detailed herein. A related aspect is drawn to (a) a nucleic aid molecule described in any one or more of the appendices detailed herein encoding one or more of the sequence of amino acids as set forth in one or more of the appendices described herein; or (b) the nucleotide sequence that hybridizes to the nucleotide sequence of (a) under moderately stringent conditions, or (e) a nucleic acid molecule comprising a nucleotide sequence that is a degenerate sequence with respect to either (a) or (b) above, or (d) splice variant cDNA sequences thereof or (e) a nucleic acid of at least 18 nucleotides capable of hybridizing under conditions of great stringency with at least one of the CDRs of nucleic acid sequence described in one or more of the appendices described herein or with a sequence having at least 80%, preferably 85%, 90%, 95% and 98%, identity after optimum alignment with the sequence as set forth in one or more of the appendices detailed herein.

A vector comprising the nucleic acid molecule described above, optionally, operably linked to control sequences recognized by a host cell transformed with the vector is also provided as is a host cell transformed with the vector. The cells transformed according to the invention can be used in processes for preparation of recombinant antibody disclosed herein. A variety of host cells can be transformed with the nucleic acid molecules encoding the antibody or a fragment thereof. The host cell can be chosen from prokaryotic or eukaryotic systems, for example bacterial cells but likewise yeast cells or animal cells, in particular mammalian cells. It is likewise possible to use insect cells or plant cells. Methods of producing a recombinant protein are well known.

The invention likewise concerns animals, except man, which comprise at least one cell transformed according to the invention. Thus, non-human transgenic animals that express the heavy and/or light chain or antigen-binding portions thereof of an anti-Her3 antibody are also provided.

The present invention further provides a pharmaceutical composition comprising the monoclonal antibody, or binding fragment thereof, according to the invention, and a pharmaceutically acceptable carrier, excipient, or diluent. The pharmaceutical composition may further comprise another component, such as an anti-tumor agent or an imaging reagent. Certain embodiments of the invention relate to the use of the invention antibodies as targeted delivery systems for cytotoxic agents such as chemotherapeutic drugs, peptides or radionuclides, for immunological response promoters such as cytokines, for pro-drugs or for gene therapies. As well, the antibodies described herein may also transport/deliver payloads such as RNAi or shRNA. These payloads may be naked or chemically modified. Immunoliposomes as potential delivery vehicles are also included.

As will be appreciated by one skilled in the art, the antibodies of the invention or binding fragments thereof will also find use in various medical or research purposes, including staging of various pathologies associated with expression of Her3. Indeed, laboratory research may also be facilitated through use of such antibodies. Identifying patients at risk of a pathological effect of an oncogenic disorder associated with expression of Her3, particularly hyperproliferative oncogenic disorders such as, but not limited to, cerebral autosomal dominant arteriopathy with subcortical infarcts and leukoencephalopathy (CADASIL), T-cell acute lymphoblastic leukemia, lymphoma, Alagille syndrome, liver disease involving aberrant vascularization; diabetes, ovarian cancer, diseases involving vascular cell fate, rheumatoid arthritis, pancreatic cancer, plasma cell neoplasms (such as multiple myeloma, plasma cell leukemia, and extramedullary plasmacytoma), and neuroblastoma is also encompassed. As would be recognized by one of ordinary skill in this art, the level of antibody expression associated with a particular disorder will vary depending on the nature and/or the severity of the pre-existing condition.

As a consequence, additional embodiments of the invention pertain to the use of the invention antibodies for detecting dysplastic or neoplastic Her3 bearing cells as well as diagnosing, assessing and treating disorders associated with expression of Her3 receptor protein or aberrant activation of the Her3 cascade.

As used herein, the term “an oncogenic disorder associated with expression of Her3” is intended to include diseases and other disorders in which the presence of high levels or abnormally low levels of Her3 receptor protein (aberrant) in a subject suffering from the disorder has been shown to be or is suspected of being either responsible for the pathophysiology of the disorder or a factor that contributes to a worsening of the disorder. Thus, “neoplastic cells” or “neoplasia associated with expression of Her3” or “dysplastic cells associated with expression of Her3” which are used interchangeably refer to abnormal cells or cell growth characterized by increased or decreased expression levels of Her3 relative to normal. Such transformed cells proliferate without normal homeostatic growth control resulting in a condition marked by abnormal proliferation of cells of a tissue—cancer. Alternatively, such disorders may be evidenced by an increase in the levels of Her3 on the cell surface or in increased ICD levels in the affected cells or tissues of a subject suffering from the disorder. The increase in Her3 levels may be detected, for example, using an anti-Her3 antibody as described above. More, it refers to cells which exhibit relatively autonomous growth, so that they exhibit an aberrant growth phenotype characterized by a significant loss of control of cell proliferation. Alternatively, the cells may express normal levels of Her3 but are marked by abnormal proliferation. Not all neoplastic cells are necessarily replicating cells at a given time point. The cells defined as neoplastic cells consist of cells in benign neoplasms and cells in malignant (or frank) neoplasms. Frankly, neoplastic cells are frequently referred to as cancer, typically termed carcinoma if originating from cells of endodermal or ectodermal histological origin, or sarcoma if originating from cell types derived from mesoderm.

In certain embodiments, “increased expression” as it relates to Her3 refers to protein or gene expression levels that demonstrate a statistically significant increase in expression (as measured by RNA expression or protein expression) relative to a control. As well “increased expression” is also used to encompass “increased activation of the Her3 cascade”. Thus, in some disorders associated with expression of Her3, the level of expression of Her3 may not be increased relative to a control but the level of activation of the Her3 cascade may be increased relative to a control or a patient without the disease.

Administration of the antibodies of the present invention in any of the conventional ways known to one skilled in the art (e.g., topical, parenteral, intramuscular, IV, subcutaneous etc.), will provide an extremely useful method of detecting dysplastic cells in a sample as well as allowing a clinician to monitor the therapeutic regiment of a patient undergoing treatment for a hyperproliferative disorder associated with or mediated by expression of Her3.

It is known in the art to use antibodies to detect the presence or expression of a specific protein. Because Her3 may be overexpressed in certain hyperproliferative disorders including, for example, cancer, Her3-specific antibodies of this invention may be used to detect the overexpression and, thus, to detect metastatic disease. As well, the immunodetection methods of the present invention may be of utility in the diagnosis of various disease states. As well, the invention antibodies may be exploited to detect differential expression of Her3 in metastatic cells. As will be apparent to the skilled artisan human Her3 or ICD or any other downstream target may be detected in a number of ways such as by various assays.

Immunodetection techniques include but are not limited to immunohistological staining, western blotting, dot blotting, precipitation, agglutination, ELISA assays, immunohistochemistry, in situ hybridization, flow cytometry or radio-immunoassay (RIA) technique or equivalent on a variety of tissues and a variety of sandwich assays. These techniques are well known in the art. See, for example, U.S. Pat. No. 5,876,949, hereby incorporated by reference.

When used with suitable labels or other appropriate detectable biomolecule or chemicals, the antibodies described herein are particularly useful for in vitro and in vivo diagnostic and prognostic applications. Suitable conditions for which the antibody of the invention will find particular use for include the detection and diagnosis of neoplasias, such as, but not limited to cancer of the ovary, prostate, colon and skin. Inflammatory responses or disorders triggered by Her3 receptor activation or cascade area also included.

Labels for use in immunoassays are generally known to those skilled in the art and include enzymes, radioisotopes, and fluorescent, luminescent and chromogenic substances, including colored particles such as colloidal gold or latex beads. Various types of labels and methods of conjugating the labels to the antibodies of the invention are well known to those skilled in the art, such as the ones set forth below.

In accordance with the above, an illustrative embodiment provides a method for detecting normal, benign, hyperplastic, and/or cancerous cells or a portion thereof in a biological sample comprising: providing a Her3 antibody or binding portion thereof which recognizes an antigen (Her3) on the surface of the cells, wherein the antibody or binding portion thereof binds to an epitope of Her3 which is also recognized by any one or more monoclonal antibodies detailed herein and wherein the antibody or binding portion thereof is bound to a label effective to permit detection of the cells or a portion thereof upon binding of the antibody or binding portion thereof to the antigen; contacting the biological sample with the antibody or binding portion thereof having a label under conditions effective to permit binding of the antibody or binding portion thereof to the antigen on any of the cells or a portion thereof in the biological sample; and detecting the presence of any of the cells or a portion thereof in the biological sample by detecting the label.

In certain embodiments, the step of contacting the antibody is carried out in a living mammal and comprises: administering the Nocth1 antibody or binding portion thereof to the mammal under conditions effective to permit binding of the antibody or binding portion thereof to the antigen on any of the cells or a portion thereof in the mammal.

In certain embodiments, the invention antibodies may be labeled with a detectable moiety, such as a fluorophore, a chromophore, a radionuclide, a chemiluminescent agent, a bioluminescent agent and an enzyme.

Yet another embodiment of the invention provides a method of diagnosis, preferably in vitro, of illnesses connected with an overexpression or under expression, preferably overexpression of the Her3 receptor. Samples are taken from the patient and subject to any suitable immunoassay with Her3 specific antibodies to detect the presence of Her3. Preferably, the biological sample is preferably tissue sample or biopsies of human origin which can be conveniently assayed for the presence of a pathological hyperproliferative oncogenic disorder associated with expression of Her3.

Once a determination is made of the amount of Her3 present in the test sample, the results can be compared with those of control samples, which are obtained in a manner similar to the test samples but from individuals that do not have or present with a hyperproliferative oncogenic disorder associated with expression of Her3, e.g., ovarian cancer. If the level of the Her3 receptor polypeptide is significantly elevated in the test sample, it may be concluded that there is an increased likelihood of the subject from which it was derived has or will develop said disorder, e.g., T-ALL, ovarian cancer etc. The diagnostic uses of the antibodies according to the present invention embrace primary tumors and cancers, as well as metastases. Preferably, the antibody, or one of its functional fragments, can be present in the form of an immunoconjugate or of a labeled antibody so as to obtain a detectable and/or quantifiable signal.

An exemplary in vitro method of diagnosing pathological hyperproliferative oncogenic disorder comprises: (a) determining the presence or absence of Her3 bearing cells in a sample; and (b) diagnosing a pathological condition or a susceptibility to a pathological condition based on the presence or absence of said Her3 bearing cells. In the clinical diagnosis or monitoring of patients with an Her3 mediated neoplastic disease, the detection of Her3 expressing cells or an increase in the levels of Her3, in comparison to the levels in a corresponding biological sample from a normal subject or noncancerous tissue is generally indicative of a patient with or suspected of presenting with an Her3 mediated disorder.

A representative in vitro method of diagnosing the presence of cancer in a patient or a susceptibility to a pathological condition associated therewith in a subject, proposes: (a) measuring the levels of Her3 receptor protein in cells or tissues of the patient; and (b) comparing the measured levels of the antigen of (a) with levels of the antigen (Her3 receptor protein) in cells or tissues from a normal human control, wherein an increase in the measured levels of the antigen in the patient versus the normal control is associated with the presence of the cancer. In certain embodiments, decreased Her3 expression may be diagnostic of a pathological condition such as disorders of the skin. Alternatively, one may measure the level of the ICD as a measure of Her3 receptor activation.

Alternatively, the above method may be practiced over several time points. A representative embodiment thus provides a method of diagnosing a pathological oncogenic disorder associated with aberrant expression of Her3 or increased Her3 receptor activation (Her3 cascade) comprising the steps of: a) detecting the presence and level of Her3 in a biological sample obtained from the mammal at a plurality of time points, wherein Her3 is detected by a method selected from the group consisting of immunoblotting, western blotting, immunoperoxidase staining, fluorescein labeling, diaminobenzadine and biotinylation; and b) correlating change in Her3 expression with said diagnosis. It is understood that other conventional assays may be used instead of or in addition to those described herein.

A method of monitoring metastatic potential of an oncogenic disorder associated with Her3 expression in a mammal is also encompassed. In accordance therewith, provided herein is a method for screening for metastatic potential of solid tumors comprising: a) obtaining a sample of tumor tissue from an individual in need of screening for metastatic potential of a solid tumor; b) reacting an antibody to Her3 with tumor tissue from the patient; c) detecting the extent of binding of the Her3 antibody to the tissue and d) correlating the extent of binding of the antibody with its metastatic potential. Preferably, the tumor is cancer arising from large bowel (colorectal cancer), prostate, breast or skin (ovarian cancer or T-ALL). In certain embodiments, step c) may be performed over a plurality of time points. As well, in certain embodiments, Her3 expression is detected by a method selected from the group consisting of immunohistochemical staining, immunoblotting, western blotting, immunoperoxidase staining, fluorescein labeling, diaminobenzadine and biotinylation.

The invention further provides for a method for predicting susceptibility to cancer comprising detecting the expression level of Her3 in a tissue sample, its presence indicating susceptibility to cancer, wherein the degree of Her3 expression correlates to the degree of susceptibility. In certain embodiments, the expression of Her3 in, for example, breast tissue, prostate tissue, colon tissue, or any other tissue suspected of cells expressing Her3 is examined, with the presence of Her3 in the sample providing an indication of cancer susceptibility or the emergence or existence of a tissue specific tumor.

Stage determination has potential prognostic value and provides criteria for designing optimal therapy. Simpson et al., J. Clin. Oncology 18:2059 (2000). Generally, pathological staging of breast cancer for example, is preferable to clinical staging because the former gives a more accurate prognosis. However, clinical staging would be preferred if it were as accurate as pathological staging because it does not depend on an invasive procedure to obtain tissue for pathological evaluation. Thus, methods for gauging tumor aggressiveness are also provided as are methods for observing the progression of a malignancy in an individual over time.

Accordingly, the invention provides an in vivo imaging reagent comprising an antibody according to the invention, or one of its functional fragments, preferably labeled, especially radiolabeled, and its use in medical imaging, in particular for the detection of Her3 mediated disorders e.g., cancer characterized by over expressing Her3 or other pathologies in which cells over express Her3.

The imaging reagents, e.g., diagnostic reagents can be administered by intravenous injection into the body of the patient, or directly into a tissue suspected of harboring Her3 bearing cells, e.g., colon or ovary or the pancreas. The dosage of reagent should be within the same ranges as for treatment methods. Typically, the reagent is labeled, although in some methods, the primary reagent with affinity for Her3 is unlabelled and a secondary labeling agent is used to bind to the primary reagent. The choice of label depends on the means of detection. For example, a fluorescent label is suitable for optical detection. Use of paramagnetic labels is suitable for tomographic detection without surgical intervention. Radioactive labels can also be detected using PET or SPECT.

Diagnosis is performed by comparing the number, size, and/or intensity of labeled loci, to corresponding baseline values. The baseline values can, as an example, represent the mean levels in a population of undiseased individuals. Baseline values can also represent previous levels determined in the same patient. For example, baseline values can be determined in a patient before beginning treatment, and measured values thereafter compared with the baseline values. A decrease in values relative to baseline signals a positive response to treatment.

Thus, a general method embodied by the invention works by administering to a patient an imaging-effective amount of an imaging reagent such as the above described monoclonal antibodies or antigen-binding fragments which are labeled and a pharmaceutically effective carrier and then detecting the agent after it has bound to Her3 present in the sample.

In certain embodiments, the method works by administering an imaging-effective amount of an imaging agent comprising a targeting moiety and an active moiety. The targeting moiety may be an antibody, Fab, Fab′2, a single chain antibody or other binding agent that interacts with an epitope present in Her3. The active moiety may be a radioactive agent, such as radioactive technetium, radioactive indium, or radioactive iodine. The imaging agent is administered in an amount effective for diagnostic use in a mammal such as a human and the localization and accumulation of the imaging agent is then detected. The localization and accumulation of the imaging agent may be detected by radionuclide imaging, radioscintigraphy, nuclear magnetic resonance imaging, computed tomography, positron emission tomography, computerized axial tomography, X-ray or magnetic resonance imaging method, fluorescence detection, and chemiluminescent detection.

The in vivo imaging methods of the present invention are also useful for providing prognoses to cancer patients. For example, the presence of Her3 indicative of an aggressive cancer likely to metastasize or likely to respond to a certain treatment can be detected. Thus, in one aspect, the invention provides a method for observing the progression of a malignancy in an individual over time comprising determining the level of Her3 expressed by cells in a sample of the tumor, comparing the level so determined to the level of Her3 expressed in an equivalent tissue sample taken from the same individual at a different time, wherein the degree of Her3 expression in the tumor sample over time provides information on the progression of the cancer.

The in vivo imaging methods of the present invention can further be used to detect Her3 mediated cancers e.g., one that has metastasized in other parts of the body.

A related embodiment relates to a pharmaceutical composition for in vivo imaging of an oncogenic disorder associated with expression of Her3 comprising the invention antibodies or binding fragment thereof which is labeled and which binds Her3 in vivo; and a pharmaceutically acceptable carrier.

The antibodies disclosed herein may also be used in methods of identifying human tumors that can escape anti-Her3 treatment by observing or monitoring the growth of the tumor implanted into a rodent or rabbit after treatment with a conventional anti-Her3 antibody.

The antibodies of the invention can also be used to study and evaluate combination therapies with anti-Her3 antibodies of this invention and other therapeutic agents. The antibodies and polypeptides of this invention can be used to study the role of Her3 in other diseases by administering the antibodies or polypeptides to an animal suffering from the disease of a similar disease and determining whether one or more symptoms of the disease are alleviated.

Those of skill in the art are very familiar with differentiating between significant expression of a target antigen, e.g., Her3, which represents a positive identification, and low level or background expression of the antigen. Indeed, background expression levels are often used to form a “cut-off” above which increased staining will be scored as significant or positive. Significant expression may be represented by high levels of antigens in target cells or tissues or alternatively, by a high proportion of cells from within a tissue that each give a positive signal.

The above diagnostic approaches can be combined with any one of a wide variety of prognostic and diagnostic protocols known in the art. For example, another embodiment of the invention is directed to methods for observing a coincidence between the expression of Her3 and a factor that is associated with malignancy, as a means for diagnosing and prognosticating the status of a tissue sample. A wide variety of factors associated with malignancy can be utilized, such as the expression of genes or gene products associated with malignancy as well as gross cytological observations. Methods for observing a coincidence between the expression of Her3 and another factor that is associated with malignancy are useful, for example, because the presence of a set of specific factors that coincide with disease provides information crucial for diagnosing and prognosticating the status of a tissue sample. The methods proposed herein can be useful to diagnose or confirm diagnosis of an oncogenic disorder associated with expression of Her3 or susceptibility thereof. For example, the methods can be used on a patient presenting with symptoms of an oncogenic disorder. If the patient has, for example increased expression levels of Her3 or aberrant Her3 receptor activation as evidenced by increased expression levels of any one or more downstream targets effected by or related to Her3 receptor activation or increased expression levels of ICD, then the patient is likely suffering from a cancerous disorder. The methods can also be used in asymptomatic patients. Presence of higher than normal Her3 may indicate for example susceptibility to future symptomatic disease. As well, the methods are useful for monitoring progression and/or response to treatment in patients who have been previously diagnosed with an Her3 mediated cancer.

Generally speaking, malignancies are characterized by either increased Her3 receptor expression, increased or aberrant Her3 receptor activation or mutations resident in the Her3 receptor protein. Malignancies characterized by aberrant or increased Her3 receptor activation may be confirmed via determination of expression levels of ICD, whose expression level may be increased in the cytoplasm following activation of the Her3 cascade. Thus, in those cases where a malignancy is characterized by increased Her3 receptor activation, one is expected to find increased expression of ICD in the cytoplasm. This increase in ICD expression can be traced to the translocation of the ICD into the cytoplasm upon Her3 receptor activation. A similar effect should be observed for downstream targets effected by aberrant Her3 receptor activation—a decrease or increase relative to normal of a specific downstream Her3 target occurring as a result of Her3 receptor activation. Thus, measurement of Her3 in biopsied tissue or other biological sample can be corroborated by determining expression of downstream target expression as a means of identifying high risk patients. In certain embodiments, single or multiple determinations of increased Her3 expression and/or ICD expression over time may serve as a marker for illness indicative of intervening medical intervention. A positive test can therefore supplement the clinicians judgment.

Increased Her3 levels also add prognostic accuracy to established severity of illness scores. Such clinical judgments will benefit by a method of scoring diseased cells. As discussed herein, measurement of Her3 expression in a tissue sample can also be used as an indicator for additional monitoring or testing, or consideration for more aggressive treatment, especially when patients are found to have increased Her3 expression levels or increased Her3 receptor activation as reflected by increased cytoplasmic ICD levels or any other downstream target.

Thus, for example, one would expect that a patient at risk of developing a Her3 mediated cancer or presenting with such a cancer would likely have increased Her3 expression relative to a control sample. As such, in certain embodiments, for such patients, a semiquantitative estimation of Her3 immunoreactivity can be performed. Towards this end, a score can be given to each slide, considering the intensity of the stain. The slides may be examined and scored independently by two investigators, and discordances may be reconciled by re-examination of the slide, and the scores then averaged. The intensity of immunostaining of individual cells may be scored on a scale of 0 (no staining) to 4 (strongest intensity) and the percentage of cells with staining at each intensity estimated. If there is no staining, a 0 score can be given. A +1 score indicates weak staining, while a +4 score indicating strong intensity of staining. As will be appreciated, any scoring scheme used to compare staining intensities may be used as long as it takes into account the relative intensity of cytoplasmic staining and allows differentiation among degrees of intensity of staining, thus providing a way to grade the malignancies. Because of the novel staining aspects of the present invention which results in highly differentiated staining, the scoring or grading can be done visually, thus allowing the technique of the present invention to be widely used clinically without sophisticated equipment. It will be understood that the staining results can be analyzed by appropriate sensitive optical equipment and analyzed by computer.

In furtherance of the above objective, the invention provides a method for diagnosing an oncogenic disorder associated with expression of Her3 comprising: a) measuring by radioimmunoassay, competitive-binding assay, Western blot analysis, ELISA assay, or sandwich assay the amount of Her3 protein in a biological sample, e.g., biopsied tissue obtained from a patient, using an antibody that specifically binds to Her3; and b) comparing the amount of antibody bound to said Her3 protein to a normal control tissue sample, wherein increased expression or over-expression of Her3 in the sample obtained from the patient relative to the normal control tissue sample is diagnostic of an oncogenic disorder associated with expression of Her3. Preferably, the Her3-specific antibody comprises sat least one antibody detailed herein.

In certain embodiment, the same scoring criteria e.g., score of 0 to 4 may be used to score cytoplasmic ICD staining as a means of corroborating the initial diagnosis. Consequently, cells may be stained with an antibody specific for ICD and the intensity level scored using the above criteria, where the intensity of immunostaining of individual cells may be scored on a scale of 0 (no staining) to 4 (strongest intensity) and the percentage of cells with staining at each intensity estimated. If there is no staining, a 0 score can be given. A +1 score indicates weak staining, while a +4 score indicating strong intensity of staining.

In additional embodiments, a prognostic index is produced by preparing a weighted scale of expression levels of the tumor markers, Her3 related to progression observed in a representative sample of a particular tumor type, wherein the different values in the weighted scale are related to increased invasiveness or metastatic spread in the representative sample.

The methods of the invention are also useful for identifying a human cancer patient at risk for additional neoplastic disease, for staging malignant disease in a human cancer patient and assessing the relative risk of metastatic disease versus the risk of toxicity (such as leukocytopenia, for example) from chemotherapeutic treatment.

The invention thus provides methods wherein the results of the determination of the level of cell surface Her3 expression and the extent of cytoplasmic localization of ICD are used to prepare a prognostic or “risk” index for making a prognostic determination. In this aspect of the invention, a prognostic index is prepared using the above criteria, wherein a value of 0 signifies a control, a value of +1 indicates weak staining etc, wherein a prognosis of a likelihood of progressing to metastatic disease is made when the staining intensity s scoffed at +4.

An illustrative embodiment of the invention provides a diagnostic or monitoring method comprising: a) obtaining a sample of tissue from an individual in need of diagnosis or monitoring for cancer; b) detecting levels of Her3 protein in said sample, c) scoring said sample for Her3 protein levels; and d) comparing said scoring to that obtained from a control tissue sample to determine the prognosis associated with said cancer. Samples may be scored using a scale of 0 to 4, where 0 is negative (no detectable Her3 expression or level comparable to a control level), and 4 is high intensity staining in the majority of cells and wherein a score of 1 to 4 indicates a poor prognosis while a score of 0 indicates a good prognosis.

A related aspect of the invention pertains to a method for screening for metastatic potential of a Her3 mediated hyperproliferative disorder comprising: a) obtaining a sample of diseased or target tissue from an individual in need of screening for metastatic potential of a Notch1 mediated tumor, b) reacting an antibody to Her3 with tumor tissue from the patient, c) detecting the extent of binding of the Her3 antibody to said tissue and d) correlating the extent of binding of said antibody with its metastatic potential. In general, any of the methods of the invention involving analysis of the levels of Her3 or ICD may be used in conjunction with additional cancer markers readily known to those of skill in the art.

Also provided is a method of detecting the presence and extent of cancer in a patient, comprising: determining the level of the antigen (Her3) in a sample of cells or a tissue section from the patient and correlating the quantity of the antigen with the presence and extent of the cancer disease in the patient relative to a normal or control patient.

One of the major challenges facing the pharmaceutical industry in drug development is to show efficacy associated with a potential therapeutic candidate. This drawback applies equally to the numerous efforts underway in the pharmaceutical industry to generate anti-Her3 inhibitory antibodies as anti-cancer therapeutics. One way to do this is to have a suitable marker that indicates when Her3 activity is inhibited. Ideally, where a candidate Her3 antagonist moiety is effective, one should observe a decrease in the expression levels of Her3 following treatment with the Her3 antagonist moiety. Alternatively, one might expect an increase in the levels of phosphorylated Her3, which signals activation of the kinase domain resident in Her3. It thus follows that favorable treatment with an Her3 antagonistic moiety would predict a decrease in Her3 expression levels on tumor cells or any other cells that express this cell surface receptor, while an unfavorable outcome would predict either no change in the expression levels or an increase in expression levels of Her3. Thus, by measuring Her3 protein expression on a tumor cell, for example, with a suitable marker, decreased expression levels may be detected as an indicator of suppressed Her3 activity. The present invention exploits the ability of the Her3 antibodies of the invention to bind Her3 with high affinity to be utilized in a “biomarker strategy” for measuring Her3 activity and/or expression or tumorigenic status by specifically measuring the expression levels of Her3 on tumor/cancer cells. As a consequence, the present invention provides a rapid means, e.g., high affinity anti-Her3 antibodies, for assessing the nature, severity and progression of a pathological hyperproliferative oncogenic disorder associated with expression of Her3.

In furtherance of the “biomarker strategy” noted above, the invention provides a method for determining onset, progression, or regression, of neoplasias associated with expression of Her3 in a subject, comprising: obtaining from a subject a first biological sample at a first time point, contacting the first sample with a effective amount of an antibody described herein under conditions allowing for binding of the antibody or a fragment thereof to Her3 suspected to be contained in the sample and determining specific binding between the antibody in the first sample and Her3 bearing cells to thereby obtain a first value, obtaining subsequently from the subject a second biological sample at a second time point, and contacting the second biological sample with the Her3 antibody and determining specific binding between the antibody and Her3 in said sample to obtain a second value, and comparing the determination of binding in the first sample to the determination of specific binding in the second sample as a determination of the onset, progression, or regression of the colon cancer, wherein an increase in expression level of Her3 in said second or subsequent sample relative to the first sample indicative of the progression of said neoplasias, and wherein decrease in indicative of regression of neoplasias in said sample.

In one embodiment, Her3 is detected by (1) adding an antibody of the invention to the sample or tissue section; (2) adding goat anti-mouse IgG antibody conjugated with peroxidase; (3) fixing with diaminobenzidene and peroxide, and (4) examining the sample or section, wherein reddish brown color indicates that the cells bear the antigen. According to the method, the effectiveness of a cancer treatment may be monitored by periodically measuring changes in the level of the antigen in a tissue sample taken from a patient undergoing the therapy, and correlating the change in level of the antigen with the effectiveness of the therapy, wherein a lower level of Her3 expression determined at a later time point relative to the level of Her3 determined at an earlier time point during the course of therapy indicates effectiveness of the therapy for the cancer disease.

In yet another embodiment, the application provides methods for determining the appropriate therapeutic protocol for a subject. Specifically, the antibodies of the invention will be very useful for monitoring the course of amelioration of malignancy in an individual, especially in those circumstances where the subject is being treated with a Her3 antibody that does not compete with the antibodies of the invention for binding Her3. Essentially, presence or absence or a change in the level of Her3 expression may be indicative as to whether a subject is likely to have a relapse or a progressive neoplasia or persistent neoplasias such as cancer associated with Her3. Thus, by measuring an increase in the number of cells expressing Her3 or changes in the concentration of Her3 present in various tissues or cells, it is possible to determine whether a particular therapeutic regimen aimed at ameliorating a malignancy associated with Her3 is effective. For those patients receiving conventional therapy, and whose Nocth1 expression has not changed over time, for example, these may, instead be treated with the Her3 antibodies of the invention and the change in Nocth1 observed over time. If a change if readily evident over a period of time, then it may be possible to switch the patient from conventional therapy to therapy with one or more of the antibodies disclosed herein.

An antibody fragment of the invention is capable of specifically binding to a target molecule of interest. For example, in some embodiments, an antibody fragment specifically binds a tumor antigen. In some embodiments, the antibody fragment specifically binds a cell surface receptor that is activated upon receptor multimerization (e.g., dimerization). In some embodiments, binding of an antibody of the invention to a target molecule inhibits binding of another molecule (such as a ligand, where the target molecule is a receptor) to said target molecule.

Thus, in one example, an antibody fragment of the invention when bound to a target molecule inhibits binding of a cognate binding partner to the target molecule. A cognate binding partner can be a ligand, or a hetero or homodimerizing molecule. In one embodiment, an antibody fragment of the invention when bound to a target molecule inhibits target molecule receptor activation. For example, in some embodiments wherein an antibody or a fragment thereof is an antagonist, binding of the antibody fragment to a cell surface receptor may inhibit dimerization of the receptor with another unit of the receptor, whereby activation of the receptor is inhibited (due at least in part to a lack of receptor dimerization). In one embodiment, an antibody fragment of the invention is capable of competing with a native Her3 receptor binding partner, e.g., delta or Serrate to the Her3 receptor. In another embodiment, an antibody of the invention or a fragment thereof is capable of competing with an endogenous Notch receptor ligand for binding to a Her3 receptor.

In certain embodiments, the herein described antibodies antagonize, or inhibit, Her3 mediated signaling by either blocking or inhibiting Her3 binding to its endogenous ligand or preventing or delaying Her3 cascade activation (hereinafter “Antagonist Therapeutics Antibodies”) are administered for therapeutic effect. Disorders which can thus be treated can be identified by in vitro assays such as those described herein or known to one skilled in the art. Such antagonist antibodies include anti-Her3 neutralizing antibodies, and competitive inhibitors of EGFR protein-protein interactions as detailed infra. In furtherance of the above objective, an antibody of the invention is administered to treat a cancerous condition, or to prevent progression from a pre-neoplastic or non-malignant state into a neoplastic or a malignant state.

Another embodiment of the preset invention is the use of any of these antibodies for the preparation of a medicament or composition for the treatment of diseases and disorders associated with Her3 receptor activation.

Another embodiment of the preset invention is the use of any of these antibodies in the treatment of disorders associated with Her3 activation comprising the inhibition of said activation by, e.g., inhibiting Her3 signaling, or neutralization of the receptor by blocking ligand binding. Her3 related disorders may include, but are not limited to cancers, lethal congenital contractural syndrome type 2 (LCCS2).

Thus, in certain aspects, the invention provides a method for treating a disease comprising administering to a subject in need of such a treatment an effective amount for treating the disease of at least one antibody or antigenic or binding fragment thereof (“fragment”) disclosed herein that binds to native human Her3 (hHer3) and abolishes or attenuates the function of the native hHer3 or Her3/Her2 heterodimer. A variety of diseases may be treated with the above described methods including cancer, in particular T-cell acute lymphoblasticleukemia/lymphoma, human breast cancer, human colorectal cancer, melanoma, human lung cancer, human head and neck cancers and human prostate cancer, an immune or inflammatory disorder an angiogenesis disorder or any other disorder mediated by Her3 signaling (G. Sithanandam & L M Anderson (2008) Cancer Gene Therapy 15:413; Jiang et al. (2007) MC 282:32689; Grivas et al. (2007) Eur J. Cancer 43:2602).

Yet another objective resides in the proposed use of constitutively active Her3 receptor or an antagonist thereof, for the purpose of developing a medicament that may find use in the treatment of a condition which is responsive to constitutively active Her3 receptor.

In a further embodiment, the present invention provides a method of inhibiting or killing cancer cells, comprising: providing to a patient in need thereof the monoclonal antibody, or binding fragment thereof of the present invention, under conditions and in an amount sufficient for the binding to the cancer cells, thereby causing inhibition or killing of the cancer cells by the immune cells of the patient. Preferably, the method is for the treatment of T-cell acute lymphoblastic leukemia/lymphoma, human colon cancer, melanoma, human lung cancer and human prostate cancer. The monoclonal antibody is preferably conjugated with a cytotoxic moiety, such as a chemotherapeutic agent, a photoactivated toxin, an RNAi molecule or a radioactive agent. Preferably, the cytotoxic moiety may be a Ricin. An alternative method proposes treatment of a Her3 mediated disorder comprising the steps as outlined above. Representative disorders include an immune or inflammatory disorder like colitis or asthma, an infectious disease, an angiogenesis disorder, atherosclerosis, or a disorder of the kidney or any other disorder mediated by Her3 signaling.

In one embodiment, an oligonucleotide, such as an RNAi molecule inhibiting Her3 expression may be conjugated to, or form the therapeutic agent portion of an immunoconjugate or antibody fusion protein of the present invention. Alternatively, the oligonucleotide may be administered concurrently or sequentially with a naked or conjugated anti-Her3 antibody or antibody fragment of the present invention. In one embodiment, the oligonucleotides are an antisense oligonucleotide (RNAi) that preferably is directed against Her3 expression.

An alternative embodiment provides a method of treating a Her3 mediated disorder by administering a pharmaceutical composition comprising at least one Nocth1 inhibitor wherein the inhibitor is an anti-Her3 antibody conjugated to a Her3 specific RNA inhibitor, and a pharmaceutically acceptable carrier. RNA inhibition (RNAi) is based on antisense modulation of Her3 in cells and tissues comprising contacting the cells and tissues with at least one Her3 antibody conjugated to a nucleic acid molecule that modulated transcription or translation of Her3 receptor protein, including but not limited to double stranded RNA, (dsRNA), small interfering RNA (siRNA), ribozymes and locked nucleic acids (LNAs), and a pharmaceutically acceptable carrier.

The present invention further provides a method for localizing cancer cells in a patient, comprising: (a) administering to the patient a detectably-labeled monoclonal antibody of the invention, or binding fragment thereof; (b) allowing the detectably-labeled (e.g. radiolabeled; fluorochrome labeled, or enzyme labeled, especially via ELISA) monoclonal antibody, or binding fragment thereof, to bind to the cancer cells within the patient; and (c) determining the location of the labeled monoclonal antibody or binding fragment thereof, within the patient.

Another embodiment of the invention relates to the use of invention antibodies, and VRs, FRs and CDRs thereof, in directed molecular evolution technologies such as phage display or bacterial or yeast cell surface display technologies in order to generate polypeptides with enhanced affinity, specificity, stability or other desired characteristics.

Another embodiment of the present invention is a cancer cell targeting diagnostic immunoconjugate comprising an antibody component that comprises an antibody or fragment thereof of any one of the antibodies or fragments thereof of the present invention, wherein the antibody, or fragment thereof is bound to at least one diagnostic/detection agent.

Preferably, the diagnostic/detection agent is selected from the group comprising a radionuclide, a contrast agent, and a photoactive diagnostic/detection agent. Still preferred, the diagnostic/detection agent is a radionuclide with an energy between 20 and 4,000 keV or is a radionuclide selected from the group consisting of ¹¹⁰In, ¹¹¹In, ¹⁷⁷Lu, ¹¹⁸F, ⁵²Fe, ⁶²Cu, ⁶⁴Cu, ⁶⁷Cu, ⁶⁷Ga, ⁶⁸Ga, ⁸⁶Y, ⁹⁰Y, ⁸⁹Zr, ⁹⁴mTc, ⁹⁴Tc, ⁹⁹mTc, ¹²⁰I, ¹²³I, ¹²⁵I, ¹³¹I, ¹⁵⁴⁻¹⁵⁸Gd, ³²P, ¹¹C, ¹³N, ¹⁵O, ¹⁸⁶Re, ¹⁸⁸Re, ⁵¹Mn, ⁵²Mn, ⁵⁵Co, ⁷²As, ⁷⁵Br, ⁷⁶Br, ⁸²mRb, ⁸³Sr, or other gamma-, beta-, or positron-emitters. Also preferred, the diagnostic/detection agent is a paramagnetic ion, such as the a metal comprising chromium (III), manganese (II), iron (III), iron (II), cobalt (II), nickel (II), copper (II), neodymium (III), samarium (III), ytterbium (III), gadolinium (III), vanadium (H), terbium (III), dysprosium (III), holmium (III) and erbium (III), or a radioopaque material, such as barium, diatrizoate, ethiodized oil, gallium citrate, meglumine, metrizamide, metrizoate, propyliodone, and thallous chloride.

Also preferred, the diagnostic/detection agent is a fluorescent labeling compound selected from the group comprising fluorescein isothiocyanate, rhodamine, phycoerytherin, phycocyanin, allophycocyanin, o-phthaldehyde and fluorescamine, a chemiluminescent labeling compound selected from the group comprising luminol, isoluminol, an aromatic acridinium ester, an imidazole, an acridinium salt and an oxalate ester, or a bioluminescent compound selected from the group comprising luciferin, luciferase and aequorin. In another embodiment, the diagnostic immunoconjugates of the present invention are used in intraoperative, endoscopic, or intravascular tumor diagnosis.

Another embodiment of the present invention is a cancer cell targeting therapeutic immunoconjugate comprising an antibody component that comprises an antibody or fragment thereof of any one of the antibodies, fusion proteins, or fragments thereof of the present invention, wherein the antibody, fusion protein, or fragment thereof is bound to at least one therapeutic agent.

Preferably, the therapeutic agent is selected from the group consisting of a radionuclide, an immunomodulator, a hormone, a hormone antagonist, an enzyme, oligonucleotide, an enzyme inhibitor, a photoactive therapeutic agent, a cytotoxic agent, an angiogenesis inhibitor, and a combination thereof.

In one embodiment, the therapeutic agent is a cytotoxic agent, such as a drug or a toxin. Also preferred, the drug is selected from the group consisting of nitrogen mustards, ethylenimine derivatives, alkyl sulfonates, nitrosoureas, gemcitabine, triazenes, folic acid analogs, anthracyclines, taxanes, COX-2 inhibitors, pyrimidine analogs, purine analogs, antibiotics, enzymes, enzyme inhibitors, epipodophyllotoxins, platinum coordination complexes, vinca alkaloids, substituted ureas, methyl hydrazine derivatives, adrenocortical suppressants, hormone antagonists, endostatin, taxols, camptothecins, SN-38, doxorubicins and their analogs, antimetabolites, alkylating agents, antimitotics, antiangiogenic, apoptotic agents, methotrexate, CPT-11, and a combination thereof.

In another embodiment, the therapeutic agent is an oligonucleotide. For example, the oligonucleotide may be an antisense oligonucleotide such as an antisense oligonucleotide against Her3 or an RNAi molecule against Her3 receptor expression.

In another embodiment, the therapeutic agent is a toxin selected from the group consisting of ricin, abrin, alpha toxin, saporin, ribonuclease (RNase), DNase I, Staphylococcal enterotoxin-A, pokeweed antiviral protein, gelonin, diphtheria toxin, Pseudomonas exotoxin, and Pseudomonas endotoxin and combinations thereof, an immunomodulator is selected from the group consisting of a cytokine, a stem cell growth factor, a lymphotoxin, a hematopoietic factor, a colony stimulating factor (CSF), an interferon (IFN), a stem cell growth factor, erythropoietin, thrombopoietin and a combinations thereof, a radionuclide selected from the group consisting of ³²P, ³³P, ⁴⁷SC, ⁶⁴Cu, ⁶⁷Cu, ⁶⁷Ga, ⁸⁶Y, ⁹⁰Y, ¹¹¹Ag, ¹¹¹In, ¹²⁵U, ¹³¹I, ¹⁴²Pr, ¹⁵³Sm, ¹⁶¹Tb, ¹⁶⁶Dy, ¹⁶⁶Ho, ¹⁷⁷Lu, ¹⁸⁶Re, ¹⁸⁸Re, ¹⁸⁹Re, ²¹²Pb, ²¹²Bi, ²¹³Bi, ²¹¹At, ²²³Ra and ²²⁵Ac, and combinations thereof; or a photoactive therapeutic agent selected from the group comprising chromogens and dyes.

Still preferred, the therapeutic agent is an enzyme selected from the group comprising malate dehydrogenase, staphylococcal nuclease, delta-V-steroid isomerase, yeast alcohol dehydrogenase, .alpha.-glycerophosphate dehydrogenase, triose phosphate isomerase, horseradish peroxidase, alkaline phosphatase, asparaginase, glucose oxidase, .beta.-galactosidase, ribonuclease, urease, catalase, glucose-6-phosphate dehydrogenase, glucoamylase and acetylcholinesterase.

A further aspect of the invention pertains to kits. The invention is a kit comprising a container housing Her3 antibody or fragment thereof or antibody containing composition and instructions for administering the components in the kit to a subject at risk of, or in need of, treatment of a disease. The kit may further comprise a container housing a pharmaceutical preparation diluent.

The kit may also be used for determining whether an embedded biological sample contains human Her3 protein comprising: (a) an Her3-binding agent that specifically binds with an embedded human Her3 protein to form a binding complex; and (b) an indicator capable of signaling the formation of said binding complex, wherein the Her3 binding agent is a monoclonal antibody or a binding fragment thereof as set forth in the application. Diagnostic procedures using anti-Her3 antibody of the invention can be performed by diagnostic laboratories, experimental laboratories, practitioners, or private individuals. The clinical sample is optionally pre-treated for enrichment of the target being tested for. The user then applies a reagent contained in the kit in order to detect the changed level or alteration in the diagnostic component

In a further embodiment, the invention concerns an article of manufacture, comprising: a container; a label on the container; and composition comprising an active agent contained within the container; wherein the composition is effective for the detection, diagnosis or prognosis of neoplasia associated with expression of Her3 and the label on the container indicates that the composition can be used for the diagnosis or the prognosis of conditions characterized by overexpression of the Her3 protein receptor.

The invention further pertains to an article of manufacture comprising a container and a composition contained within said container, wherein the composition includes an antibody as described herein.

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways.

Other characteristics and advantages of the invention appear in the continuation of the description with the examples and the Figures whose legends are represented below.

### BRIEF DESCRIPTION OF THE DRAWINGS

FIG. 1. Titer of anti-Her3 sera binding of Her3 determined by series of dilutions of serum in ELISA.

FIG. 2. Binding of human and mouse Her3/ErbB3 by anti-Her3 antibodies on heterologous Her3 expressing CHO cells

FIGS. 3 (A&B). Dose response of purified anti-Her3 IgGs in inhibition of Her3 phosphorylation assay.

A) a set of mouse IgGs prepared by Strategic Diagnostic Inc. (SDI, 111 Pencader Drive Newark, Del. 19702);
B) mouse IgGs produced by Genovac GmbH (Waltershofener Str 17, Freiburg 79111, Germany)

FIGS. 4 (A&B). pAKT inhibition of Her3 IgGs in MCF7 cells

FIGS. 5 (A&B). pERK inhibition of Her3 IgGs in MCF7 cells

FIGS. 6 (A&B). Inhibition of NRG-induced cell growth in MCF7 cells by anti Her3 antibodies and data are shown as percentage of reduction by the antibodies relative to no antibody control.

FIGS. 7 (A&B) ligand blocking assay using alpha screening format and graphs show dose response of two sets of mouse anti-Her3 antibodies.

FIG. 8. PCR primer sets for cloning variable heavy and light chain cDNA sequences from mouse hybridoma cell lines.

### DETAILED DESCRIPTION OF THE INVENTION

### Overview of the Invention

Provided herein are various human Her3 specific antibodies, preferably monoclonal antibodies. Included are antagonist, inhibitory and neutralizing anti-Her3 antibodies that inhibit or decrease cancer cell growth or proliferation. Compositions comprising one or more of the herein described antibodies effective for use in the treating Her3 mediated hyper-proliferative disorders are also included. An Her3 receptor antagonist includes antigen-binding fragments thereof that bind the Her3 receptor extracellularly and is effective in blocking cleavage of the receptor or activating the Her3 receptor mediated signaling cascade. The compositions can be provided in an article of manufacture or a kit.

Another aspect of the invention is an isolated nucleic acid encoding any one or more of the anti-Her3 antibodies of the invention, as well as a vector comprising the nucleic acid. The human Her3 DNA sequence can be found using GenBank Accession Number (GenBank accession number—NM_{—}001982). Methods of recombinant production of the invention antibodies are also within the scope of the invention. Another aspect of the invention is a method of inhibiting or decreasing the proliferation of cancer cells by administering a Her3 antibody which results in blocking of the endogenous ligand to the Her3 receptor or inactivation/deactivation of Her3 signaling. Another aspect of the invention is a method of destroying cancer and tumor cells which express a Her3 receptor by administering to a patient in need thereof, a therapeutically effective amount of a composition comprising a Her3 receptor binding partner, e.g., any one or more of the Her3 specific antibodies disclosed herein effective for that purpose. A further aspect of the invention is a method of alleviating cancer by administering an agonist or antagonist of Her3 receptor. For therapeutic applications, the modulators of Her3 signaling can be used alone, or in combination therapy with, e.g., hormones, antiangiogens, or radiolabeled compounds, or with surgery, cryotherapy, and/or radiotherapy.

The Her3 receptor binding partner useful in destroying cancer cells includes soluble ligands of the receptor, antibodies and fragments thereof that bind the Her3 receptor. The binding partners can be conjugated to a cytotoxic agent. The antibodies are preferably growth inhibitory antibodies. The cytotoxic agent can be a toxin, antibiotic, radioactive isotope or nucleolytic enzyme. A preferred cytotoxic agent is a toxin, preferably a small molecule toxin such as calicheamicin or a maytansinoid.

The antagonists and binding partners of Her3 receptor can be synthetically or recombinantly produced or otherwise isolated.

The mention of particular references, patent application and patents throughout this application should be read as being incorporated by reference into the text of the specification herein in their entirety.

### DEFINITIONS

Before the present proteins, nucleotide sequences, and methods are described, it is to be understood that the present invention is not limited to the particular methodologies, protocols, cell lines, vectors, and reagents described, as these may vary. It is also understood that the terminology used herein is for the purpose of describing particular embodiments only, and is not to limit the scope of the present invention.

The singular forms “a,” “an,” and “the” include plural reference unless the context clearly dictates otherwise.

All technical and scientific terms used herein have the same meanings as commonly understood by one of ordinary skill in the art to which this invention pertains. The practice of the present invention will employ, unless otherwise indicated, conventional techniques of protein chemistry and biochemistry, molecular biology, microbiology and recombinant DNA technology, which are within the skill of the art. Such techniques are explained fully in the literature.

Although any machines, materials, and methods similar or equivalent to those described herein can be used to practice or test the present invention, the preferred machines, materials, and methods are now described. All patents, patent applications, and publications mentioned herein, whether supra or infra, are each incorporated by reference in its entirety.

Terms used throughout this application are to be construed with ordinary and typical meaning to those of ordinary skill in the art. However, Applicants desire that the following terms be given the particular definition as defined below:

Also, the phraseology and terminology used herein is for the purpose of description and should not be regarded as limiting. The use of “including,” “comprising,” or “having,” “containing”, “involving”, and variations thereof herein, is meant to encompass the items listed thereafter and equivalents thereof as well as additional items.

“Nucleic acid” or a “nucleic acid molecule” “nucleic acid molecule encoding Her3” have been used for convenience to encompass DNA encoding Her3, RNA (including pre-mRNA and mRNA or portions thereof) transcribed from such DNA, and also cDNA derived from such RNA. As well it encompasses any DNA or RNA molecule, either single- or double-stranded and, if single-stranded, the molecule of its complementary sequence in either linear or circular form. As used herein, the terms “target nucleic acid” and. In discussing nucleic acid molecules, a sequence or structure of a particular nucleic acid molecule may be described herein according to the normal convention of providing the sequence in the 5′ to 3′ direction. In some embodiments of the invention, nucleic acids are “isolated.” This term, when applied to DNA, refers to a DNA molecule that is separated from sequences with which it is immediately contiguous in the naturally occurring genome of the organism in which it originated. For example, an “isolated nucleic acid” may comprise a DNA molecule inserted into a vector, such as a plasmid or virus vector, or integrated into the genomic DNA of a prokaryotic or eukaryotic cell or host organism. When applied to RNA, the term “isolated nucleic acid” refers primarily to an RNA molecule encoded by an isolated DNA molecule as defined above. Alternatively, the term may refer to an RNA molecule that has been sufficiently separated from other nucleic acids with which it would be associated in its natural state (i.e., in cells or tissues). An isolated nucleic acid (either DNA or RNA) may further represent a molecule produced directly by biological or synthetic means and separated from other components present during its production.

Similarly, “amino acid sequence” as used herein refers to an oligopeptide, peptide, polypeptide, or protein sequence and fragments or portions thereof, of a naturally occurring or synthetic molecule.

The terms “isolated”, “purified”, or “biologically pure” refer to material that is substantially or essentially free from components that normally accompany it as found in its native state. Purity and homogeneity are typically determined using analytical chemistry techniques such as polyacrylamide gel electrophoresis or high performance liquid chromatography. A protein or nucleic acid that is the predominant species present in a preparation is substantially purified. In particular, an isolated nucleic acid is separated from some open reading frames that naturally flank the gene and encode proteins other than protein encoded by the gene. The term “purified” in some embodiments denotes that a nucleic acid or protein gives rise to essentially one band in an electrophoretic gel. Preferably, it means that the nucleic acid or protein is at least 85% pure, more preferably at least 95% pure, and most preferably at least 99% pure. “Purify” or “purification” in other embodiments means removing at least one contaminant from the composition to be purified. In this sense, purification does not require that the purified compound be homogenous, e.g., 100% pure

Nucleic acid is “operably linked” when it is placed into a functional relationship with another nucleic acid sequence. This can be a gene and a regulatory sequence(s) which are connected in such a way as to permit gene expression when the appropriate molecules (e.g., transcriptional activator proteins) are bound to the regulatory sequences(s). For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist; the synthetic oligonucleolide adaptors or linkers are used in accordance with conventional practice.

The term “cell”, “cell line” and “cell culture” are used interchangeably, and all such designations include progeny. It is also understood that all progeny may not be precisely identical in DNA content, due to deliberate or inadvertent mutations. Mutant progeny that have the same function or biological property, as screened for in the originally transformed cell, are included.

The “host cells” used in the present invention generally are prokaryotic or eukaryotic hosts. Examples of suitable host cells are described in Section B. Vectors, Host Cells and Recombinant Methods: (vii) Selection and transformation of host cells.

“Transformation” means introducing DNA into an organism so that the DNA is replicable, either as an extrachromosomal element or by chromosomal integration.

“Transfection” refers to the taking up of an expression vector by a host cell whether or not any coding sequences are in fact expressed.

The terms “transfected host cell” and “transformed” refer to the introduction of DNA into a cell. The cell is termed “host cell” and it may be either prokaryotic or eukaryotic. Typical prokaryotic host cells include various strains of E. coli. Typical eukaryotic host cells are mammalian, such as Chinese hamster ovary or cells of human origin. The introduced DNA sequence may be from the same species as the host cell or a different species from the host cell, or it may be a hybrid DNA sequence, containing some foreign and some homologous DNA.

In the context of the present invention, “modulation” and “modulation of expression” mean either an increase (stimulation) or a decrease (inhibition) in the amount or levels of a nucleic acid molecule encoding the Her3 receptor or the level of the protein—Her3 or modulation of the activity attendant the native Her3 receptor—signaling cascade etc. Inhibition is often the preferred form of modulation of expression and the protein receptor is often a preferred target nucleic acid.

The terms “replicable expression vector” and “expression vector” refer to a piece of DNA, usually double-stranded, which may have inserted into it a piece of foreign DNA. Foreign DNA is defined as heterologous DNA, which is DNA not naturally found in the host cell. The vector is used to transport the foreign or heterologous DNA into a suitable host cell. Once in the host cell, the vector can replicate independently of the host chromosomal DNA and several copies of the vector and its inserted (foreign) DNA may be generated.

The term “vector” means a DNA construct containing a DNA sequence which is operably linked to a suitable control sequence capable of effecting the expression of the DNA in a suitable host. Such control sequences include a promoter to effect transcription, an optional operator sequence to control such transcription, a sequence encoding suitable mRNA ribosome binding sites, and sequences which control the termination of transcription and translation. The vector may be a plasmid, a phage particle, or simply a potential genomic insert. Once transformed into a suitable host, the vector may replicate and function independently of the host genome, or may in some instances, integrate into the genome itself. In the present specification, “plasmid” and “vector” are sometimes used interchangeably, as the plasmid is the most commonly used form of vector at present. However, the invention is intended to include such other form of vector which serve equivalent function as and which are, or become, known in the art. Typical expression vectors for mammalian cell culture expression, for example, are based on pRK5 (EP 307,247), pSV16B (WO 91/08291), and pVL1392 (Pharmingen).

The expression “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

The terms “protein” or “polypeptide” are intended to be used interchangeably. They refer to a chain of two (2) or more amino acids which are linked together with peptide or amide bonds, regardless of post-translational modification (eg., glycosylation or phosphorylation). Antibodies are specifically intended to be within the scope of this definition. The polypeptides of this invention may comprise more than one subunit, where each subunit is encoded by a separate DNA sequence.

Amino acids may be referred to herein either by their commonly known three letter symbols or by the one-letter symbols recommended by the IUPAC-IUB Biochemical Nomenclature Commission. Nucleotides, likewise, may be referred to by their commonly accepted single-letter codes.

The phrase “substantially identical” with respect to an antibody polypeptide sequence shall be construed as an antibody exhibiting at least 70%, preferably 80%, more preferably 90% and most preferably 95% sequence identity to the reference polypeptide sequence. The term with respect to a nucleic acid sequence shall be construed as a sequence of nucleotides exhibiting at least about 85%, preferably 90%, more preferably 95% and most preferably 97% sequence identity to the reference nucleic acid sequence. For polypeptides, the length of the comparison sequences will generally be at least 25 amino acids. For nucleic acids, the length will generally be at least 75 nucleotides. Methods and computer programs for the alignment are well known in the art. Sequence identity may be measured using sequence analysis software (e.g, Sequence Analysis Software Package, Genetics Computer Group, University of Wisconsin Biotechnology Center, 1710 University Ave., Madison, Wis. 53705). This software matches similar sequences by assigning degrees of homology to various substitutions, deletions, and other modifications.

The term “identity” or “homology” shall be construed to mean the percentage of amino acid residues in the candidate sequence that are identical with the residue of a corresponding sequence to which it is compared, after aligning the sequences and introducing gaps, if necessary to achieve the maximum percent identity for the entire sequence, and not considering any conservative substitutions as part of the sequence identity. A molecule is “substantially similar” to another molecule if both molecules have substantially similar structures or biological activity. Thus, provided that two molecules possess a similar activity, they are considered variants as that term is used herein even if the structure of one of the molecules is not found in the other, or if the sequence of amino acid residues is not identical. Neither N- or C-terminal extensions nor insertions shall be construed as reducing identity or homology

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site located at www.ncbi.nlm.nih.gov/BLAST/ or the like). Such sequences are then said to be “substantially identical.” This definition also refers to, or may be applied to, the compliment of a test sequence. The definition also includes sequences that have deletions and/or additions, as well as those that have substitutions, as well as naturally occurring, e.g., polymorphic or allelic variants, and man-made variants. As described below, the preferred algorithms can account for gaps and the like. Preferably, identity exists over a region that is at least about 25 amino acids or nucleotides in length, or more preferably over a region that is 50-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of one of the number of contiguous positions selected from the group consisting typically of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Preferred examples of algorithms that are suitable for determining percent sequence identity and sequence similarity include the BLAST and BLAST 2.0 algorithms. BLAST and BLAST 2.0 are used, with the parameters described herein, to determine percent sequence identity for the nucleic acids and proteins of the invention.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, e.g., where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequences.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical or associated, e.g., naturally contiguous, sequences. Because of the degeneracy of the genetic code, a large number of functionally identical nucleic acids encode most proteins. For instance, the codons GCA, GCC, GCG, and GCU all encode the amino acid alanine. Thus, at every position where an alanine is specified by a codon, the codon can be altered to another of the corresponding codons described without altering the encoded polypeptide. Such nucleic acid variations are “silent variations,” which are one species of conservatively modified variations. Every nucleic acid sequence herein which encodes a polypeptide also describes silent variations of the nucleic acid. One of skill will recognize that in certain contexts each codon in a nucleic acid (except AUG, which is ordinarily the only codon for methionine, and TGG, which is ordinarily the only codon for tryptophan) can be modified to yield a functionally identical molecule. Accordingly, often silent variations of a nucleic acid which encodes a polypeptide is implicit in a described sequence with respect to the expression product, but not with respect to actual probe sequences.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention. Typically conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

The term “amino acid sequence variant” refers to a polypeptide that has amino acid sequences that differ to some extent from a native sequence polypeptide.

“Substitutional” “amino acid variant” refers to molecules with some differences in their amino acid sequences as compared to a native amino acid sequence. The substitutions may be single, where only one amino acid in the molecule as been substituted, or they may be multiple, where two or more amino acids have been substituted in the same molecule. Ordinarily, amino acid sequence variants of Her3 receptor will possess at least about 70% homology with the native sequence Her3 receptor, preferably, at least about 80%, more preferably at least about 85%, even more preferably at least about 90% homology, and most preferably at least 95%. The amino acid sequence variants can possess substitutions, deletions, and/or insertions at certain positions within the amino acid sequence of the native amino acid sequence. “Insertional” variants are those with one or more amino acids inserted immediately adjacent to an amino acid at a particular position in a native sequence. Immediately adjacent to an amino acid means connected to either the .alpha.-carboxyl or .alpha.-amino functional group of the amino acid. “Deletional” variants are those with one or more amino acids in the native amino acid sequence removed. Ordinarily, deletional variants will have one or two amino acids deleted in a particular region of the molecule.

The term “antibody” (Ab) as used herein includes monoclonal antibodies, polyclonal antibodies, multispecific antibodies (e.g. bispecific antibodies), and antibody fragments, so long as they exhibit the desired biological activity. The term “immunoglobulin” (Ig) is used interchangeably with “antibody” herein. An “isolated antibody” is one which has been identified and separated and/or recovered from a component of its natural environment. Contaminant components of its natural environment are materials which would interfere with diagnostic or therapeutic uses for the antibody, and may include enzymes, hormones, and other proteinaceous or nonproteinaceous solutes. In certain embodiments, the antibody will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or non-reducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

The term “immunoglobulin” or “antibody” (used interchangeably herein) is used in the broadest sense and is meant to encompass an immunoglobulin molecule obtained by in vitro or in vivo generation of an immunogenic response. A broad scope refers to an antigen-binding protein having a basic four-polypeptide chain structure consisting of two heavy and two light chains, said chains being stabilized, for example, by interchain disulfide bonds, which has the ability to specifically bind antigen. Both heavy and light chains are folded into domains. The term “domain” refers to a globular region of a heavy or light chain polypeptide comprising peptide loops (e.g., comprising 3 to 4 peptide loops) stabilized, for example, by .beta.-pleated sheet and/or intrachain disulfide bond. Domains are further referred to herein as “constant” or “variable”, based on the relative lack of sequence variation within the domains of various class members in the case of a “constant” domain, or the significant variation within the domains of various class members in the case of a “variable” domain. “Constant” domains on the light chain are referred to interchangeably as “light chain constant regions”, “light chain constant domains”, “CL” regions or “CL” domains) “Constant” domains on the heavy chain are referred to interchangeably as “heavy chain constant regions”, “heavy chain constant domains”, “CH” regions or “CH” domains) “Variable” domains on the light chain are referred to interchangeably as “light chain variable regions”, “light chain variable domains”, “VL” regions or “VL” domains). “Variable” domains on the heavy chain are referred to interchangeably as “heavy chain variable regions”, “heavy chain variable domains”, “V_{H}” regions or “V_{H}” domains). Particular amino acid residues are believed to form an interface between the light and heavy chain variable domains (Clothia et al., J. Mol. Biol. 186, 651-66, 1985); Novotny and Haber, Proc. Natl. Acad. Sci. USA 82, 4592-4596 (1985). The term includes polyclonal, monoclonal, single chain and multivalent antibodies. Fragments e.g., Fab, Fab′, F(ab′)₂, Fv etc. are also included. Representative members include for example, single anti-Her3 monoclonal antibodies (including agonist, antagonist, and neutralizing antibodies), anti-Her3 antibody compositions with polyepitopic specificity (e.g. bispecific antibodies so long as they exhibit the desired biological activity), polyclonal antibodies, single chain anti-Her3 antibodies, and fragments of anti-Her3 antibodies as long as they exhibit the desired biological or immunological activity. The antibodies may be genetically engineered antibodies and/or produced by recombinant DNA techniques. Fully human antibodies can also be produced by phage display, gene and chromosome transfection methods, as well as by other means. The L chain from any vertebrate species can be assigned to one of two clearly distinct types, called kappa and lambda, based on the amino acid sequences of their constant domains. Depending on the amino acid sequence of the constant domain of their heavy chains (C_{H}), immunoglobulins can be assigned to different classes or isotypes. There are five classes of immunoglobulins: IgA, IgD, IgE, IgG, and IgM, having heavy chains designated α, δ, ε, γ, and .mu., respectively. The γ and α classes are further divided into subclasses on the basis of relatively minor differences in C_{H} sequence and function, e.g., humans express the following subclasses: IgG1, IgG2, IgG3, IgG4, IgA1, and IgA2.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. In addition to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler and Milstein, Nature 256, 495 (1975), or may be made by recombinant methods, e.g., as described in U.S. Pat. No. 4,816,567. The monoclonal antibodies for use with the present invention may also be isolated from phage antibody libraries using the techniques described in Clackson el at. Nature 352: 624-628 (1991), as well as in Marks et al., J. Mol. Biol. 222: 581-597 (1991).

An “intact” antibody is one which comprises an antigen-binding site as well as a C_{L} and at least heavy chain constant domains, C_{H1}, C_{H2} and C_{H3}. The constant domains may be native sequence constant domains (e.g. human native sequence constant domains) or amino acid sequence variant thereof. Preferably, the intact antibody has one or more effector functions.

The term “region” refers to a part or portion of an antibody chain and includes constant or variable domains as defined herein, as well as more discrete parts or portions of said domains. For example, light chain variable domains or regions include “complementarity determining regions” or “CDRs” interspersed among “framework regions” or “FRs”, as defined herein.

The term “antigen” as used herein, means a molecule which is reactive with a specific antibody.

“Epitope” or “antigenic determinant” refers to a site on an antigen to which an antibody binds. Epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5 or 8-10 amino acids in a unique spatial conformation. Methods of determining spatial conformation of epitopes include, for example, x-ray crystallography and 2-dimensional nuclear magnetic resonance.

Antibodies of “IgG class” refers to antibodies of IgG1, IgG2, IgG3, and IgG4. The numbering of the amino acid residues in the heavy and light chains is that of the EU index (Kabat, et al., “Sequences of Proteins of Immunological Interest”, 5^{th} ed., National Institutes of Health, Bethesda, Md. (1991); the EU numbering scheme is used herein).

An “immunogenic response” or “antigenic response” is one that results in the production of antibodies directed to a compound after the appropriate cells have been contacted therewith. The compound that is used to elicit an immunogenic response is referred to as an immunogen or antigen. The antibodies produced in the immunogenic response specifically bind the immunogen used to elicit the response.

The term “antibody mutant” refers to an amino acid sequence variant of an antibody wherein one or more of the amino acid residues have been modified. Such mutant necessarily have less than 100% sequence identity or similarity with the amino acid sequence having at least 75% amino acid sequence identity or similarity with the amino acid sequence of either the heavy or light chain variable domain of the antibody, more preferably at least 80%, more preferably at least 85%, more preferably at least 90%, and most preferably at least 95%. Since the method of the invention applies equally to both polypeptides, antibodies and fragments thereof, these terms are sometimes employed interchangeably.

Alternatively or additionally, the word “mutant”, as used herein, is interchangeable with “mutationally-altered” and “glycosylation site altered”. The terms refer to an antibody that comprises at least one immunoglobulin variable region containing at least one mutation that modifies a V region glycosylation site. A mutant immunoglobulin refers to an immunoglobulin (e.g., F(ab′)₂, Fv, Fab, bifunctional antibodies, antibodies, etc.) comprising at least one immunoglobulin variable region containing at least one mutation that modifies a V region glycosylation site. A mutant immunoglobulin chain has at least one mutation that modifies a V region glycosylation site, typically in the V region framework. Thus, the pattern (i.e., frequency and or location(s) of occurrence) of V region glycosylation sites is altered in a mutant immunoglobulin.

The term “variable” in the context of variable domain of antibodies, refers to the fact that certain portions of the variable domains differ extensively in sequence among antibodies and are used in the binding and specificity of each particular antibody for its particular antigen. The V domain mediates antigen binding and define specificity of a particular antibody for its particular antigen. However, the variability is not evenly distributed across the 110-amino acid span of the variable domains. It is concentrated in three segments called complementarity determining regions (CDRs) also known as hypervariable regions both in the light chain and the heavy chain variable domains. There are at least two techniques for determining CDRs: (1) an approach based on cross-species sequence variability (i.e., Kabat et al., Sequences of Proteins of Immunological Interest (National Institute of Health, Bethesda, Md. 1987); and (2) an approach based on crystallographic studies of antigen-antibody complexes (Chothia, C. el at. (1989), Nature 342: 877). The more highly conserved portions of variable domains are called the framework (FR) of 15-30 amino acids separated by shorter “hypervariable regions” (9-12 amino acids long). The variable domains of native heavy and light chains each comprise four FR regions, largely adopting a .beta.-sheet configuration, connected by three CDRs, which form loops connecting, and in some cases forming part of, the .beta.-sheet structure. The CDRs in each chain are held together in close proximity by the FR regions and, with the CDRs from the other chain, contribute to the formation of the antigen binding site of antibodies. See Kabat et al., Sequences of Proteins of Immunological Interest, 5th Ed. Public Health Service, National Institutes of Health, Bethesda, Md. (1991). The constant domains are not involved directly in binding an antibody to an antigen, but exhibit various effector function, such as participation of the antibody in antibody-dependent cellular toxicity.

The term “hypervariable region” when used herein refers to the amino acid residues of an antibody which are responsible for antigen-binding. The hypervariable region generally comprises amino acid residues from a “complementarity determining region” or “CDR” (e.g. around about residues 24-34 (Li), 50-56 (L2) and 89-97 (L3) in the V_{L}, and around about 1-35 (H1), 50-65 (H2) and 95-102 (H3) in the V_{H}; Kabate et al., Sequences of Proteins of Immunological Interest, 5th Ed. Public Health Service, National Institutes of Health, Bethesda, Md. (1991)) and/or those residues from a “hypervariable loop” (e.g. residues 26-32 (L1), 50-52 (L2) and 91-96 (L3) in the V_{L}, and 26-32 (H1), 53-55 (H2) and 96-101 (H3) in the V_{H}; Chothia and Lesk J. Mol. Biol. 196:901-917 (1987)).

Immunoglobulins or antibodies can exist in monomeric or polymeric form. The term “antigen-binding fragment” refers to a polypeptide fragment of an immunoglobulin or antibody binds antigen or competes with intact antibody (i.e., with the intact antibody from which they were derived) for antigen binding (i.e., specific binding). The term “conformation” refers to the tertiary structure of a protein or polypeptide (e.g., an antibody, antibody chain, domain or region thereof). For example, the phrase “light (or heavy) chain conformation” refers to the tertiary structure of a light (or heavy) chain variable region, and the phrase “antibody conformation” or “antibody fragment conformation” refers to the tertiary structure of an antibody or fragment thereof. Preferably, the fragment exhibits qualitative biological activity in common with a full-length antibody. For example, a functional fragment or analog of an anti-IgE antibody is one which can bind to an IgE immunoglobulin in such a manner so as to prevent or substantially reduce the ability of such molecule from having the ability to bind to the high affinity receptor, Fc.epsilon.RI. Antibody fragments can be prepared by in vitro manipulation of antibodies (e.g., by limited proteolysis of an antibody), or via recombinant DNA technology (e.g., the preparation of single-chain antibodies from phage display libraries). Examples of antibody fragments include Fab, Fab′, F(ab′)2, and Fv fragments; diabodies; linear antibodies (U.S. Pat. No. 5,641,870, Example 2; Zapata et al., Protein Eng. 8(10): 1057-1062 (1995)); single-chain antibody molecules; and multispecific antibodies formed from antibody fragments.

Binding fragments are produced by recombinant DNA techniques, or by enzymatic or chemical cleavage of intact immunoglobulins. Binding fragments include Fab, Fab′, F(ab′)2, Fabc, Fv, single chains, and single-chain antibodies. Other than “bispecific” or “bifunctional” immunoglobulins or antibodies, an immunoglobulin or antibody is understood to have each of its binding sites identical.

“A functional variant” of the antibody molecule according to the invention is an antibody molecule which possesses a biological activity (either functional or structural) that is substantially similar to the antibody molecule according to the invention, i.e. a substantially similar substrate specificity or cleavage of the substrate. The term “functional variant” also includes “a fragment”, “an allelic variant”, “variant based on the degenerative nucleic acid code” or “chemical derivatives”. Such a “functional variant” e.g. may carry one or several point mutations, one or several nucleic acid exchanges, deletions or insertions or one or several amino acid exchanges, deletions or insertions. Said functional variant is still retaining its biological activity such as antibody binding activity, at least in part or even going along with an improvement said biological activity.

An “allelic variant” is a variant due to the allelic variation, e.g. differences in the two alleles in humans. Said variant is still retaining its biological activity such as antibody target binding activity, at least in part or even going along with an improvement said biological activity.

A “variant based on the degenerative of the genetic code” is a variant due to the fact that a certain amino acid may be encoded by several different nucleotide triplets. Said variant is still retaining its biological activity such as antibody binding activity, at least in part or even going along with an improvement said biological activity.

A “fusion molecule” may be the antibody molecule according to the invention fused to e.g. a reporter such as a radiolabel, a chemical molecule such as a toxin or a fluorescent label or any other molecule known in the art.

As used herein, a “chemical derivative” according to the invention is an antibody molecule according to the invention chemically modified or containing additional chemical moieties not normally being part of the molecule. Such moieties may improve the molecule's activity such as target destruction (e.g. killing of tumor cells) or may improve its solubility, absorption, biological half life, etc.

Papain digestion of antibodies produces two identical antigen-binding fragments, called “Fab” fragments, and a residual “Fc” fragment, a designation reflecting the ability to crystallize readily. The Fab fragment consists of an entire L chain along with the variable region domain of the H chain (V_{H}), and the first constant domain of one heavy chain (CH 1). Each Fab fragment is monovalent with respect to antigen binding, i.e., it has a single antigen-binding site. Pepsin treatment of an antibody yields a single large F(ab′)2 fragment which roughly corresponds to two disulfide linked Fab fragments having divalent antigen-binding activity and is still capable of cross-linking antigen. Fab′ fragments differ from Fab fragments by having additional few residues at the carboxy terminus of the CH1 domain including one or more cysteines from the antibody hinge region. Fab′-SH is the designation herein for Fab′ in which the cysteine residue(s) of the constant domains bear a free thiol group. F(ab′)2 antibody fragments originally were produced as pairs of Fab′ fragments which have hinge cysteines between them. Other chemical couplings of antibody fragments are also known.

The Fc fragment comprises the carboxy-terminal portions of both H chains held together by disulfides. The effector functions of antibodies are determined by sequences in the Fc region, which region is also the part recognized by Fc receptors (FcR) found on certain types of cells. By “Fc” or “Fc region” as used herein is meant the polypeptides comprising the last two constant region immunoglobulin domains of IgA, IgD, and IgG, and the last three constant region immunoglobulin domains of IgE and IgM, and part of the flexible hinge N-terminal to these domains. Although the boundaries of the Fc region may vary, the human IgG heavy chain Fc region is usually defined to comprise residues C226 or P230 to its carboxyl-terminus, wherein the numbering is according to the EU numbering scheme. Fc may refer to this region in isolation, or this region in the context of a full length antibody or antibody fragment. Ergo, by “outside the Fc region” as used herein is meant the region of an antibody that does not comprise the Fc region of the antibody. In accordance with the aforementioned definition of Fc region, “outside the Fc region” for an IgG1 antibody is herein defined to be from the N-terminus up to and including residue T225 or C229, wherein the numbering is according to the EU numbering scheme. Thus the Fab region and part of the hinge region of an antibody are outside the Fc region.

“Fc receptor” or “FcR” describes a receptor that binds to the Fc region of an antibody. The preferred FcR is a native sequence human FcR. Moreover, a preferred FcR is one which binds an IgG antibody (a γ receptor) and includes receptors of the FcγRI, FcγRII and FcγRIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain. [M. in Daeron, Annu. Rev. Immunol. 15:203-234 (1997)]. FcRs are reviewed in Ravetch and Kinet, Annu. Rev. Immunol. 9:457-492 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein. The term also includes the neonatal receptor, FcRn, which is responsible for the transfer of maternal IgGs to the fetus (Guyer et al., J. Immunol. 117:587 (1976) and Kim et al., J. Immunol. 24:249 (1994)).

An “Fv” fragment is the minimum antibody fragment which comprises the variable domains of its heavy chain and light chain and thus contains a complete antigen recognition and binding site. This region consists of a dimer of one heavy and one light chain variable domain in a tight, non-covalent association (V_{H}-V_{L} dimer). It is in this configuration that the three CDRs of each variable domain interact to define an antigen binding site on the surface of the V_{H}-V_{L} dimer. Collectively, the six CDRs confer antigen binding specificity to the antibody. However, even a single variable domain (or half of an Fv comprising only three CDRs specific for an antigen) has the ability to recognize and bind antigen, although at a lower affinity than the entire binding site. One or more scFv fragments may be linked to other antibody fragments (such as the constant domain of a heavy chain or a light chain) to form antibody constructs having one or more antigen recognition sites.

The term “single chain variable fragment or scFv” refers to an Fv fragment in which the heavy chain domain and the light chain domain are linked. “Single-chain Fv” also abbreviated as “sFv” or “scFv” are antibody fragments that comprise the V_{H} and V_{L} antibody domains connected into a single polypeptide chain. Generally, the sFv polypeptide further comprises a polypeptide linker between the V_{H} and V_{L} domains which enables the sFv to form the desired structure for antigen binding. For a review of sFv, see Pluckthun in The Pharmacology of Monoclonal Antibodies, vol. 113, Rosenburg and Moore eds., Springer-Verlag, New York, pp. 269-315 (1994); Borrebaeck 1995, infra.

The skilled person will also be familiar with so-called miniantibodies which have a bi-, tri- or tetravalent structure and are derived from scFv. The multimerization is carried out by di-, tri- or tetrameric coiled coil structures (Pack et al., 1993 Biotechnology II:, 1271-1277; Lovejoy et al. 1993 Science 259: 1288-1293; Pack et al., 1995 J. Mol. Biol. 246: 28-34).

By minibody the skilled person means a bivalent, homodimeric scFv derivative. It consists of a fusion protein which contains the CH3 region of an immunoglobulin, preferably IgG, most preferably IgG1 as the dimerization region which is connected to the scFv via a hinge region (e.g. also from IgG1) and a linker region. The disulphide bridges in the hinge region are mostly formed in higher cells and not in prokaryotes. In some embodiments an antibody according to the invention is a Her3-specific minibody antibody fragment. Examples of minibody-antibody proteins from the prior art can be found in Hu et al. (1996, Cancer Res. 56: 3055-61).

The term “diabodies” refers to a small antibody fragments with two antigen-binding sites, which fragments comprise a heavy chain variable domain (V_{H}) connected to a light chain variable domain (V_{L}) in the same polypeptide chain (V_{H}-V_{L}). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA 90: 6444-6448 (1993). For the structure and properties of the different classes of antibodies, see, e.g., Basic and Clinical Immunology, 8th edition, Daniel P. Stites, Abba I. Terr and Tristram G. Parslow (eds.), Appleton & Lange, Norwalk, Conn., 1994, page 71 and Chapter 6.

By triabody the skilled person means a: trivalent homotrimeric scFv derivative (Kortt et al. 1997 Protein Engineering 10: 423-433). ScFv derivatives wherein V_{H}-V_{L} are fused directly without a linker sequence lead to the formation of trimers.

The Fab fragment [also designated as F(ab)] also contains the constant domain of the light chain and the first constant domain (CH1) of the heavy chain. Fab′ fragments differ from Fab fragments by the addition of a few residues at the carboxyl terminus of the heavy chain CH1 domain including one or more cysteines from the antibody hinge region. Fab′-SH is the designation herein for Fab′ in which the cysteine residue(s) of the constant domains have a free thiol group. F(ab′) fragments are produced by cleavage of the disulfide bond at the hinge cysteines of the F(ab′)₂ pepsin digestion product. Additional chemical couplings of antibody fragments are known to those of ordinary skill in the art.

A “bispecific” or “bifunctional antibody” is an artificial hybrid antibody having two different heavy/light chain pairs and two different binding sites. Bispecific antibodies can be produced by a variety of methods including fusion of hybridomas or linking of Fab′ fragments. See, e.g., Songsivilai & Lachmann, Clin. Exp. Immunol. 79:315-321 (1990); Kostelny et al., J. Immunol. 148, 1547-1553 (1992).

An antibody “which binds” an antigen of interest, e.g. a tumor-associated polypeptide antigen target., e.g., Her3, is one that binds the antigen with sufficient affinity such that the antibody is useful as a diagnostic and/or therapeutic agent in targeting a cell or tissue expressing the antigen, and does not significantly cross-react with other proteins. In such embodiments, the extent of binding of the antibody to a “non-target” protein will be less than about 10% of the binding of the antibody, oligopeptide or other organic molecule to its particular target protein as determined by fluorescence activated cell sorting (FACS) analysis or radioimmunoprecipitation (RIA). With regard to the binding of an antibody to a target molecule, the term “specific binding” or “specifically binds to” or is “specific for” a particular polypeptide or an epitope on a particular polypeptide target means binding that is measurably different from a non-specific interaction. Specific binding can be measured, for example, by determining binding of a molecule compared to binding of a control molecule, which generally is a molecule of similar structure that does not have binding activity. For example, specific binding can be determined by competition with a control molecule that is similar to the target, for example, an excess of non-labeled target. In this case, specific binding is indicated if the binding of the labeled target to a probe is competitively inhibited by excess unlabeled target.

“Humanized” forms of non-human (e.g., murine) antibodies are chimeric immunoglobulins, immunoglobulin chains or fragments thereof (such as Fv, Fab, Fab′, F(ab′)₂ or other antigen-binding subsequences of antibodies) which contain minimal sequence derived from non-human immunoglobulin. For the most part, humanized antibodies are human immunoglobulins (recipient antibody) in which residues from a complementarity-determining region (CDR) of the recipient are replaced by residues from a CDR of a non-human species (donor antibody) such as mouse, rat or rabbit having the desired specificity, affinity, and capacity. Thus, a humanized antibody is a recombinant protein in which the CDRs from an antibody from one species; e.g., a rodent antibody, is transferred from the heavy and light variable chains of the rodent antibody into human heavy and light variable domains. The constant domains of the antibody molecule is derived from those of a human antibody. In some instances, Fv framework region (FR) residues of the human immunoglobulin are replaced by corresponding non-human residues. Furthermore, humanized antibodies may comprise residues which are found neither in the recipient antibody nor in the imported CDR or framework sequences. These modifications are made to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin sequence. The humanized antibody optimally also will comprise at least a portion of an immunoglobulin constant region (Fc), typically that of a human immunoglobulin. For further details, see Jones et al., Nature, 321:522-525 (1986); Reichmann et al., Nature, 332:323-329 (1988); and Presta, Curr. Op. Struct. Biol., 2:593-596 (1992). The humanized antibody includes a Primatized™ antibody wherein the antigen-binding region of the antibody is derived from an antibody produced by immunizing macaque monkeys with the antigen of interest.

Completely “human” antibodies may be desirable for therapeutic treatment of human patients. Human antibodies can be made by a variety of methods known in the art including phage display methods described above using antibody libraries derived from human immunoglobulin sequences. See U.S. Pat. Nos. 4,444,887 and 4,716,111; and PCT publications WO 98/46645; WO 98/50433; WO 98/24893; WO 98/16654; WO 96/34096; WO 96/33735; and WO 91/10741, each of which is incorporated herein by reference in its entirety. Human antibodies can also be produced using transgenic mice which are incapable of expressing functional endogenous immunoglobulins, but which can express human immunoglobulin genes., see, e.g., PCT publications WO 98/24893; WO 92/01047; WO 96/34096; WO 96/33735; European Patent No. 0 598 877; U.S. Pat. Nos. 5,413,923; 5,625,126; 5,633,425; 5,569,825; 5,661,016; 5,545,806; 5,814,318; 5,885,793; 5,916,771; and 5,939,598, which are incorporated by reference herein in their entireties. In addition, companies such as Abgenix, Inc. (Fremont, Calif.) and Medarex (Princeton, N.J.) can be engaged to provide human antibodies directed against a selected antigen using technology similar to that described above.

Completely human antibodies that recognize a selected epitope can be generated using a technique referred to as “guided selection.” In this approach a selected non-human monoclonal antibody, e.g., a mouse antibody, is used to guide the selection of a completely human antibody recognizing the same epitope (Jespers et al., Biotechnology 12:899-903 (1988).

A chimeric antibody is a recombinant protein that contains the variable domains including the complementarity determining regions (CDRs) of an antibody derived from one species, preferably a rodent antibody, while the constant domains of the antibody molecule is derived from those of a human antibody. For veterinary applications, the constant domains of the chimeric antibody may be derived from that of other species, such as a cat or dog.

A “species-dependent antibody,” e.g., a mammalian anti-human Her3 antibody, is an antibody which has a stronger binding affinity for an antigen from a first mammalian species than it has for a homologue of that antigen from a second mammalian species. Normally, the species-dependent antibody “bind specifically” to a human antigen (i.e., has a binding affinity (Kd) value of no more than about 1×10⁻⁷ M, preferably no more than about 1×10⁻⁸ and most preferably no more than about 1×10⁻⁹ M) but has a binding affinity for homologue of the antigen from a second non-human mammalian species which is at least about 500 fold, or at least about 1000 fold, weaker than its binding affinity for the human antigen. The species-dependent antibody can be of any of the various types of antibodies as defined above, but preferably is a human antibody.

Antibody “effector functions” refer to those biological activities attributable to the Fc region (a native sequence Fc region or amino acid sequence variant Fc region) of an antibody, and vary with the antibody isotype. Examples of antibody effector functions include: C1q binding and complement dependent cytotoxicity; Fe receptor binding; antibody-dependent cell-mediated cytotoxicity (ADCC); phagocytosis; down regulation of cell surface receptors (e.g., B cell receptor); and B cell activation.

“Antibody-dependent cell-mediated cytotoxicity” or “ADCC” refers to a form of cytotoxicity in which secreted Ig bound onto Fc receptors (FcRs) present on certain cytotoxic cells (e.g., Natural Killer (NK) cells, neutrophils, and macrophages) enable these cytotoxic effector cells to bind specifically to an antigen-bearing target cell and subsequently kill the target cell with cytotoxins. The antibodies “arm” the cytotoxic cells and are absolutely required for such killing. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol. 9:457-92 (1991). To assess ADCC activity of a molecule of interest, an in vitro ADCC assay, such as that described in U.S. Pat. No. 5,500,362 or 5,821,337 may be performed. Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. Proc. Natl. Acad. Sci. U.S.A. 95:652-656 (1998).

“Complement dependent cytotoxicity” or “CDC” refers to the lysis of a target cell in the presence of complement. Activation of the classical complement pathway is initiated by the binding of the first component of the complement system (C1q) to antibodies (of the appropriate subclass) which are bound to their cognate antigen. To assess complement activation, a CDC assay, e.g., as described in Gazzano-Santoro et al., J. Immunol. Methods 202:163 (1996), may be performed.

“Human effector cells” are leukocytes which express one or more FcRs and perform effector functions. Preferably, the cells express at least FcγRIII and perform ADCC effector function. Examples of human leukocytes which mediate ADCC include peripheral blood mononuclear cells (PBMC), natural killer (NK) cells, monocytes, cytotoxic T cells and neutrophils; with PBMCs and NK cells being preferred. The effector cells may be isolated from a native source, e.g., from blood.

The term “cytotoxic agent” as used herein refers to a substance that inhibits or prevents the function of cells and/or causes destruction of cells. The term is intended to include radioactive isotopes (e.g. At 211, I 131, I 125, Y 90, Re 186, Re 188, Sm 153, Bi 212, P 32 and radioactive isotopes of Lu), chemotherapeutic agents e.g. methotrexate, adriamycin, vinca alkaloids (vincristine, vinblastine, etoposide), doxorubicin, melphalan, mitomycin C, chlorambucil, daunorubicin or other intercalating agents, enzymes and fragments thereof such as nucleolytic enzymes, antibiotics, and toxins such as small molecule toxins or enzymatically active toxins of bacterial, fungal, plant or animal origin, including fragments and/or variants thereof, and the various anti-tumor or anticancer agents disclosed below. Other cytotoxic agents are described below.

A “chemotherapeutic agent” is a chemical compound useful in the treatment of cancer. Examples of chemotherapeutic agents include adriamycin, doxorubicin, epirubicin, 5-fluorouracil, cytosine arabinoside (“Ara-C”), cyclophosphamide, thiotepa, busulfan, cytoxin, taxoids, e.g., paclitaxel (Taxol™, Bristol-Myers Squibb Oncology, Princeton, N.J.), and docetaxel (Taxotere™, Rhone-Poulenc Rorer, Antony, France), toxotere, methotrexate, cisplatin, melphalan, vinblastine, bleomycin, etoposide, ifosfamide, mitomycin C, mitoxantrone, vincristine, vinorelbine, carboplatin, teniposide, daunomycin, caminomycin, aminopterin, dactinomycin, mitomycins, esperamicins (see U.S. Pat. No. 4,675,187), 6-thioguanine, 6-mercaptopurine, actinomycin D, VP-16, chlorambucil, melphalan, and other related nitrogen mustards. Also included in this definition are hormonal agents that act to regulate or inhibit hormone action on tumors such as tamoxifen and onapristone. A “glycosylation variant” “Glycoform variant” antibody herein is an antibody with one or more carbohydrate moieties attached thereto which differ from one or more carbohydrate moieties attached to a main species antibody. Examples of glycosylation variants herein include antibodies with a G1 or G2 oligosaccharide structure, instead of a G0 oligosaccharide structure, attached to an Fc region thereof, antibody with one or two carbohydrate moieties attached to one or two light chains thereof, antibody with no carbohydrate attached to one or two heavy chains of the antibody, etc, as well as combinations of such glycosylation alterations.

“Glycosylation sites” refer to amino acid residues which are recognized by a eukaryotic cell as locations for the attachment of sugar residues. The amino acids where carbohydrate, such as oligosaccharide, is attached are typically asparagine (N-linkage), serine (O-linkage), and threonine (O-linkage) residues. The specific site of attachment is typically signaled by a sequence of amino acids, referred to herein as a “glycosylation site sequence”. The glycosylation site sequence for N-linked glycosylation is: -Asn-X-Ser- or -Asn-X-Thr-, where X may be any of the conventional amino acids, other than proline. The predominant glycosylation site sequence for O-linked glycosylation is: -(Thr or Ser)-X-X-Pro-, where X is any conventional amino acid. The recognition sequence for glycosaminoglycans (a specific type of sulfated sugar) is -Ser-Gly-X-Gly-, where X is any conventional amino acid. The terms “N-linked” and “O-linked” refer to the chemical group that serves as the attachment site between the sugar molecule and the amino acid residue. N-linked sugars are attached through an amino group; O-linked sugars are attached through a hydroxyl group. However, not all glycosylation site sequences in a protein are necessarily glycosylated; some proteins are secreted in both glycosylated and nonglycosylated forms, while others are fully glycosylated at one glycosylation site sequence but contain another glycosylation site sequence that is not glycosylated. Therefore, not all glycosylation site sequences that are present in a polypeptide are necessarily glycosylation sites where sugar residues are actually attached. The initial N-glycosylation during biosynthesis inserts the “core carbohydrate” or “core oligosaccharide” (Proteins, Structures and Molecular Principles, (1984) Creighton (ed.), W.H. Freeman and Company, New York, which is incorporated herein by reference).

A “V region glycosylation site” is a position in a variable region where a carbohydrate, typically an oligosaccharide, is attached to an amino acid residue in the polypeptide chain via an N-linked or O-linked covalent bond. Since not all glycosylation site sequences are necessarily glycosylated in a particular cell, a glycosylation site is defined operationally by reference to a designated cell type in which glycosylation occurs at the site, and is readily determined by one of ordinary skill in the art. Thus, a mutant antibody has at least one mutation that adds, subtracts, or relocates a V region glycosylation site, such as, for example, an N-linked glycosylation site sequence. Preferably, the mutation(s) are substitution mutations that introduce conservative amino acid substitutions, where possible, to modify a glycosylation site. Preferably, when the parent immunoglobulin sequence contains a glycosylation site in a V region framework, particularly in a location near the antigen binding site (for example, near a CDR), the glycosylation site sequence is mutated (e.g., by site-directed mutagenesis) to abolish the glycosylation site sequence, typically by producing a conservative amino acid substitution of one or more of the amino acid residues comprising the glycosylation site sequence. When the parent immunoglobulin sequence contains a glycosylation site in a CDR, and where the parent immunoglobulin specifically binds an epitope that contains carbohydrate, that glycosylation site is preferably retained. If the parent immunoglobulin specifically binds an epitope that comprises only polypeptide, glycosylation sites occurring in a CDR are preferably eliminated by mutation (e.g., site-directed mutation).

“Glycosylation-reduced antibodies” and “glycosylation-reduced immunoglobulin chains” are mutant antibodies and mutant immunoglobulin chains, respectively, in which at least one glycosylation site that is present in the parent sequence has been destroyed by mutation and is absent in the mutant sequence.

“Glycosylation-supplemented antibodies” and “glycosylation-supplemented immunoglobulin chains” are mutant antibodies and mutant immunoglobulin chains, respectively, in which at least one glycosylation site is present in the mutant sequence at a position where no glycosylation site occurs in the parent sequence. Typically, glycosylation-supplemented antibodies that have a higher binding affinity for a carbohydrate-containing epitope than does the parent antibody have a glycosylation site present in a CDR where the parent antibody does not. Typically, a glycosylation-supplemented antibody that specifically binds an epitope that contains polypeptide sequence but no carbohydrate have a lower affinity that the parental antibody.

“Parent immunoglobulin sequence” (or “parent immunoglobulin”) and “parent polynucleotide sequence” refer herein to a reference amino acid sequence or polynucleotide sequence, respectively. A parent polynucleotide sequence may encode a naturally-occurring immunoglobulin chain of a fragment thereof wherein glycosylation site sequences, if any, present in the V region occur about at the same relative amino acid residue position(s) at which glycosylation site sequence(s) are present in naturally-occurring immunoglobulin sequence(s) from which the parent sequence(s) were derived. When mutations, such as site-directed mutations, are introduced into a parent immunoglobulin sequence, the resultant sequence is referred to as a mutant immunoglobulin sequence (or a mutated immunoglobulin sequence).

As used herein, the twenty conventional amino acids and their abbreviations follow conventional usage (Immunology—A Synthesis, 2nd Edition, E. S. Golub and D. R. Gren, Eds., Sinauer Associates, Sunderland, Mass. (1991) which is incorporated herein by reference).

An antibody drug-conjugate (ADCs) is one approach for the treatment of cancer. (Braslawsky et al., Cancer Res, 1990; 50: 6608-14; Liu et al., Proc Natl Acad Sci USA, 1996; 93: 8618-23; Bernstein et al., Leukemia, 2000; 14: 474-5; Ross et al., Cancer Res, 2002; 62: 2546-53; Bhaskar et al., Cancer Res, 2003; 63: 6387-94; Doronina et al., Nat Biotechnol, 2003; 21: 778-84; Francisco et al., Blood, 2003; 102: 1458-65). The strategy of this approach is to deliver a toxic payload to the cancer cell via an antibody that targets a cancer-specific antigen. This strategy requires that the potent drug is internalized via the antibody-antigen complex, released within the cell and specifically kills the cancer cells (Bhaskar et al., Cancer Res, 2003; 63: 6387-94; Doronina et al., Nat Biotechnol, 2003; 21: 778-84; Francisco et al., Blood, 2003; 102: 1458-65). Ideally, the potent drug is internalized via the antibody-antigen complex, released within the cell and specifically kills the cancer cells. In order to minimize toxic side effects it is critical that the molecular target is not expressed in essential organs that are accessible to circulating antibodies. In addition, the target must be at the plasma membrane of cancer cells to allow antibody access.

The same criteria that make a target attractive for an ADC approach to cancer therapy are also desirable for an antibody dependent cellular cytotoxicity (ADCC) approach. In an ADCC approach, a naked antibody to the target is used to recruit immune effector cells (cytotoxic T lymphocytes, natural killer cells, activated macrophages) to the tumor. These effector cells then specifically kill the targeted tumor cells.

As used herein, the term “antibody phage library” refers to the phage library used in the affinity maturation process described above and in Hawkins et at., J. Mol Biol. 254: 889-896 (1992), and in Lowman et al., Biochemistry 30(45): 10832-10838 (1991). Each library comprises a hypervariable region (eg. 6-7 sites) for which all possible amino acid substitutions are generated. The antibody mutants thus generated are displayed in a monovalent fashion from filamentous phage particles as fusions to the gene III product of M13 packaged within each particle and expressed on the exterior of the phage.

“Single-chain Fv” also abbreviated as “sFv” or “scFv” are antibody fragments that comprise the V H and V L antibody domains connected into a single polypeptide chain. Preferably, the sFv polypeptide further comprises a polypeptide linker between the V H and V L domains which enables the sFv to faun the desired structure for antigen binding. For a review of sFv, see Pluckthun in The Pharmacology of Monoclonal Antibodies, vol. 113, Rosenburg and Moore eds., Springer-Verlag, New York, pp. 269-315 (1994); Borrebaeck 1995, infra.

The term “diabodies” refers to small antibody fragments prepared by constructing sFv fragments (see preceding paragraph) with short linkers (about 5-10 residues) between the V H and V L domains such that inter-chain but not intra-chain pairing of the V domains is achieved, resulting in a bivalent fragment, i.e., fragment having two antigen-binding sites. Bispecific diabodies are heterodimers of two “crossover” sFv fragments in which the V H and V L domains of the two antibodies are present on different polypeptide chains. Diabodies are described more fully in, for example, EP 404,097; WO 93/11161; and Hollinger et al., Proc. Natl. Acad. Sci. USA, 90:6444-6448 (1993).

As used herein, “target molecule” means any molecule, not necessarily a protein, for which it is desirable to produce an antibody or ligand. Preferably, however, the target will be a protein and most preferably the target will be an antigen—EGFR or human her3 or a Her2/Her3 dimer.

“Her3” encompasses all members of the Her3 receptor family including a Her2/Her3 dimer. A “full length” Her3 receptor protein or nucleic acid refers to a polypeptide or polynucleotide sequence, or a variant thereof, that contains all of the elements normally contained in one or more naturally occurring, wild type Her3 polynucleotide or polypeptide sequences. For example, a full length Her3 nucleic acid will typically comprise all of the exons that encode for the full length, naturally occurring protein. The “full length” may be prior to, or after, various stages of post-translation processing.

The term “epitope tagged” when used herein refers to a chimeric polypeptide comprising a polypeptide fused to a “tag polypeptide”. The tag polypeptide has enough residues to provide an epitope against which an antibody can be made, yet is short enough such that it does not interfere with activity of the polypeptide to which it is fused. The tag polypeptide preferably also is fairly unique so that the antibody does not substantially cross-react with other epitopes. Suitable tag polypeptides generally have at least six amino acid residues and usually between about 8 and 50 amino acid residues (preferably, between about 10 and 20 amino acid residues).

By “solid phase” is meant a non-aqueous matrix to which the antibody of the present invention can adhere. Examples of solid phases encompassed herein include those formed partially or entirely of glass (e.g., controlled pore glass), polysaccharides (e.g., agarose), polyacrylamides, polystyrene, polyvinyl alcohol and silicones. In certain embodiments, depending on the context, the solid phase can comprise the well of an assay plate; in others it is a purification column (e.g., an affinity chromatography column). This term also includes a discontinuous solid phase of discrete particles, such as those described in U.S. Pat. No. 4,275,149.

A “liposome” is a small vesicle composed of various types of lipids, phospholipids and/or surfactant which is useful for delivery of a drug (such as an IL-17A/F polypeptide or antibody thereto) to a mammal. The components of the liposome are commonly arranged in a bilayer formation, similar to the lipid arrangement of biological membranes.

A “small molecule” is defined herein to have a molecular weight below about 500 Daltons.

The term “prodrug” as used in this application refers to a precursor or derivative of a pharmaceutically active substance that is less cytotoxic to tumor cells compared to the parent drug and is capable of being enzymatically activated or converted into the more active parent form. See, e.g., Wilman, “Prodrugs in Cancer Chemotherapy,” Biochemical Society Transactions, 14, pp. 375-382, 615 Meeting, Belfast (1986) and Stella et al., (ed.), “Prodrugs: A Chemical Approach to Targeted Drug Delivery,” Directed Drug Delivery, Borchardt et al., (ed.), pp. 247-267, Human Press (1985. Examples of cytotoxic drugs that can be derivatized into a prodrug form for use in this invention include, but are not limited to, those chemotherapeutic agents described above.

An “antibody that inhibits the growth of cancer cells expressing Her3 receptor or a “growth inhibitory” antibody is one which binds to and results in measurable growth inhibition of cancer cells expressing or overexpressing Her3 receptor. Growth inhibition of tumor cells in vivo can be determined in various ways. The antibody is growth inhibitory in vivo if administration of the anti-Her3 antibody at a therapeutically effective dose results in reduction in tumor size or tumor cell proliferation within a measurable period of time from the first administration of the antibody. Growth inhibition can be measured at an antibody concentration of about 0.1 to 30 μg/ml or about 0.5 nM to 200 nM in cell culture where the growth inhibition is determined 1-10 days after exposure of the tumor cells to the antibody. An antibody that binds to “Her3” includes an antibody that preferably binds Her3 and prevents dimerization with Her2.

The terms “cancer” “neoplasia” and “cancerous” refer to or describe any malignant neoplasm or spontaneous growth or proliferation of cells. The term as used herein encompasses both fully developed malignant neoplasms, as well as premalignant lesions. A subject having “cancer”, for example, may have a tumor.

A “Her3 receptor-expressing cancer” is a cancer comprising cells that have Her3 receptor protein present on the cell surface. A “Her3 receptor-expressing cancer” produces sufficient levels of Her3 receptor on the surface of cells thereof, such that a Her3 receptor agonist/antagonist or antibody can bind thereto and have a therapeutic effect with respect to the cancer. A cancer which “overexpresses” Her3 receptor is one which has significantly higher levels of Her3 receptor at the cell surface thereof, compared to a noncancerous cell of the same tissue type. Her3 receptor overexpression may be determined in a diagnostic or prognostic assay by evaluating increased levels of the Her3 receptor protein present on the surface of a cell (e.g. via an immunohistochemistry assay; FACS analysis). Alternatively, or additionally, one may measure levels of Her3 receptor-encoding nucleic acid or mRNA in the cell, e.g. via fluorescent in situ hybridization; (FISH; see WO98/45479 published October, 1998), Southern blotting, Northern blotting, or polymerase chain reaction (PCR) techniques, such as real time quantitative PCR (RT-PCR). One may also study Her3 receptor overexpression by measuring shed antigen in a biological fluid such as serum, e.g, using antibody-based assays (see also, e.g., U.S. Pat. No. 4,933,294 issued Jun. 12, 1990; WO91/05264 published Apr. 18, 1991: U.S. Pat. No. 5,401,638 issued Mar. 28, 1995; and Sias et. al. J. Immunol. Methods 132: 73-80 (1990)). Aside from the above assays, various in vivo assays are available to the skilled practitioner. For example, one may expose cells within the body of the patient to an antibody which is optionally labeled with a detectable label, e.g. a radioactive isotope, and binding of the antibody to cells in the patient can be evaluated, e.g. by external scanning for radioactivity or by analyzing a biopsy taken from a patient previously exposed to the antibody.

“Alleviation of cancer” refers to both therapeutic treatment and prophylactic or preventative measures, wherein the object is to prevent or slow down (lessen) the targeted pathologic condition or disorder. Those in need of alleviation include those already with the disorder as well as those prone to have the disorder or those in whom the disorder is to be prevented. A subject or mammal is “alleviated” for a Her3 receptor-expressing cancer if, after receiving a therapeutic amount of a Her3 receptor agonist according to the methods of the present invention, the patient shows observable and/or measurable reduction in or absence of one or more of the following: reduction in the number of cancer cells or absence of the cancer cells, reduction in the tumor size; inhibition (i.e., slow to some extent and preferably stop) of cancer cell infiltration into peripheral organs; inhibition (i.e., slow to some extent and preferably stop) of tumor metastasis; inhibition, to some extent, of tumor growth; and/or relief to some extent, one or more of the symptoms associated with the specific cancer, and reduced morbidity and mortality. To the extent the Her3 receptor antagonist or antibody may prevent growth and/or kill existing cancer cells, it may be cytostatic and/or cytotoxic. Reduction of these signs or symptoms may also be felt by the patient. Detection and measurement of these above indicators are known to those of skill in the art, including, but not limited for example, reduction in tumor burden, inhibition of tumor size, reduction in proliferation of secondary tumors, expression of genes in tumor tissue, presence of biomarkers, lymph node involvement, histologic grade, and nuclear grade.

The term “therapeutically effective amount” refers to an amount of an agonist and/or antagonist antibody effective to “alleviate” a disease or disorder in a subject or mammal. A “therapeutically effective amount”, in reference to the treatment of tumor, refers to an amount capable of invoking one or more of the following effects: (1) inhibition, to some extent, of tumor growth, including, slowing down and complete growth arrest; (2) reduction in the number of tumor cells; (3) reduction in tumor size; (4) inhibition (i.e., reduction, slowing down or complete stopping) of tumor cell infiltration into peripheral organs; (5) inhibition (i.e., reduction, slowing down or complete stopping) of metastasis; (6) enhancement of anti-tumor immune response, which may, but does not have to, result in the regression or rejection of the tumor; and/or (7) relief, to some extent, of one or more symptoms associated with the disorder. A “therapeutically effective amount” of a Her3 antibody for purposes of treatment of tumor may be determined empirically and in a routine manner.

The term “inhibition of tumor volume” refers to any decrease or reduction in a tumor volume. The term “tumor volume” refers to the total size of the tumor, which includes the tumor itself plus affected lymph nodes if applicable. Tumor volume may be determined by a variety of methods known in the art, such as, e.g. by measuring the dimensions of the tumor using calipers, computed tomography (CT) or magnetic resonance imaging (MRI) scans, and calculating the volume using equations based on, for example, the z-axis diameter, or on standard shapes such as the sphere, ellipsoid, or cube.

The term “biologically active” (synonymous with “bioactive”) indicates that a composition or compound itself has a biological effect, or that it modifies, causes, promotes, enhances, blocks, reduces, limits the production or activity of, or reacts with or binds to an endogenous molecule that has a biological effect. A “biological effect” may be but is not limited to one that stimulates or causes an immunoreactive response; one that impacts a biological process in an animal; one that impacts a biological process in a pathogen or parasite; one that generates or causes to be generated a detectable signal; and the like. Biologically active compositions, complexes or compounds may be used in therapeutic, prophylactic and diagnostic methods and compositions. Biologically active compositions, complexes or compounds act to cause or stimulate a desired effect upon an animal. Non-limiting examples of desired effects include, for example, preventing, treating or curing a disease or condition in an animal suffering therefrom; limiting the growth of or killing a pathogen in an animal infected thereby; augmenting or altering the phenotype or genotype of an animal; and stimulating a prophylactic immunoreactive response in an animal.

In the context of therapeutic applications of the invention, the term “biologically active” indicates that the composition, complex or compound has an activity that impacts an animal suffering from a disease or disorder in a positive sense and/or impacts a pathogen or parasite in a negative sense. Thus, a biologically active composition, complex or compound may cause or promote a biological or biochemical activity within an animal that is detrimental to the growth and/or maintenance of a pathogen or parasite; or of cells, tissues or organs of an animal that have abnormal growth or biochemical characteristics, such as cancer cells, or cells affected by autoimmune or inflammatory disorders.

In the context of prophylactic applications of the invention, the term “biologically active” indicates that the composition or compound induces or stimulates an immunoreactive response. In some embodiments, the immunoreactive response is designed to be prophylactic, i.e., prevents infection by a pathogen. In other embodiments, the immunoreactive response is designed to cause the immune system of an animal to react to the detriment of cells of an animal, such as cancer cells, that have abnormal growth or biochemical characteristics. In this application of the invention, compositions, complexes or compounds comprising antigens are formulated as a vaccine.

It will be understood by those skilled in the art that a given composition, complex or compound may be biologically active in therapeutic, diagnostic and prophylactic applications. A composition, complex or compound that is described as being “biologically active in a cell” is one that has biological activity in vitro (i.e., in a cell culture) or in vivo (i.e., in the cells of an animal). A “biologically active portion” of a compound or complex is a portion thereof that is biologically active once it is liberated from the compound or complex. It should be noted, however, that such a component may also be biologically active in the context of the compound or complex.

In order to achieve a biological effect, invention constructs may comprise an additional moiety to facilitate internalization and/or uptake by a target cell.

A “patient” or “subject” or “host” refers to either a human or non-human animal.

“Treatment” refers to both therapeutic treatment and prophylactic or preventative measures. Those in need of treatment include those already with the disorder as well as those in which the disorder is to be prevented. The above parameters for assessing successful treatment and improvement in the disease are readily measurable by routine procedures familiar to a physician. For cancer therapy, efficacy can be measured, for example, by assessing the time to disease progression (TTP) and/or determining the response rate (RR). For prostate cancer, the progress of therapy can be assessed by routine methods, usually by measuring serum PSA (prostate specific antigen) levels; the higher the level of PSA in the blood, the more extensive the cancer. Commercial assays for detecting PSA are available, e.g, Hybritech Tandem-E® and Tandem-R® PSA assay kits, the Yang ProsCheck® polyclonal assay (Yang Labs, Bellevue, Wash.), Abbott Imx® (Abbott Labs, Abbott Park, Ill.), etc. Metastasis can be determined by staging tests and by bone scan and tests for calcium level and other enzymes to determine spread to the bone. CT scans can also be done to look for spread to the pelvis and lymph nodes in the area. Chest X-rays and measurement of liver enzyme levels by known methods are used to look for metastasis to the lungs and liver, respectively. Other routine methods for monitoring the disease include transrectal ultrasonography (TRUS) and transrectal needle biopsy (TRNB).

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

An “antagonist” of Her3 receptor in addition to binding Her3 receptor, has a direct effect on a Her3 receptor bearing cell. The Her3 receptor agonist will bind Her3 receptor, and as well, initiate or mediate the signaling event associated with the Her3 receptor or the Her2/Her3 dimer. The ability to induce Her3 receptor activation can be quantified using techniques known in the art such as reporter constructs such as Beta-galactosidase, chloramphenicol acetyl transferase (CAT) or luciferase. The Her3 receptor antagonist will inhibit signaling transmitted from the Her3 receptor or the Her2/Her3 dimer or prevent Her3 from associating with Her2 or another member of the EGFR family.

The Her3 receptor “antagonist” will inhibit signaling transmitted from the Her3 receptor through various mechanisms including blocking formation of a Her2/Her3 dimer formation or stimulating receptor degradation. The term “antagonist” is used in the broadest sense, and includes any molecule that partially or fully blocks, inhibits, or neutralizes a biological activity of a native Her3 receptor protein.

Suitable antagonist molecules specifically include antagonist antibodies or antibody fragments, fragments or amino acid sequence variants thereof etc. Methods for identifying agonists or antagonists of a Her3 receptor polypeptide are known in the art. An exemplary method proposes contacting a Her3 bearing cells or tissue with a candidate antagonist molecule and measuring a detectable change in one or more biological activities normally associated with the Her3 receptor.

“Mammal” for purposes of treatment refers to any animal classified as a mammal, including humans, domestic and farm animals, and zoo, sports, or pet animals, such as dogs, cats, cattle, horses, sheep, pigs, goats, rabbits, etc. Preferably, the mammal is human.

The term “administering” includes any method of delivery of a compound of the present invention, including but not limited to, a pharmaceutical composition or therapeutic agent, into a subject's system or to a particular region in or on a subject. The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration. “Parenteral administration” and “administered parenterally” means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intra-articular, subcapsular, subarachnoid, intraspinal and intrasternal injection and infusion.

Administration “in combination with” one or more further therapeutic agents includes simultaneous (concurrent) and consecutive administration in any order.

The term “modulate” means to affect (e.g., either upregulate, downregulate or otherwise control) the level of a signaling pathway. Cellular processes under the control of signal transduction include, but are not limited to, transcription of specific genes, normal cellular functions, such as metabolism, proliferation, differentiation, adhesion, apoptosis and survival, as well as abnormal processes, such as transformation, blocking of differentiation and metastasis.

A “disorder” is any condition that would benefit from treatment with the polypeptide. This includes chronic and acute disorders or diseases including those pathological conditions which predispose the mammal to the disorder in question.

“Chronic” administration refers to administration of the agent(s) in a continuous mode as opposed to an acute mode, so as to maintain the initial therapeutic effect (activity) for an extended period of time. “Intermittent” administration is treatment that is not consecutively done without interruption, but rather is cyclic in nature.

An “effector” or “effector moiety” or “effector component” is a molecule that is bound (or linked, or conjugated), either covalently, through a linker or a chemical bond, or noncovalently, through ionic, van der Waals, electrostatic, or hydrogen bonds, to an antibody. The “effector” can be a variety of molecules including, e.g., detection moieties including radioactive compounds, fluorescent compounds, an enzyme or substrate, tags such as epitope tags, a toxin, activatable moieties, a chemotherapeutic or cytotoxic agent, a chemoattractant, a lipase; an antibiotic; or a radioisotope emitting “hard” e.g., beta radiation.

The same criteria that make a target attractive for an ADC approach to cancer therapy are also desirable for an antibody dependent cellular cytotoxicity (ADCC) approach. In an ADCC approach, a naked antibody to the target is used to recruit immune effector cells (cytotoxic T lymphocytes, natural killer cells, activated macrophages) to the tumor. These effector cells then specifically kill the targeted tumor cells.

“Biological sample” as used herein is a sample of biological tissue or cells that contains nucleic acids or polypeptides, e.g., Her3 or Her3 protein, polynucleotide or transcript. Such samples include, but are not limited to, tissue isolated from primates (e.g., humans) or from rodents (e.g., mice, and rats). Biological samples may also include sections of tissues such as biopsy and autopsy samples, frozen sections taken for histologic purposes, skin, etc. Biological samples also include explants and primary and/or transformed cell cultures derived from patient tissues. A biological sample is typically obtained from a eukaryotic organism, most preferably a mammal such as a primate e.g., human.

“Providing a biological sample” means to obtain a biological sample for use in methods described in this invention. Most often, this will be done by removing a sample of cells from a human, but can also be accomplished by using previously isolated cells (e.g., isolated by another person, at another time, and/or for another purpose), or by performing the methods of the invention in vivo.

“Tumor”, as used herein, refers to all neoplastic cell growth and proliferation, whether malignant or benign, and all pre-cancerous and cancerous cells and tissues.

“Her3” encompasses all members of the Her3 receptor family and in particular, Her3 and Her3.

The Her3 ligands include Jagged1, Jagged2, Delta1, Delta3, and Delta4. “Her3” cDNA and deduced amino acid sequence is as set forth in SEQ ID NOS. 1 and 2.

A “label” or a “detectable moiety” is a composition detectable by spectroscopic, photochemical, biochemical, immunochemical, chemical, or other physical means. The word “label” when used herein refers to a detectable compound or composition which is conjugated directly or indirectly to the antibody so as to generate a “labeled” antibody. The label may be detectable by itself (e.g. radioisotope labels or fluorescent labels) or, in the case of an enzymatic label, may catalyze chemical alteration of a substrate compound or composition which is detectable. For example, useful labels include fluorescent dyes, electron-dense reagents, enzymes (e.g., as commonly used in an ELISA), biotin, digoxigenin, colloidal gold, luminescent nanocrystals (e.g. quantum dots), haptens and proteins or other entities which can be made detectable, e.g., by incorporating a radiolabel into the peptide or used to detect antibodies specifically reactive with the peptide. The radioisotope may be, for example, ³H, ¹⁴C, ³²P, ³⁵S, or ¹²⁵I. In some cases, particularly using antibodies against the proteins of the invention, the radioisotopes are used as toxic moieties, as described below. Any method known in the art for conjugating the antibody to the label may be employed. The lifetime of radiolabeled peptides or radiolabeled antibody compositions may be extended by the addition of substances that stabilize the radiolabeled peptide or antibody and protect it from degradation. Any substance or combination of substances that stabilize the radiolabeled antibody may be used including those substances disclosed in U.S. Pat. No. 5,961,955.

During last few years, it has been show that the targeting of growth factor receptors, like EGFR or Her2/neu over-expressed on the tumoral cell surface, with respectively humanized (Herceptin™) or chimeric (C225) antibodies results in an significant inhibition of the tumoral growth on patients and in a significant increase of the efficacy of classical chemotherapy treatments (Carter P., Nature Rev. Cancer, 2001, 1(2):118; Hortobagyi G. N., Semin. Oncol., 2001, 28:43; Herbst R. S. et al., Semin. Oncol., 2002, 29:27). Other receptors like IGF-IR or VEGF-R (for vascular endothelial growth factor receptor) have been identified as potential target in several preclinical studies.

### Preparation of Chimeric, Humanized and Human Anti-Her3 Antibodies

The monoclonal antibodies herein include chimeric, hybrid and recombinant antibodies produced by splicing a variable (including hypervariable) domain of the antibody of interest with a constant domain (e.g. “humanized” antibodies), or a light chain with a heavy chain, or a chain from one species with a chain from another species, or fusions with heterologous proteins, regardless of species of origin or immunoglobulin class or subclass designation, as well as antibody fragments (e.g., Fab, F(ab′)₂, and Fv), so long as they exhibit the desired biological activity or properties. See, e.g. U.S. Pat. No. 4,816,567 and Mage et al., in Monoclonal Antibody Production Techniques and Applications, pp. 79-97 (Marcel Dekker, Inc.: New York, 1987). Thus, for the purposes of obtaining a chimeric Her3 antibody, the CDR's from the murine antibodies disclosed herein can be grafted onto a human “Y” framework. The resulting “chimeric” Her3 antibodies, in turn, can be humanized by techniques known to one skilled in the art. The affinity of a chimeric, humanized or human anti-Her3 antibody may be evaluated using a direct binding assay or a competitive binding assay, as exemplified below.

### Antibody Structure

Naturally occurring (wildtype) antibody molecules are Y-shaped molecules consisting of four polypeptide chains, two identical heavy chains and two identical light chains, which are covalently linked together by disulfide bonds. Both types of polypeptide chains have constant regions, which do not vary or vary minimally among antibodies of the same class (i.e., IgA, IgM, etc.), and variable regions. The variable regions are unique to a particular antibody and comprise a recognition element for an epitope. The carboxy-terminal regions of both heavy and light chains are conserved in sequence and are called the constant regions (also known as C-domains). The amino-terminal regions (also known as V-domains) are variable in sequence and are responsible for antibody specificity. The antibody specifically recognizes and binds to an antigen mainly through six short complementarity-determining regions (CDRs) located in their V-domains.

Each light chain of an antibody is associated with one heavy chain, and the two chains are linked by a disulfide bridge formed between cysteine residues in the carboxy-terminal region of each chain, which is distal from the amino terminal region of each chain that constitutes its portion of the antigen binding domain. Antibody molecules are further stabilized by disulfide bridges between the two heavy chains in an area known as the hinge region, at locations nearer the carboxy terminus of the heavy chains than the locations where the disulfide bridges between the heavy and light chains are made. The hinge region also provides flexibility for the antigen-binding portions of an antibody.

An antibody's specificity is determined by the variable regions located in the amino terminal regions of the light and heavy chains. The variable regions of a light chain and associated heavy chain form an “antigen binding domain” that recognizes a specific epitope; an antibody thus has two antigen binding domains. The antigen binding domains in a wild type antibody are directed to the same epitope of an immunogenic protein, and a single wild type antibody is thus capable of binding two molecules of the immunogenic protein at the same time. Thus, a wild type antibody is monospecific (i.e., directed to a unique antigen) and divalent (i.e., capable of binding two molecules of antigen).

### Types of Antibodies

“Polyclonal antibodies” are generated in an immunogenic response to a protein having many epitopes. A composition (e.g., serum) of polyclonal antibodies thus includes a variety of different antibodies directed to the same and to different epitopes within the protein. Methods for producing polyclonal antibodies are known in the art (see, e.g., Cooper et al., Section III of Chapter 11 in: Short Protocols in Molecular Biology, 2nd Ed., Ausubel et al., eds., John Wiley and Sons, New York, 1992, pages 11-37 to 11-41).

“Antipeptide antibodies” (also known as “monospecific antibodies”) are generated in a humoral response to a short (typically, 5 to 20 amino acids) immunogenic polypeptide that corresponds to a few (preferably one) isolated epitopes of the protein from which it is derived. A plurality of antipeptide antibodies includes a variety of different antibodies directed to a specific portion of the protein, i.e, to an amino acid sequence that contains at least one, preferably only one, epitope. Methods for producing antipeptide antibodies are known in the art (see, e.g., Cooper et al., Section III of Chapter 11 in: Short Protocols in Molecular Biology, 2nd Ed., Ausubel et al., eds., John Wiley and Sons, New York, 1992, pages 11-42 to 11-46).

A “Monoclonal antibody” is a specific antibody that recognizes a single specific epitope of an immunogenic protein. In a plurality of a monoclonal antibody, each antibody molecule is identical to the others in the plurality. In order to isolate a monoclonal antibody, a clonal cell line that expresses, displays and/or secretes a particular monoclonal antibody is first identified; this clonal cell line can be used in one method of producing the antibodies of the invention. Methods for the preparation of clonal cell lines and of monoclonal antibodies expressed thereby are known in the art (see, for example, Fuller et al., Section II of Chapter 11 in: Short Protocols in Molecular Biology, 2nd Ed., Ausubel et al., eds., John Wiley and Sons, New York, 1992, pages 11-22 to 11-11-36).

A “Naked antibody” is an antibody that lacks the Fc portion of a wildtype antibody molecule. The Fc portion of the antibody molecule provides effector functions, such as complement fixation and ADCC (antibody dependent cell cytotoxicity), which set mechanisms into action that may result in cell lysis. See, e.g., Markrides, Therapeutic inhibition of the complement system, Pharmacol. Rev. 50:59-87, 1998. In some systems, it appears that the therapeutic action of an antibody depends upon the effector functions of the Fc region (see, e.g., Golay et al., Biologic response of B lymphoma cells to anti-CD20 monoclonal antibody rituximab in vitro: CD55 and CD59 regulate complement-mediated cell lysis, Blood 95:3900-3908, 2000).

However, it is possible that the Fc portion is not required for therapeutic function in every instance, as other mechanisms, such as apoptosis, can come into play. Moreover, the Fc region may be deleterious in some applications as antibodies comprising an Fc region are taken up by Fc receptor-bearing cells, thereby reducing the amount of therapeutic antibody taken up by targeted cells. Vaswani and Hamilton, Humanized antibodies as potential therapeutic drugs, Ann. Allergy Asthma Immunol. 81:105-119, 1998. Components of the immune system may recognize and react to antibodies that are clumped together on the surface of tumor cells. It is thus envisioned that the resulting immune response will target and destroy, or at least limit the proliferation of, the tumor cells.

One way to get naked antibodies delivered to surfaces where they will clump together is to use a targetable construct or complex to bring different naked antibodies together on a targeted cellular surface. By way of non-limiting example, an anti-C20 antibody (e.g., Rituxan) and an anti-C22 antibody might be administered separately or together, allowed to clear so that unbound antibodies are removed from the system.

Naked antibodies are also of interest for therapy of diseases caused by parasites, such as malaria. Vukovic et al., Immunoglobulin G3 antibodies specific for the 19-kilodalton carboxyl-terminal fragment of Plasmodium yoelii merozoite surface protein 1 transfer protection to mice deficient in Fc-RI receptors, Infect. Immun. 68:3019-22, 2000.

“Single chain antibodies (scFv)” generally do not include portions of the Fc region of antibodies that are involved in effector functions and are thus naked antibodies, although methods are known for adding such regions to known scFv molecules if desired. See Helfrich et al., A rapid and versatile method for harnessing scFv antibody fragments with various biological functions, J. Immunol. Meth. 237:131-145, 2000; and de Haard et al., Creating and engineering human antibodies for immunotherapy, Adv. Drug Delivery Rev. 31:5-31, 1998.

### Antibody Fragments

Proteolytic Antibody Fragments

Antibody fragments produced by limited proteolysis of wild type antibodies are called proteolytic antibody fragments. These include, but are not limited to, the following.

“F(ab′)₂ fragments” are released from an antibody by limited exposure of the antibody to a proteolytic enzyme, e.g., pepsin or ficin. An F(ab′)₂ fragment comprises two “anus,” each of which comprises a variable region that is directed to and specifically binds a common antigen. The two Fab′ molecules are joined by interchain disulfide bonds in the hinge regions of the heavy chains; the Fab′ molecules may be directed toward the same (bivalent) or different (bispecific) epitopes.

“Fab′ fragments” contain a single anti-binding domain comprising a Fab and an additional portion of the heavy chain through the hinge region.

“Fab′-SH fragments” are typically produced from F(ab′)₂ fragments, which are held together by disulfide bond(s) between the H chains in an F(ab′)₂ fragment. Treatment with a mild reducing agent such as, by way of non-limiting example, beta-mercaptoethylamine, breaks the disulfide bond(s), and two Fab′ fragments are released from one F(ab′)₂ fragment. Fab′-SH fragments are monovalent and monospecific.

“Fab fragments” (i.e., an antibody fragment that contains the antigen-binding domain and comprises a light chain and part of a heavy chain bridged by a disulfide bond) are produced by papain digestion of intact antibodies. A convenient method is to use papain immobilized on a resin so that the enzyme can be easily removed and the digestion terminated. Fab fragments do not have the disulfide bond(s) between the H chains present in an F(ab′)₂ fragment.

### Recombinant Antibody Fragments

“Single-chain antibodies” are one type of antibody fragment. The term single chain antibody is often abbreviated as “scFv” or “sFv.” These antibody fragments are produced using molecular genetics and recombinant DNA technology. A single-chain antibody consists of a polypeptide chain that comprises both a V_{H} and a V_{L} portion. Unlike wildtype antibodies, wherein two separate heavy and light polypeptide chains are conjoined to form a single antigen-binding variable region, a single-chain antibody is a single polypeptide that comprises an antigen-binding variable region. That is, a single-chain antibody comprises the variable, antigen-binding determinative region of a single light and heavy chain of an antibody linked together by a chain of 10-25 amino acids.

The term “single-chain antibody” includes but is not limited to a disulfide-linked Fv (dsFv) in which two single-chain antibodies linked together by a disulfide bond; a bispecific sFv (a sFv or a dsFv molecule having two antigen-binding domains, each of which may be directed to a different epitope); a diabody (a dimerized sFv formed when the V_{H} domain of a first sFv assembles with the V_{L}, domain of a second sFv and the V_{L} domain of the first sFv assembles with the V_{H} domain of the second sFv; the two antigen-binding regions of the diabody may be directed towards the same or different epitopes); and a triabody (a trimerized sFv, formed in a manner similar to a diabody, but in which three antigen-binding domains are created in a single complex; the three antigen binding domains may be directed towards the same or different epitopes).

“Fully human antibodies” are human antibodies that can be produced in transgenic animals such as Xenomice. XenoMouse strains are genetically engineered mice in which the murine IgH and Igk loci have been functionally replaced by their human Ig counterparts on yeast artificial YAC transgenes. These human Ig transgenes can carry the majority of the human variable repertoire and can undergo class switching from IgM to IgG isotypes. The immune system of the xenomouse recognizes administered human antigens as foreign and produces a strong humoral response. The use of XenoMouse in conjunction with well-established hybridomas techniques, results in fully human IgG mAbs with sub-nanomolar affinities for human antigens (see U.S. Pat. No. 5,770,429, entitled “Transgenic non-human animals capable of producing heterologous antibodies”, U.S. Pat. No. 6,162,963, entitled “Generation of xenogenetic antibodies”; U.S. Pat. No. 6,150,584, entitled “Human antibodies derived from immunized xenomice”, U.S. Pat. No. 6,114,598, entitled “Generation of xenogeneic antibodies”; and U.S. Pat. No. 6,075,181, entitled “Human antibodies derived from immunized xenomice”; for reviews, see Green, Antibody engineering via genetic engineering of the mouse: XenoMouse strains are a vehicle for the facile generation of therapeutic human monoclonal antibodies, J. Immunol. Meth. 231:11-23, 1999; Wells, Eek, a XenoMouse: Abgenix, Inc., Chem. Biol. 7:R185-6, 2000; and Davis et al., Transgenic mice as a source of filly human antibodies for the treatment of cancer, Cancer Metastasis Rev. 18:421-5, 1999).

“Complementary determining region peptides” or “CDR peptides” are another faun of an antibody fragment. A CDR peptide (also known as “minimal recognition unit”) is a peptide corresponding to a single complementarity-determining region (CDR), and can be prepared by constructing genes encoding the CDR of an antibody of interest. Such genes are prepared, for example, by using the polymerase chain reaction to synthesize the variable region from RNA of antibody-producing cells. See, for example, Larrick et al., Methods: A Companion to Methods in Enzymology 2:106, 1991.

### Compositions of the Invention and Methods of Making Same

This invention encompasses a plurality of substantially pure, isolated anti-Her3 isolated antibodies and polynucleotide embodiments. Explicitly included are compositions, including pharmaceutical compositions, comprising an anti-Her3 antibody; and polynucleotides comprising sequences encoding an anti-Her3 antibody. As used herein, compositions comprise one or more antibodies that bind to Her3, and/or one or more polynucleotides comprising sequences encoding one or more antibodies that bind to Her3. These compositions may further comprise suitable carriers, such as pharmaceutically acceptable excipients including buffers, which are well known in the art.

The anti-Her3 antibodies of the invention are preferably monoclonal. Also encompassed within the scope of the invention are Fab, Fab′, Fab′-SH and F(ab′)₂ fragments of the anti-Her3 antibodies provided herein. Single chain anti-Her3 antibodies as well as multispecific and multivariant Her3 specific antibodies are also included. These antibody fragments can be created by traditional means, such as enzymatic digestion, or may be generated by recombinant techniques. These fragments are useful for the diagnostic and therapeutic purposes set forth below.

Monoclonal antibodies are obtained from a population of substantially homogeneous antibodies, i.e., the individual antibodies comprising the population are identical except for possible naturally occurring mutations that may be present in minor amounts. Thus, the modifier “monoclonal” indicates the character of the antibody as not being a mixture of discrete antibodies.

The anti-Her3 monoclonal antibodies of the invention are preferably made by recombinant DNA methods (U.S. Pat. No. 4,816,567).

The binding specificity of monoclonal antibodies produced by recombinant means is determined by immunoprecipitation or by an in vitro binding assay, such as radioimmunoassay (RIA) or enzyme-linked immunoadsorbent assay (ELISA).

The binding affinity of the monoclonal antibody can, for example, be determined by the Scatchard analysis of Munson et al., Anal. Biochem., 107:220 (1980).

The anti-Her3 antibodies of the invention can be made by using combinatorial libraries to screen for synthetic antibody clones with the desired activity or activities. In principle, synthetic antibody clones are selected by screening phage libraries containing phage that display various fragments of antibody variable region (Fv) fused to phage coat protein. Such phage libraries are panned by affinity chromatography against the desired antigen. Clones expressing Fv fragments capable of binding to the desired antigen are adsorbed to the antigen and thus separated from the non-binding clones in the library. The binding clones are then eluted from the antigen, and can be further enriched by additional cycles of antigen adsorption/elution. Any of the anti-Her3 antibodies of the invention can be obtained by designing a suitable antigen screening procedure to select for the phage clone of interest followed by construction of a full length anti-Her3 antibody clone using the Fv sequences from the phage clone of interest and suitable constant region (Fc) sequences described in Kabat et al., Sequences of Proteins of Immunological Interest, Fifth Edition, NIH Publication 91-3242, Bethesda Md. (1991), vols. 1-3.

The antigen-binding domain of an antibody is formed from two variable (V) regions of about 110 amino acids, one each from the light (V_{L}) and heavy (V_{H}) chains, that both present three hypervariable loops or complementarity-determining regions (CDRs). Variable domains can be displayed functionally on phage, either as single-chain Fv (scFv) fragments, in which V_{H} and V_{L} are covalently linked through a short, flexible peptide, or as Fab fragments, in which they are each fused to a constant domain and interact non-covalently, as described in Winter et al., Ann. Rev. Immunol., 12: 433-455 (1994). As used herein, scFv encoding phage clones and Fab encoding phage clones are collectively referred to as “Fv phage clones” or “Fv clones”.

Repertoires of V_{H} and V_{L} genes can be separately cloned by polymerase chain reaction (PCR) and recombined randomly in phage libraries, which can then be searched for antigen-binding clones as described in Winter et al., Ann. Rev. Immunol., 12: 433-455 (1994). Libraries from immunized sources provide high-affinity antibodies to the immunogen without the requirement of constructing hybridomas. Alternatively, the naive repertoire can be cloned to provide a single source of human antibodies to a wide range of non-self and also self antigens without any immunization as described by Griffiths et al., EMBO J. 12: 725-734 (1993). Finally, naive libraries can also be made synthetically by cloning the unrearranged V-gene segments from stem cells, and using PCR primers containing random sequence to encode the highly variable CDR3 regions and to accomplish rearrangement in vitro as described by Hoogenboom and Winter, J. Mol. Biol., 227: 381-388 (1992).

Filamentous phage is used to display antibody fragments by fusion to the minor coat protein pIII. The antibody fragments can be displayed as single chain Fv fragments, in which V_{H} and V_{L} domains are connected on the same polypeptide chain by a flexible polypeptide spacer, e.g. as described by Marks et al., J. Mol. Biol., 222: 581-597 (1991), or as Fab fragments, as described in Hoogenboom et al., Nucl. Acids Res., 19: 4133-4137 (1991).

In general, nucleic acids encoding antibody gene fragments are obtained from immune cells harvested from humans. If a library biased in favor of anti-Her3 clones is desired, the subject is immunized with Her3 to generate an antibody response, and spleen cells and/or circulating B cells other peripheral blood lymphocytes (PBLs) are recovered for library construction. In another embodiment, a human antibody gene fragment library biased in favor of anti-Her3 clones is obtained by generating an anti-Her3 antibody response in transgenic mice carrying a functional human immunoglobulin gene array (and lacking a functional endogenous antibody production system) such that Her3 immunization gives rise to B cells producing human antibodies against Her3. The generation of human antibody-producing transgenic mice is described below.

Additional enrichment for anti-Her3 reactive cell populations can be obtained by using a suitable screening procedure to isolate B cells expressing Her3-specific membrane bound antibody, e.g., by cell separation with Her3 affinity chromatography or adsorption of cells to fluorochrome-labeled Her3 followed by flow-activated cell sorting (FACS).

Alternatively, the use of spleen cells and/or B cells or other PBLs from an unimmunized donor provides a better representation of the possible antibody repertoire, and also permits the construction of an antibody library using any animal (human or non-human) species in which Her3 is not antigenic. For libraries incorporating in vitro antibody gene construction, stem cells are harvested from the subject to provide nucleic acids encoding unrearranged antibody gene segments. The immune cells of interest can be obtained from a variety of animal species, such as human, mouse, rat, lagomorpha, lupine, canine, feline, porcine, bovine, equine, and avian species, etc.

Nucleic acid encoding antibody variable gene segments (including V_{H} and V_{L} segments) are recovered from the cells of interest and amplified. In the case of rearranged V_{H} and V_{L} gene libraries, the desired DNA can be obtained by isolating genomic DNA or mRNA from lymphocytes followed by polymerase chain reaction (PCR) with primers matching the 5′ and 3′ ends of rearranged V_{H} and V_{L} genes as described in Orlandi et al., Proc. Natl. Acad. Sci. (USA), 86: 3833-3837 (1989), thereby making diverse V gene repertoires for expression. The V genes can be amplified from cDNA and genomic DNA, with back primers at the 5′ end of the exon encoding the mature V-domain and forward primers based within the J-segment as described in Orlandi et al. (1989) and in Ward et al., Nature, 341: 544-546 (1989). However, for amplifying from cDNA, back primers can also be based in the leader exon as described in Jones et al., Biotechnol., 9: 88-89 (1991), and forward primers within the constant region as described in Sastry et al., Proc. Natl. Acad. Sci. (USA), 86: 5728-5732 (1989). To maximize complementarity, degeneracy can be incorporated in the primers as described in Orlandi et al. (1989) or Sastry et al. (1989). Preferably, the library diversity is maximized by using PCR primers targeted to each V-gene family in order to amplify all available V_{H} and V_{L} arrangements present in the immune cell nucleic acid sample, e.g. as described in the method of Marks et al., J. Mol. Biol., 222: 581-597 (1991) or as described in the method of Orum et al., Nucleic Acids Res., 21: 4491-4498 (1993). For cloning of the amplified DNA into expression vectors, rare restriction sites can be introduced within the PCR primer as a tag at one end as described in Orlandi et al. (1989), or by further PCR amplification with a tagged primer as described in Clackson et al., Nature, 352: 624-628 (1991).

Repertoires of synthetically rearranged V genes can be derived in vitro from V gene segments. Most of the human V_{H}-gene segments have been cloned and sequenced (reported in Tomlinson et al., J. Mol. Biol., 227: 776-798 (1992)), and mapped (reported in Matsuda et al., Nature Genet., 3: 88-94 (1993); these cloned segments (including all the major conformations of the H1 and H2 loop) can be used to generate diverse V_{H} gene repertoires with PCR primers encoding H3 loops of diverse sequence and length as described in Hoogenboom and Winter, J. Mol. Biol., 227: 381-388 (1992). V_{H} repertoires can also be made with all the sequence diversity focused in a long H3 loop of a single length as described in Barbas et al., Proc. Natl. Acad. Sci. USA, 89: 4457-4461 (1992). Human V.kappa. and V.lamda. segments have been cloned and sequenced (reported in Williams and Winter, Eur. J. Immunol., 23: 1456-1461 (1993)) and can be used to make synthetic light chain repertoires. Synthetic V gene repertoires, based on a range of V_{H} and V_{L} folds, and L3 and H3 lengths, will encode antibodies of considerable structural diversity. Following amplification of V-gene encoding DNAs, germline V-gene segments can be rearranged in vitro according to the methods of Hoogenboom and Winter, J. Mol. Biol., 227: 381-388 (1992).

Repertoires of antibody fragments can be constructed by combining V_{H} and V_{L} gene repertoires together in several ways. Each repertoire can be created in different vectors, and the vectors recombined in vitro, e.g., as described in Hogrefe et al., Gene, 128: 119-126 (1993), or in vivo by combinatorial infection, e.g., the loxP system described in Waterhouse et al., Nucl. Acids Res., 21: 2265-2266 (1993). The in vivo recombination approach exploits the two-chain nature of Fab fragments to overcome the limit on library size imposed by E. coli transformation efficiency. Naive V_{H} and V_{L} repertoires are cloned separately, one into a phagemid and the other into a phage vector. The two libraries are then combined by phage infection of phagemid-containing bacteria so that each cell contains a different combination and the library size is limited only by the number of cells present (about 10¹² clones). Both vectors contain in vivo recombination signals so that the V_{H} and V_{L} genes are recombined onto a single replicon and are co-packaged into phage virions. These huge libraries provide large numbers of diverse antibodies of good affinity (K_{d}⁻¹ of about 10⁻⁸ M).

Alternatively, the repertoires may be cloned sequentially into the same vector, e.g. as described in Barbas et al., Proc. Natl. Acad. Sci. USA, 88: 7978-7982 (1991), or assembled together by PCR and then cloned, e.g. as described in Clackson et al., Nature, 352: 624-628 (1991). PCR assembly can also be used to join V_{H} and V_{L} DNAs with DNA encoding a flexible peptide spacer to form single chain Fv (scFv) repertoires. In yet another technique, “in cell PCR assembly” is used to combine V_{H} and V_{L} genes within lymphocytes by PCR and then clone repertoires of linked genes as described in Embleton et al., Nucl. Acids Res., 20: 3831-3837 (1992).

The antibodies produced by naive libraries (either natural or synthetic) can be of moderate affinity (K_{d}⁻¹ of about 10⁶ to 10⁷ M⁻¹), but affinity maturation can also be mimicked in vitro by constructing and reselecting from secondary libraries as described in Winter et al. (1994), supra. For example, mutation can be introduced at random in vitro by using error-prone polymerase (reported in Leung et al., Technique, 1: 11-15 (1989)) in the method of Hawkins et al., J. Mol. Biol., 226: 889-896 (1992) or in the method of Gram et al., Proc. Natl. Acad. Sci. USA, 89: 3576-3580 (1992). Additionally, affinity maturation can be performed by randomly mutating one or more CDRs, e.g. using PCR with primers carrying random sequence spanning the CDR of interest, in selected individual Fv clones and screening for higher affinity clones. WO 9607754 (published 14 Mar. 1996) described a method for inducing mutagenesis in a complementarity determining region of an immunoglobulin light chain to create a library of light chain genes. Another effective approach is to recombine the V_{H} or V_{L} domains selected by phage display with repertoires of naturally occurring V domain variants obtained from unimmunized donors and screen for higher affinity in several rounds of chain reshuffling as described in Marks et al., Biotechnol., 10: 779-783 (1992). This technique allows the production of antibodies and antibody fragments with affinities in the 10⁻⁹ M range.

Her3 nucleic acid and amino acid sequences are known in the art. A representative nucleic acid and amino acid sequence of Her3 is detailed in SEQ ID NOS. 1 and 2 respectively. Nucleic acid sequence encoding the Her3 can be designed using the amino acid sequence of the desired region of Her3. Alternatively, the cDNA sequence (or fragments thereof) of GenBank Accession Nos. NM_{—019074}. Her3 is a transmembrane protein. The extracellular region contains 36 EGF-like repeats, as well as a DSL domain that is conserved among all Her3 ligands and is necessary for receptor binding. The predicted protein also contains a transmembrane region, and a cytoplasmic tail lacking any catalytic motifs. Human Her3 protein is a 685 amino acid protein. The accession number of human Her3 is NM_{—019074}. See Sarah J. Bray, “Her3 signaling: a simple pathway becomes complex” Nature Reviews Molecular Cell Biology, 7: 678-689 (2006), the entire content of which is incorporated by reference herein.

DNAs encoding Her3 can be prepared by a variety of methods known in the art. These methods include, but are not limited to, chemical synthesis by any of the methods described in Engels et al., Agnew. Chem. Int Ed. Engl., 28: 716-734 (1989), such as the triester, phosphite, phosphoramidite and H-phosphonate methods. In one embodiment, codons preferred by the expression host cell are used in the design of the Her3 encoding DNA. Alternatively, DNA encoding the Her3 can be isolated from a genomic or cDNA library.

Following construction of the DNA molecule encoding the Her3, the DNA molecule is operably linked to an expression control sequence in an expression vector, such as a plasmid, wherein the control sequence is recognized by a host cell transformed with the vector. In general, plasmid vectors contain replication and control sequences which are derived from species compatible with the host cell. The vector ordinarily carries a replication site, as well as sequences which encode proteins that are capable of providing phenotypic selection in transformed cells. Suitable vectors for expression in prokaryotic and eukaryotic host cells are known in the art and some are further described herein. Eukaryotic organisms, such as yeasts, or cells derived from multicellular organisms, such as mammals, may be used.

Optionally, the DNA encoding the Her3 is operably linked to a secretory leader sequence resulting in secretion of the expression product by the host cell into the culture medium. Examples of secretory leader sequences include stII, ecotin, lamB, herpes GD, Ipp, alkaline phosphatase, invertase, and alpha factor. Also suitable for use herein is the 36 amino acid leader sequence of protein A (Abrahmsen et al., EMBO J., 4: 3901 (1985)).

Host cells are transfected and preferably transformed with the above-described expression or cloning vectors of this invention and cultured in conventional nutrient media modified as appropriate for inducing promoters, selecting transformants, or amplifying the genes encoding the desired sequences.

Transfection refers to the taking up of an expression vector by a host cell whether or not any coding sequences are in fact expressed. Numerous methods of transfection are known to the ordinarily skilled artisan, for example, CaPO₄ precipitation and electroporation. Successful transfection is generally recognized when any indication of the operation of this vector occurs within the host cell. Methods for transfection are well known in the art, and some are further described herein.

Transformation means introducing DNA into an organism so that the DNA is replicable, either as an extrachromosomal element or by chromosomal integrant. Depending on the host cell used, transformation is done using standard techniques appropriate to such cells.

Methods for transformation are well known in the art, and some are further described herein.

Prokaryotic host cells used to produce the Her3 can be cultured as described generally in Sambrook et al., supra.

The mammalian host cells used to produce the Her3 can be cultured in a variety of media, which is well known in the art and some of which is described herein.

The host cells referred to in this disclosure encompass cells in in vitro culture as well as cells that are within a host animal.

Purification of Her3 may be accomplished using art-recognized methods, some of which are described herein.

The purified Her3 can be attached to a suitable matrix such as agarose beads, acrylamide beads, glass beads, cellulose, various acrylic copolymers, hydroxyl methacrylate gels, polyacrylic and polymethacrylic copolymers, nylon, neutral and ionic carriers, and the like, for use in the affinity chromatographic separation of phage display clones. Attachment of the Her3 protein to the matrix can be accomplished by the methods described in Methods in Enzymology, vol. 44 (1976). A commonly employed technique for attaching protein ligands to polysaccharide matrices, e.g. agarose, dextran or cellulose, involves activation of the carrier with cyanogen halides and subsequent coupling of the peptide ligand's primary aliphatic or aromatic amines to the activated matrix.

Alternatively, Her3 can be used to coat the wells of adsorption plates, expressed on host cells affixed to adsorption plates or used in cell sorting, or conjugated to biotin for capture with streptavidin-coated beads, or used in any other art-known method for panning phage display libraries.

The phage library samples are contacted with immobilized Her3 under conditions suitable for binding of at least a portion of the phage particles with the adsorbent. Normally, the conditions, including pH, ionic strength, temperature and the like are selected to mimic physiological conditions. The phages bound to the solid phase are washed and then eluted by acid, e.g. as described in Barbas et al., Proc. Natl. Acad. Sci. USA, 88: 7978-7982 (1991), or by alkali, e.g. as described in Marks et al., 3. Mal. Biol., 222: 581-597 (1991), or by Her3 antigen competition, e.g. in a procedure similar to the antigen competition method of Clackson et al., Nature, 352: 624-628 (1991). Phages can be enriched 20-1,000-fold in a single round of selection. Moreover, the enriched phages can be grown in bacterial culture and subjected to further rounds of selection.

The efficiency of selection depends on many factors, including the kinetics of dissociation during washing, and whether multiple antibody fragments on a single phage can simultaneously engage with antigen. Antibodies with fast dissociation kinetics (and weak binding affinities) can be retained by use of short washes, multivalent phage display and high coating density of antigen in solid phase. The high density not only stabilizes the phage through multivalent interactions, but favors rebinding of phage that has dissociated. The selection of antibodies with slow dissociation kinetics (and good binding affinities) can be promoted by use of long washes and monovalent phage display as described in Bass et al., Proteins, 8: 309-314 (1990) and in WO 92/09690, and a low coating density of antigen as described in Marks et al., Biotechnol., 10: 779-783 (1992).

It is possible to select between phage antibodies of different affinities, even with affinities that differ slightly, for Her3. However, random mutation of a selected antibody (e.g. as performed in some of the affinity maturation techniques described above) is likely to give rise to many mutants, most binding to antigen, and a few with higher affinity. With limiting Her3, rare high affinity phage could be competed out. To retain all the higher affinity mutants, phages can be incubated with excess biotinylated Her3, but with the biotinylated Her3 at a concentration of lower molarity than the target molar affinity constant for Her3. The high affinity-binding phages can then be captured by streptavidin-coated paramagnetic beads. Such “equilibrium capture” allows the antibodies to be selected according to their affinities of binding, with sensitivity that permits isolation of mutant clones with as little as two-fold higher affinity from a great excess of phages with lower affinity. Conditions used in washing phages bound to a solid phase can also be manipulated to discriminate on the basis of dissociation kinetics.

Anti-Her3 clones may be activity selected. In one embodiment, the invention provides anti-Her3 antibodies that block the binding between a Her3 receptor, preferably one of a Her3 and/or Her3 receptor and its binding partner. Fv clones corresponding to such anti-Her3 antibodies can be selected by (1) isolating anti-Her3 clones from a phage library as described above, and optionally amplifying the isolated population of phage clones by growing up the population in a suitable bacterial host; (2) selecting Her3 and a second protein against which blocking and non-blocking activity, respectively, is desired; (3) adsorbing the anti-Her3 phage clones to immobilized Her3; (4) using an excess of the second protein to elute any undesired clones that recognize Her3-binding determinants which overlap or are shared with the binding determinants of the second protein; and (5) eluting the clones which remain adsorbed following step (4). Optionally, clones with the desired blocking/non-blocking properties can be further enriched by repeating the selection procedures described herein one or more times.

DNA encoding, for example, phage display Fv clones of the invention is readily isolated and sequenced using conventional procedures (e.g. by using oligonucleotide primers designed to specifically amplify the heavy and light chain coding regions of interest from hybridoma or phage DNA template). Once isolated, the DNA can be placed into expression vectors, which are then transfected into host cells such as E. coli cells, simian COS cells, Chinese hamster ovary (CHO) cells, or myeloma cells that do not otherwise produce immunoglobulin protein, to obtain the synthesis of the desired monoclonal antibodies in the recombinant host cells. Review articles on recombinant expression in bacteria of antibody-encoding DNA include Skerra et al., Curr. Opinion in Immunol., 5: 256 (1993) and Pluckthun, Immunol. Revs, 130: 151 (1992).

DNA encoding the Fv clones of the invention can be combined with known DNA sequences encoding heavy chain and/or light chain constant regions (e.g. the appropriate DNA sequences can be obtained from Kabat et al., supra) to form clones encoding full or partial length heavy and/or light chains. It will be appreciated that constant regions of any isotype can be used for this purpose, including IgG, IgM, IgA, IgD, and IgE constant regions, and that such constant regions can be obtained from any human or animal species. A Fv clone derived from the variable domain DNA of one animal (such as human) species and then fused to constant region DNA of another animal species to form coding sequence(s) for “hybrid”, full length heavy chain and/or light chain is included in the definition of “chimeric” and “hybrid” antibody as used herein. In a preferred embodiment, a Fv clone derived from human variable DNA is fused to human constant region DNA to form coding sequence(s) for all human, full or partial length heavy and/or light chains.

### Antibody Fragments

In certain circumstances there are advantages of using antibody fragments, rather than whole antibodies. The smaller size of the fragments allows for rapid clearance, and may lead to improved access to solid tumors.

An antibody functional fragment refers to a portion of an antibody which retains some or all of its target-specific binding activity. Such functional fragments can include, for example, antibody functional fragments such as Fv, Fab, F(ab′), F(ab′)₂, F(ab′)₂, single chain Fv (scFv), diabodies, triabodies, tetrabodies and minibody. Other functional fragments can include, for example, heavy (H) or light (L) chain polypeptides, variable heavy (V_{H}) and variable light (V_{L}) chain region polypeptides, complementarity determining region (CDR) polypeptides, single domain antibodies, and polypeptides that contain at least a portion of an immunoglobulin that is sufficient to retain target-specific binding activity. The present invention encompasses antibody fragments. In certain circumstances there are advantages of using antibody fragments, rather than whole antibodies. The smaller size of the fragments allows for rapid clearance, and may lead to improved access to solid tumors.

Various techniques have been developed for the production of antibody fragments. Traditionally, these fragments were derived via proteolytic digestion of intact antibodies (see, e.g., Morimoto et al., Journal of Biochemical and Biophysical Methods 24:107-117 (1992); and Brennan et al., Science, 229:81 (1985)). However, these fragments can now be produced directly by recombinant host cells. Fab, Fv and ScFv antibody fragments can all be expressed in and secreted from E. coli, thus allowing the facile production of large amounts of these fragments. Antibody fragments can be isolated from the antibody phage libraries discussed above. Alternatively, Fab′-SH fragments can be directly recovered from E. coli and chemically coupled to form F(ab′)₂ fragments (Carter et al., Bio/Technology 10: 163-167 (1992)). According to another approach, F(ab′)₂ fragments can be isolated directly from recombinant host cell culture. Fab and F(ab′)₂ fragment with increased in vivo half-life comprising a salvage receptor binding epitope residues are described in U.S. Pat. No. 5,869,046. Other techniques for the production of antibody fragments will be apparent to the skilled practitioner. In other embodiments, the antibody of choice is a single chain Fv fragment (scFv). See WO 93/16185; U.S. Pat. Nos. 5,571,894; and 5,587,458. Fv and sFv are the only species with intact combining sites that are devoid of constant regions; thus, they are suitable for reduced nonspecific binding during in vivo use. sFv fusion proteins may be constructed to yield fusion of an effector protein at either the amino or the carboxy terminus of an sFv. See Antibody Engineering, ed. Borrebaeck, supra. The antibody fragment may also be a “linear antibody”, e.g., as described in U.S. Pat. No. 5,641,870 for example. Such linear antibody fragments may be monospecific or bispecific.

With respect to antibodies and functional fragments thereof that exhibit beneficial binding characteristics to a target molecule, various forms, alterations and modifications are well known in the art. Target-specific monoclonal antibodies for use in a biopharmaceutical formulation of the invention can include any of such various monoclonal antibody forms, alterations and modifications. Examples of such various forms and terms as they are known in the art are set forth below.

### Polyclonal Antibodies

Polyclonal antibodies are preferably raised in animals by multiple subcutaneous (sc) or intraperitoneal (ip) injections of the relevant antigen and an adjuvant. It may be useful to conjugate the relevant antigen (especially when synthetic peptides are used) to a protein that is immunogenic in the species to be immunized. For example, the antigen can be conjugated to keyhole limpet hemocyanin (KLH), serum albumin, bovine thyroglobulin, or soybean trypsin inhibitor, using a bifunctional or derivatizing agent, e.g., maleimidobenzoyl sulfosuccinimide ester (conjugation through cysteine residues). N-hydroxysuccinimide (through lysine residues), glutaraldehyde, succinic anhydride, SOCl₂, or R^{I}N═C═NR, where R and R¹ are different alkyl groups.

Animals are immunized against the antigen, immunogenic conjugates, or derivatives by combining, e.g., 100 μg or 5 μg of the protein or conjugate (for rabbits or mice, respectively) with 3 volumes of Freund's complete adjuvant and injecting the solution intradermally at multiple sites. One month later, the animals are boosted with ⅕ to 1/10 the original amount of peptide or conjugate in Freund's complete adjuvant by subcutaneous injection at multiple sites. Seven to 14 days later, the animals are bled and the serum is assayed for antibody titer. Animals are boosted until the titer plateaus. Conjugates also can be made in recombinant cell culture as protein fusions. Also, aggregating agents such as alum are suitably used to enhance the immune response.

### Monoclonal Antibodies

Monoclonal antibodies may be made using the hybridoma method first described by Kohler et al., Nature, 256:495 (1975), or may be made by recombinant DNA methods (U.S. Pat. No. 4,816,567).

In the hybridoma method, a mouse or other appropriate host animal, such as a hamster, is immunized as described above to elicit lymphocytes that produce or are capable of producing antibodies that will specifically bind to the protein used for immunization. Alternatively, lymphocytes may be immunized in vitro. After immunization, lymphocytes are isolated and then fused with a myeloma cell line using a suitable fusing agent, such as polyethylene glycol, to form a hybridoma cell (Goding, Monoclonal Antibodies: Principles and Practice, pp. 59-103 (Academic Press, 1986)).

The hybridoma cells thus prepared are seeded and grown in a suitable culture medium which medium preferably contains one or more substances that inhibit the growth or survival of the unfused, parental myeloma cells (also referred to as fusion partner). For example, if the parental myeloma cells lack the enzyme hypoxanthine guanine phosphoribosyl transferase (HGPRT or HPRT), the selective culture medium for the hybridomas typically will include hypoxanthine, aminopterin, and thymidine (HAT medium), which substances prevent the growth of HGPRT-deficient cells.

Preferred fusion partner myeloma cells are those that fuse efficiently, support stable high-level production of antibody by the selected antibody-producing cells, and are sensitive to a selective medium that selects against the unfused parental cells. Preferred myeloma cell lines are murine myeloma lines, such as those derived from MOPC-21 and MPC-11 mouse tumors available from the Salk Institute Cell Distribution Center, San Diego, Calif. USA, and SP-2 and derivatives e.g., X63-Ag8-653 cells available from the American Type Culture Collection, Rockville, Md. USA. Human myeloma and mouse-human heteromyeloma cell lines also have been described for the production of human monoclonal antibodies (Kozbor, J. Immunol., 133:3001 (1984); and Brodeur et al., Monoclonal Antibody Production Techniques and Applications, pp. 51-63 (Marcel Dekker, Inc., New York, 1987)).

Culture medium in which hybridoma cells are growing is assayed for production of monoclonal antibodies directed against the antigen. Preferably, the binding specificity of monoclonal antibodies produced by hybridoma cells is determined by immunoprecipitation or by an in vitro binding assay, such as radioimmunoassay (RIA) or enzyme-linked immunosorbent assay (ELISA).

The binding affinity of the monoclonal antibody can, for example, be determined by the Scatchard analysis described in Munson et al., Anal, Biochem., 107:220 (1980).

Once hybridoma cells that produce antibodies of the desired specificity, affinity, and/or activity are identified, the clones may be subcloned by limiting dilution procedures and grown by standard methods (Goding, Monoclonal Antibodies: Principles and Practice, pp. 59-103 (Academic Press, 1986)). Suitable culture media for this purpose include, for example, D-MEM or RPMI-1640 medium. In addition, the hybridoma cells may be grown in vivo as ascites tumors in an animal e.g, by i.p. injection of the cells into mice.

The monoclonal antibodies secreted by the subclones are suitably separated from the culture medium, ascites fluid, or serum by conventional antibody purification procedures such as, for example, affinity chromatography (e.g., using protein A or protein G-Sepharose®) or ion-exchange chromatography, hydroxylapatite chromatography, gel electrophoresis, dialysis, etc.

DNA encoding the monoclonal antibodies is readily isolated and sequenced using conventional procedures (e.g., by using oligonucleotide probes that are capable of binding specifically to genes encoding the heavy and light chains of murine antibodies). The hybridoma cells serve as a preferred source of such DNA. Once isolated, the DNA may be placed into expression vectors, which are then transfected into host cells such as E. coli cells, simian COS cells, Chinese Hamster Ovary (CHO) cells, or myeloma cells that do not otherwise produce antibody protein, to obtain the synthesis of monoclonal antibodies in the recombinant host cells. Review articles on recombinant expression in bacteria of DNA encoding the antibody include Skerra et al., Curr. Opinion in Immunol., 5:256-262 (1993) and Pluckthun, Immunol. Revs., 130:151-188 (1992).

In a further embodiment, monoclonal antibodies or antibody fragments can be isolated from antibody phage libraries generated using the techniques described in McCafferty et al., Nature, 348:552-554 (1990). Clackson et al., Nature, 352:624-628 (1991) and Marks et al., J. Mol. Biol., 222:581-597 (1991) describe the isolation of murine and human antibodies, respectively, using phage libraries. Subsequent publications describe the production of high affinity (nM range) human antibodies by chain shuffling (Marks et al., Bio/Technology, 10:779-783 (1992)), as well as combinatorial infection and in viva recombination as a strategy for constructing very large phage libraries (Waterhouse et al., Nuc. Acids. Res., 21:2265-2266 (1993)). Thus, these techniques are viable alternatives to traditional monoclonal antibody hybridoma techniques for isolation of monoclonal antibodies.

The DNA that encodes the antibody may be modified, for example, by substituting human heavy chain and light chain constant domain (C_{H} and C_{L}) sequences for the homologous murine sequences (U.S. Pat. No. 4,816,567; and Morrison, et al., Proc. Natl. Acad. Sci. USA, 81:6851 (1984)), or by fusing the immunoglobulin coding sequence with all or part of the coding sequence for a non-immunoglobulin polypeptide. The non-immunoglobulin polypeptide sequences can substitute for the constant domains of an antibody, or they are substituted for the variable domains of one antigen-combining site of an antibody to create a chimeric bivalent antibody comprising one antigen-combining site having specificity for an antigen and another antigen-combining site having specificity for a different antigen.

### Humanized Antibodies

Methods for humanizing non-human antibodies are well known in the art. Generally, a humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. Accordingly, such “humanized” antibodies are chimeric antibodies (U.S. Pat. No. 4,816,567) wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

The choice of human variable domains, both light and heavy, to be used in making the humanized antibodies is very important to reduce antigenicity. According to the so-called “best-fit” method, the sequence of the variable domain of a rodent antibody is screened against the entire library of known human variable-domain sequences. The human sequence which is closest to that of the rodent is then accepted as the human framework (FR) for the humanized antibody (Sims et al., J. Immunol., 151:2296 (1993); Chothia et al., J. Mol. Biol., 196:901 (1987)). Another method uses a particular framework derived from the consensus sequence of all human antibodies of a particular subgroup of light or heavy chains. The same framework may be used for several different humanized antibodies (Carter et al., Proc. Natl. Acad. Sci. USA, 89:4285 (1992); Presta et al., J. Immunol., 151:2623 (1993)).

It is further important that antibodies be humanized with retention of high affinity for the antigen and other favorable biological properties. To achieve this goal, according to a preferred method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the CDR residues are directly and most substantially involved in influencing antigen binding.

### Human Antibodies

Human anti-Her3 antibodies of the invention can be constructed by combining Fv clone variable domain sequence(s) selected from human-derived phage display libraries with known human constant domain sequences(s) as described above. Alternatively, human monoclonal anti-Her3 antibodies of the invention can be made by the hybridoma method. Human myeloma and mouse-human heteromyeloma cell lines for the production of human monoclonal antibodies have been described, for example, by Kozbor J. Immunol., 133: 3001 (1984); Brodeur et al, Monoclonal Antibody Production Techniques and Applications, pp. 51-63 (Marcel Dekker, Inc., New York, 1987); and Boerner et al, J. Immunol., 147: 86 (1991). Human antibodies can also be derived from phage-display libraries (Hoogenboom et al., 3. Mal. Biol., 227:381 (1991); Marks et al., J. Mol. Biol., 222:581-597 (1991)).

Alternatively, it is now possible to produce transgenic animals (e.g., mice) that are capable, upon immunization, of producing a full repertoire of human antibodies in the absence of endogenous immunoglobulin production. For example, it has been described that the homozygous deletion of the antibody heavy-chain joining region (J_{H}) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, e.g., Jakobovits et al., Proc. Natl. Acad. Sci. USA, 90:2551 (1993); Jakobovits et al., Nature, 362:255-258 (1993); Bruggermann et al., Year in Immuno., 7:33 (1993); U.S. Pat. Nos. 5,545,806, 5,569,825, 5,591,669 (all of GenPharm); 5,545,807; and WO 97/17852.

Alternatively, phage display technology (McCafferty et al., Nature 348:552-553 (1990) can be used to produce human antibodies and antibody fragments in vitro, from immunoglobulin variable (V) domain gene repertoires from unimmunized donors. According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B-cell. Phage display can be performed in a variety of formats, reviewed in, e.g., Johnson, Kevin S. and Chiswell, David J., Current Opinion in Structural Biology 3:564-571 (1993). Several sources of V-gene segments can be used for phage display. Clackson et al., Nature, 352:624-628 (1991) isolated a diverse array of anti-oxazolone antibodies from a small random combinatorial library of V genes derived from the spleens of immunized mice. A repertoire of V genes from unimmunized human donors can be constructed and antibodies to a diverse array of antigens (including self-antigens) can be isolated essentially following the techniques described by Marks et al., J. Mol, Biol. 222:581-597 (1991), or Griffith et al., EMBO J. 12:725-734 (1993). See, also, U.S. Pat. Nos. 5,565,332 and 5,573,905.

Human antibodies may also be generated by in vitro activated B cells (see U.S. Pat. Nos. 5,567,610 and 5,229,275).

Gene shuffling can also be used to derive human antibodies from non-human, e.g. rodent, antibodies, where the human antibody has similar affinities and specificities to the starting non-human antibody. According to this method, which is also called “epitope imprinting”, either the heavy or light chain variable region of a non-human antibody fragment obtained by phage display techniques as described above is replaced with a repertoire of human V domain genes, creating a population of non-human chain/human chain scFv or Fab chimeras. Selection with antigen results in isolation of a non-human chain/human chain chimeric scFv or Fab wherein the human chain restores the antigen binding site destroyed upon removal of the corresponding non-human chain in the primary phage display clone, i.e. the epitope governs (imprints) the choice of the human chain partner. When the process is repeated in order to replace the remaining non-human chain, a human antibody is obtained (see PCT WO 93/06213 published Apr. 1, 1993). Unlike traditional humanization of non-human antibodies by CDR grafting, this technique provides completely human antibodies, which have no FR or CDR residues of non-human origin.

### Bispecific Antibodies

Bispecific antibodies are monoclonal antibodies that have binding specificities for at least two different antigens. In the present case, one of the binding specificities is for Her3 and the other is for any other antigen. Exemplary bispecific antibodies may bind to two different epitopes of the Her3 protein. Bispecific antibodies may also be used to localize cytotoxic agents to cells which express Her3. These antibodies possess a Her3-binding arm and an arm which binds the cytotoxic agent (e.g. saporin, anti-interferon-.alpha., vinca alkaloid, ricin A chain, methotrexate or radioactive isotope hapten). Bispecific antibodies can be prepared as full length antibodies or antibody fragments (e.g. F(ab′)₂ bispecific antibodies).

Methods for making bispecific antibodies are known in the art. Traditionally, the recombinant production of bispecific antibodies is based on the co-expression of two immunoglobulin heavy chain-light chain pairs, where the two heavy chains have different specificities (Milstein and Cuello, Nature, 305: 537 (1983)). Because of the random assortment of immunoglobulin heavy and light chains, these hybridomas (quadromas) produce a potential mixture of 10 different antibody molecules, of which only one has the correct bispecific structure. The purification of the correct molecule, which is usually done by affinity chromatography steps, is rather cumbersome, and the product yields are low. Similar procedures are disclosed in WO 93/08829 published May 13, 1993, and in Traunecker et al., EMBO J., 10: 3655 (1991).

According to a different and more preferred approach, antibody variable domains with the desired binding specificities (antibody-antigen combining sites) are fused to immunoglobulin constant domain sequences. The fusion preferably is with an immunoglobulin heavy chain constant domain, comprising at least part of the hinge, CH2, and CH3 regions. It is preferred to have the first heavy-chain constant region (CH1), containing the site necessary for light chain binding, present in at least one of the fusions. DNAs encoding the immunoglobulin heavy chain fusions and, if desired, the immunoglobulin light chain, are inserted into separate expression vectors and are co-transfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance.

In one embodiment of this approach, the bispecific antibodies are composed of a hybrid immunoglobulin heavy chain with a first binding specificity in one arm, and a hybrid immunoglobulin heavy chain-light chain pair (providing a second binding specificity) in the other arm. It was found that this asymmetric structure facilitates the separation of the desired bispecific compound from unwanted immunoglobulin chain combinations, as the presence of an immunoglobulin light chain in only one half of the bispecific molecule provides for a facile way of separation. This approach is disclosed in WO 94/04690. For further details of generating bispecific antibodies see, for example, Suresh et al., Methods in Enzymology, 121:210 (1986).

According to another approach, the interface between a pair of antibody molecules can be engineered to maximize the percentage of heterodimers which are recovered from recombinant cell culture. The preferred interface comprises at least a part of the C_{H3} domain of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Techniques for generating bispecific antibodies from antibody fragments have also been described in the literature. For example, bispecific antibodies can be prepared using chemical linkage. Brennan et al., Science, 229: 81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Various techniques for making and isolating bispecific antibody fragments directly from recombinant cell culture have also been described. For example, bispecific antibodies have been produced using leucine zippers. See Kostelny et al., J. Immunol., 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. Another strategy for making bispecific antibody fragments by the use of single-chain Fv (scFv) dimers has also been reported. See Gruber et al., J. Immunol., 152:5368 (1994).

### Heteroconjugate

Bispecific antibodies include cross-linked or “heteroconjugate” antibodies. As such, heteroconjugate antibodies are also within the scope of the present invention. Heteroconjugate antibodies are composed of two covalently joined antibodies. Such antibodies have, for example, been proposed to target immune system cells to unwanted cells, e.g., U.S. Pat. No. 4,676,980, and for treatment of HIV infection, e.g., WO 91/00360; WO 92/200373; EP 03089. Heteroconjugate antibodies may be made using any convenient cross-linking methods. It is contemplated that the antibodies may be prepared in vitro using known methods in synthetic protein chemistry, including those involving cross-linking agents. For example, immunotoxins may be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

### Diabody

The “diabody” technology described by Hollinger et al., Proc. Natl. Acad. Sci. USA, 90:6444-6448 (1993) has provided an alternative mechanism for making bispecific antibody fragments. A diabody is a bivalent dimer formed by the non-covalent association of two scFvs, yielding two Fv binding sites. Briefly, a diabody refers to an engineered antibody construct prepared by isolating the binding domains (both heavy and light chain) of a binding antibody, and supplying a linking moiety which joins or operably links the heavy and light chains on the same polypeptide chain thereby preserving the binding function (see, Holliger et al. (1993) Proc. Natl. Mad. Sci. USA 90:6444; Poljak (1994) Structure 2:1121-1123). This forms, in essence, a radically abbreviated antibody, having only the variable domain necessary for binding the antigen. By using a linker that is too short to allow pairing between the two domains on the same chain, the domains are forced to pair with the complementary domains of another chain and create two antigen-binding sites. These dimeric antibody fragments, or diabodies, are bivalent and bispecific. Thus, diabodies are dimers of two scFv molecules that cannot fold properly into one scFv molecule. Diabodies are built like scFv molecules, but usually have a short (less than 10, preferably 1-5 amino acids) peptide linker connecting both V-domains, whereby both domains can not interact intramolecular, and are forced to interact intermolecular (Holliger et al., 1993) (U.S. Pat. No. 5,837,242). A diabody thus may consist of a V_{H}-V_{L} chain that interacts with a similar V_{H}-V_{L} chain to form a dimer of the formula V_{H}-V_{L}:V_{H}-V_{L}. The diabody chain dimers bind the antigen specified by V_{H} and V_{L} bivalent. Winter described the construction of bispecific diabodies by coupling the V_{H} domain of a chosen antibody A to the V_{L} domain of a chosen antibody B, using a peptide linker sufficiently short to inhibit the interaction of V_{H}(A) with V_{L}(B). Also the reverse molecule V_{H}(B)-V_{L}(A) is made the same way (Holliger, Griffiths, Hoogenboom, Malmqvist, Marks, McGuinness, Pope, Prospero and Winter: “Multivalent and multispecific binding proteins, their manufacture and use”, U.S. Pat. No. 5,837,242, 1998). The skilled artisan will appreciate that any method to generate diabodies can be used. Suitable methods are described by Holliger, et al. (1993) supra, Poljak (1994) supra, Zhu, et al. (1996) Biotechnology 14:192-196, and U.S. Pat. No. 6,492,123, incorporated herein by reference.

### Fab′-SH

Recent progress has facilitated the direct recovery of Fab′-SH fragments from E. coli, which can be chemically coupled to form bispecific antibodies. Shalaby et al., J. Exp. Med., 175: 217-225 (1992) describe the production of a fully humanized bispecific antibody F(ab′)₂ molecule. Each Fab′ fragment was separately secreted from E. coli and subjected to directed chemical coupling in vitro to form the bispecific antibody. The bispecific antibody thus fowled was able to bind to cells overexpressing the Her2 receptor and normal human T cells, as well as trigger the lytic activity of human cytotoxic lymphocytes against human breast tumor targets.

### Trispecific

Antibodies with more than two valences are contemplated. For example, trispecific antibodies can be prepared. Tuft et al. J. Immunol. 147: 60 (1991).

### Tetravalent

Tetravalent bispecific antibodies can be created by chemical cross-linking of two monoclonal antibodies (Bs(IgG)2) (Karpovsky et al., 1984) (U.S. Pat. No. 4,676,980). Problems related to their rapid clearance in vivo via the kidney due to their small size may be circumvented by, for example, increasing their molecular weight size thereby increasing their serum permanence and product efficacy. (Wu, A. M., Chen, W., Raubitschek, A., Williams, L. E., Neumaier, M., Fischer, R., Hu, S. Z., Odom-Maryon, T., Wong, J. Y. and Shively, J. E.: Tumor localization of anti-CEA single-chain Fvs: improved targeting by non-covalent dimers. Immunotechnology 2 (1996) 21-36).

### Peptibodies

Peptibodies, which consist of an immunoglobulin constant region domain (Fc) linked to two binding peptides through either the carboxyl- or amino termini of the Fc domain, also are included herein as an antibody functional fragment. Such antibody binding fragments can be found described in, for example, Harlow and Lane, supra; Molec. Biology and Biotechnology: A Comprehensive Desk Reference (Myers, R. A. (ed.), New York: VCH Publisher, Inc.); Huston et al., Cell Biophysics, 22:189-224 (1993); Pluckthun and Skerra, Meth. Enzymol., 178:497-515 (1989) and in Day, E. D., Advanced. Immunochemistry, Second Ed., Wiley-Liss, Inc., New York, N.Y. (1990).

### Multivalent Antibodies

A multivalent antibody may be internalized (and/or catabolized) faster than a bivalent antibody by a cell expressing an antigen to which the antibodies bind. The antibodies of the present invention can be multivalent antibodies (which are other than of the IgM class) with three or more antigen binding sites (e.g. tetravalent antibodies), which can be readily produced by recombinant expression of nucleic acid encoding the polypeptide chains of the antibody. The multivalent antibody can comprise a dimerization domain and three or more antigen binding sites. The preferred dimerization domain comprises (or consists of) an Fc region or a hinge region. In this scenario, the antibody will comprise an Fc region and three or more antigen binding sites amino-terminal to the Fc region. The preferred multivalent antibody herein comprises (or consists of) three to about eight, but preferably four, antigen binding sites. The multivalent antibody comprises at least one polypeptide chain (and preferably two polypeptide chains), wherein the polypeptide chain(s) comprise two or more variable domains. For instance, the polypeptide chain(s) may comprise VD1-(X1)ₙ-VD2-(X2)ₙ-Fc, wherein VD1 is a first variable domain, VD2 is a second variable domain, Fc is one polypeptide chain of an Fc region, X1 and X2 represent an amino acid or polypeptide, and n is 0 or 1. For instance, the polypeptide chain(s) may comprise: V_{H}-CH1-flexible linker-V_{H}-CH1-Fc region chain; or V_{H}-CH1-V_{H}-CH1-Fc region chain. The multivalent antibody herein preferably further comprises at least two (and preferably four) light chain variable domain polypeptides. The multivalent antibody herein may, for instance, comprise from about two to about eight light chain variable domain polypeptides. The light chain variable domain polypeptides contemplated here comprise a light chain variable domain and, optionally, further comprise a CL domain.

### Antibody Variants

In some embodiments, amino acid sequence modification(s) of the antibodies described herein are contemplated. For example, it may be desirable to improve the binding affinity and/or other biological properties of the antibody. Amino acid sequence variants of the antibody are prepared by introducing appropriate nucleotide changes into the antibody nucleic acid, or by peptide synthesis. Such modifications include, for example, deletions from, and/or insertions into and/or substitutions of residues within the amino acid sequences of the antibody. Any combination of deletion, insertion, and substitution is made to arrive at the final construct, provided that the final construct possesses the desired characteristics. The amino acid alterations may be introduced in the subject antibody amino acid sequence at the time that sequence is made.

A useful method for identification of certain residues or regions of the antibody that are preferred locations for mutagenesis is called “alanine scanning mutagenesis” as described by Cunningham and Wells (1989) Science, 244:1081-1085. Here, a residue or group of target residues are identified (e.g., charged residues such as arg, asp, his, lys, and glu) and replaced by a neutral or negatively charged amino acid (most preferably alanine or polyalanine) to affect the interaction of the amino acids with antigen. Those amino acid locations demonstrating functional sensitivity to the substitutions then are refined by introducing further or other variants at, or for, the sites of substitution. Thus, while the site for introducing an amino acid sequence variation is predetermined, the nature of the mutation per se need not be predetermined. For example, to analyze the performance of a mutation at a given site, ala scanning or random mutagenesis is conducted at the target codon or region and the expressed immunoglobulins are screened for the desired activity.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue or the antibody fused to a cytotoxic polypeptide. Other insertional variants of the antibody molecule include the fusion of the N- or C-terminus of the antibody to an enzyme (e.g. for ADEPT) or a polypeptide which increases the serum half-life of the antibody.

Glycosylation of polypeptides is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-acetylgalactosamine, galactose, or xylose to a hydroxyamino acid, most commonly serine or threonine, although 5-hydroxyproline or 5-hydroxylysine may also be used. Addition of glycosylation sites to the antibody is conveniently accomplished by altering the amino acid sequence such that it contains one or more of the above-described tripeptide sequences (for N-linked glycosylation sites). The alteration may also be made by the addition of, or substitution by, one or more serine or threonine residues to the sequence of the original antibody (for O-linked glycosylation sites). See section marked “Effector Function Engineering”, infra.

Where the antibody comprises an Fc region, the carbohydrate attached thereto may be altered. For example, antibodies with a mature carbohydrate structure that lacks fucose attached to an Fc region of the antibody are described in US Pat Appl No US 2003/0157108 (Presta, L.). See also US 2004/0093621 (Kyowa Hakko Kogyo Co., Ltd). Antibodies with a bisecting N-acetylglucosamine (GlcNAc) in the carbohydrate attached to an Fc region of the antibody are referenced in WO 2003/011878, Jean-Mairet et al. and U.S. Pat. No. 6,602,684, Umana et al. Antibodies with at least one galactose residue in the oligosaccharide attached to an Fc region of the antibody are reported in WO 1997/30087, Patel et al. See, also, WO 1998/58964 (Raju, S.) and WO 1999/22764 (Raju, S.) concerning antibodies with altered carbohydrate attached to the Fc region thereof. See also US 2005/0123546 (Umana et al.) on antigen-binding molecules with modified glycosylation.

At least one glycosylation variant herein comprises an Fc region, wherein a carbohydrate structure attached to the Fc region lacks fucose. Such variants have improved ADCC function. Optionally, the Fc region further comprises one or more amino acid substitutions therein which further improve ADCC, for example, substitutions at positions 298, 333, and/or 334 of the Fc region (Eu numbering of residues). Examples of publications related to “defucosylated” or “fucose-deficient” antibodies include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; Okazaki et al. J. Mol. Biol. 336:1239-1249 (2004); Yamane-Ohnuki et al. Biotech. Bioeng. 87: 614 (2004). Examples of cell lines producing defacosylated antibodies include Lec13 CHO cells deficient in protein fucosylation (Ripka et al. Arch. Biochem. Biophys. 249:533-545 (1986); US Pat Appl No US 2003/0157108 A1, Presta, L; and WO 2004/056312 A1, Adams et al., especially at Example 11), and knockout cell lines, such as alpha-1,6-fucosyltransferase gene, FUT8, knockout CHO cells (Yamane-Ohnuki et al. Biotech. Bioeng. 87: 614 (2004)). For further details, see “Effector Function Engineering” infra.

Another type of variant is an amino acid substitution variant. These variants have at least one amino acid residue in the antibody molecule replaced by a different residue. The sites of greatest interest for substitutional mutagenesis include the hypervariable regions, but FR alterations are also contemplated. Conservative substitutions are shown in Table 1 under the heading of “preferred substitutions”. If such substitutions result in a change in biological activity, then more substantial changes, denominated “exemplary substitutions” in Table 1, or as further described below in reference to amino acid classes, may be introduced and the products screened.

Exemplary Preferred Residue Substitutions Ala (A) Val; Leu; Ile Val Arg (R) Lys; Gln; Asn Lys Asn (N) Gln; His; Asp, Lys; Arg Gln Asp (D) Glu; Asn Glu Cys (C) Ser; Ala Ser Gln (O) Asn; Glu Asn Glu (E) Asp; Gln Asp Gly (G) Ala Ala His (H) Asn; Gln; Lys; Arg Arg Ile (I) Leu; Val; Met; Ala; Leu Phe; Norleucine Leu (L) Norleucine; Ile; Val; Ile Met; Ala; Phe Lys (K) Arg; Gln; Asn Arg Met (M) Leu; Phe; Ile Leu Phe (F) Trp; Leu; Val; Ile; Ala; Tyr Tyr Pro (P) Ala Ala Ser (S) Thr Thr Thr (T) Val; Ser Ser Trp (W) Tyr; Phe Tyr Tyr (Y) Trp; Phe; Thr; Ser Phe Val (V) Ile; Len; Met; Phe; Leu Ala; Norleucine

Substantial modifications in the biological properties of the antibody are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side-chain properties:

(1) hydrophobic: Norleucine, Met, Ala, Val, Leu, Ile;

(2) neutral hydrophilic: Cys, Ser, Thr, Asn, Gln;

(3) acidic: Asp, Glu;

(4) basic: His, Lys, Arg;

(5) residues that influence chain orientation: Gly, Pro; and

(6) aromatic: Trp, Tyr, Phe.

Non-conservative substitutions will entail exchanging a member of one of these classes for another class.

One type of substitutional variant involves substituting one or more hypervariable region residues of a parent antibody (e.g. human antibody). Generally, the resulting variant(s) selected for further development will have improved biological properties relative to the parent antibody from which they are generated. A convenient way for generating such substitutional variants involves affinity maturation using phage display. Briefly, several hypervariable region sites (e.g. 6-7 sites) are mutated to generate all possible amino acid substitutions at each site. The antibodies thus generated are displayed from filamentous phage particles as fusions to the gene III product of M13 packaged within each particle. The phage-displayed variants are then screened for their biological activity (e.g. binding affinity) as herein disclosed. In order to identify candidate hypervariable region sites for modification, alanine scanning mutagenesis can be performed to identify hypervariable region residues contributing significantly to antigen binding. Alternatively, or additionally, it may be beneficial to analyze a crystal structure of the antigen-antibody complex to identify contact points between the antibody and antigen. Such contact residues and neighboring residues are candidates for substitution according to the techniques elaborated herein. Once such variants are generated, the panel of variants is subjected to screening as described herein and antibodies with superior properties in one or more relevant assays may be selected for further development.

Nucleic acid molecules encoding amino acid sequence variants of the antibody are prepared by a variety of methods known in the art. These methods include, but are not limited to, isolation from a natural source (in the case of naturally occurring amino acid sequence variants) or preparation by oligonucleotide-mediated (or site-directed) mutagenesis, PCR mutagenesis, and cassette mutagenesis of an earlier prepared variant or a non-variant version of the antibody.

### Effector Function Engineering

(A) Anti-Her3 Antibodies with Variant Fc Regions

Most immune functions of antibodies depend on their ability to act as flexible adaptor molecules, linking pathogen with appropriate elimination mechanisms. This ‘bridging’ role entails two types of recognition, each involving contributions from particular antibody domains. The first involves highly specific recognition of the antigen target, and is mediated through the amino-terminal variable domains of the two Fab regions of the antibody. The second involves interaction of the constant domains of the Fc region of the molecule with various effector molecules, including complement and, perhaps most importantly, Fc receptors (FcRs) present on phagocytes and other immune cells. The dual recognition of target and FcR by immunoglobulin molecules has a key role in eliciting effector mechanisms to rid the body of bacteria, viruses, and parasites.

Briefly, therapeutic antibodies can exert potent biological functions through two major non-exclusive mechanisms: (i) they can block interactions between receptors and their ligands due to the exquisite epitope specificity of their variable domains (“neutralizing/antagonist antibodies”) or trigger potent biological responses such as apoptosis or cell proliferation once they are bound to surface molecules (“agonist antibodies”); (ii) induce effector functions against pathogens and tumor cells following their interactions with the complement component C1q and/or with receptors for Fc region (FcγR). See Cragg et al., Curr Opin Immunol 11:541-547 (1999); Glennie et al., Immunol Today 21:403-410 (2000).

The effector functions of immunoglobulins e.g., IgG, which is the most common immunoglobulin, are mediated by the antibody Fc region through two major mechanisms: (1) binding to the cell surface Fc receptors (FcγRs) can lead to ingestion of pathogens by phagocytosis or lysis by killer cells via the antibody-dependent cellular cytotoxicity (ADCC) pathway, or (2) binding to the C1q part of the first complement component C1 initiates the complement-dependent cytotoxicity (CDC) pathway, resulting in the lysis of pathogens. Reviewed in Daeron, Annu. Rev. Immunol. 15:203-234 (1997); Ward and Ghetie, Therapeutic Immunol. 2:77-94 (1995); Ravetch and Kinet, Annu. Rev. Immunol. 9:457-492 (1991); Uananue and Benacerraf, Textbook of Immunology, 2nd Edition, Williams & Wilkins, p. 218 (1984)). There are three known FcγRs, designated FcγRI (CD64), FcγRII (CD32), and FcγRIII (CD16). Anti-tumor efficacy can be due to a combination of these mechanisms, and their relative importance in clinical therapy appears to be cancer dependent. Notwithstanding this arsenal of anti-tumor weapons, the potency of antibodies as anti-cancer agents is unsatisfactory, particularly given their high cost. Currently for anti-cancer therapy, any small improvement in mortality rate defines success.

Thus, it may be desirable to introduce one or more amino acid modifications in an Fc region of the immunoglobulin polypeptides of the invention, thereby generating an Fc region variant. The Fc region variant may comprise a human Fc region sequence (e.g., a human IgG1, IgG2, IgG3 or IgG4 Fc region) comprising an amino acid modification (e.g. a substitution) at one or more amino acid positions including that of a hinge cysteine.

ADCC involves the recognition of the antibody by immune cells that engage the antibody-marked cells and either through their direct action, or through the recruitment of other cell types, leads to the tagged-cell's death. CDC is a process where a cascade of different complement proteins become activated, usually when several IgGs are in close proximity to each other, either with one direct outcome being cell lysis, or one indirect outcome being attracting other immune cells to this location for effector cell function.

A promising means for enhancing the anti-tumor potency of antibodies is via enhancement of their ability to mediate cytotoxic effector functions such as ADCC, ADCP, and CDC. The importance of ADCC as a cytotoxic mechanism of anti-tumor mAbs has been demonstrated in animal studies. Ravetch et al., Annu. Rev. Immunol. 16:421-432 (1998) showed that the tumoricidal effect of a humanized anti-Her2/neu mAb (epithelial growth factor receptor 2; Trastuzumab) was significantly reduced in FcγR knockout nude mice as compared to wild-type nude mice. Similarly, the tumor regression activity of a chimeric anti-CD20 mAb (Rituximab) was significantly reduced in FcγR deficient mice as compared to wild-type mice. Ravetch, supra; Clynes et al., Inhibitory Fc receptors modulate in vivo cytotoxicity against tumor targets. Nat. Med. 6:443-446 (2000). Further support for an important role for ADCC was provided by a study of Cartron et al., who found that in patients with a polymorphism in FcγRIIIa leading to increased binding of IgG1, therapy with an anti-CD20 mAb produced a 90% response rate (patients with complete remission or partial response) at 12 months, compared to a 51% response rate in individuals not expressing this polymorphism of FcγRIIIa. Cartron et al., Therapeutic activity of humanized anti-CD20 monoclonal antibody and polymorphism in IgG Fc receptor FcγRIIIa gene, Blood 99:754-758 (2002). Others have shown that this FcγRIIIa polymorphism and also a polymorphism in FcγRIIa are associated with the response rate to therapeutic mAbs. W. K. Weng and R. Levy, Two immunoglobulin G fragment C receptor polymorphisms independently predict response to rituximab in patients with follicular lymphoma. J. Clin. Oncol. 21:3940-3947 (2003). The importance of FcγR-mediated effector functions for the anti-cancer activity of antibodies has also been demonstrated in mice (Clynes et al., 1998, Proc Natl Acad Sci USA 95:652-656; Clynes et al., 2000, Nat Med 6:443-446), and the affinity of interaction between Fc and certain FcγRs correlates with targeted cytotoxicity in cell-based assays (Shields et al., 2001, J Biol Chem 276:6591-6604; Presta et al., 2002, Biochem Soc Trans 30:487-490; Shields et al., 2002, J Biol Chem 277:26733-26740). Additionally, a correlation has been observed between clinical efficacy in humans and their allotype of high (V158) or low (F158) affinity polymorphic forms of FcγRIIIa (Cartron et al., 2002, Blood 99:754-758). Together these data suggest that an antibody that is optimized for binding to certain FcγRs may better mediate effector functions and thereby destroy cancer cells more effectively in patients. The balance between activating and inhibiting receptors is an important consideration, and optimal effector function may result from an antibody that has enhanced affinity for activation receptors, for example FcγRI, FcγRIIa/c, and FcγRIIIa, yet reduced affinity for the inhibitory receptor FcγRIIb. Furthermore, because FcγRs can mediate antigen uptake and processing by antigen presenting cells, enhanced FcγR affinity may also improve the capacity of antibody therapeutics to elicit an adaptive immune response. Fc variants have been successfully engineered with selectively enhanced binding to FcγRs, and furthermore these Fc variants provide enhanced potency and efficacy in cell-based effector function assays. See for example U.S. Ser. No. 10/672,280, U.S. Ser. No. 10/822,231, entitled “Optimized Fc Variants and Methods for their Generation”, U.S. Ser. No. 60/627,774, entitled “Optimized Fc Variants”, and U.S. Ser. No. 60/642,477, entitled “Improved Fc Variants”, and references cited therein. 7,276,585 Xencor, Inc. (Monrovia, Calif.) See also Patent No. 6,821,505.

Most mAbs that mediate antibody-dependent cellular cytotoxicity (ADCC) also activate the complement system 1. A. Gorter and S. Meri, “Immune evasion of tumor cells using membrane-bound complement regulatory proteins.” Immunol. Today, pp. 576-582 (1999).

Complement initiates three mechanisms that can be used against mAb-coated tumor cells. The first is direct complement killing of tumor cells by the membrane attack complex (MAC), a process usually called ‘complement-dependent cytotoxicity’ (CDC). The second mechanism is complement receptor-dependent enhancement of ADCC. In this case, CR3 binds to iC3b, thus enhancing FcγR-mediated effector cell binding. A third mechanism used for killing microorganisms, CR3-dependent cellular cytotoxicity (CR3-DCC), is usually not activated with tumors.

Based upon the results of chemical modifications and crystallographic studies, Burton et al. (Nature, 288:338-344 (1980)) proposed that the binding site for the complement subcomponent C1q on IgG involves the last two (C-terminal).beta.-strands of the CH2 domain. Burton later suggested (Molec. Immunol., 22(3):161-206 (1985)) that the region comprising amino acid residues 318 to 337 might be involved in complement fixation.

Duncan and Winter (Nature 332:738-40 (1988)), using site directed mutagenesis, reported that Glu318, Lys320 and Lys322 form the binding site to C1q. The data of Duncan and Winter were generated by testing the binding of a mouse IgG2b isotype to guinea pig C1q. The role of Glu318, Lys320 and Lys322 residues in the binding of C1q was confirmed by the ability of a short synthetic peptide containing these residues to inhibit complement mediated lysis. Similar results are disclosed in U.S. Pat. No. 5,648,260 issued on Jul. 15, 1997, and U.S. Pat. No. 5,624,821 issued on Apr. 29, 1997.

The residue Pro331 has been implicated in C1q binding by analysis of the ability of human IgG subclasses to carry out complement mediated cell lysis. Mutation of Ser331 to Pro331 in IgG4 conferred the ability to activate complement. (Tao et al., J. Exp. Med., 178:661-667 (1993); Brekke et al., Eur. J. Immunol., 24:2542-47 (1994)).

From the comparison of the data of the Winter group, and the Tao et al. and Brekke et al. papers, Ward and Ghetie concluded in their review article that there are at least two different regions involved in the binding of C1q: one on the .beta.-strand of the CH2 domain bearing the Glu318, Lys320 and Lys322 residues, and the other on a turn located in close proximity to the same .beta.-strand, and containing a key amino acid residue at position 331.

Other reports suggest that human IgG1 residues Leu235, and Gly237, located in the lower hinge region, play a critical role in complement fixation and activation. Xu et al., J. Immunol. 150:152 A (Abstract) (1993). WO94/29351 published Dec. 22, 1994 reports that amino acid residues necessary for C1q and FcR binding of human IgG1 are located in the N-terminal region of the CH2 domain, i.e. residues 231 to 238.

It has further been proposed that the ability of IgG to bind C1q and activate the complement cascade also depends on the presence, absence, or modification of the carbohydrate moiety positioned between the two CH2 domains (which is normally anchored at Asn297). Ward and Ghetie, Therapeutic Immunology 2:77-94 (1995) at page 81.

The binding site on human and murine antibodies for FcγR have been previously mapped to the so-called “lower hinge region” consisting of residues 233-239 (EU index numbering as in Kabat et al., Sequences of Proteins of Immunological Interest, 5th Ed. Public Health Service, National Institutes of Health, Bethesda, Md. (1991)). Woof et al. Molec. Immunol. 23:319-330 (1986); Duncan et al. Nature 332:563 (1988); Canfield and Morrison, J. Exp. Med. 173:1483-1491 (1991); Chappel et al., Proc. Natl. Acad. Sci. USA 88:9036-9040 (1991). Of residues 233-239, P238 and S239 have been cited as possibly being involved in binding, but these two residues have never been evaluated by substitution or deletion.

Other previously cited areas possibly involved in binding to FcγR: G316-K338 (human IgG) for human FcγRI (by sequence comparison only; no substitution mutants were evaluated) (Woof et al. Molec. Immunol 23:319-330 (1986)); K274-R301 (human IgG1) for human FcγRIII (based on peptides) (Sarmay et al. Molec. Immunol. 21:43-51 (1984)); Y407-R416 (human IgG) for human FcγRIII (based on peptides) (Gergely et al. Biochem. Soc. Trans. 12:739-743 (1984)).

U.S. Pat. No. 6,165,745 discloses a method of producing an antibody with a decreased biological half-life by introducing a mutation into the DNA segment encoding the antibody. The mutation includes an amino acid substitution at position 253, 310, 311, 433, or 434 of the Fc-hinge domain. The full disclosure of U.S. Pat. No. 6,165,745, as well as the full disclosure of all other U.S. patent references cited herein, are hereby incorporated by reference.

U.S. Patent Application No. 20020098193 A1 and PCT Publication No. WO 97/34621 disclose mutant IgG molecules having increased serum half-lives relative to IgG wherein the mutant IgG molecule has at least one amino acid substitution in the Fc-hinge region. However, no experimental support is provided for mutations at positions 250, 314, or 428.

U.S. Pat. No. 6,277,375 B1 discloses a composition comprising a mutant IgG molecule having an increased serum half-life relative to the wild-type IgG, wherein the mutant IgG molecule comprises the amino acid substitutions: threonine to leucine at position 252, threonine to serine at position 254, or threonine to phenylalanine at position 256. A mutant IgG with an amino acid substitution at position 433, 435, or 436 is also disclosed.

U.S. Pat. No. 6,528,624 discloses a variant of an antibody comprising a human IgG Fc region, which variant comprises an amino acid substitution at one or more of amino acid positions 270, 322, 326, 327, 329, 331, 333, and 334 of the human IgG Fc region.

In accordance with the above description and the teachings of the art, it is contemplated that in some embodiments, an antibody used in methods of the invention may comprise one or more alterations as compared to the wild type counterpart antibody, e.g. in the Fc region. These antibodies would nonetheless retain substantially the same characteristics required for therapeutic utility as compared to their wild type counterpart. For example, it is thought that certain alterations can be made in the Fc region that would result in altered (i.e., either improved or diminished) C1q binding and/or Complement Dependent Cytotoxicity (CDC), e.g., as described in WO99/51642. See also Duncan & Winter Nature 322:738-40 (1988); U.S. Pat. No. 5,648,260; U.S. Pat. No. 5,624,821; and WO94/29351 concerning other examples of Fc region variants. WO00/42072 (Presta) and WO 2004/056312 (Lowman) describe antibody variants with improved or diminished binding to FcRs. The content of these patent publications are specifically incorporated herein by reference. See, also, Shields et al. J. Biol. Chem. 9(2): 6591-6604 (2001). Antibodies with increased half lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934 μl (Hinton et al.). These antibodies comprise an Fc region with one or more substitutions therein which improve binding of the Fc region to FcRn. Polypeptide variants with altered Fc region amino acid sequences and increased or decreased C1q binding capability are described in U.S. Pat. No. 6,194,551B1, WO99/51642. The contents of those patent publications are specifically incorporated herein by reference. See, also, Idusogie et al. J. Immunol. 164:4178-4184 (2000).

Functional Assays of Molecules with Variant Fc Regions

A. Variant Fc Regions

The ability of any particular antibody e.g., any one or more of the anti-antibodies disclosed herein, to mediate lysis of the target cell by complement activation and/or ADCC can be assayed. Functional assays for identifying potent Fc variants of any one or more of the anti-Her3 antibodies of the invention are well known to one skilled in the art. See, for example, U.S Patent Application Publications 2005/0037000 and 2005/0064514, and International Patent Application Publication WO 04/063351 (each of which is hereby incorporated by reference in its entirety); that describe yeast display technology for characterizing an antibody with a variant Fc region. Likewise, R-Fc binding assays are disclosed in U.S Patent Application Publications 2005/0037000 and 2005/0064514, and International Patent Application Publication WO 04/063351 (each of which is hereby incorporated by reference in its entirety).

Examples of effector cell functions that can be assayed in accordance with the invention, include but are not limited to, antibody-dependent cell mediated cytotoxicity, phagocytosis, opsonization, opsonophagocytosis, C1q binding, and complement dependent cell mediated cytotoxicity. Any cell-based or cell free assay known to those skilled in the art for determining effector cell function activity can be used (For effector cell assays, see Perussia et al., 2000, Methods Mal. Biol. 121: 179-92; Baggiolini et al., 1998 Experientia, 44(10): 841-8; Lehmann et al., 2000 J. Immunol. Methods, 243(1-2): 229-42; Brown E J. 1994, Methods Cell Biol., 45: 147-64; Munn et al., 1990 J. Exp. Med., 172: 231-237, Abdul-Majid et al., 2002 Scand. J. Immunol. 55: 70-81; Ding et al., 1998, Immunity 8:403-411, each of which is incorporated by reference herein in its entirety).

Generally, the cells of interest are grown and labeled in vitro; the target antibody is added to the cell culture in combination with either serum complement or immune cells which may be activated by the antigen-antibody complexes. Cytolysis of the target cells is detected by the release of label from the lysed cells. In fact, antibodies can be screened using the patient's own serum as a source of complement and/or immune cells. The antibody that is capable of activating complement or mediating ADCC in the in vitro test can then be used therapeutically in that particular patient.

Preferably, the effector cells used in the ADCC assays of the invention are peripheral blood mononuclear cells (PBMC) that are preferably purified from normal human blood, using standard methods known to one skilled in the art, e.g. using Ficoll-Paque density gradient centrifugation.

An exemplary assay for determining ADCC activity of such anti-Her3 antibodies with variant Fc regions is based on a 51Cr release assay comprising of: labeling target cells with [51Cr]Na2CrO4 (this cell-membrane permeable molecule is commonly used for labeling since it binds cytoplasmic proteins and although spontaneously released from the cells with slow kinetics, it is released massively following target cell necrosis); opsonizing the target cells with the anti-antibodies with variant Fc region(s) of the invention; combining the opsonized radiolabeled target cells with effector cells in a microtitre plate at an appropriate ratio of target cells to effector cells; incubating the mixture of cells for 16-18 hours at 37° C.; collecting supernatants; and analyzing radioactivity. The cytotoxicity of the anti-antibodies with variant Fc regions can then be determined using known formulae, for example using the following formula: % lysis=(experimental cpm−target leak cpm)/(detergent lysis cpm−target leak cpm)×100%. Alternatively, % lysis=(ADCC−AICC)/(maximum release−spontaneous release). Specific lysis can be calculated using the formula: specific lysis=% lysis with the anti-antibodies with variant Fc region(s) of the invention−% lysis in the absence of the anti-antibodies with variant Fc region(s) of the invention. A graph can be generated by varying either the target:effector cell ratio or antibody concentration. Perussia et al., 2000, Methods Mol. Biol. 121: 179-92.

The affinities and binding properties of anti-antibodies with variant Fc regions for an FcγR may initially be determined using in vitro assays (biochemical or immunological based assays) known in the art for determining Fc-FcγR interactions, i.e., specific binding of an Fc region to an FcγR including but not limited to ELISA assay, surface plasmon resonance assay, immunoprecipitation assays. Preferably, the binding properties of the anti-antibodies with variant Fc regions in accordance with the invention may also be characterized by in vitro functional assays for determining one or more FcγR mediator effector cell functions. In some embodiments, the anti-Her3 Fc variants of the invention have similar binding properties in in vivo models as those in in vitro based assays. However, the present invention does not exclude molecules of the invention that do not exhibit the desired phenotype in in vitro based assays but do exhibit the desired phenotype in vivo.

Methods for generating anti-antibodies with variant Fc regions are known. DNA encoding an amino acid sequence variant of any one or more of the herein disclosed starting anti-Her3 antibodies may be prepared by a variety of methods known in the art. These methods include, but are not limited to, preparation by site-directed (or oligonucleotide-mediated) mutagenesis, PCR mutagenesis, and cassette mutagenesis of an earlier prepared DNA encoding the antibody. In an alternative embodiment of the invention, however, a nucleic acid encoding an Fc region of a parent antibody is available and this nucleic acid sequence is altered to generate a variant nucleic acid sequence encoding the Fc region variant.

Site-directed mutagenesis is a preferred method for preparing substitution variants. This technique is well known in the art (see, e.g., Carter et al. Nucleic Acids Res. 13:4431-4443 (1985) and Kunkel et al, Proc. Natl. Acad. Sci. USA 82:488 (1985)). Briefly, in carrying out site-directed mutagenesis of DNA, the starting DNA is altered by first hybridizing an oligonucleotide encoding the desired mutation to a single strand of such starting DNA. After hybridization, a DNA polymerase is used to synthesize an entire second strand, using the hybridized oligonucleotide as a primer, and using the single strand of the starting DNA as a template. Thus, the oligonucleotide encoding the desired mutation is incorporated in the resulting double-stranded DNA.

PCR mutagenesis is also suitable for making amino acid sequence variants of the starting polypeptide. See Higuchi, in PCR Protocols, pp. 177-183 (Academic Press, 1990); and Vallette et al., Nuc. Acids Res. 17:723-733 (1989). Briefly, when small amounts of template DNA are used as starting material in a PCR, primers that differ slightly in sequence from the corresponding region in a template DNA can be used to generate relatively large quantities of a specific DNA fragment that differs from the template sequence only at the positions where the primers differ from the template.

Another method for preparing variants, cassette mutagenesis, is based on the technique described by Wells et al., Gene 34:315-323 (1985). The starting material is the plasmid (or other vector) comprising the starting polypeptide DNA to be mutated. The codon(s) in the starting DNA to be mutated are identified. There must be a unique restriction endonuclease site on each side of the identified mutation site(s). If no such restriction sites exist, they may be generated using the above-described oligonucleotide-mediated mutagenesis method to introduce them at appropriate locations in the starting polypeptide DNA. The plasmid DNA is cut at these sites to linearize it. A double-stranded oligonucleotide encoding the sequence of the DNA between the restriction sites but containing the desired mutation(s) is synthesized using standard procedures, wherein the two strands of the oligonucleotide are synthesized separately and then hybridized together using standard techniques. This double-stranded oligonucleotide is referred to as the cassette. This cassette is designed to have 5′ and 3′ ends that are compatible with the ends of the linearized plasmid, such that it can be directly ligated to the plasmid. This plasmid now contains the mutated DNA sequence.

Alternatively, or additionally, the desired amino acid sequence encoding an anti-Fc variant can be determined, and a nucleic acid sequence encoding such amino acid sequence variant can be generated synthetically.

In certain embodiments, the modification entails one or more amino acid substitutions. The substitution may, for example, be a “conservative substitution”.

In some embodiments, the molecules of the invention with altered affinities for activating and/or inhibitory receptors having variant Fc regions, have one or more amino acid modifications.

The Fc regions of any one or more of the herein disclosed anti-antibodies may be optimized for a variety of properties. Properties that may be optimized include but are not limited to enhanced or reduced affinity for an FcγR. In one embodiment, the Fc variants are optimized to possess enhanced affinity for a human activating FcγR, preferably FcγRI, FcγRIIa, FcγRIIc, FcγRIIIa, and FcγRIIIb, most preferably FcγRIIIa. In an alternative embodiment, the Fc region is optimized to possess reduced affinity for the human inhibitory receptor FcγRIIb. These embodiments are anticipated to provide anti-antibodies with enhanced therapeutic properties in humans, for example enhanced effector function and greater anti-cancer potency.

In an alternate embodiment, the Fc variants of the present invention are optimized to have reduced or ablated affinity for a human FcγR, including but not limited to FcγRI, FcγRIIa, FcγRIIb, FcγRIIc, FcγRIIIa, and FcγRIIIb. These embodiments are anticipated to provide anti-Her3 antibodies with enhanced therapeutic properties in humans, for example reduced effector function and reduced toxicity. As is known in the art, cancer cells can be grafted or injected into mice to mimic a human cancer, a process referred to as xenografting. Testing of any one or more of the anti-Her3 antibodies that comprise Fc variants that are optimized for one or more mouse FcγR, may provide valuable information with regard to the efficacy of the antibody, its mechanism of action, and the like.

The Fc variants of the present invention may also be optimized for enhanced functionality and/or solution properties in aglycosylated form. In one embodiment, the aglycosylated Fc variants of the present invention bind an Fc ligand with greater affinity than the aglycosylated form of the parent Fc polypeptide. Exemplary Fc ligands include but are not limited to FcγRs, C1q, FcRn, and proteins A and G, and may be from any source, preferably human. In an alternative embodiment, the Fc variants of the invention are optimized to be more stable and/or more soluble than the aglycosylated form of the parent Fc polypeptide.

Certain aspects of this invention thus involve antibodies which are (a) directed against a particular antigen and (b) belong to a subclass or isotype that is capable of mediating the lysis of cells to which the antibody molecule binds. More specifically, these antibodies should belong to a subclass or isotype that, upon complexing with cell surface proteins, activates serum complement and/or mediates antibody dependent cellular cytotoxicity (ADCC) by activating effector cells such as natural killer cells or macrophages. Towards this end, it may be desirable to modify the antibody of the invention with respect to effector function, so as to enhance the effectiveness of the antibody in treating cancer, for example. For example, cysteine residue(s) may be introduced in the Fc region, thereby allowing interchain disulfide bond formation in this region. The homodimeric antibody thus generated may have improved internalization capability and/or increased complement-mediated cell killing and antibody-dependent cellular cytotoxicity (ADCC). See Caron et al., J. Exp Med. 176:1191-1195 (1992) and Shopes, B. J. Immunol. 148:2918-2922 (1992). Homodimeric antibodies with enhanced anti-tumor activity may also be prepared using heterobifunctional cross-linkers as described in Wolff et al. Cancer Research 53:2560-2565 (1993). Alternatively, any one or more of the anti-antibodies of the invention can be engineered with dual Fc regions and may thereby have enhanced complement lysis and ADCC capabilities. See Stevenson et al. Anti-Cancer Drug Design 3:219-230 (1989).

In yet another embodiment, the antibodies with variant Fc region(s) of the invention are characterized for antibody dependent cellular cytotoxicity (ADCC) see, e.g., Ding et al, Immunity, 1998, 8:403-11; which is incorporated herein by reference in its entirety.

In another example, one or more amino acids in the Fc region can be replaced with a different amino acid residue such that the antibody has altered C1q binding and/or reduced or abolished complement dependent cytotoxicity (CDC). This approach is described in further detail in U.S. Pat. No. 6,194,551 by Idusogie et al.

In another example, one or more amino acid residues within amino acid positions 231 and 239 are altered to thereby alter the ability of the antibody to fix complement. This approach is described further in PCT Publication WO 94/29351 by Bodmer et al.

A broad aspect of the invention thus relates to immunoglobulins (e.g., anti-antibodies disclosed herein), comprising a variant Fc region, having one or more amino acid modifications (e.g., substitutions, but also including insertions or deletions) in one or more regions, which modifications alter, e.g., increase or decrease, the affinity of the variant Fc region for an FcγR. As binding to FcγRIIb decreases ADCC, it is important to increase binding to FcγRIIIA and decrease binding to FcγRIIB. Thus, in certain embodiments, said one or more amino acid modification increases the affinity of the variant Fc region for FcγRIIIA and/or FcγRIIA.

In certain embodiments, the herein described anti-antibodies with a variant Fc region further specifically bind FcγRIIB (via the Fc region) with a lower affinity than a comparable antibody molecule (i.e., having the same amino acid sequence as the antibody with a variant Fc region except for the one or more amino acid modifications in the Fc region) comprising the wild-type Fc region binds FcγRIIB.

In some embodiments, the invention encompasses molecules comprising a variant Fc region, wherein said variant Fc region comprises at least one amino acid modification relative to a wild type Fc region, which variant Fc region does not bind any FcγR or binds with a reduced affinity, relative to a comparable molecule comprising the wild-type Fc region, as determined by standard assays (e.g., in vitro assays) known to one skilled in the art. In a specific embodiment, the invention encompasses molecules comprising a variant Fc region, wherein said variant Fc region comprises at least one amino acid modification relative to a wild type Fc region, which variant Fc region only binds one FcγR, wherein the FcγR is FcγRIIIA. In another specific embodiment, the invention encompasses molecules comprising a variant Fc region, wherein said variant Fc region comprises at least one amino acid modification relative to a wild type Fc region, which variant Fc region only binds one FcγR wherein the FcγR is FcγRIIA. In yet another embodiment, the invention encompasses an anti-antibody molecule comprising a variant Fc region, wherein the variant Fc region comprises at least one amino acid modification relative to a wild type Fc region, which variant Fc region only binds one FcγR wherein the FcγR is FcγRIIB.

In yet another embodiment, provided herein is at least one or more anti-antibodies which comprises an antigen binding region and a variant Fc region, wherein the variant Fc region: (A) differs from a wild-type Fc region by comprising at least one amino acid modification according to the EU index as in Kabat, relative to the wild-type Fc region (unmodified), e.g., any one or more corresponding anti-antibodies disclosed herein that include the wild type Fc polypeptide; and (B) binds an FcγR with an increased affinity relative to a said wild-type Fc region.

The Fc variants of the present invention may be combined with other Fc modifications, including but not limited to modifications that alter effector function or interaction with one or more Fc ligands. Such combination may provide additive, synergistic, or novel properties in antibodies or Fc fusions. In one embodiment, the Fc variants of the present invention may be combined with other known Fc variants (Duncan et al., 1988, Nature 332:563-564; Lund et al., 1991, J Immunol 147:2657-2662; Lund et al., 1992, Mol Immunol 29:53-59; Alegre et al., 1994, Transplantation 57:1537-1543; Hutchins et al., 1995, Proc Natl Acad Sci USA 92:11980-11984; Jefferis et al., 1995, Immunol Left 44:111-117; Lund et al., 1995, Faseb J 9:115-119; Jefferis et al., 1996, Immunol Left 54:101-104; Lund et al., 1996, J Immunol 157:4963-4969; Armour et al., 1999, Eur J Immunol 29:2613-2624; Idusogie et al., 2000, J Immunol 164:4178-4184; Reddy et al., 2000, J Immunol 164:1925-1933; Xu et al., 2000, Cell Immunol 200:16-26; Idusogie et al., 2001, J Immunol 166:2571-2575; Shields et al., 2001, J Biol Chem 276:6591-6604; Jefferis et al., 2002, Immunol Left 82:57-65; Presta et al., 2002, Biochem Soc Trans 30:487-490; Hinton et al., 2004, J Biol Chem 279:6213-6216) (U.S. Pat. No. 5,624,821; U.S. Pat. No. 5,885,573; U.S. Pat. No. 6,194,551; PCT WO 00/42072; PCT WO 99/58572; US 2004/0002587 A1). In an alternate embodiment, the Fc variants of the present invention are incorporated into an antibody or Fc fusion that comprises one or more engineered glycoforms (infra). Thus combinations of the Fc variants of the present invention with other Fc modifications, as well as undiscovered Fc modifications, are contemplated with the goal of generating novel antibodies or Fc fusions with optimized properties.

B. Anti-Her3 Engineered Glycoforms

The invention additionally, encompasses anti-antibodies including fragments thereof which are differentially modified during or after translation, e.g., by glycosylation, proteolytic cleavage etc. Any of numerous chemical modifications may be carried out by known techniques, including but not limited to, specific chemical cleavage by trypsin, papain, metabolic synthesis in the presence of tunicamycin etc.

Antibodies are glycoproteins containing carbohydrate structures at conserved positions in the heavy chain constant regions, with each isotype possessing a distinct array of N-linked carbohydrate structures, which variably affect protein assembly, secretion or functional activity. The structure of the attached N-linked carbohydrate varies considerably and can include high-mannose, multiply-branched as well as biantennary complex oligosaccharides. (Wright, A., and Morrison, S. L., Trends Biotech. 15:26-32 (1997)). The major carbohydrate units are attached to amino acid residues of the constant region of the antibody. Carbohydrate is also known to attach to the antigen binding sites of some antibodies and may affect the antibody-binding characteristics by limiting access of the antigen to the antibody binding site. Typically, there is heterogeneous processing of the core oligosaccharide structures attached at a particular glycosylation site such that even monoclonal antibodies exist as multiple glycoforms. Likewise, it has been shown that major differences in antibody glycosylation occur between cell lines, and even minor differences are seen for a given cell line grown under different culture conditions. (Lifely, M. R. et al., Glycobiology 5(8):813-22 (1995)).

Monoclonal antibodies often achieve their therapeutic benefit through two binding events. First, the variable domain of the antibody binds to a specific tumor receptor on the surface of the target cell. This is followed by recruitment of effector cells such as natural killer (NK) cells that bind to the constant region (Fc) of the antibody and destroy cells to which the antibody has bound. This process, known as antibody-dependent cell cytotoxicity (ADCC), partially depends on a specific N-glycosylation event at Asn 297 in the Fc domain of the heavy chain of IgG1s. In general, antibodies that lack this N-glycosylation structure still bind antigen but cannot mediate ADCC, apparently as a result of reduced affinity of the Fc domain of the antibody for the Fc receptor FcγRIIIa on the surface of NK cells. Interestingly, there is a linear increase of in vitro complement activation with increasing terminal galactosylation of the carbohydrate moiety in the Fc domain. There are a number of roles associated with the carbohydrate units. Glycosylation may affect overall solubility and the rate of catabolism of the antibody. It is also known that carbohydrate is necessary for cellular secretion of some antibody chains. It has been demonstrated that glycosylation of the constant region plays a vital role in the effector functioning of an antibody; without this glycosylation in its correct configuration, the antibody may be able to bind to the antigen but may not be able to bind for example to macrophages, helper and suppressor cells or complement, to carry out its role of blocking or lysing the cell to which it is bound. Hyperglycosylated proteins have been shown to exhibit increased serum half-life, are less sensitive to proteolysis and more heat-stable compared with the non-glycosylated forms. (Leatherbarrow et al., Mol. Immunol. 22:407 (1985)).

IgG1 type antibodies, which represent the most commonly used antibodies in cancer immunotherapy, are glycoproteins that have a conserved N-linked glycosylation site at Asn297 in each CH2 domain. The two complex bi-antennary oligosaccharides attached to Asn297 are buried between the CH2 domains, forming extensive contacts with the polypeptide backbone, and their presence is essential for the antibody to mediate effector functions such as antibody dependent cellular cytotoxicity (ADCC) (Lifely, M. R., et al., Glycobiology 5:813-822 (1995); Jefferis, R., et al., Immunol Rev. 163:59-76 (1998); Wright, A. and Morrison, S. L., Trends Biotechnol. 15:26-32 (1997)). Glycosylation of IgG at asparagine 297 in the C_{H2} domain is also required for full capacity of IgG to activate the classical pathway of complement-dependent cytolysis (Tao and Morrison, J. Immunol. 143:2595 (1989)).

More, glycosylation of IgM at asparagine 402 in the CH 3 domain is necessary for proper assembly and cytolytic activity of the antibody (Muraoka and Shulman, J. Immunol. 142:695 (1989)). Likewise, removal of glycosylation sites as positions 162 and 419 in the CH1 and CH3 domain of an IgA antibody has been shown to lead to intracellular degradation and at least 90% inhibition of secretion (Taylor and Wall, Mol. Cell. Biol. 8:4197 (1988)).

Glycosylation of immunoglobulins in the variable (V) region has also been observed. Sox and Hood, Proc. Natl. Acad. Sci. USA 66:975 (1970), reported that about 20% of human antibodies are glycosylated in the V region. Glycosylation of the V domain is believed to arise from fortuitous occurrences of the N-linked glycosylation signal Asn-Xaa-Ser/Thr in the V region sequence and has not been recognized in the art as playing an important role in immunoglobulin function.

It has also been reported that glycosylation at CDR2 of the heavy chain, in the antigen binding site, of a murine antibody specific for .alpha.-(1-6)dextran increases its affinity for dextran (Wallick et al., J. Exp. Med. 168:1099 (1988) and Wright et al., EMBO J. 10:2717 (1991)). See U.S. Pat. No. 6,933,368. Some classes and subclasses also have O-linked sugars, often in the hinge region, eg. IgD and IgA from some species.

For example, the absence of fucose or the presence of a bisecting N-acetylglucosamine in the carbohydrate structure of the monoclonal antibody, has been positively correlated with the potency of ADCC. Specifically, defucosylated carbohydrate residues on monoclonal antibodies have been shown to enhance the ADCC capability of the target antibody more than threefold. “Glycosylation of therapeutic proteins in different production systems” Acta Paediatrica, 96: 17-22 (2007); Shields et al., “Lack of Fucose on Human IgG1 N-Linked Oligosaccharide Improves Binding to Human FcγRIII and Antibody-dependent Cellular Toxicity”, J. Biol. Chem., 277: 26733-26740 (2002). Likewise, specific engineered glycoforms of monoclonal antibodies, which interact solely with the FcγRIIIa receptor of natural killer cells, exhibit superior ADCC compared with heterogeneous glycoforms that interact with different Fc receptors. The collective data impel the conclusion that glycoengineering for directed glycosylation of therapeutic proteins can improve the therapeutic effect in vivo. See Umaa, P. et al., Nature Biotechnol. 17:176-180 (1999)). See also U.S. Pat. No. 5,624,821; U.S. Pat. No. 6,602,684; WO 00/42072 and WO 07/048,122, the content of each of which is incorporated in its entirety by reference herein. See also US serial No. 2006/0182744. Not only is ADCC dependent on glycosylation of the Fc domain, but the degree of cell-mediated killing is also sensitive to the composition of the glycans in the Fc region of the antibody.

As a consequence, the present invention, in related embodiments, provides “Engineered Glycoforms” of any one or more of the anti-antibodies disclosed herein including fragments thereof, wherein the glycosylation profiles of the antibody are altered to enhance their use in the treatment of specific types of cancers or other disease states.

By “engineered glycoform” as used herein is meant a carbohydrate composition that is covalently attached to an Fc polypeptide, wherein the carbohydrate composition differs chemically from that of a parent Fc polypeptide. Engineered glycoforms may be useful for a variety of purposes, including but not limited to enhancing or reducing effector function. Engineered glycoforms may be generated by a variety of methods known in the art (Umana et al., 1999, Nat Biotechnol 17:176-180; Davies et al., 2001, Biotechnol Bioeng 74:288-294; Shields et al., 2002, J Biol Chem 277:26733-26740; Shinkawa et al., 2003, J Biol Chem 278:3466-3473); (U.S. Pat. No. 6,602,684; U.S. Ser. No. 10/277,370; U.S. Ser. No. 10/113,929; PCT WO 00/61739A1; PCT WO 01/29246A1; PCT WO 02/31140A1; PCT WO 02/30954A1); (Potelligent™ technology [Biowa, Inc., Princeton, N.J.]; GlycoMAb™ glycosylation engineering technology [GLYCART biotechnology AG, Zurich, Switzerland)). Many of these techniques are based on controlling the level of fucosylated and/or bisecting oligosaccharides that are covalently attached to the Fc region, for example by expressing an Fc polypeptide in various organisms or cell lines, engineered or otherwise (for example Lec-13 CHO cells or rat hybridoma YB2/0 cells), by regulating enzymes involved in the glycosylation pathway (for example FUT8 [.alpha.1,6-fucosyltranserase] and/or .beta.1-4-N-acetylglucosaminyltransferase III [GnTIII]), or by modifying carbohydrate(s) after the Fc polypeptide has been expressed. Engineered glycoform typically refers to the different carbohydrate or oligosaccharide; thus an Fc polypeptide, for example an antibody or Fc fusion, may comprise an engineered glycoform. Alternatively, engineered glycoform may refer to the Fc polypeptide that comprises the different carbohydrate or oligosaccharide.

Covalent modification of the target antibody included within the scope of this invention comprises altering the native glycosylation pattern of the polypeptide. “Altering the native glycosylation pattern” is intended for purposes herein to mean deleting one or more carbohydrate moieties found in the native target antibody, and/or adding one or more glycosylation sites that are not present in the native target antibody.

In still another embodiment, the glycosylation of an antibody is modified. For example, an aglycoslated antibody can be made (i.e., the antibody lacks glycosylation). Glycosylation can be altered to, for example, increase the affinity of the antibody for antigen. Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such aglycosylation may increase the affinity of the antibody for antigen. Such an approach is described in further detail in U.S. Pat. Nos. 5,714,350 and 6,350,861 by Co et al.

Additionally or alternatively, an antibody can be made that has an altered type of glycosylation, such as a hypofucosylated antibody having reduced amounts of fucosyl residues or an antibody having increased bisecting GlcNac structures. Such altered glycosylation patterns have been demonstrated to increase the ADCC ability of antibodies. Such carbohydrate modifications can be accomplished by, for example, expressing the antibody in a host cell with altered glycosylation machinery. Cells with altered glycosylation machinery have been described in the art and can be used as host cells in which to express recombinant antibodies of the invention to thereby produce an antibody with altered glycosylation. For example, EP 1,176,195 by Hanai et al. describes a cell line with a functionally disrupted FUT8 gene, which encodes a fucosyl transferase, such that antibodies expressed in such a cell line exhibit hypofucosylation. PCT Publication WO 03/035835 by Presta describes a variant CHO cell line, Lec13 cells, with reduced ability to attach fucose to Asn(297)-linked carbohydrates, also resulting in hypofucosylation of antibodies expressed in that host cell (see also Shields, R. L. et al. (2002) J. Biol. Chem. 277:26733-26740). PCT Publication WO 99/54342 by Umana et al. describes cell lines engineered to express glycoprotein-modifying glycosyl transferases (e.g., beta(1,4)—N-acetylglucosaminyltransferase III (GnTIII)) such that antibodies expressed in the engineered cell lines exhibit increased bisecting GlcNac structures which results in increased ADCC activity of the antibodies (see also Umana et al. (1999) Nat. Biotech. 17:176-180).

Antibodies disclosed herein can be glycosylated in both the C-regions and in the V-regions. Obviously C-region glycosylation is dependent on the particular sequence which by definition defines the class and subclass of the antibody. As noted elsewhere, many classes of antibody have conserved N-linked glycosylation sites in the constant domains. For example all IgG antibodies have a conserved N-linked glycosylation site in the CH2 domain at residue Asn297.

Two basic types of glycosylation of therapeutic proteins are known: O-linked and N-linked glycosylation. O-linked glycosylation is initiated by the attachment of N-acetyl-galactosamine to a serine or threonine residue in the peptide backbone of the therapeutic protein. The proximal carbohydrate is the target for glycosyltransferases to form a mature O-glycan structure. It is difficult to predict where O-linked glycosylation will occur in the protein as there is no clear consensus amino acid sequence for O-linked glycosylation (3, 4). However, O-linked glycosylation is affected by secondary structural elements such as an extended β-turn. In contrast, consensus amino acid sequences are known for N-glycosylation. N-glycosylation occurs at a specific sequence motif, Asn-X-Thr/Ser (sequon or consensus sequence; where X is any amino acid except proline), and this consensus sequence must be accessible to the precursor transferring the enzyme. In the case where X=Pro, no glycosylation takes place. Asn-X-Thr/Ser sequences in n-turns can influence the protein conformation by N-linked glycosylation. As glycosylation precedes final protein folding, the structure of the resultant therapeutic protein may be altered, resulting in differences in activity or stability compared with the non-glycosylated form. Thus, recombinant antibodies of the invention can be modified to recreate or create additional glycosylation sites if desired, which is simply achieved by engineering the appropriate amino acid sequences (such as Asn-X-Ser, Asn-X-Thr, Ser, or Thr) into the primary sequence of the antibody. Glycosylation of polypeptides is typically either N-linked or O-linked. N-linked refers to the attachment of the carbohydrate moiety to the side chain of an asparagine residue. The tri-peptide sequences asparagine-X-serine and asparagine-X-threonine, where X is any amino acid except proline, are the recognition sequences for enzymatic attachment of the carbohydrate moiety to the asparagine side chain. Thus, the presence of either of these tri-peptide sequences in a polypeptide creates a potential glycosylation site. O-linked glycosylation refers to the attachment of one of the sugars N-acetylgalactosamine, galactose, or xylose, to a hydroxyamino acid, most commonly serine or threonine, although 5-hydroxyproline or 5-hydroxylysine may also be used.

Thus, in certain embodiments of the invention a mutant anti-antibody is provided that exhibits a higher affinity for its antigen e.g., receptor or endogenous binding partner, than a parent antibody that comprises a parent immunoglobulin chain, wherein the mutant immunoglobulin chain comprises an amino acid substitution that eliminates a variable region glycosylation site of the parent immunoglobulin chain, said elimination having the effect of increasing the affinity of the mutant antibody relative to the parent antibody. Alternative embodiments contemplate variants that are “aglycosylated.”

“Glycosylation sites” refer to amino acid residues which are recognized by a eukaryotic cell as locations for the attachment of sugar residues. The amino acids where carbohydrate, such as oligosaccharide, is attached are typically asparagine (N-linkage), serine (O-linkage), and threonine (O-linkage) residues. In order to identify potential glycosylation sites within an antibody or antigen-binding fragment, the sequence of the antibody is examined, for example, by using publicly available databases such as the website provided by the Center for Biological Sequence Analysis (see http://www.cbs.dtu.dk/services/NetNGlyc/ for predicting N-linked glycosylation sites) and http://www.cbs.dtu.dk/services/NetOGlyc/ for predicting O-linked glycosylation sites). Additional methods for altering glycosylation sites of antibodies are described in U.S. Pat. Nos. 6,350,861 and 5,714,350.

Several approaches have been attempted to alter the glycosylation state of IgG antibodies: inhibition of glycosylation by culturing cells in the presence of the drug tunicamycin (Leatherbarrow et al. 1985; Walker et al. 1989; Pound et al. 1993); treatment of glycoproteins with specific glycosidases that remove the entire oligosaccharide or specific residues (Tsuchiya et al. 1989; Boyd et al. 1995); or site-directed mutagenesis to remove either the carbohydrate addition site (Tao, Smith et al. 1993) or residues within the CH2 region that contact the core oligosaccharide residues (Lund, Takahashi et al. 1996). These studies have confirmed that the presence of carbohydrate is essential to antibody function.

Glycosylation can be achieved by methods known in the art, e.g., by producing the antibody in a mammalian host cell such as Chinese Hamster Ovary (CHO) cell or in yeast. Addition of glycosylation sites to the target antibody is conveniently accomplished by altering the amino acid sequence such that it contains one or more of the above-described tri-peptide sequences (for N-linked glycosylation sites). The alteration may also be made by the addition of, or substitution by, one or more serine or threonine residues to the native target antibody sequence (for O-linked glycosylation sites). For ease, the target antibody amino acid sequence is preferably altered through changes at the DNA level, particularly by mutating the DNA encoding the target antibody at preselected bases such that codons are generated that will translate into the desired amino acids. The DNA mutation(s) may be made using methods described above under the heading of “Amino Acid Sequence Variants of Target antibody”.

Yeast provides substantial advantages over bacteria for the production of immunoglobulin H and L chains. Yeasts carry out post-translational peptide modifications including glycosylation. A number of recombinant DNA strategies now exist which utilize strong promoter sequences and high copy number plasmids which can be used for production of the desired proteins in yeast. Yeast recognizes leader sequences of cloned mammalian gene products and secretes peptides bearing leader sequences (i.e., pre-peptides) (Hitzman, et al., 11th International Conference on Yeast, Genetics and Molecular Biology, Montpelier, France, Sep. 13-17, 1982).

Another means of increasing the number of carbohydrate moieties on the target antibody is by chemical or enzymatic coupling of glycosides to the polypeptide. These procedures are advantageous in that they do not require production of the polypeptide in a host cell that has glycosylation capabilities for N- and O-linked glycosylation. Depending on the coupling mode used, the sugar(s) may be attached to (a) arginine and histidine, (b) free carboxyl groups, (c) free sulfhydryl groups such as those of cysteine, (d) free hydroxyl groups such as those of serine, threonine, or hydroxyproline, (e) aromatic residues such as those of phenylalanine, tyrosine, or tryptophan, or (f) the amide group of glutamine. These methods are described in WO 87/05330 published Sep. 11, 1987, and in Aplin and Wriston (CRC Crit. Rev. Biochem., pp. 259-306 [1981]).

Moreover, the main species antibody or variant thereof may further comprise glycosylation variations, non-limiting examples of which include antibody comprising a G1 or G2 oligosaccharide structure attached to the Fc region thereof, antibody comprising a carbohydrate moiety attached to a light chain thereof (e.g. one or two carbohydrate moieties, such as glucose or galactose, attached to one or two light chains of the antibody, for instance attached to one or more lysine residues), antibody comprising one or two non-glycosylated heavy chains, or antibody comprising a sialylated oligosaccharide attached to one or two heavy chains thereof etc.

Immune effector functions are unnecessary or even deleterious in certain clinical settings. In another embodiment of the invention, antibodies or fragments thereof are altered wherein the constant region of the antibody is modified to reduce at least one constant region-mediated biological effector function relative to an unmodified antibody. Such modified antibodies are often referred to as “aglycosylated” antibodies. In order to improve the binding affinity of an antibody of the invention or antigen-binding fragment thereof to the antigen while minimizing or reducing its binding to the Fc receptor, the antibody can be mutated at particular regions necessary for Fc receptor (FcR) interactions (see e.g., Canfield, S. M. and S. L. Morrison (1991) J. Exp. Med. 173:1483-1491; and Lund, J. et al. (1991) J. of Immunol. 147:2657-2662). Reduction in FcR binding ability of the antibody may also reduce other effector functions which rely on FcR interactions, such as opsonization and phagocytosis and antigen-dependent cellular cytotoxicity, glycosylation sites of the antibody can be altered, for example, by mutagenesis (e.g., site-directed mutagenesis). As a consequence, such antibodies do not exhibit substantial immune effector functions that are dependent on glycosylation of the Fc region. Generally and preferably, an aglycosylated antibody of the invention does not exhibit substantial immune effector functions except for binding to FcRn. In some embodiments, an antibody of the invention or a fragment thereof does not possess substantial or completely lacks effector functions other than FcRn binding. In one embodiment, said effector function is complement lysis. In one embodiment, said effector function is antibody dependent cell cytotoxicity (ADCC). In one embodiment, the antibody fragment binds FcRn.

Aglycosylated antibodies can be produced by a variety of methods known in the art. A convenient method comprises expressing the antibody in a prokaryotic host cell such as E. coli. The amino acids where carbohydrate, such as oligosaccharide, is attached are typically asparagine (N-linkage), serine (O-linkage), and threonine (O-linkage) residues. In order to identify potential glycosylation sites within an antibody or antigen-binding fragment, the sequence of the antibody is examined, for example, by using publicly available databases such as the website provided by the Center for Biological Sequence Analysis (see http://www.cbs.dtu.dk/services/NetNGlyc/ for predicting N-linked glycosylation sites) and http://www.cbs.dtu.dk/services/NetOGlyc/ for predicting O-linked glycosylation sites). Additional methods for altering glycosylation sites of antibodies are described in U.S. Pat. Nos. 6,350,861 and 5,714,350.

Removal of carbohydrate moieties present on the native target antibody may also be accomplished chemically or enzymatically. Chemical deglycosylation requires exposure of the polypeptide to the compound trifluoromethanesulfonic acid, or an equivalent compound. This treatment results in the cleavage of most or all sugars except the linking sugar (N-acetylglucosamine or N-acetylgalactosamine), while leaving the polypeptide intact. Chemical deglycosylation is described by Hakimuddin et al. (Arch. Biochem. Biophys., 259:52 [1987]) and by Edge et al. (Anal. Biochem., 118:131 [1981]). Enzymatic cleavage of carbohydrate moieties on polypeptides can be achieved by the use of a variety of endo- and exo-glycosidases as described by Thotakura et al. (Meth. Enzymol. 138:350 [1987]). Glycosylation at potential glycosylation sites may be prevented by the use of the compound tunicamycin as described by Duskin et al. (J. Biol. Chem., 257:3105 [1982]). Tunicamnycin blocks the formation of protein-N-glycoside linkages.

Thus, in some embodiments, the antibodies of the invention or an antigen-binding fragment thereof is modified to reduce or eliminate potential glycosylation sites. In still another embodiment, the constant region of the antibody, or fragment thereof of the invention is modified to reduce at least one constant region-mediated biological effector function relative to an unmodified antibody.

C. Antibody-Salvage Receptor Binding Epitope Fusions”

In certain embodiments of the invention, it may be desirable to use an antibody fragment, rather than an intact antibody, to increase tumor penetration, for example. In this case, it may be desirable to modify the antibody fragment in order to increase its serum half life. This may be achieved, for example, by incorporation of a salvage receptor binding epitope into the antibody fragment (e.g. by mutation of the appropriate region in the antibody fragment or by incorporating the epitope into a peptide tag that is then fused to the antibody fragment at either end or in the middle, e.g., by DNA or peptide synthesis).

A systematic method for preparing such an antibody variant having an increased in vivo half-life comprises several steps. The first involves identifying the sequence and conformation of a salvage receptor binding epitope of an Fc region of an IgG molecule. Once this epitope is identified, the sequence of the antibody of interest is modified to include the sequence and conformation of the identified binding epitope. After the sequence is mutated, the antibody variant is tested to see if it has a longer in vivo half-life than that of the original antibody. If the antibody variant does not have a longer in vivo half-life upon testing, its sequence is further altered to include the sequence and conformation of the identified binding epitope. The altered antibody is tested for longer in vivo half-life, and this process is continued until a molecule is obtained that exhibits a longer in vivo half-life.

The salvage receptor binding epitope being thus incorporated into the antibody of interest is any suitable such epitope as defined above, and its nature will depend, e.g., on the type of antibody being modified. The transfer is made such that the antibody of interest still possesses the biological activities described herein.

The epitope generally constitutes a region wherein any one or more amino acid residues from one or two loops of a Fc domain are transferred to an analogous position of the antibody fragment. Even more preferably, three or more residues from one or two loops of the Fc domain are transferred. Still more preferred, the epitope is taken from the CH2 domain of the Fc region (e.g., of an IgG) and transferred to the CH1, CH3, or V_{H} region, or more than one such region, of the antibody. Alternatively, the epitope is taken from the CH2 domain of the Fc region and transferred to the C_{L} region or V_{L} region, or both, of the antibody fragment.

D. Antibody Derivatives

The antibodies of the present invention can be further modified to contain additional nonproteinaceous moieties that are known in the art and readily available. Preferably, the moieties suitable for derivatization of the antibody are water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer is attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

Other modifications of the antibody are contemplated herein. For example, the antibody may be linked to one of a variety of nonproteinaceous polymers, e.g., polyethylene glycol, polypropylene glycol, polyoxyalkylenes, or copolymers of polyethylene glycol and polypropylene glycol. The antibody also may be entrapped in microcapsules prepared, for example, by coacervation techniques or by interfacial polymerization (for example, hydroxymethylcellulose or gelatin-microcapsules and poly-(methylmethacylate) microcapsules, respectively), in colloidal drug delivery systems (for example, liposomes, albumin microspheres, microemulsions, nano-particles and nanocapsules), or in macroemulsions. Such techniques are disclosed in Remington's Pharmaceutical Sciences, 16th edition, Oslo, A., Ed., (1980). In vivo Stabilization Using Polymeric Stabilizing Moieties—PEGylation:

Another type of covalent modification of the target antibody, e.g., any one or more of the anti-Her3 antibodies of the invention comprises linking the target antibody to various nonproteinaceous polymers, e.g. polyethylene glycol, polypropylene glycol or polyoxyalkylenes, in the manner set forth in U.S. Pat. No. 4,640,835; 4,496,689; 4,301,144; 4,670,417; 4,791,192 or 4,179,337. Thus, in some embodiments, the antibodies and antibody fragments of the invention may be chemically modified to provide a desired effect such as increased solubility, stability and circulating time of the polypeptide, or decreased immunogenicity.

The antibody or fragments thereof polypeptides may be modified at random positions within the molecule, or at predetermined positions within the molecule and may include one, two, three or more attached chemical moieties. For example, PEGylation of antibodies and antibody fragments of the invention may be carried out by any of the PEGylation reactions known in the art, See for example, EP 0 154 316 by Nishimura et al. and EP 0 401 384 by Ishikawa et al. Methods for determining the degree of substitution are discussed, for example, in Delgado et al., Crit. Rev. Thera. Drug Carrier Sys. 9:249-304 (1992). (each of which is incorporated by reference herein in its entirety). To PEGylate an antibody, the antibody, or fragment thereof, typically is reacted with polyethylene glycol (PEG), such as a reactive ester or aldehyde derivative of PEG, under conditions in which one or more PEG groups become attached to the antibody or antibody fragment. Preferably, the PEGylation is carried out via an acylation reaction or an alkylation reaction with a reactive PEG molecule (or an analogous reactive water-soluble polymer). As used herein, the term “polyethylene glycol” is intended to encompass any of the forms of PEG that have been used to derivatize other proteins, such as mono(C1-C10)alkoxy- or aryloxy-polyethylene glycol or polyethylene glycol-maleimide. In certain embodiments, the antibody to be PEGylated is an aglycosylated antibody

In another aspect, a single immunoglobulin variable domain derived from an invention antibody containing composition is stabilized in vivo by linkage or association with a (non-polypeptide) polymeric stabilizing moiety. Examples of this type of stabilization are described, for example, in WO99/64460 (Chapman et al.) and EP1,160,255 (King et al.), each of which is incorporated herein by reference. Specifically, these references describe the use of synthetic or naturally-occurring polymer molecules, such as polyalkylene, polyalkenylenes, polyoxyalkylenes or polysaccharides, to increase the in vivo half-life of immunoglobulin polypeptides. A typical example of a stabilizing moiety is polyethylene glycol, or PEG, a polyalkylene. The process of linking PEG to an immunoglobulin polypeptide is described in these references and is referred to herein as “PEGylation.” As described therein, an immunoglobulin polypeptide can be PEGylated randomly, as by attachment of PEG to lysine or other amino acids on the surface of the protein, or site-specifically, e.g., through PEG attachment to an artificially introduced surface cysteine residue. Depending upon the immunoglobulin, it may be preferred to use a non-random method of polymer attachment, because random attachment, by attaching in or near the antigen-binding site or sites on the molecule often alters the affinity or specificity of the molecule for its target antigen. Polyethylene glycol can also be attached to proteins using a number of different intervening linkers. For example, U.S. Pat. No. 5,612,460, the entire disclosure of which is incorporated herein by reference, discloses urethane linkers for connecting polyethylene glycol to proteins. Protein-polyethylene glycol conjugates wherein the polyethylene glycol is attached to the protein by a linker can also be produced by reaction of proteins with compounds such as MPEG-succinimidylsuccinate, MPEG activated with 1,1′-carbonyldiimidazole, MPEG-2,4,5-trichloropenylcarbonate, MPEG-p-nitrophenolcarbonate, and various MPEG-succinate derivatives. A number of additional polyethylene glycol derivatives and reaction chemistries for attaching polyethylene glycol to proteins are described in WO 98/32466, the entire disclosure of which is incorporated herein by reference. PEGylated protein products produced using the reaction chemistries set out herein are included within the scope of the invention.

One system for attaching polyethylene glycol directly to amino acid residues of proteins without an intervening linker employs tresylated MPEG, which is produced by the modification of monmethoxy polyethylene glycol (MPEG) using tresylchloride (C1SO₂CH₂CF₃). Upon reaction of protein with tresylated MPEG, polyethylene glycol is directly attached to amine groups of the protein. Thus, the invention includes protein-polyethylene glycol conjugates produced by reacting proteins of the invention with a polyethylene glycol molecule having a 2,2,2-trifluoroethane sulphonyl group.

A general method for preparing PEGylated antibodies and antibody fragments of the invention will generally comprise the steps of (a) reacting the antibody or antibody fragment with polyethylene glycol, such as a reactive ester or aldehyde derivative of PEG, under conditions whereby the antibody or antibody fragment becomes attached to one or more PEG groups, and (b) obtaining the reaction products. It will be apparent to one of ordinary skill in the art to select the optimal reaction conditions or the acylation reactions based on known parameters and the desired result.

There are a number of attachment methods available to those skilled in the art, e.g., EP 0 401 384, herein incorporated by reference (coupling PEG to G-CSF), see also Malik et al., Exp. Hematol. 20:1028-1035 (1992) (reporting PEGylation of GM-CSF using tresyl chloride). For example, polyethylene glycol may be covalently bound through amino acid residues via a reactive group, such as, a free amino or carboxyl group. Reactive groups are those to which an activated polyethylene glycol molecule may be bound. The amino acid residues having a free amino group may include lysine residues and the N-terminal amino acid residues; those having a free carboxyl group may include aspartic acid residues, glutamic acid residues and the C-terminal amino acid residue. Sulfhydryl groups may also be used as a reactive group for attaching the polyethylene glycol molecules. Preferred for therapeutic purposes is attachment at an amino group, such as attachment at the N-terminus or lysine group.

As suggested above, polyethylene glycol may be attached to proteins via linkage to any of a number of amino acid residues. As indicated above, pegylation of the proteins of the invention may be accomplished by any number of means. For example, polyethylene glycol may be attached to the protein either directly or by an intervening linker. Linkerless systems for attaching polyethylene glycol to proteins are described in Delgado et al., Crit. Rev. Thera. Drug Carrier Sys. 9:249-304 (1992); Francis et al., Intern. J. of Hematol. 68:1-18 (1998); U.S. Pat. No. 4,002,531; U.S. Pat. No. 5,349,052; WO 95/06058; and WO 98/32466, the disclosures of each of which are incorporated herein by reference. For example, polyethylene glycol can be linked to a protein via covalent bonds to lysine, histidine, aspartic acid, glutamic acid, or cysteine residues. One or more reaction chemistries may be employed to attach polyethylene glycol to specific amino acid residues (e.g., lysine, histidine, aspartic acid, glutamic acid, or cysteine) of the protein or to more than one type of amino acid residue (e.g, lysine, histidine, aspartic acid, glutamic acid, cysteine and combinations thereof) of the protein.

One may specifically desire proteins chemically modified at the N-terminus. Using polyethylene glycol as an illustration of the present composition, one may select from a variety of polyethylene glycol molecules (by molecular weight, branching, etc.), the proportion of polyethylene glycol molecules to protein (or peptide) molecules in the reaction mix, the type of PEGylation reaction to be performed, and the method of obtaining the selected N-terminally PEGylated protein. The method of obtaining the N-terminally PEGylated preparation (i.e., separating this moiety from other mono-PEGylated moieties if necessary) may be by purification of the N-terminally PEGylated material from a population of PEGylated protein molecules. Selective proteins chemically modified at the N-terminus may be accomplished by reductive alkylation which exploits differential reactivity of different types of primary amino groups (lysine versus the N-terminal) available for derivatization in a particular protein. Under the appropriate reaction conditions, substantially selective derivatization of the protein at the N-terminus with a carbonyl group containing polymer is achieved.

The polymer may be of any molecular weight, and may be branched or unbranched. Branched polyethylene glycols are described, for example, in U.S. Pat. No. 5,643,575; Morpurgo et al., Appl. Biochem. Biotechnol. 56:59-72 (1996); Vorobjev et al., Nucleosides Nucleotides 18:2745-2750(1999); and Caliceti et al., Bioconjug. Chem. 10:638-646 (1999), the disclosures of each of which are incorporated herein by reference. For polyethylene glycol, the preferred molecular weight is between about 1 kDa and about 100 kDa (the term “about” indicating that in preparations of polyethylene glycol, some molecules will weigh more, some less, than the stated molecular weight) for ease in handling and manufacturing. Other sizes may be used, depending on the desired therapeutic profile (e.g., the duration of sustained release desired, the effects, if any on biological activity, the ease in handling, the degree or lack of antigenicity and other known effects of the polyethylene glycol to a therapeutic protein or analog).

It is preferred that the addition of PEG or another polymer does not interfere with the antigen-binding affinity or specificity of the antibody variable domain polypeptide. By “does not interfere with the antigen-binding affinity or specificity” is meant that the PEG-linked antibody single variable domain has an IC₅₀ or ND₅₀ which is no more than 10% greater than the IC₅₀ or ND₅₀, respectively, of a non-PEG-linked antibody variable domain having the same antibody single variable domain. In the alternative, the phrase “does not interfere with the antigen-binding affinity or specificity” means that the PEG-linked form of an antibody single variable domain retains at least 90% of the antigen binding activity of the non-PEGylated form of the polypeptide.

PEGylated antibodies and antibody fragments may generally be used to treat conditions that may be alleviated or modulated by administration of the antibodies and antibody fragments described herein. Generally the PEGylated antibodies and antibody fragments have increased half-life, as compared to the non-PEGylated antibodies and antibody fragments. The PEGylated antibodies and antibody fragments may be employed alone, together, or in combination with other pharmaceutical compositions.

The target antibody may also be entrapped in microcapsules prepared, for example, by coacervation techniques or by interfacial polymerization (for example, hydroxymethylcellulose or gelatin-microcapsules and poly-[methylmethacylate]microcapsules, respectively), in colloidal drug delivery systems (for example, liposomes, albumin microspheres, microemulsions, nano-particles and nanocapsules), or in macroemulsions. Such techniques are disclosed in Remington's Pharmaceutical Sciences, 16th edition, Osol, A., Ed., (1980).

### Antibody-Coated Liposomes and Therapeutics (Immunoliposomes)

Liposomal formulations are often used in therapeutics and pharmaceuticals. However, the biodistribution of liposomes in initial studies meant that such formulations were not widely applicable for use in humans. The technology of “stealth or stealthed” liposomes and formulations was thus developed, which allows liposomes to circulate for longer durations. A preferred agent for use in stealthing liposomes is polyethylene glycol (PEG), and the resultant liposomes are also termed PEGylated liposomes.

Any one of the antibodies or fragments thereof disclosed herein may also be formulated as immunoliposomes. Liposomes containing the antibody are prepared by methods known in the art, such as described in Epstein et al., Proc. Natl. Acad. Sci. USA 82: 3688 (1985); Hwang et al., Proc. Natl. Acad. Sci. USA 77: 4030 (1980); and U.S. Pat. Nos. 4,485,045 and 4,544,545. Liposomes with enhanced circulation time are disclosed in U.S. Pat. No. 5,013,556.

Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter. Fab′ fragments of the antibody of the present invention can be conjugated to the liposomes as described in Martin et al., J. Biol. Chem. 257: 286-288 (1982) via a disulfide interchange reaction. A chemotherapeutic agent (such as Doxorubicin) is optionally contained within the liposome. See Gabizon et al., J. National Cancer Inst. 81 (19): 1484 (1989).

Stealth liposomes have also been proposed for use in delivering cytotoxic agents to tumors in cancer patients. A range of drugs have been incorporated into stealth liposomes, including cisplatin (Rosenthal et al., 2002), TNF.alpha. (Kim et al., 2002), doxorubicin (Symon et al., 1999) and adriamycin (Singh et al., 1999), each reference being specifically incorporated herein by reference. However, recent reports have indicated unexpected low efficacy of stealth liposomal doxorubicin and vinorelbine in the treatment of metastatic breast cancer (Rimassa et al., 2003). See also U.S Patent Application No. 20040170620, the content of which is incorporated in its entirety by reference herein.

Thus, in certain embodiments, the invention provides improved stealthed liposome formulations, in which the stealthed liposomes are functionally associated or “coated” with an antibody that binds to an aminophospholipid or anionic phospholipid, preferably to PS or PE. The 9D2, 304 (ATCC 4545) and like, competing antibodies of the invention are preferred for such uses, although any antibody, or antigen binding region thereof, which binds to an aminophospholipid or anionic phospholipid may be used.

Any stealthed liposome may form the basis of the new liposomal formulations, and preferably a PEGylated liposome will be employed. The stealthed liposomes are “coated”, i.e., operatively or functionally associated with the antibody that binds to an aminophospholipid or anionic phospholipid. The operative or functional association is made such that the antibody retains the ability to specifically bind to the target aminophospholipid or anionic phospholipid, preferably PS or PE, thereby delivering or targeting the stealthed liposome and any contents thereof to PS- and/or PE-positive cells, such as tumor cells and tumor vascular endothelial cells.

The antibody-coated stealthed liposomes of the invention may be used alone. Preferably, however, such liposomes will also contain one or more second therapeutic agents, such as anti-cancer or chemotherapeutic agents (the first therapeutic agent being the antibody itself). The second therapeutic agents are generally described as being within the “core” of the liposome. Any one or more of the second, anti-cancer or chemotherapeutic agents known in the art and/or described herein for conjugation to antibodies, or for combination therapies, may be used in the antibody-coated stealthed liposomes of the invention, for example, any chemotherapeutic or radiotherapeutic agent, cytokine, anti-angiogenic agent or apoptosis-inducing agent. In certain embodiments, preferred chemotherapeutic agents are anti-tubulin drugs, docetaxel and paclitaxel.

Moreover, the antibody-coated stealthed liposomes of the invention may also be loaded with one or more anti-viral drugs for use in treating viral infections and diseases. As with the anti-cancer agents, any one or more of the second, anti-viral drugs known in the art and/or described herein for conjugation to antibodies, or for combination therapies, may be used in the antibody-coated stealthed liposomes of the invention.

In other embodiments of the invention the invention antibodies or antigen-binding fragments thereof are conjugated to albumen using art recognized techniques.

To increase the serum half life of the antibody, one may incorporate a salvage receptor binding epitope into the antibody (especially an antibody fragment) as described in U.S. Pat. No. 5,739,277, for example. As used herein, the term “salvage receptor binding epitope” refers to an epitope of the Fc region of an IgG molecule (e.g., IgG₁, IgG₂, IgG₃, or IgG₄) that is responsible for increasing the in vivo serum half-life of the IgG molecule.

### Species and Molecule Selectivity

The anti-Her3 antibody of the invention including binding fragments thereof demonstrates both species and molecule selectivity. In one aspect, the anti-Her3 antibody of the invention binds to human Her3. Following the teachings of the specification, one may determine the species selectivity for the anti-Her3 antibody using methods well known in the art. For instance, one may determine species selectivity using Western blot, FACS, ELISA or MA. In a preferred embodiment, one may determine the species selectivity using Western blot.

Likewise, one may determine the selectivity of an anti-Her3 antibody for Her3 using methods well known in the art following the teachings of the specification. For instance, one may determine the molecule selectivity using Western blot, FACS, ELISA or RIA. In a preferred embodiment, one may determine the molecular selectivity using Western blot.

### Naked Antibody Therapy

A therapeutically effective amount of a naked fully human anti-Her3 antibody, or fragments thereof can be formulated in a pharmaceutically acceptable excipient. The efficacy of the naked fully human Her3 antibodies and their fragments can also be enhanced by supplementing these naked antibodies with one or more other naked antibodies, with one or more immunoconjugates of fully human invention Her3 antibodies, conjugated with one or more therapeutic agents, including drugs, toxins, immunomodulators, hormones, oligonucleotides, hormone antagonists, enzymes, enzyme inhibitors, therapeutic radionuclides, an angiogenesis inhibitor, etc., administered concurrently or sequentially or according to a prescribed dosing regimen, with the Her3 antibodies or fragments thereof.

### Immunoliposomes

The anti-Her3 antibodies disclosed herein may also be formulated as immunoliposomes. Liposomes containing the antibody are prepared by methods known in the art, such as described in Epstein et al., Proc. Natl. Acad. Sci. USA, 82:3688 (1985); Hwang et al., Proc. Natl. Acad. Sci. USA, 77:4030 (1980); and U.S. Pat. Nos. 4,485,045 and 4,544,545. Liposomes with enhanced circulation time are disclosed in U.S. Pat. No. 5,013,556.

Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter. Fab′ fragments of the antibody of the present invention can be conjugated to the liposomes as described in Martin et al. J. Biol. Chem. 257: 286-288 (1982) via a disulfide interchange reaction. A chemotherapeutic agent (such as Doxorubicin) is optionally contained within the liposome. See Gabizon et al. J. National Cancer Inst. 81(19)1484 (1989)

### Antibody Dependent Enzyme Mediated Prodrug Therapy (ADEPT)

The antibody of the present invention may also be used in ADEPT by conjugating the antibody to a prodrug-activating enzyme which converts a prodrug (e.g., a peptidyl chemotherapeutic agent, see WO81/01145) to an active anti-cancer drug. See, for example, WO 88/07378 and U.S. Pat. No. 4,975,278.

The enzyme component of the immunoconjugate useful for ADEPT includes any enzyme capable of acting on a prodrug in such a way so as to convert it into its more active, cytotoxic form.

Enzymes that are useful in the method of this invention include, but are not limited to, alkaline phosphatase useful for converting phosphate-containing prodrugs into free drugs; arylsulfatase useful for converting sulfate-containing prodrugs into free drugs; cytosine deaminase useful for converting non-toxic 5-fluorocytosine into the anti-cancer drug, 5-fluorouracil; proteases, such as serratia protease, thermolysin, subtilisin, carboxypeptidases and cathepsins (such as cathepsins B and L), that are useful for converting peptide-containing prodrugs into free drugs; D-alanylcarboxylpeptidascs, useful for converting prodrugs that contain D-amino acid substituents; carbohydrate-cleaving enzymes such as beta-galactosidase and neuraminidase useful for converting glycosylated prodrugs into free drugs; beta-lactamase useful for converting drugs derivatized with beta-lactams into free drugs; and penicillin amidases, such as penicillin V amidase or penicillin G amidase, useful for converting drugs derivatized at their amine nitrogens with phenoxylacetyl or phenylacetyl groups, respectively, into free drugs. Alternatively, antibodies with enzymatic activity, also known in the art as “abzymes”, can be used to convert to prodrugs of the invention into free active drugs (Massey, Nature 328: 457-458 (1987)). Antibody-abzyme conjugates can be prepared as described herein for delivery of the abzyme to a tumor cell population.

The enzymes of this invention can be covalently bound to the antibody mutant by techniques well known in the art such as the use of the heterobifunctional crosslinking reagents discussed above. Alternatively, fusion proteins comprising at least the antigen binding region of an antibody of the invention linked to at least a functionally active portion of an enzyme of the invention can be constructed using recombinant DNA techniques well known in the art (Neubergeret et al., Nature 312: 604-608 (1984)).

Antibodies with enzymatic activity, known as catalytic antibodies or “abzymes”, can also be employed to convert prodrugs into active drugs. Abzymes based upon the antibodies of the invention, preferably the 9D2 and 3G4 and like antibodies, thus form another aspect of the present invention. The technical capacity to make abzymes also exists within those of ordinary skill in the art, as exemplified by Massey et al. (1987), specifically incorporated herein by reference for purposes of supplementing the abzyme teaching. Catalytic antibodies capable of catalyzing the breakdown of a prodrug at the carbamate position, such as a nitrogen mustard aryl carbamate, are further contemplated, as described in EP 745,673, specifically incorporated herein by reference.

Screening for Antibodies with Desired Properties

Techniques for generating antibodies have been described above. The antibodies of the present invention can be characterized for their physical/chemical properties and biological functions by various assays known in the art. In some embodiments, antibodies are characterized for any one or more of binding to Her3 receptor protein, and/or reduction or blocking of Her3 receptor activation; and/or reduction or blocking of Her3 receptor downstream molecular signaling; and/or disruption or blocking of Her3 receptor binding to its native ligand, e.g, serrate or delta etc ; and/or promotion of endothelial cell proliferation; and/or inhibition of endothelial cell differentiation; and/or inhibition of arterial differentiation; and/or inhibition of tumor vascular perfusion; and/or treatment and/or prevention of a tumor, cell proliferative disorder or a cancer; and/or treatment or prevention of a disorder associated with Her3 expression and/or activity; and/or treatment or prevention of a disorder associated with Her3 receptor expression and/or activity.

In certain embodiments, antibodies may be selected based upon certain biological characteristics such as for example assessing the growth inhibitory effects of an anti-Her3 antibody of the invention. This property may be assessed by methods known in the art, e.g., using cells which express Her3 receptor either endogenously or following transfection with the Her3 receptor gene. For example, tumor cell lines and Her3 receptor-transfected cells may be treated with an anti-Her3 receptor monoclonal antibody of the invention at various concentrations for a few days (e.g., 2-7) days and stained with crystal violet or MTT or analyzed by some other colorimetric assay. Another method of measuring proliferation would be by comparing ³H-thymidine uptake by the cells treated in the presence or absence of an anti-Her3 receptor antibody of the invention. After antibody treatment, the cells are harvested and the amount of radioactivity incorporated into the DNA quantitated in a scintillation counter. Appropriate positive controls include treatment of a selected cell line with a growth inhibitory antibody known to inhibit growth of that cell line. Preferably, the Her3 receptor agonist will inhibit cell proliferation of a Her3 receptor-expressing tumor cell in vitro or in vivo by about 25-100% compared to the untreated tumor cell, more preferably, by about 30-100%, and even more preferably by about 50-100% or 70-100%, at an antibody concentration of about 0.5 to 30 μg/ml. Growth inhibition can be measured at an antibody concentration of about 0.5 to 30 μg/ml or about 0.5 nM to 200 nM in cell culture, where the growth inhibition is determined 1-10 days after exposure of the tumor cells to the antibody. The antibody is growth inhibitory in vivo if administration of the anti-Her3 receptor antibody at about 1 μg/kg to about 100 mg/kg body weight results in reduction in tumor size or tumor cell proliferation within about 5 days to 3 months from the first administration of the antibody.

The purified antibodies can be further characterized by a series of assays including, but not limited to, N-terminal sequencing, amino acid analysis, non-denaturing size exclusion high pressure liquid chromatography (HPLC), mass spectrometry, ion exchange chromatography and papain digestion.

The assaying method for detecting Her3 using the antibodies of the invention or binding fragments thereof are not particularly limited. Any assaying method can be used, so long as the amount of antibody, antigen or antibody-antigen complex corresponding to the amount of antigen (e.g., the level of Her3) in a fluid to be tested can be detected by chemical or physical means and the amount of the antigen can be calculated from a standard curve prepared from standard solutions containing known amounts of the antigen. Representative immunoassays encompassed by the present invention include, but are not limited to, those described in U.S. Pat. Nos. 4,367,110 (double monoclonal antibody sandwich assay); Wide et al., Kirkham and Hunter, eds. Radioimmunoassay Methods, E. and S. Livingstone, Edinburgh (1970); U.S. Pat. No. 4,452,901 (western blot); Brown et al., J. Biol. Chem. 255: 4980-4983 (1980) (immunoprecipitation of labeled ligand); and Brooks et al., Clin. Exp. Immunol. 39:477 (1980) (immunocytochemistry); immunofluorescence techniques employing a fluorescently labeled antibody, coupled with light microscopic, flow cytometric, or fluorometric detection etc. See also Immunoassays for the 80's, A. Voller et al., eds., University Park, 1981, Zola, Monoclonal Antibodies: A Manual of Techniques, pp. 147-158 (CRC Press, Inc. 1987).

(1) Sandwich assays involve the use of two antibodies, each capable of binding to a different immunogenic portion, or epitope, of the protein to be detected. In a sandwich assay, the test sample analyte is bound by a first antibody which is immobilized on a solid support, and thereafter a second antibody binds to the analyte, thus forming an insoluble three-part complex. See, e.g., U.S. Pat. No. 4,376,110. The second antibody may itself be labeled with a detectable moiety (direct sandwich assays) or may be measured using an anti-immunoglobulin antibody that is labeled with a detectable moiety (indirect sandwich assay). For example, one type of sandwich assay is an ELISA assay, in which case the detectable moiety is an enzyme.

In the sandwich assay, the immobilized antibody of the present invention is reacted with a test fluid (primary reaction), then with a labeled form of antibody of the present invention (secondary reaction), and the activity of the labeling agent on the immobilizing carrier is measured, whereby the Her3 level in the test fluid can be quantified. The primary and secondary reactions may be performed simultaneously or with some time intervals. The methods of labeling and immobilization can be performed by modifications of those methods described above. In the immunoassay by the sandwich assay, the antibody used for immobilized or labeled antibody is not necessarily from one species, but a mixture of two or more species of antibodies may be used to increase the measurement sensitivity, etc. In the method of assaying Her3 by the sandwich assay, for example, when the antibodies used in the primary reaction recognize the partial peptides at the C-terminal region of Her3, the antibodies used in the secondary reaction are preferably those recognizing partial peptides other than the C-terminal region (i.e., the N-terminal region). When the antibodies used for the primary reaction recognize partial peptides at the N-terminal region of Her3, the antibodies used in the secondary reaction, antibodies recognizing partial peptides other than the N-terminal region (i.e., the C-terminal region) are preferably employed.

Other types of “sandwich” assays, which can also be useful for detecting Her3, are the so-called “simultaneous” and “reverse” assays. A simultaneous assay involves a single incubation step wherein the antibody bound to the solid support and labeled antibody are both added to the sample being tested at the same time. After the incubation is completed, the solid support is washed to remove the residue of fluid sample and uncomplexed labeled antibody. The presence of labeled antibody associated with the solid support is then determined as it would be in a conventional “forward” sandwich assay.

In the “reverse” assay, stepwise addition first of a solution of labeled antibody to the fluid sample followed by the addition of unlabeled antibody bound to a solid support after a suitable incubation period, is utilized. After a second incubation, the solid phase is washed in conventional fashion to free it of the residue of the sample being tested and the solution of unreacted labeled antibody. The determination of labeled antibody associated with a solid support is then determined as in the “simultaneous” and “forward” assays. In one embodiment, a combination of antibodies of the present invention specific for separate epitopes can be used to construct a sensitive three-site immunoradiometric assay.

This type of assays may also be used to quantify Her3 expression in whatever “sample” it may present itself. Thus, in certain aspects, the sandwich assay includes:

(i) a method for quantifying expression levels of Her3 in a test fluid, comprising reacting the antibody specifically reacting with a partial peptide at the N-terminal region of the Her3 immobilized on a carrier, a labeled form of the antibody specifically reacting with a partial peptide at the C-terminal region and the test fluid, and measuring the activity of the label; or

(ii) a method for quantifying Her3 expression in a test fluid, comprising reacting the antibody specifically reacting with a partial peptide at the C-terminal region of the Her3 immobilized onto a carrier, the antibody specifically reacting with a partial peptide at the N-terminal region of a labeled form of the Her3 and the test fluid, and measuring the activity of the label; etc.

(2) Competitive Assay

Competitive binding assays rely on the ability of a labeled standard to compete with the test sample analyte for binding with a limited amount of antibody. The amount of Her3 protein in the test sample is inversely proportional to the amount of standard that becomes bound to the antibodies. To facilitate determining the amount of standard that becomes bound, the antibodies generally are insolubilized before or after the competition, so that the standard and analyte that are bound to the antibodies may conveniently be separated from the standard and analyte which remain unbound.

For quantifying the level of Her3 expression, one skilled in the art may combine and/or competitively react antibodies of the invention or fragments thereof a test fluid and a labeled form of Her3, measure a ratio of the labeled Her3 bound to the antibodies or fragments thereof b to thereby quantify the Her3 in the test fluid.

(3) Immunometric Assay

In the immunometric assay, an antigen in a test fluid and a solid phase antigen are competitively reacted with a given amount of a labeled form of the antibody of the present invention followed by separating the solid phase from the liquid phase; or an antigen in a test fluid and an excess amount of labeled form of the antibody of the present invention are reacted, then a solid phase antigen is added to bind an unreacted labeled form of the antibody of the present invention to the solid phase and the solid phase is then separated from the liquid phase. Thereafter, the labeled amount of any of the phases is measured to determine the antigen level in the test fluid.

Typical, and preferred, immunometric assays include “forward” assays in which the antibody bound to the solid phase is first contacted with the sample being tested to extract the Her3 from the sample by formation of a binary solid phase antibody-Her3 complex. After a suitable incubation period, the solid support is washed to remove the residue of the fluid sample, including unreacted Her3, if any, and then contacted with the solution containing a known quantity of labeled antibody (which functions as a “reporter molecule”). After a second incubation period to permit the labeled antibody to complex with the Her3 bound to the solid support through the unlabeled antibody, the solid support is washed a second time to remove the unreacted labeled antibody. This type of forward sandwich assay can be a simple “yes/no” assay to determine whether Her3 is present or can be made quantitative by comparing the measure of labeled antibody with that obtained for a standard sample containing known quantities of Her3. Such “two-site” or “sandwich” assays are described by Wide (Radioimmune Assay Method, Kirkham, ed., Livingstone, Edinburgh, 1970, pp. 199 206).

(4) Nephrometry

In the nephrometry, the amount of insoluble sediment, which is produced as a result of the antigen-antibody reaction in a gel or in a solution, is measured. Even when the amount of an antigen in a test fluid is small and only a small amount of the sediment is obtained, a laser nephrometry utilizing laser scattering can be suitably used.

Examples of labeling agents, which may be used in the above referenced assay methods (1) to (4) using labeling agents, include radioisotopes (e.g., ¹²⁵I, ¹³¹I, ³H, ¹⁴C, ³²P, ³³P, ³⁵S, etc., fluorescent substances, e.g., cyanine fluorescent dyes (e.g., Cy2, Cy3, Cy5, Cy5.5, Cy7), fluorescamine, fluorescein isothiocyanate, etc., enzymes (e.g., .beta.-galactosidase, .beta.-glucosidase, alkaline phosphatase, peroxidase, malate dehydrogenase, etc.), luminescent substances (e.g., luminol, a luminol derivative, luciferin, lucigenin, etc.), biotin, lanthanides, etc. In addition, a biotin-avidin system may be used as well for binding an antibody to a labeling agent.

In the immobilization of antigens or antibodies, physical adsorption may be used. Alternatively, chemical binding that is conventionally used for immobilization of proteins, enzymes, etc. may be used as well. Examples of the carrier include insoluble polysaccharides such as agarose, dextran, cellulose, etc.; synthetic resins such as polystyrene, polyacrylamide, silicone, etc.; or glass; and the like.

In certain embodiments, the antibodies of the present invention are tested for their antigen binding activity. The antigen binding assays that are known in the art and can be used herein include without limitation any direct or competitive binding assays using techniques such as western blots, radioimmunoassays, ELISA (enzyme linked immunosorbent assay), “sandwich” immunoassays, immunoprecipitation assays, fluorescent immunoassays, and protein A immunoassays. Illustrative antigen binding assay are provided herein.

In some embodiments, the binding affinity of anti Her3 antibodies is determined. Antibodies of the invention preferably have a binding affinity (K_{D}) to Her3 of at least about 1×10⁻⁷ M, more preferably at least about 1×10⁻⁸ M, more preferably at least about 1×10⁻⁹ M, and most preferably at least about 1×10⁻¹° M. Preferred antibody-producing cells of the invention produce substantially only antibodies having a binding affinity to Her3 of at least about 1×10⁻⁷ M, more preferably at least about 1×10⁻⁸ M, more preferably at least about 1×10⁻⁹ M, and most preferably at least about 1×10⁻¹⁰ M. Preferred compositions of the invention comprise substantially only antibodies having a binding affinity to Her3 of at least about 1×10⁻⁷M, more preferably at least about 1×10⁻⁸ M, more preferably at least about 1×10⁻⁹ M, and most preferably at least about 1×10⁻¹° M.

In another aspect of the invention, the antibodies of the invention bind to Her3 with substantially the same K_{D} as an antibody that comprises one of the amino acid sequences selected from the group as set forth in one of Appendix I or II. In another embodiment, the antibody binds to Her3 with substantially the same K_{D} as an antibody that comprises one or more CDRs from an antibody that comprises one of the amino acid sequences set forth herein. Anti-Her3 antibodies according to the invention or identified using the methods disclosed herein have a low dissociation rate. In one embodiment, the anti-Her3 antibody has a K_{off} of 1×10⁻⁴ or lower, preferably a K_{off} that is 5×10⁻⁵ or lower.

In another embodiment, the antibodies of the invention or those identified or produced using the methods of the invention bind to Her3 with substantially the same K_{off} as an antibody that comprises one or more CDRs disclosed herein. Illustrative assays for affinity analysis are described herein.

### Affinity Analysis for Epitope(s)

Affinity can be either absolute or relative. By absolute affinity, it is meant that the assay for affinity gives defined numerical determinations of the affinity of one compound for another. Comparison of the affinity of the complex being tested to that of a reference compound whose binding affinity is known allows for the determination of relative binding affinity of the test ligand.

Whether absolute or relative, affinity of one molecule for another can be measured by any method known in the art. By way of non-limiting example, such methods include competition assays, surface plasmon resonance, half-maximal binding assays, competition assays, Scatchard analysis, direct force techniques (Wong et al., Direct force measurements of the streptavidin-biotin interaction, Biomol. Eng. 16:45-55, 1999), and mass spectrometry (Downard, Contributions of mass spectrometry to structural immunology, J. Mass Spectrom. 35:493-503, 2000).

The binding affinity and dissociation rate of an antibody to Her3 may be determined by any method known in the art. For example, the binding affinity can be measured by competitive ELISAs, RIAs or surface plasmon resonance, such as BIAcore. The dissociation rate can also be measured by surface plasmon resonance. Alternatively, the binding affinity and dissociation rate is measured by surface plasmon resonance. More, the binding affinity and dissociation rate is measured using a BIAcore. See below for a brief description, it being understood the invention is not limited to the specific assays detailed herein.

1. Absolute Affinity

As regards absolute affinity, “low affinity” refers to binding wherein the dissociation constant (K_{D}) between two molecules is about 10⁻⁵ M to 10⁻⁷ M. “Moderate affinity” refers to binding wherein the dissociation constant (K_{D}) between two molecules is at least about 10⁻⁷M to 10⁻⁸ M. “High affinity” refers to a binding wherein the association constant between the two molecules is at least about 10⁻⁸ M to about 10⁻¹⁴ M, and preferably about 10⁻⁹ M to about 10⁻¹⁴ M, more preferably about 10⁻¹⁰ M to about 10⁻¹⁴ M, and most preferably greater than about 10⁻¹⁴ M.

The dissociation constant, K_{D}, is an equilibrium constant for the dissociation of one species into two, e.g., the dissociation of a complex of two or more molecules into its components, for example, dissociation of a substrate from an enzyme. Exemplary K_{D} values for compositions of the present invention are from about 10⁻⁷ M (100 nM) to about 10⁻¹² M (0.001 nM). The stability constant is an equilibrium constant that expresses the propensity of a species to form from its component parts. The larger the stability constant, the more stable is the species. The stability constant (formation constant) is the reciprocal of the instability constant (dissociation constant).

The affinity of an invention antibody for a target epitope, or the affinity of a bi-specific antibody for a carrier epitope, is driven by non-covalent interactions. There are four main non-covalent attractive forces between molecules: (i) electrostatic forces, which occur between oppositely charged molecules such as amino groups and carboxylic groups; (ii) hydrogen bonds, which are formed when hydrogen atoms are shared between electronegative atoms such as nitrogen and oxygen; (iii) Van der Waals forces, which are generated between electron clouds around molecules oppositely polarized by neighboring atoms; and (iv) hydrophobic interactions, which are formed when water is excluded from the interface allowing hydrophobic molecules to interact in a waterless environment.

Non-covalent interactions can, but rarely do, have the strength of a covalent linkage (i.e., a chemical bond). In some instances, the affinity of the invention antibody for a target epitope, although driven by non-covalent interactions, is so high as to approach the strength of a covalent bond. This provides for invention antibodies that are very stable relative to other Her3 receptor antibodies of the invention.

Preferably, the affinity of an invention antibody for its cognate target epitope, is a K_{D} of about 100 nM to about 0.01 nM; more preferably, greater than about 100 nM, or greater than about 10 nM; most preferably, greater than about 1 nM, or greater than about 0.1 nM. Typical K_{D} for target epitopes are from about 0.1 nM to 100 nM, preferably from about 0.1 nM to 10 nM, more preferably from about 0.5 nM to 5 nM, or about 1 nM.

In the invention, when multiple copies of a carrier epitope are present on the antibody, the affinity of an antibody for its cognate carrier epitope may be greater than the affinity of an antibody for a free carrier epitope or for a monovalent antibody comprising the carrier epitope. Additionally or alternatively, a multivalent targetable construct having x carrier epitopes has a greater affinity for its target epitope than would x number of constructs. Put another way, the compositions of the invention also provides for synergistic, rather than merely additive, binding effects.

2. Surface Plasmon Resonance

Binding parameters such as K_{D} may be measured using surface plasmon resonance on a chip, for example, with a BIAcore™ chip coated with immobilized binding components. Surface plasmon resonance is used to characterize the microscopic association and dissociation constants of reaction between an antibody or antibody fragment and its ligand. Such methods are generally described in the following references which are incorporated herein by reference. (Vely et al., BIAcore analysis to test phosphopeptide-SH2 domain interactions, Meth. Mol. Biol. 121:313-21, 2000; Liparoto et al., Biosensor analysis of the interleukin-2 receptor complex, J. Mol. Recog. 12:316-21, 1999; Lipschultz et al., Experimental design for analysis of complex kinetics using surface plasmon resonance, Methods 20:310-8, 2000; Malmqvist, BIACORE: an affinity biosensor system for characterization of biomolecular interactions, Biochem. Soc. Transactions 27:33540, 1999; Alfthan, Surface plasmon resonance biosensors as a tool in antibody engineering, Biosensors & Bioelectronics 13:653-63, 1998; Fivash et al., BIAcore for macromolecular interaction, Curr. Opin. Biotech. 9:97-101, 1998; Price et al., Summary report on the ISOBM TD-4 Workshop: analysis of 56 monoclonal antibodies against the MUC1 mucin, Tumour Biol. 19 Suppl 1:1-20, 1998; Malmqvist et al., Biomolecular interaction analysis: affinity biosensor technologies for functional analysis of proteins, Curr. Opin. Chem. Biol. 1:378-83, 1997; O'Shannessy et al., Interpretation of deviations from pseudo-first-order kinetic behavior in the characterization of ligand binding by biosensor technology, Anal. Biochem. 236:275-83, 1996; Malmborg et al., BIAcore as a tool in antibody engineering, J. Immunol. Meth. 183:7-13, 1995; Van Regenmortel, Use of biosensors to characterize recombinant proteins, Dev. Biol. Standardization 83:143-51, 1994; O'Shannessy, Determination of kinetic rate and equilibrium binding constants for macromolecular interactions: a critique of the surface plasmon resonance literature, Curr. Opin. Biotechnol. 5:65-71, 1994). Models using BIAcore to examine the binding of fixed ligands to multivalent compounds have been described (Muller et al., Model and simulation of multivalent binding to fixed ligands, Anal. Biochem. 261:149-158, 1998).

BIAcore™ uses the optical properties of surface plasmon resonance (SPR) to detect alterations in protein concentration bound within to a dextran matrix lying on the surface of a gold/glass sensor chip interface, a dextran biosensor matrix. In brief, proteins are covalently bound to the dextran matrix at a known concentration and a ligand for the protein (e.g., antibody) is injected through the dextran matrix. Near infrared light, directed onto the opposite side of the sensor chip surface is reflected and also induces an evanescent wave in the gold film, which in turn, causes an intensity dip in the reflected light at a particular angle known as the resonance angle. If the refractive index of the sensor chip surface is altered (e.g., by ligand binding to the bound protein) a shift occurs in the resonance angle. This angle shift can be measured and is expressed as resonance units (RUs) such that 1000 RUs is equivalent to a change in surface protein concentration of 1 ng/mm 2. These changes are displayed with respect to time along the y-axis of a sensorgram, which depicts the association and dissociation of any biological reaction.

Additional details may be found in Jonsson et al., Introducing a biosensor based technology for real-time biospecific interaction analysis, Ann. Biol. Clin. 51:19-26, 1993; Jonsson et al., Real-time biospecific interaction analysis using surface plasmon resonance and a sensor chip technology, Biotechniques 11:620-627, 1991; Johnsson et al., Comparison of methods for immobilization to carboxymethyl dextran sensor surfaces by analysis of the specific activity of monoclonal antibodies, J. Mal. Recog. 8:125-131, 1995; and Johnsson, Immobilization of proteins to a carboxymethyldextran-modified gold surface for biospecific interaction analysis in surface plasmon resonance sensors, Anal. Biochem. 198:268-277, 1991; Karlsson et al., Kinetic analysis of monoclonal antibody-antigen interactions with a new biosensor based analytical system, J. Immunol. Meth. 145:229, 1991; Weinberger et al., Recent trends in protein biochip technology, Pharmacogenomics 1:395-416, 2000; Lipschultz et al., Experimental design for analysis of complex kinetics using surface plasmon resonance, Methods 20:310-8, 2000.

3. Relative Affinity

Affinity may also be defined in relative terms, e.g., by IC₅₀. In the context of affinity, the IC₅₀ of a compound is the concentration of that compound at which 50% of a reference ligand is displaced from a target epitope in vitro or targeted tissue in vivo. Typically, IC₅₀ is determined by competitive ELISA. In still another embodiment, the invention provides anti-Her3 monoclonal antibodies that compete with a conventional anti-Her3 antibody for binding to Her3 receptor protein. Such competitor antibodies include antibodies that recognize a Her3 epitope that is the same as or overlaps with the Her3 epitope recognized by any one of a conventional antibody. Such competitor antibodies can be obtained by assay well known to one skilled in the art. For example, they can b obtained by screening anti-Her3 hybridoma supernatants for binding to immobilized Her3 in competition with labeled 26.6, 26.14, 26.20, 26.34, and/or 26.82 antibodies. Alternatively, they can be used in a binding assay A hybridoma supernatant containing competitor antibody will reduce the amount of bound, labeled antibody detected in the subject competition binding mixture as compared to the amount of bound, labeled antibody detected in a control binding mixture containing irrelevant (or no) antibody. Any of the competition binding assays described herein are suitable for use in the foregoing procedure.

Anti-Her3 antibodies of the invention possessing the unique properties described herein can be obtained by screening anti-Her3 hybridoma clones for the desired properties by any convenient method. For example, if an anti-Her3 monoclonal antibody that blocks or does not block the binding of Her3 receptors to its binding partner e.g., a Her3 ligand is desired, the candidate antibody can be tested in a binding competition assay, such as a competitive binding ELISA, wherein plate wells are coated with the binding partner, and a solution of antibody in an excess of the Her3 receptor of interest is layered onto the coated plates, and bound antibody is detected enzymatically, e.g. contacting the bound antibody with HRP-conjugated anti-Ig antibody or biotinylated anti-Ig antibody and developing the HRP color reaction., e.g. by developing plates with streptavidin-HRP and/or hydrogen peroxide and detecting the HRP color reaction by spectrophotometry at 490 nm with an ELISA plate reader.

In one embodiment, the present invention contemplates an altered antibody that possesses some but not all effector functions, which make it a desired candidate for many applications in which the half life of the antibody in vivo is important yet certain effector functions (such as complement and ADCC) are unnecessary or deleterious. In certain embodiments, the Fc activities of the produced immunoglobulin are measured to ensure that only the desired properties are maintained. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol 9:457-92 (1991). An example of an in vitro assay to assess ADCC activity of a molecule of interest is described in U.S. Pat. No. 5,500,362 or 5,821,337. Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. Proc. Natl. Acad. Sci. A 95:652-656 (1998). C1q binding assays may also be carried out to confirm that the antibody is unable to bind C1q and hence lacks CDC activity. To assess complement activation, a CDC assay, e.g. as described in Gazzano-Santoro et al., J. Immunol. Methods 202:163 (1996), may be performed. FcRn binding and in vivo clearance/half life determinations can also be performed using methods known in the art, e.g. those described in the Examples section.

### Identification of Her3 Epitopes Recognized by Anti-Her3 Antibody

One may determine whether an anti-Her3 antibody derived from the antibodies of the invention or produced in accordance with the methods described above binds to the same antigen as another antibody, e.g., conventional antibody using a variety of methods known in the art. For instance, one may determine whether a test anti-Her3 antibody binds to the same antigen by using an anti-Her3 antibody to capture an antigen that is known to bind to the anti-Her3 antibody, eluting the antigen from the antibody, and then determining whether the test antibody will bind to the eluted antigen.

One may determine whether a test antibody binds to the same epitope as an anti-Her3 antibody by binding the anti-Her3 antibody to Her3 receptor protein under saturating conditions, and then measuring the ability of the test antibody to bind to Her3. If the test antibody, e.g., anti-Her3 antibodies derived from the invention antibodies or in accordance with the methods of the invention is able to bind to the Her3 receptor protein at the same time as the reference anti-Her3 antibody, then the test antibody binds to a different epitope as the anti-Her3 antibody. However, if the test antibody is not able to bind to Her3 receptor protein at the same time, then the test antibody binds to the same epitope as the human anti-Her3 antibody. This experiment may be performed using ELISA, RIA or surface plasmon resonance. In certain embodiments, the experiment is performed using surface plasmon resonance, supra. In another embodiment, BIAcore is used, see supra. One may also determine whether an anti-Her3 antibody cross-competes with a reference anti-Her3 antibody. For example, one may determine whether a test anti-Her3 antibody cross-competes with another by using the same method that is used to measure whether the anti-Her3 antibody is able to bind to the same epitope as another anti-Her3 antibody.

The diagnostic method may also be used to determine whether a tumor is potentially cancerous, if it expresses high levels of Her3, or benign, if it expresses low levels of Her3. Thus, for example, biological samples obtained from patients suspected of exhibiting an oncogenic disorder mediated by Her3 may be assayed for the presence of Her3 expressing cells.

As noted, the anti-Her3 antibodies of the invention may be used to determine the levels of Her3 receptor protein in a tissue or in cells derived from the tissue. In one embodiment, the tissue is a diseased tissue. In a more preferred embodiment, the tissue is a tumor or a biopsy thereof. In a preferred embodiment of the method, a tissue or a biopsy thereof is excised from a patient. The tissue or biopsy is then used in an immunoassay to determine, e.g., Her3 levels, cell surface levels of Her3, levels of tyrosine phosphorylation of Her3, or localization of Her3 by the methods discussed herein. The method can be used to determine tumors that express Her3.

In a related embodiment, the present invention provides methods for diagnosing cancers by assaying for changes in the level of Her3 in cells, tissues or body fluids compared with the levels in cells, tissues, or body fluids, preferably of the same type in a control sample. A change, especially an increase, in levels of Her3 in the patient versus the control is associated with the presence of cancer. Typically, for a quantitative diagnostic assay, a positive result indicating that the patient being tested has cancer is one in which levels of Her3 in or on cells, tissues or body fluid are at least two times higher, and preferably three to five times higher, or greater, than the levels of the antigens in or on the same cells, tissues, or body fluid of the control. Normal controls include a human without cancer and/or non-cancerous samples from the patient.

The in vitro diagnostic methods may include any method known to one skilled in the art including immunohistological or immunohistochemical detection of tumor cells (e.g., on human tissue, or on cells dissociated from excised tumor specimens), or serological detection of tumor associated antigens (e.g., in blood samples or other biological fluids). Immunohistochemical techniques involve staining a biological specimen, such as a tissue specimen, with one or more of the antibodies of the invention and then detecting the presence on the specimen of antibody-antigen complexes comprising antibodies bound to the cognate antigen. The formation of such antibody-antigen complexes with the specimen indicates the presence of cancer in the tissue.

Detection of the antibody on the specimen can be accomplished using techniques known in the art such as immunoenzymatic techniques, e.g., immunoperoxidase staining technique, or the avidin-biotin technique, or immunofluorescence techniques (see, e.g., Ciocca et al., 1986, “Immunohistochemical Techniques Using Monoclonal Antibodies”, Meth. Enzymol., 121:562 79 and Introduction to Immunology, Ed. Kimball, (2^{nd} Ed), Macmillan Publishing Company, 1986, pp. 113 117). Those skilled in the art can determine operative and optimal assay conditions by routine experimentation.

In another embodiment, the present invention assists in the diagnosis of cancers and tumors by the identification and measurement of the Her3 receptor protein levels in biological samples. If Her3 receptor protein is normally present, and the development of the oncogenic disorder is caused by an abnormal quantity of the cell surface receptor (Her3), e.g., expression relative to normal, the assay should compare Her3 levels in the biological sample to the range expected in normal, non-oncogenic tissue of the same cell type. Thus, a statistically significant increase in the amount of Her3 bearing cells or Her3 expression level in the subject relative to the control subject or subject's baseline, can be a factor that may lead to a diagnosis of an oncogenic disorder that is progressing or at risk for such a disorder. Likewise, the presence of high levels of Her3 indicative of cancers likely to metastasize can also be detected. For those cancers that express the antigen recognized by the antibodies of the invention, e.g., Her3, the ability to detect the antigen provides early diagnosis, thereby affording the opportunity for early treatment. Early detection is especially important for cancers difficult to diagnose in their early stages.

Moreover, the level of antigen detected and measured in a body fluid sample such as for example diseased tissue provides a means for monitoring the course of therapy for the cancer or tumor, including, but not limited to, surgery, chemotherapy, radiation therapy, the therapeutic methods of the present invention, and combinations thereof. By correlating the level of the antigen in the tissue sample with the severity of disease, the level of such antigen can be used to indicate successful removal of the primary tumor, cancer, and/or metastases, for example, as well as to indicate and/or monitor the effectiveness of other therapies over time. For example, a decrease in the level of the cancer or tumor-specific antigen over time indicates a reduced tumor burden in the patient. By contrast, no change, or an increase, in the level of antigen over time indicates ineffectiveness of therapy, or the continued growth of the tumor or cancer.

A typical in vitro immunoassay for detecting Her3 comprises incubating a biological sample in the presence of a detectably labeled anti-Her3 antibody or antigen binding fragment of the present invention capable of selectively binding to Her3, and detecting the labeled fragment or antibody which is bound in a sample. The antibody is bound to a label effective to permit detection of the cells or portions (e.g., Her3 or fragments thereof liberated from hyperplastic, dysplastic and/or cancerous cells) thereof upon binding of the antibody to the cells or portions thereof. The presence of any cells or portions thereof in the biological sample is detected by detection of the label.

The biological sample may be brought into contact with, and immobilized onto, a solid phase support or carrier, such as nitrocellulose, or other solid support or matrix, which is capable of immobilizing cells, cell particles, membranes, or soluble proteins. The support may then be washed with suitable buffers, followed by treatment with the detectably-labeled anti-Her3 antibody. The solid phase support may then be washed with buffer a second time to remove unbound antibody. The amount of bound label on the solid support may then be detected by conventional means. Accordingly, in another embodiment of the present invention, compositions are provided comprising the monoclonal antibodies, or binding fragments thereof, bound to a solid phase support, such as described herein.

In vitro assays in accordance with the present invention also include the use of isolated membranes from cells expressing a recombinant Her3, soluble fragments comprising the ligand binding segments of Her3, or fragments attached to solid phase substrates. These assays allow for the diagnostic determination of the effects of either binding segment mutations and modifications, or ligand mutations and modifications, e.g., ligand analogues.

In certain embodiments the monoclonal antibodies and binding fragments thereof of the present invention may be used in in vitro assays designed to screen compounds for binding affinity to Her3, See Fodor et al. Science 251: 767-773 (1991), incorporated herein by reference. In accordance with this objective, the invention contemplates a competitive drug screening assay, where the monoclonal antibodies or fragments thereof of the invention compete with a test compound for binding to Her3. In this manner the monoclonal antibodies and fragments thereof are used to detect the presence of any polypeptide which shares one or more binding sites of the Her3 and can be used to occupy binding sites on the receptor which might otherwise be occupied by the antibody.

In certain embodiments, the anti-Her3 antibodies of the invention may be used to determine or quantify the amount of Her3 on the cell surface after treatment of the cells with various compounds. This method can be used to test compounds that may be used to activate or inhibit Her3. In this method, one sample of cells is treated with a test compound for a period of time while another sample is left untreated. If the total level of Her3 is to be measured, the cells are lysed and the total Her3 level is measured using one of the immunoassays described herein.

A preferred immunoassay for measuring total Her3 receptor protein levels is an ELISA or Western blot. If only the cell surface level of Her3 is to be measured, the cells are not lysed, and the cell surface levels of Her3 are measured using any one or more of the assays known to the skilled artisan, e.g., one of the immunoassays described herein. A preferred immunoassay for determining cell surface levels of Her3 includes the steps of labeling the cell surface proteins with a detectable label, such as biotin or ¹²⁵I, immunoprecipitating the Her3 with an anti-Her3 antibody and then detecting the labeled Her3. Another preferred immunoassay for determining the localization of Her3, e.g., cell surface levels, is by using immunohistochemistry.

As well, provided herein is a method to determine whether a conventional anti-Her3 antibody decreases Her3 expression on a target tumor tissue or cell. The term “conventional Her3 antagonist” “conventional treatment with a Her3 moiety” is used interchangeably to mean Her3 specific monoclonal antibodies currently available that specifically target Her3 expression and do not bind to the same epitope as the antibodies of the invention.

A further aspect of the invention is an assessment of the susceptibility that an individual has for developing cancer mediated by Her3. The method comprises the steps of measuring the level of expression of Her3 in a cell or tissue of interest, incubating the cell or tissue with an anti-Her3 antibody or antigen-binding portion thereof, then re-measuring the level of Her3 expression with an anti-Her3 antibody or antigen binding fragment of the invention in the cell or tissue. Alternatively, ICD expression levels may be measured in the above example. A diagnosis that levels of Her3 are low could be used for predicting that the patient is responding to treatment with the conventional anti-Her3 antibody regiment. On the contrary, no change in the level of Her3 or an increase in expression of Her3 after treatment with a conventional anti-Her3 antibody indicate that the patient is either unresponsive to the current treatment protocol or unlikely to respond to further treatment with the conventional anti-Her3 antibody, thereby allowing for earlier intervention. The anti-Her3 antibodies of the invention may be used in the above diagnostic assays either simultaneously with administration of the conventional Her3 antibody or after treatment with the conventional anti-Her3 antibody. Preferably, the conventional Her3 antibody does not compete with the anti-Her3 antibody of the invention for binding Her3 protein. The above assays can be performed iteratively over a period of time to assess the therapeutic efficacy of a conventional anti-Her3 antibody based therapeutic protocol.

In this way, the anti-Her3 antibody of the invention can be used as a “negative biomarker” allowing it to be used to assess the treatment and therapeutic protocol of a conventional anti-Her3 antibody based therapy.

### Vectors, Host Cells and Recombinant Methods

The invention also includes nucleic acids encoding the heavy chain and/or light chain of the anti-Her3 antibodies of the invention. Nucleic acids of the invention also include fragments of the nucleic acids of the invention. A “fragment” refers to a nucleic acid sequence that is preferably of sufficient length to encode a functionally active fragment of the invention antibodies, e.g., light or heavy chain. A “fragment” can also mean the whole coding sequence of a gene and may include 5′ and 3′ untranslated regions.

Constructs of any one or more polynucleotides having sequences as set forth herein can be generated synthetically. Alternatively, single-step assembly of a gene and entire plasmid from large numbers of oligodeoxyribonucleotides is described by, e.g., Stemmer et al., Gene (Amsterdam) (1995) 164(1):49-53. In this method, assembly PCR (the synthesis of long DNA sequences from large numbers of oligodeoxyribonucleotides) is derived from DNA shuffling (Stemmer, Nature (1994) 370:389-391).

Appropriate polynucleotide constructs are purified using standard recombinant DNA techniques as described in, for example, Sambrook et al., Molecular Cloning: A Laboratory Manual, 2nd Ed., (1989) Cold Spring Harbor Press, Cold Spring Harbor, N.Y. The gene product encoded by a polynucleotide of the invention is expressed in any expression system, including, for example, bacterial, yeast, insect, amphibian and mammalian systems. Vectors, host cells and methods for obtaining expression in same are well known in the art. Suitable vectors and host cells are described in U.S. Pat. No. 5,654,173.

Polynucleotide molecules comprising a polynucleotide sequence provided herein are generally propagated by placing the molecule in a vector. Viral and non-viral vectors are used, including plasmids. The choice of plasmid will depend on the type of cell in which propagation is desired and the purpose of propagation. Certain vectors are useful for amplifying and making large amounts of the desired DNA sequence. Other vectors are suitable for expression in cells in culture. Still other vectors are suitable for transfer and expression in cells in a whole animal or person. The choice of appropriate vector is well within the skill of the art. Many such vectors are available commercially. Methods for preparation of vectors comprising a desired sequence are well known in the art.

The polynucleotides set forth in any one or more of SEQ ID NOs set forth in one or more appendices disclosed herein or their corresponding full-length polynucleotides are linked to regulatory sequences as appropriate to obtain the desired expression properties. These can include promoters (attached either at the 5′ end of the sense strand or at the 3′ end of the antisense strand), enhancers, terminators, operators, repressors, and inducers. The promoters can be regulated or constitutive. In some situations it may be desirable to use conditionally active promoters, such as tissue-specific or developmental stage-specific promoters. These are linked to the desired nucleotide sequence using the techniques described above for linkage to vectors. Any techniques known in the art can be used.

When any appropriate host cells or organisms are used to replicate and/or express the polynucleotides or nucleic acids of the invention, the resulting replicated nucleic acid, RNA, expressed protein or polypeptide, is within the scope of the invention as a product of the host cell or organism. The product is recovered by any appropriate means known in the art.

Expression of a target gene, e.g., corresponding to any one or more of the nucleic acid molecules set forth herein can be regulated in the cell to which the gene is native. For example, an endogenous gene of a cell can be regulated by an exogenous regulatory sequence as disclosed in U.S. Pat. No. 5,641,670.

The encoded antibody heavy chain preferably comprises an amino acid sequence selected from the group consisting of SEQ ID Nos as set forth in one of Appendix I-III. The encoded antibody light chain preferably comprises an amino acid sequence a set forth in one of the appendices set forth herein.

In some embodiments, the invention provides nucleic acids encoding both a heavy chain and a light chain of an antibody of the invention. For example, a nucleic acid of the invention may comprise a nucleic acid sequence (Appendix I) encoding an amino acid sequence as set forth in one of Appendix I or III and a nucleic acid sequence (Appendix I) encoding an amino acid sequence as set forth in one of Appendix II or III.

Nucleic acids of the invention include nucleic acids having at least 80%, more preferably at least about 90%, more preferably at least about 95%, and most preferably at least about 98% homology to nucleic acids of the invention. The terms “percent similarity”, “percent identity” and “percent homology” when referring to a particular sequence, are used as set forth in the University of Wisconsin GCG software program. Nucleic acids of the invention also include complementary nucleic acids. In some instances, the sequences will be fully complementary (no mismatches) when aligned. In other instances, there may be up to about a 20% mismatch in the sequences.

The invention also provides a nucleic acid molecule encoding the variable region of the light chain (V_{L}) as described herein as well as an amino acid sequence that is at least 70%, 75%, 80%, 85%, 90%, 95%, 96%, 97%, 98% or 99% identical to one of the amino acid sequences encoding a V_{L} as described herein, particularly to a V_{L} that comprises an amino acid sequence of one of the sequences as set forth in Appendix II or III. The invention also provides a nucleic acid sequence that is at least 70%, 75%, 80%, 85%, 90%, 95%, 96%, 97%, 98% or 99% identical to a nucleic acid sequence of one of the sequences as set forth in Appendix I. In another embodiment, the nucleic acid molecule encoding a V_{L} is one that hybridizes under highly stringent conditions to a nucleic acid sequence encoding a V_{L} as described above.

The invention also provides a nucleic acid molecule encoding the variable region of the heavy chain (V_{H}) as described herein as well as an amino acid sequence that is at least 70%, 75%, 80%, 85%, 90%, 95%, 96%, 97%, 98% or 99% identical to one of the amino acid sequences encoding a V_{H} as described herein, particularly to a V_{H} that comprises an amino acid sequence of one of the sequences set forth in Appendix II or III. The invention also provides a nucleic acid sequence that is at least 70%, 75%, 80%, 85%, 90%, 95%, 96%, 97%, 98% or 99% identical to a nucleic acid sequence of any one or more of the sequences set forth in Appendix I. In another embodiment, the nucleic acid molecule encoding a V_{H} is one that hybridizes under highly stringent conditions to a nucleic acid sequence encoding a V_{H} as described above.

In the context of this invention, “hybridization” means the pairing of complementary strands of oligomeric compounds. In the present invention, the preferred mechanism of pairing involves hydrogen bonding, which may be Watson-Crick, Hoogsteen or reversed Hoogsteen hydrogen bonding, between complementary nucleoside or nucleotide bases (nucleobases) of the strands of oligomeric compounds. For example, adenine and thymine are complementary nucleobases which pair through the formation of hydrogen bonds. Hybridization can occur under varying circumstances.

The term “selectively hybridize” referred to herein means to detectably and specifically bind. Polynucleotides, oligonucleotides and fragments thereof in accordance with the invention selectively hybridize to nucleic acid strands under hybridization and wash conditions that minimize appreciable amounts of detectable binding to nonspecific nucleic acids. “High stringency” or “highly stringent” conditions can be used to achieve selective hybridization conditions as known in the art and discussed herein. An example of “high stringency” or “highly stringent” conditions is a method of incubating a polynucleotide with another polynucleotide, wherein one polynucleotide may be affixed to a solid surface such as a membrane, in a hybridization buffer of 6×. SSPE or SSC, 50% formamide, 5×. Denhardt's reagent, 0.5% SDS, 100 μg/ml denatured, fragmented salmon sperm DNA at a hybridization temperature of 42° C. for 12-16 hours, followed by twice washing at 55° C. using a wash buffer of 1×SSC, 0.5% SDS. See also Sambrook et al., supra, pp. 9.50-9.55.

The nucleic acid molecule encoding either or both of the entire heavy and light chains of an anti-Her3 antibodies or the variable regions thereof may be obtained from any source that produces an anti-Her3 antibody. Methods of isolating mRNA encoding an antibody are well-known in the art (See, e.g., Sambrook et al.) The mRNA may be used to produce cDNA for use in the polymerase chain reaction (PCR) or cDNA cloning of antibody genes.

A nucleic acid molecule encoding the entire heavy chain of an anti-Her3 antibody disclosed herein, may be constructed by fusing a nucleic acid molecule encoding the variable domain of a heavy chain or an antigen-binding domain thereof with a constant domain of a heavy chain. Similarly, a nucleic acid molecule encoding the light chain of the anti-Her3 antibody of the invention, may be constructed by fusing a nucleic acid molecule encoding the variable domain of a light chain or an antigen-binding domain thereof with a constant domain of a light chain. The nucleic acid molecules encoding the V_{H} and V_{L} chain may be converted to full-length antibody genes by inserting them into expression vectors already encoding heavy chain constant and light chain constant regions, respectively, such that the V_{H} segment is operatively linked to the heavy chain constant region (C_{H}) segment(s) within the vector and the V_{L} segment is operatively linked to the light chain constant region (C_{L}) segment within the vector. Alternatively, the nucleic acid molecules encoding the V_{H} or V_{L} chains are converted into full-length antibody genes by linking, e.g., ligating, the nucleic acid molecule encoding a V_{H} chain to a nucleic acid molecule encoding a C_{H} chain using standard molecular biological techniques. The same may be achieved using nucleic acid molecules encoding V_{L} and C_{L} chains. The sequences of human heavy and light chain constant region genes are known in the art. See, e.g., Kabat et al., Sequences of Proteins of Immunological Interest, 5th Ed., NTH Publ. No. 913242, 1991. Nucleic acid molecules encoding the full-length heavy and/or light chains may then be expressed from a cell into which they have been introduced and the anti-Her3 antibody isolated.

The nucleic acid molecules may be used to recombinantly express large quantities of anti-Her3 antibodies using techniques known to one skilled in the art of recombinant biologist. Likewise, the herein described nucleic acid molecules can also be used to recombinantly produce any one of the anti-Her3 antibody variants, mutants, fragments thereof or derivatives, including single chain antibodies, bispecific, scFv etc immunoadhesins, diabodies, mutated antibodies and antibody derivatives, as described further below.

In another embodiment, the nucleic acid molecules of the invention may be used as probes or PCR primers for specific antibody sequences. For instance, a nucleic acid molecule probe may be used in diagnostic methods or a nucleic acid molecule PCR primer may be used to amplify regions of DNA that could be used, inter alia, to isolate nucleic acid sequences for use in producing variable domains of anti-Her3 antibodies. In a preferred embodiment, the nucleic acid molecules are oligonucleotides. In a more preferred embodiment, the oligonucleotides are from highly variable regions of the heavy and light chains of the antibody of interest. In an even more preferred embodiment, the oligonucleotides encode all or a part of one or more of the CDRs.

A “vector” is a replicon, such as a plasmid, cosmid, bacmid, phage, artificial chromosome (BAC, YAC) or virus, into which another genetic sequence or element (either DNA or RNA) may be inserted so as to bring about the replication of the attached sequence or element. A “replicon” is any genetic element, for example, a plasmid, cosmid, bacmid, phage, artificial chromosome (BAC, YAC) or virus, which is capable of replication largely under its own control. A replicon may be either RNA or DNA and may be single or double stranded. In some embodiments, the expression vector contains a constitutively active promoter segment (such as but not limited to CMV, SV40, Elongation Factor or LTR sequences) or an inducible promoter sequence such as the steroid inducible pIND vector (Invitrogen), where the expression of the nucleic acid can be regulated. The expression vector can be introduced into a cell by transfection,

In addition to the antibody chain genes, the recombinant expression vectors of the invention carry regulatory sequences that control the expression of the antibody chain genes in a host cell. It will be appreciated by those skilled in the art that the design of the expression vector, including the selection of regulatory sequences may depend on such factors as the choice of the host cell to be transformed, the level of expression of protein desired, etc. Preferred regulatory sequences for mammalian host cell expression include viral elements that direct high levels of protein expression in mammalian cells, such as promoters and/or enhancers derived from retroviral LTRs, cytomegalovirus (CMV) (such as the CMV promoter/enhancer), Simian Virus 40 (SV40) (such as the SV40 promoter/enhancer), adenovirus, (e.g., the adenovirus major late promoter (AdMLP)), polyoma and strong mammalian promoters such as native immunoglobulin and actin promoters. For further description of viral regulatory elements, and sequences thereof, see e.g., U.S. Pat. No. 5,168,062 by Stinski, U.S. Pat. No. 4,510,245 by Bell et al. and U.S. Pat. No. 4,968,615 by Schaffner et al.

In addition to the antibody chain genes and regulatory sequences, the recombinant expression vectors of the invention may carry additional sequences, such as sequences that regulate replication of the vector in host cells (e.g., origins of replication) and selectable marker genes. The selectable marker gene facilitates selection of host cells into which the vector has been introduced (see e.g., U.S. Pat. Nos. 4,399,216, 4,634,665 and 5,179,017, all by Axel et al.).

For example, typically the selectable marker gene confers resistance to drugs, such as G418, hygromycin or methotrexate, on a host cell into which the vector has been introduced. Preferred selectable marker genes include the dihydrofolate reductase (DHFR) gene (for use in dhfr-host cells with methotrexate selection/amplification) and the neo gene (for G418 selection). For recombinant production of an antibody of the invention, the nucleic acid encoding it is isolated and inserted into a replicable vector for further cloning (amplification of the DNA) or for expression. DNA encoding the antibody is readily isolated and sequenced using conventional procedures (e.g., by using oligonucleotide probes that are capable of binding specifically to genes encoding the heavy and light chains of the antibody). Many vectors are available. The choice of vector depends in part on the host cell to be used. Generally, preferred host cells are of either prokaryotic or eukaryotic (generally mammalian) origin. It will be appreciated that constant regions of any isotype can be used for this purpose, including IgG, IgM, IgA, IgD, and IgE constant regions, and that such constant regions can be obtained from any human or animal species.

a. Generating Antibodies Using Prokaryotic Host Cells:
i. Vector Construction

Polynucleotide sequences encoding polypeptide components of the antibody of the invention can be obtained using standard recombinant techniques. Desired polynucleotide sequences may be isolated and sequenced from antibody producing cells such as hybridoma cells. Alternatively, polynucleotides can be synthesized using nucleotide synthesizer or PCR techniques. Once obtained, sequences encoding the polypeptides are inserted into a recombinant vector capable of replicating and expressing heterologous polynucleotides in prokaryotic hosts. Many vectors that are available and known in the art can be used for the purpose of the present invention. Selection of an appropriate vector will depend mainly on the size of the nucleic acids to be inserted into the vector and the particular host cell to be transformed with the vector. Each vector contains various components, depending on its function (amplification or expression of heterologous polynucleotide, or both) and its compatibility with the particular host cell in which it resides. The vector components generally include, but are not limited to: an origin of replication, a selection marker gene, a promoter, a ribosome binding site (RBS), a signal sequence, the heterologous nucleic acid insert and a transcription termination sequence.

In general, plasmid vectors containing replicon and control sequences which are derived from species compatible with the host cell are used in connection with these hosts. The vector ordinarily carries a replication site, as well as marking sequences which are capable of providing phenotypic selection in transformed cells. For example, E. coli is typically transformed using pBR322, a plasmid derived from an E. coli species. pBR322 contains genes encoding ampicillin (Amp), kanamycin (Kn) and tetracycline (Tet) resistance and thus provides easy means for identifying transformed cells. pBR322, its derivatives, or other microbial plasmids or bacteriophage may also contain, or be modified to contain, promoters which can be used by the microbial organism for expression of endogenous proteins. Examples of pBR322 derivatives used for expression of particular antibodies are described in detail in Carter et al., U.S. Pat. No. 5,648,237.

A convenient vector is one that encodes a functionally complete human CH or CL immunoglobulin sequence, with appropriate restriction sites engineered so that any V_{H} or V_{L} sequence can be easily inserted and expressed, as described above. In such vectors, splicing usually occurs between the splice donor site in the inserted T region and the splice acceptor site preceding the human C region, and also at the splice regions that occur within the human CH exons. Polyadenylation and transcription termination occur at native chromosomal sites downstream of the coding regions. The recombinant expression vector can also encode a signal peptide that facilitates secretion of the antibody chain from a host cell. The antibody chain gene may be cloned into the vector such that the signal peptide is linked in-frame to the amino terminus of the antibody chain gene. The signal peptide can be an immunoglobulin signal peptide or a heterologous signal peptide (i.e., a signal peptide from a non-immunoglobulin protein).

In addition, phage vectors containing replicon and control sequences that are compatible with the host microorganism can be used as transforming vectors in connection with these hosts. For example, bacteriophage such as λGEM™-11 may be utilized in making a recombinant vector which can be used to transform susceptible host cells such as E. coli LE392.

The expression vector of the invention may comprise two or more promoter-cistron pairs, encoding each of the polypeptide components. A promoter is an untranslated regulatory sequence located upstream (5′) to a cistron that modulates its expression. Prokaryotic promoters typically fall into two classes, inducible and constitutive. Inducible promoter is a promoter that initiates increased levels of transcription of the cistron under its control in response to changes in the culture condition, e.g. the presence or absence of a nutrient or a change in temperature.

A large number of promoters recognized by a variety of potential host cells are well known. The selected promoter can be operably linked to cistron DNA encoding the light or heavy chain by removing the promoter from the source DNA via restriction enzyme digestion and inserting the isolated promoter sequence into the vector of the invention. Both the native promoter sequence and many heterologous promoters may be used to direct amplification and/or expression of the target genes. In some embodiments, heterologous promoters are utilized, as they generally permit greater transcription and higher yields of expressed target gene as compared to the native target polypeptide promoter.

Promoters suitable for use with prokaryotic hosts include the PhoA promoter, the .beta.-galactamase and lactose promoter systems, a tryptophan (trp) promoter system and hybrid promoters such as the tac or the trc promoter. However, other promoters that are functional in bacteria (such as other known bacterial or phage promoters) are suitable as well. Their nucleotide sequences have been published, thereby enabling a skilled worker operably to ligate them to cistrons encoding the target light and heavy chains (Siebenlist et al. (1980) Cell 20: 269) using linkers or adaptors to supply any required restriction sites.

In one aspect of the invention, each cistron within the recombinant vector comprises a secretion signal sequence component that directs translocation of the expressed polypeptides across a membrane. In general, the signal sequence may be a component of the vector, or it may be a part of the target polypeptide DNA that is inserted into the vector. The signal sequence selected for the purpose of this invention should be one that is recognized and processed (i.e. cleaved by a signal peptidase) by the host cell. For prokaryotic host cells that do not recognize and process the signal sequences native to the heterologous polypeptides, the signal sequence is substituted by a prokaryotic signal sequence selected, for example, from the group consisting of the alkaline phosphatase, penicillinase, Ipp, or heat-stable enterotoxin (STII) leaders, LamB, PhoE, PelB, OmpA and MBP. In one embodiment of the invention, the signal sequences used in both cistrons of the expression system are STII signal sequences or variants thereof.

In another aspect, the production of the immunoglobulins according to the invention can occur in the cytoplasm of the host cell, and therefore does not require the presence of secretion signal sequences within each cistron. In that regard, immunoglobulin light and heavy chains are expressed, folded and assembled to form functional immunoglobulins within the cytoplasm. Certain host strains (e.g., the E. coli trxB-strains) provide cytoplasm conditions that are favorable for disulfide bond formation, thereby permitting proper folding and assembly of expressed protein subunits. Proba and Pluckthun. Gene, 159:203 (1995).

Prokaryotic host cells suitable for expressing antibodies of the invention include Archaebacteria and Eubacteria, such as Gram-negative or Gram-positive organisms. Examples of useful bacteria include Escherichia (e.g., E. coli), Bacilli (e.g., B. subtilis), Enterobacteria, Pseudomonas species (e.g., P. aeruginosa), Salmonella typhimurium, Serratia marcescans, Klebsiella, Proteus, Shigella, Rhizobia, Vitreoscilla, or Paracoccus. In one embodiment, gram-negative cells are used. In one embodiment, E. coli cells are used as hosts for the invention. Examples of E. coli strains include strain W3110 (Bachmann, Cellular and Molecular Biology, vol. 2 (Washington, D.C.: American Society for Microbiology, 1987), pp. 1190-1219; ATCC™ Deposit No. 27,325) and derivatives thereof, including strain 33D3 having genotype W3110 .DELTA.fhu.DELTA. (.DELTA.tonA) ptr3 lac Iq lacL8.DELTA.ompT.DELTA. (nmpc-fepE) degP41 kanR (U.S. Pat. No. 5,639,635). Other strains and derivatives thereof, such as E. coli 294 (ATCC 31,446), E. coli B, E. coli .lambda. 1776 (ATCC 31,537) and E. coli RV308 (ATCC 31,608) are also suitable. These examples are illustrative rather than limiting. Methods for constructing derivatives of any of the above-mentioned bacteria having defined genotypes are known in the art and described in, for example, Bass et al., Proteins, 8:309-314 (1990). It is generally necessary to select the appropriate bacteria taking into consideration replicability of the replicon in the cells of a bacterium. For example, E. coli, Serratia, or Salmonella species can be suitably used as the host when well known plasmids such as pBR322, pBR325, pACYC177, or pKN410 are used to supply the replicon. Typically the host cell should secrete minimal amounts of proteolytic enzymes, and additional protease inhibitors may desirably be incorporated in the cell culture.

ii. Antibody Production

Host cells are transformed with the above-described expression vectors and cultured in conventional nutrient media modified as appropriate for inducing promoters, selecting transformants, or amplifying the genes encoding the desired sequences.

Transformation means introducing DNA into the prokaryotic host so that the DNA is replicable, either as an extrachromosomal element or by chromosomal integrant. Depending on the host cell used, transformation is done using standard techniques appropriate to such cells. The calcium treatment employing calcium chloride is generally used for bacterial cells that contain substantial cell-wall barriers. Another method for transformation employs polyethylene glycol/DMSO. Yet another technique used is electroporation.

Prokaryotic cells used to produce any one or more of the anti-Her3 antibodies of the invention are grown in media known in the art and suitable for culture of the selected host cells. Examples of suitable media include Luria broth (LB) plus necessary nutrient supplements. In some embodiments, the media also contains a selection agent, chosen based on the construction of the expression vector, to selectively permit growth of prokaryotic cells containing the expression vector. For example, ampicillin is added to media for growth of cells expressing ampicillin resistant gene.

Any necessary supplements besides carbon, nitrogen, and inorganic phosphate sources may also be included at appropriate concentrations introduced alone or as a mixture with another supplement or medium such as a complex nitrogen source. Optionally the culture medium may contain one or more reducing agents selected from the group consisting of glutathione, cysteine, cystamine, thioglycollate, dithioerythritol and dithiothreitol.

The prokaryotic host cells are cultured at suitable temperatures. For E. coli growth, for example, the preferred temperature ranges from about 20° C. to about 39° C., more preferably from about 25° C. to about 37° C., even more preferably at about 30° C. The pH of the medium may be any pH ranging from about 5 to about 9, depending mainly on the host organism. For E. coli, the pH is preferably from about 6.8 to about 7.4, and more preferably about 7.0.

If an inducible promoter is used in the expression vector of the invention, protein expression is induced under conditions suitable for the activation of the promoter. In one aspect of the invention, PhoA promoters are used for controlling transcription of the polypeptides. Accordingly, the transformed host cells are cultured in a phosphate-limiting medium for induction. Preferably, the phosphate-limiting medium is the C.R.A.P medium (see, e.g., Simmons et al., J. Immunol. Methods (2002), 263:133-147). A variety of other inducers may be used, according to the vector construct employed, as is known in the art.

In one embodiment, the expressed polypeptides of the present invention are secreted into and recovered from the periplasm of the host cells. Protein recovery typically involves disrupting the microorganism, generally by such means as osmotic shock, sonication or lysis. Once cells are disrupted, cell debris or whole cells may be removed by centrifugation or filtration. The proteins may be further purified, for example, by affinity resin chromatography. Alternatively, proteins can be transported into the culture media and isolated therein. Cells may be removed from the culture and the culture supernatant being filtered and concentrated for further purification of the proteins produced. The expressed polypeptides can be further isolated and identified using commonly known methods such as polyacrylamide gel electrophoresis (PAGE) and Western blot assay.

Another aspect of the invention contemplates antibody production in large quantity by a fermentation process. Various large-scale fed-batch fermentation procedures are available for production of recombinant proteins. Large-scale fermentations have at least 1000 liters of capacity, preferably about 1,000 to 100,000 liters of capacity. These fermentors use agitator impellers to distribute oxygen and nutrients, especially glucose (the preferred carbon/energy source). Small scale fermentation refers generally to fermentation in a fermenter that is no more than approximately 100 liters in volumetric capacity, and can range from about 1 liter to about 100 liters.

In a fermentation process, induction of protein expression is typically initiated after the cells have been grown under suitable conditions to a desired density, e.g., an OD550 of about 180-220, at which stage the cells are in the early stationary phase. A variety of inducers may be used, according to the vector construct employed, as is known in the art and described above. Cells may be grown for shorter periods prior to induction. Cells are usually induced for about 12-50 hours, although longer or shorter induction time may be used.

To improve the production yield and quality of the polypeptides of the invention, various fermentation conditions can be modified. For example, to improve the proper assembly and folding of the secreted antibody polypeptides, additional vectors overexpressing chaperone proteins, such as Dsb proteins (DsbA, DsbB, DsbC, DsbD and or DsbG) or FkpA (a peptidylprolyl cis,trans-isomerase with chaperone activity) can be used to co-transform the host prokaryotic cells. The chaperone proteins have been demonstrated to facilitate the proper folding and solubility of heterologous proteins produced in bacterial host cells. Chen et al. (1999) J Bio Chem 274:19601-19605; Georgiou et al., U.S. Pat. No. 6,083,715; Georgiou et al., U.S. Pat. No. 6,027,888; Bothmann and Pluckthun (2000) J. Biol. Chem. 275:17100-17105; Ramm and Pluckthun (2000) J. Biol. Chem. 275:17106-17113; Arie et al. (2001) Mol. Microbiol. 39:199-210.

To minimize proteolysis of expressed heterologous proteins (especially those that are proteolytically sensitive) certain host strains deficient for proteolytic enzymes can be used for the present invention. For example, host cell strains may be modified to effect genetic mutation(s) in the genes encoding known bacterial proteases such as Protease III, OmpT, DegP, Tsp, Protease I, Protease Mi, Protease V, Protease VI and combinations thereof. Some E. coli protease-deficient strains are available and described in, for example, Joly et al. (1998), supra; Georgiou et al., U.S. Pat. No. 5,264,365; Georgiou et al., U.S. Pat. No. 5,508,192; Hara et al., Microbial Drug Resistance, 2:63-72 (1996).

In one embodiment, E. coli strains deficient for proteolytic enzymes and transformed with plasmids overexpressing one or more chaperone proteins are used as host cells in the expression system of the invention.

iii. Antibody Purification

Standard protein purification methods known in the art can be employed. When using recombinant techniques, the antibody can be produced intracellularly, in the periplasmic space, or directly secreted into the medium. If the antibody is produced intracellularly, as a first step, the particulate debris, either host cells or lysed fragments, are removed, for example, by centrifugation or ultrafiltration. Carter et al., Bio/Technology 10:163-167 (1992) describe a procedure for isolating antibodies which are secreted to the periplasmic space of E. coli. Briefly, cell paste is thawed in the presence of sodium acetate (pH 3.5), EDTA, and phenylmethylsulfonylfluoride (PMSF) over about 30 min. Cell debris can be removed by centrifugation. Where the antibody is secreted into the medium, supernatants from such expression systems are generally first concentrated using a commercially available protein concentration filter, for example, an Amicofin® or Millipore Pellicon® ultrafiltration unit. A protease inhibitor such as PMSF may be included in any of the foregoing steps to inhibit proteolysis and antibiotics may be included to prevent the growth of adventitious contaminants.

The following procedures are exemplary of suitable purification procedures: fractionation on immunoaffinity or ion-exchange columns, ethanol precipitation, reverse phase HPLC, chromatography on silica or on a cation-exchange resin such as DEAE, chromatofocusing, SDS-PAGE, ammonium sulfate precipitation, and gel filtration using, for example, Sephadex G-75.

The suitability of protein A as an affinity ligand depends on the species and isotype of any immunoglobulin Fc domain that is present in the antibody. Protein A can be used to purify antibodies that are based on human γ1, γ2, or γ4 heavy chains (Lindmark et al., J. Immunol. Meth. 62:1-13 (1983)). Protein G is recommended for all mouse isotypes and for human γ3 (Guss et al., EMBO J. 5:15671575 (1986)). The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the antibody comprises a C H3 domain, the Bakerbond ABX®resin (J. T. Baker, Phillipsburg, N.J.) is useful for purification. Other techniques for protein purification such as fractionation on an ion-exchange column, ethanol precipitation, Reverse Phase HPLC, chromatography on silica, chromatography on heparin SEPHAROSE® chromatography on an anion or cation exchange resin (such as a polyaspartic acid column), chromatofocusing, SDS-PAGE, and ammonium sulfate precipitation are also available depending on the antibody to be recovered.

In one aspect, Protein A immobilized on a solid phase is used for immunoaffinity purification of the full length antibody products of the invention. Protein A is a 41 kD cell wall protein from Staphylococcus aureus which binds with a high affinity to the Fc region of antibodies. Lindmark et al (1983) J. Immunol. Meth. 62:1-13. The solid phase to which Protein A is immobilized is preferably a column comprising a glass or silica surface, more preferably a controlled pore glass column or a silicic acid column. In some applications, the column has been coated with a reagent, such as glycerol, in an attempt to prevent nonspecific adherence of contaminants.

As the first step of purification, the preparation derived from the cell culture as described above is applied onto the Protein A immobilized solid phase to allow specific binding of the antibody of interest to Protein A. The solid phase is then washed to remove contaminants non-specifically bound to the solid phase. Finally the antibody of interest is recovered from the solid phase by elution.

Following any preliminary purification step(s), the mixture comprising the antibody of interest and contaminants may be subjected to low pH hydrophobic interaction chromatography using an elution buffer at a pH between about 2.5-4.5, preferably performed at low salt concentrations (e.g., from about 0-0.25M salt).

### Generating Antibodies Using Eukaryotic Host Cells:

The vector components generally include, but are not limited to, one or more of the following: a signal sequence, an origin of replication, one or more marker genes, an enhancer element, a promoter, and a transcription termination sequence.

### (i) Signal Sequence Component

A vector for use in a eukaryotic host cell may also contain a signal sequence or other polypeptide having a specific cleavage site at the N-terminus of the mature protein or polypeptide of interest. The heterologous signal sequence selected preferably is one that is recognized and processed (i.e., cleaved by a signal peptidase) by the host cell. In mammalian cell expression, mammalian signal sequences as well as viral secretory leaders, for example, the herpes simplex gD signal, are available.

The DNA for such precursor region is ligated in reading frame to DNA encoding the antibody.

### (ii) Origin of Replication

Generally, an origin of replication component is not needed for mammalian expression vectors. For example, the SV40 origin may typically be used only because it contains the early promoter.

(iii) Selection Gene Component

Expression and cloning vectors may contain a selection gene, also termed a selectable marker. Typical selection genes encode proteins that (a) confer resistance to antibiotics or other toxins, e.g., ampicillin, neomycin, methotrexate, or tetracycline, (b) complement auxotrophic deficiencies, where relevant, or (c) supply critical nutrients not available from complex media.

One example of a selection scheme utilizes a drug to arrest growth of a host cell. Those cells that are successfully transformed with a heterologous gene produce a protein conferring drug resistance and thus survive the selection regimen. Examples of such dominant selection use the drugs neomycin, mycophenolic acid and hygromycin.

Another example of suitable selectable markers for mammalian cells are those that enable the identification of cells competent to take up the antibody nucleic acid, such as DHFR, thymidine kinase, metallothionein-I and -II, preferably primate metallothionein genes, adenosine deaminase, ornithine decarboxylase, etc.

For example, cells transformed with the DHFR selection gene are first identified by culturing all of the transformants in a culture medium that contains methotrexate (Mtx), a competitive antagonist of DHFR. An appropriate host cell when wild-type DHFR is employed is the Chinese hamster ovary (CHO) cell line deficient in DHFR activity (e.g., ATCC CRL-9096).

Alternatively, host cells (particularly wild-type hosts that contain endogenous DHFR) transformed or co-transformed with DNA sequences encoding an antibody, wild-type DHFR protein, and another selectable marker such as aminoglycoside 3′-phosphotransferase (APH) can be selected by cell growth in medium containing a selection agent for the selectable marker such as an aminoglycosidic antibiotic, e.g., kanamycin, neomycin, or G418. See U.S. Pat. No. 4,965,199.

### (iv) Promoter Component

Expression and cloning vectors usually contain a promoter that is recognized by the host organism and is operably linked to the antibody polypeptide nucleic acid. Promoter sequences are known for eukaryotes. Virtually all eukaryotic genes have an AT-rich region located approximately 25 to 30 bases upstream from the site where transcription is initiated. Another sequence found 70 to 80 bases upstream from the start of transcription of many genes is a CNCAAT region where N may be any nucleotide. At the 3′ end of most eukaryotic genes is an AATAAA sequence that may be the signal for addition of the poly A tail to the 3′ end of the coding sequence. All of these sequences are suitably inserted into eukaryotic expression vectors.

Antibody polypeptide transcription from vectors in mammalian host cells is controlled, for example, by promoters obtained from the genomes of viruses such as polyoma virus, fowlpox virus, adenovirus (such as Adenovirus 2), bovine papilloma virus, avian sarcoma virus, cytomegalovirus, a retrovirus, hepatitis-B virus and Simian Virus 40 (SV40), from heterologous mammalian promoters, e.g., the actin promoter or an immunoglobulin promoter, from heat-shock promoters, provided such promoters are compatible with the host cell systems.

The early and late promoters of the SV40 virus are conveniently obtained as an SV40 restriction fragment that also contains the SV40 viral origin of replication. The immediate early promoter of the human cytomegalovirus is conveniently obtained as a HindIII E restriction fragment. A system for expressing DNA in mammalian hosts using the bovine papilloma virus as a vector is disclosed in U.S. Pat. No. 4,419,446. A modification of this system is described in U.S. Pat. No. 4,601,978. Alternatively, the Rous Sarcoma Virus long terminal repeat can be used as the promoter.

### (v) Enhancer Element Component

Transcription of DNA encoding the antibody polypeptide of this invention by higher eukaryotes is often increased by inserting an enhancer sequence into the vector. Many enhancer sequences are now known from mammalian genes (globin, elastase, albumin, .alpha.-fetoprotein, and insulin). Typically, however, one will use an enhancer from a eukaryotic cell virus. Examples include the SV40 enhancer on the late side of the replication origin (bp 100-270), the cytomegalovirus early promoter enhancer, the polyoma enhancer on the late side of the replication origin, and adenovirus enhancers. See also Yaniv, Nature 297:17-18 (1982) on enhancing elements for activation of eukaryotic promoters. The enhancer may be spliced into the vector at a position 5′ or 3′ to the antibody polypeptide-encoding sequence, but is preferably located at a site 5′ from the promoter.

### (vi) Transcription Termination Component

Expression vectors used in eukaryotic host cells will typically also contain sequences necessary for the termination of transcription and for stabilizing the mRNA. Such sequences are commonly available from the 5′ and, occasionally 3′, untranslated regions of eukaryotic or viral DNAs or cDNAs. These regions contain nucleotide segments transcribed as polyadenylated fragments in the untranslated portion of the mRNA encoding an antibody. One useful transcription termination component is the bovine growth hormone polyadenylation region. See WO94/11026 and the expression vector disclosed therein.

(vii) Selection and Transformation of Host Cells

Suitable host cells for cloning or expressing the DNA in the vectors herein include higher eukaryote cells described herein, including vertebrate host cells. Propagation of vertebrate cells in culture (tissue culture) has become a routine procedure. Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/-DHFR (CHO, Urlaub et al., Proc. Natl. Acad. Sci. USA 77:4216 (1980)); mouse sertoli cells (TM4, Mather, Biol. Reprod. 23:243-251 (1980); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci. 383:44-68 (1982)); MRC 5 cells; FS4 cells; and a human hepatoma line (Hep G2).

Host cells are transformed with the above-described expression or cloning vectors for antibody production and cultured in conventional nutrient media modified as appropriate for inducing promoters, selecting transformants, or amplifying the genes encoding the desired sequences.

(viii) Culturing the Host Cells

The Suitable host cells for producing an antibody of this invention may be cultured in a variety of media. Commercially available media such as Ham's F10 (Sigma), Minimal Essential Medium ((MEM), (Sigma), RPM-1640 (Sigma), and Dulbecco's Modified Eagle's Medium ((DMEM), Sigma) are suitable for culturing the host cells. In addition, any of the media described in Ham et al., Meth. Enz. 58:44 (1979), Barnes et al., Anal. Biochem. 102:255 (1980), U.S. Pat. No. 4,767,704; 4,657,866; 4,927,762; 4,560,655; or 5,122,469; WO 90/03430; WO 87/00195; or U.S. Pat. No. Re. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

### (ix) Purification of Antibody

When using recombinant techniques, the antibody can be produced intracellularly, or directly secreted into the medium. If the antibody is produced intracellularly, as a first step, the particulate debris, either host cells or lysed fragments, are removed, for example, by centrifugation or ultrafiltration. Where the antibody is secreted into the medium, supernatants from such expression systems are generally first concentrated using a commercially available protein concentration filter, for example, an Amicon or Millipore Pellicon™ ultrafiltration unit. A protease inhibitor such as PMSF may be included in any of the foregoing steps to inhibit proteolysis and antibiotics may be included to prevent the growth of adventitious contaminants.

The antibody composition prepared from the cells can be purified using, for example, hydroxylapatite chromatography, gel electrophoresis, dialysis, and affinity chromatography, with affinity chromatography being the preferred purification technique. The suitability of protein A as an affinity ligand depends on the species and isotype of any immunoglobulin Fc domain that is present in the antibody. Protein A can be used to purify antibodies that are based on human γ1, γ2, or γ4 heavy chains (Lindmark et al., J. Immunol. Meth. 62:1-13 (1983)). Protein G is recommended for all mouse isotypes and for human γ3 (Guss et al., EMBO J. 5:15671575 (1986)). The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the antibody comprises a CH3 domain, the Bakerbond ABX™ resin (J. T. Baker, Phillipsburg, N.J.) is useful for purification. Other techniques for protein purification such as fractionation on an ion-exchange column, ethanol precipitation, Reverse Phase HPLC, chromatography on silica, chromatography on heparin SEPHAROSE™ chromatography on an anion or cation exchange resin (such as a polyaspartic acid column), chromatofocusing, SDS-PAGE, and ammonium sulfate precipitation are also available depending on the antibody to be recovered.

Following any preliminary purification step(s), the mixture comprising the antibody of interest and contaminants may be subjected to low pH hydrophobic interaction chromatography using an elution buffer at a pH between about 2.5-4.5, preferably performed at low salt concentrations (e.g., from about 0-0.25 M salt).

### Immunoconjugates

The invention also pertains to immunoconjugates (interchangeably termed “antibody-drug conjugates” or “ADC”) comprising at least one invention antibody conjugated to a cytotoxic agent such as a chemotherapeutic agent, a growth inhibitory agent, a toxin (e.g., an enzymatically active toxin of bacterial, fungal, plant, or animal origin, or fragments thereof), or a radioactive isotope (i.e., a radioconjugate).

The use of antibody-drug conjugates for the local delivery of cytotoxic or cytostatic agents, i.e. drugs to kill or inhibit tumor cells in the treatment of cancer (Syrigos and Epenetos (1999) Anticancer Research 19:605-614; Niculescu-Duvaz and Springer (1997) Adv. Drg Del. Rev. 26:151-172; U.S. Pat. No. 4,975,278) allows targeted delivery of the drug moiety to tumors, and intracellular accumulation therein, where systemic administration of these unconjugated drug agents may result in unacceptable levels of toxicity to normal cells as well as the tumor cells sought to be eliminated (Baldwin et al., (1986) Lancet pp. (Mar. 15, 1986):603-05; Thorpe, (1985) “Antibody Carriers Of Cytotoxic Agents In Cancer Therapy: A Review,” in Monoclonal Antibodies '84: Biological And Clinical Applications, A. Pinchera et al. (ed.s), pp. 475-506). Maximal efficacy with minimal toxicity is sought thereby. Both polyclonal antibodies and monoclonal antibodies have been reported as useful in these strategies (Rowland et al., (1986) Cancer Immunol. Immunother., 21:183-87). Drugs used in these methods include daunomycin, doxorubicin, methotrexate, and vindesine (Rowland et al., (1986) supra). Toxins used in antibody-toxin conjugates include bacterial toxins such as diphtheria toxin, plant toxins such as ricin, small molecule toxins such as geldanamycin (Mandler et al (2000) Jour. of the Nat. Cancer Inst. 92(19):1573-1581; Mandler et al (2000) Bioorganic & Med. Chem. Letters 10: 1025-1028; Mandler et al (2002) Bioconjugate Chem. 13:786-791), maytansinoids (EP 1391213; Liu et al., (1996) Proc. Natl. Acad. Sci. USA 93:8618-8623), and calicheamicin (Lode et al (1998) Cancer Res. 58:2928; Hinman et al (1993) Cancer Res. 53:3336-3342). The toxins may effect their cytotoxic and cytostatic effects by mechanisms including tubulin binding, DNA binding, or topoisomerase inhibition. Some cytotoxic drugs tend to be inactive or less active when conjugated to large antibodies or protein receptor ligands.

ZEVALIN™ (ibritumomab tiuxetan, Biogen/Idec) is an antibody-radioisotope conjugate composed of a murine IgG1 kappa monoclonal antibody directed against the CD20 antigen found on the surface of normal and malignant B lymphocytes and ¹¹¹In or ⁹⁸Y radioisotope bound by a thiourea linker-chelator (Wiseman et al (2000) Eur. Jour. Nucl. Med. 27(7):766-77; Wiseman et al (2002) Blood 99(12):4336-42; Witzig et al (2002) J. Clin. Oncol. 20(10):2453-63; Witzig et al (2002) J. Clin. Oncol. 20(15):3262-69). Although ZEVALIN™ has activity against B-cell non-Hodgkin's Lymphoma (NHL), administration results in severe and prolonged cytopenias in most patients. MYLOTARG™ (gemtuzumab ozogamicin, Wyeth Pharmaceuticals), an antibody drug conjugate composed of a hu CD33 antibody linked to calicheamicin, was approved in 2000 for the treatment of acute myeloid leukemia by injection (Drugs of the Future (2000) 25(7):686; U.S. Pat. Nos. 4,970,198; 5,079,233; 5,585,089; 5,606,040; 5,693,762; 5,739,116; 5,767,285; 5,773,001). Cantuzumab mertansine (Immunogen, Inc.), an antibody drug conjugate composed of the huC242 antibody linked via the disulfide linker SPP to the maytansinoid drug moiety, DM1, is advancing into Phase II trials for the treatment of cancers that express CanAg, such as colon, pancreatic, gastric, and others. MLN-2704 (Millennium Pharm., BZL Biologics, Immunogen Inc.), an antibody drug conjugate composed of the anti-prostate specific membrane antigen (PSMA) monoclonal antibody linked to the maytansinoid drug moiety, DM1, is under development for the potential treatment of prostate tumors. The auristatin peptides, auristatin E (AE) and monomethylauristatin (MMAE), synthetic analogs of dolastatin, were conjugated to chimeric monoclonal antibodies cBR96 (specific to Lewis Y on carcinomas) and cAC10 (specific to CD30 on hematological malignancies) (Doronina et al (2003) Nature Biotechnology 21(7):778-784) and are under therapeutic development.

Chemotherapeutic agents useful in the generation of immunoconjugates are described herein (eg., above). Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes. See, e.g., WO 93/21232 published Oct. 28, 1993. A variety of radionuclides are available for the production of radioconjugated antibodies. Examples include ²¹²Bi, ¹³¹I, ¹³¹In, ⁹⁰Y, and ¹⁸⁶Re. Conjugates of the antibody and cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl)hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al., Science, 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026.

Conjugates of an antibody and one or more small molecule toxins, such as a calicheamicin, maytansinoids, dolastatins, auristatins, a trichothecene, and CC1065, and the derivatives of these toxins that have toxin activity, are also contemplated herein.

i. Maytansine and Maytansinoids

In some embodiments, the immunoconjugate comprises an antibody (full length or fragments) of the invention conjugated to one or more maytansinoid molecules.

Maytansinoids are mitotoxic inhibitors which act by inhibiting tubulin polymerization. Maytansine was first isolated from the east African shrub Maytenus serrata (U.S. Pat. No. 3,896,111). Subsequently, it was discovered that certain microbes also produce maytansinoids, such as maytansinol and C-3 maytansinol esters (U.S. Pat. No. 4,151,042). Synthetic maytansinol and derivatives and analogues thereof are disclosed, for example, in U.S. Pat. Nos. 4,137,230; 4,248,870; 4,256,746; 4,260,608; 4,265,814; 4,294,757; 4,307,016; 4,308,268; 4,308,269; 4,309,428; 4,313,946; 4,315,929; 4,317,821; 4,322,348; 4,331,598; 4,361,650; 4,364,866; 4,424,219; 4,450,254; 4,362,663; and 4,371,533.

Maytansinoid drug moieties are attractive drug moieties in antibody drug conjugates because they are: (i) relatively accessible to prepare by fermentation or chemical modification, derivatization of fermentation products, (ii) amenable to derivatization with functional groups suitable for conjugation through the non-disulfide linkers to antibodies, (iii) stable in plasma, and (iv) effective against a variety of tumor cell lines.

Immunoconjugates containing maytansinoids, methods of making same, and their therapeutic use are disclosed, for example, in U.S. Pat. Nos. 5,208,020, 5,416,064 and European Patent EP 0 425 235 B1, the disclosures of which are hereby expressly incorporated by reference. Liu et al., Proc. Natl. Acad. Sci. USA 93:8618-8623 (1996) described immunoconjugates comprising a maytansinoid designated DM1 linked to the monoclonal antibody C242 directed against human colorectal cancer. The conjugate was found to be highly cytotoxic towards cultured colon cancer cells, and showed antitumor activity in an in vivo tumor growth assay. Chari et al., Cancer Research 52:127-131 (1992) describe immunoconjugates in which a maytansinoid was conjugated via a disulfide linker to the murine antibody A7 binding to an antigen on human colon cancer cell lines, or to another murine monoclonal antibody TA.1 that binds the Her2/neu oncogene. The cytotoxicity of the TA.1-maytansinoid conjugate was tested in vitro on the human breast cancer cell line SK-BR-3, which expresses 3×10⁵ Her2 surface antigens per cell. The drug conjugate achieved a degree of cytotoxicity similar to the free maytansinoid drug, which could be increased by increasing the number of maytansinoid molecules per antibody molecule. The A7-maytansinoid conjugate showed low systemic cytotoxicity in mice.

Antibody-maytansinoid conjugates are prepared by chemically linking an antibody to a maytansinoid molecule without significantly diminishing the biological activity of either the antibody or the maytansinoid molecule. See, e.g., U.S. Pat. No. 5,208,020 (the disclosure of which is hereby expressly incorporated by reference). An average of 3-4 maytansinoid molecules conjugated per antibody molecule has shown efficacy in enhancing cytotoxicity of target cells without negatively affecting the function or solubility of the antibody, although even one molecule of toxin/antibody would be expected to enhance cytotoxicity over the use of naked antibody. Maytansinoids are well known in the art and can be synthesized by known techniques or isolated from natural sources. Suitable maytansinoids are disclosed, for example, in U.S. Pat. No. 5,208,020 and in the other patents and nonpatent publications referred to hereinabove. Preferred maytansinoids are maytansinol and maytansinol analogues modified in the aromatic ring or at other positions of the maytansinol molecule, such as various maytansinol esters.

There are many linking groups known in the art for making antibody-maytansinoid conjugates, including, for example, those disclosed in U.S. Pat. No. 5,208,020 or EP Patent 0 425 235 B1, Chari et al., Cancer Research 52:127-131 (1992), and U.S. patent application Ser. No. 10/960,602, filed Oct. 8, 2004, the disclosures of which are hereby expressly incorporated by reference. Antibody-maytansinoid conjugates comprising the linker component SMCC may be prepared as disclosed in U.S. patent application Ser. No. 10/960,602, filed Oct. 8, 2004. The linking groups include disulfide groups, thioether groups, acid labile groups, photolabile groups, peptidase labile groups, or esterase labile groups, as disclosed in the above-identified patents, disulfide and thioether groups being preferred. Additional linking groups are described and exemplified herein.

Conjugates of the antibody and maytansinoid may be made using a variety of bifunctional protein coupling agents such as N-succinimidyl-3-(2-pyridyldithio) propionate (SPDP), succinimidyl-4-(N-maleimidomethyl)cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis(p-azidobenzoyl)hexanediamine), bis-diazonium. derivatives (such as bis-(p-diazoniumbenzoyl)ethylenediamine), diisocyanates (such as toluene 2,6-diisocyanate), and bis-active fluorine compounds (such as 1,5-difluoro-2,4-dinitrobenzene). Particularly preferred coupling agents include N-succinimidyl-3-(2-pyridyldithio)propionate (SPDP) (Carlsson et al., Biochem. J. 173:723-737 (1978)) and N-succinimidyl-4-(2-pyridylthio)pentanoate (SPP) to provide for a disulfide linkage.

The linker may be attached to the maytansinoid molecule at various positions, depending on the type of the link. For example, an ester linkage may be formed by reaction with a hydroxyl group using conventional coupling techniques. The reaction may occur at the C-3 position having a hydroxyl group, the C-14 position modified with hydroxymethyl, the C-15 position modified with a hydroxyl group, and the C-20 position having a hydroxyl group. In a preferred embodiment, the linkage is formed at the C-3 position of maytansinol or a maytansinol analogue.

ii. Auristatins and Dolastatins

In some embodiments, the immunoconjugate comprises an antibody of the invention conjugated to dolastatins or dolastatin peptidic analogs and derivatives, the auristatins (U.S. Pat. Nos. 5,635,483; 5,780,588). Dolastatins and auristatins have been shown to interfere with microtubule dynamics, GTP hydrolysis, and nuclear and cellular division (Woyke et al (2001) Antimicrob. Agents and Chemother. 45(12):3580-3584) and have anticancer (U.S. Pat. No. 5,663,149) and antifungal activity (Pettit et al (1998) Antimicrob. Agents Chemother. 42:2961-2965). The dolastatin or auristatin drug moiety may be attached to the antibody through the N (amino) terminus or the C (carboxyl) terminus of the peptidic drug moiety (WO 02/088172).

Exemplary auristatin embodiments include the N-terminus linked monomethylauristatin drug moieties DE and DF, disclosed in “Monomethylvaline Compounds Capable of Conjugation to Ligands”, U.S. Ser. No. 10/983,340, filed Nov. 5, 2004, the disclosure of which is expressly incorporated by reference in its entirety.

Typically, peptide-based drug moieties can be prepared by forming a peptide bond between two or more amino acids and/or peptide fragments. Such peptide bonds can be prepared, for example, according to the liquid phase synthesis method (see E. Schroder and K. Lubke, “The Peptides”, volume 1, pp 76-136, 1965, Academic Press) that is well known in the field of peptide chemistry. The auristatin/dolastatin drug moieties may be prepared according to the methods of: U.S. Pat. No. 5,635,483; U.S. Pat. No. 5,780,588; Pettit et al (1989) J. Am. Chem. Soc. 111:5463-5465; Pettit et al (1998) Anti-Cancer Drug Design 13:243-277; Pettit, G. R., et al. Synthesis, 1996, 719-725; and Pettit et al (1996) J. Chem. Soc. Perkin Trans. 1 5:859-863. See also Doronina (2003) Nat Biotechnol 21(7):778-784; “Monomethylvaline Compounds Capable of Conjugation to Ligands”, U.S. Ser. No. 10/983,340, filed Nov. 5, 2004, hereby incorporated by reference in its entirety (disclosing, e.g., linkers and methods of preparing monomethylvaline compounds such as MMAE and MMAF conjugated to linkers).

iii. Calicheamicin

In other embodiments, the immunoconjugate comprises an antibody of the invention conjugated to one or more calicheamicin molecules. The calicheamicin family of antibiotics are capable of producing double-stranded DNA breaks at sub-picomolar concentrations. For the preparation of conjugates of the calicheamicin family, see U.S. Pat. Nos. 5,712,374, 5,714,586, 5,739,116, 5,767,285, 5,770,701, 5,770,710, 5,773,001, 5,877,296 (all to American Cyanamid Company). Structural analogues of calicheamicin which may be used include, but are not limited to, .gamma sub.1.sup.I, .alpha.sub.2.sup.I, .alpha.sub.3.sup.I, N-acetyl-.gamma.sub.1.sup.I, PSAG and .theta.sup.I.sub.1 (Hinman et al., Cancer Research 53:3336-3342 (1993), Lode et al., Cancer Research 58:2925-2928 (1998) and the aforementioned U.S. patents to American Cyanamid). Another anti-tumor drug that the antibody can be conjugated is QFA which is an antifolate. Both calicheamicin and QFA have intracellular sites of action and do not readily cross the plasma membrane. Therefore, cellular uptake of these agents through antibody mediated internalization greatly enhances their cytotoxic effects.

iv. Other Cytotoxic Agents

Other antitumor agents that can be conjugated to the antibodies of the invention include BCNU, streptozoicin, vincristine and 5-fluorouracil, the family of agents known collectively LL-E33288 complex described in U.S. Pat. Nos. 5,053,394, 5,770,710, as well as esperamicins (U.S. Pat. No. 5,877,296).

Enzymatically active toxins and fragments thereof which can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin and the tricothecenes. See, for example, WO 93/21232 published Oct. 28, 1993.

Procedures for conjugating the biological agents with the cytotoxic agents have been previously described. Procedures for conjugating chlorambucil with antibodies are described by Flechner, I, European Journal of Cancer, 9:741-745 (1973); Ghose, T. et al., British Medical Journal, 3:495-499 (1972); and Szekerke, M., et al., Neoplasma, 19:211-215 (1972), which are hereby incorporated by reference. Procedures for conjugating daunomycin and adriamycin to antibodies are described by Hurwitz, E. et al., Cancer Research, 35:1175-1181 (1975) and Arnon, R. et al. Cancer Surveys, 1:429-449 (1982), which are hereby incorporated by reference. Procedures for preparing antibody-ricin conjugates are described in U.S. Pat. No. 4,414,148 and by Osawa, T., et al. Cancer Surveys, 1:373-388 (1982) and the references cited therein, which are hereby incorporated by reference. Coupling procedures are also described in EP 86309516.2, which is hereby incorporated by reference.

The present invention further contemplates an immunoconjugate formed between an antibody and a compound with nucleolytic activity (e.g., a ribonuclease or a DNA endonuclease such as a deoxyribonuclease; DNase).

For selective destruction of the tumor, the antibodies of the invention can be coupled to high energy radiation emitters, for example, a radioisotope, such as ¹³¹I, a .gamma.-emitter, which, when localized at the tumor site, results in a killing of several cell diameters. See, e.g., S. E. Order, “Analysis, Results, and Future Prospective of the Therapeutic Use of Radiolabeled Antibody in Cancer Therapy”, Monoclonal Antibodies for Cancer Detection and Therapy, R. W. Baldwin et al. (eds.), pp 303-316 (Academic Press 1985), which is hereby incorporated by reference. A variety of radioactive isotopes are available for the production of radioconjugated antibodies. Examples include At²¹¹, I¹³¹, I¹²⁵, Y⁹⁰, Re¹⁸⁶, Sm¹⁸⁸, Sm¹⁵³, Bi²¹², P³², Pb²¹² and radioactive isotopes of Lu. When the conjugate is used for detection, it may comprise a radioactive atom for scintigraphic studies, for example tc⁹⁹m or I¹²³, or a spin label for nuclear magnetic resonance (NMR) imaging (also known as magnetic resonance imaging, MRI), such as iodine-123 again, iodine-131, indium-111, fluorine-19, carbon-13, nitrogen-15, oxygen-17, gadolinium, manganese or iron.

The radio- or other labels may be incorporated in the conjugate in known ways.

For example, the peptide may be biosynthesized or may be synthesized by chemical amino acid synthesis using suitable amino acid precursors involving, for example, fluorine-19 in place of hydrogen. Labels such as tc⁹⁹m or I¹²³_{, Re}¹⁸⁶_{, Re}¹⁸⁸ and In¹¹¹ can be attached via a cysteine residue in the peptide. Yttrium-90 can be attached via a lysine residue. The IODOGEN method (Fraker et al (1978) Biochem. Biophys. Res. Commun. 80: 49-57) can be used to incorporate iodine-123. “Monoclonal Antibodies in Immunoscintigraphy” (Chatal, CRC Press 1989) describes other methods in detail.

Conjugates of the antibody and cytotoxic agent may be made using a variety of bifunctional protein coupling agents such as N-succinimidyl-3-(2-pyridyldithio) propionate (SPDP), succinimidyl-4-(N-maleimidomethyl)cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis(p-azidobenzoyl)hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al., Science 238:1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026. The linker may be a “cleavable linker” facilitating release of the cytotoxic drug in the cell, For example, an acid-labile linker, peptidase-sensitive linker, photolabile linker, dimethyl linker or disulfide-containing linker (Chari et al., Cancer Research 52:127-131 (1992); U.S. Pat. No. 5,208,020) may be used.

The compounds of the invention expressly contemplate, but are not limited to, ADC prepared with cross-linker reagents: BMPS, EMCS, GMBS, HBVS, LC-SMCC, MBS, MPBH, SBAP, SIA, SIAB, SMCC, SMPB, SMPH, sulfo-EMCS, sulfa-GMBS, sulfo-KMUS, sulfo-MBS, sulfo-SIAB, sulfo-SMCC, and sulfo-SMPB, and SVSB (succinimidyl-(4-vinylsulfone)benzoate) which are commercially available (e.g., from Pierce Biotechnology, Inc., Rockford, Ill., U.S.A). See pages 467-498, 2003-2004 Applications Handbook and Catalog.

v. Preparation of Antibody Drug Conjugates

In the antibody drug conjugates (ADC) of the invention, an antibody (Ab) is conjugated to one or more drug moieties (D), e.g. about 1 to about 20 drug moieties per antibody, through a linker (L). The ADC of Formula I may be prepared by several routes, employing organic chemistry reactions, conditions, and reagents known to those skilled in the art, including: (1) reaction of a nucleophilic group of an antibody with a bivalent linker reagent, to form Ab-L, via a covalent bond, followed by reaction with a drug moiety D; and (2) reaction of a nucleophilic group of a drug moiety with a bivalent linker reagent, to form D-L, via a covalent bond, followed by reaction with the nucleophilic group of an antibody. Additional methods for preparing ADC are described herein.

### Ab-(L-D)ₚI

The linker may be composed of one or more linker components. Exemplary linker components include 6-maleimidocaproyl (“MC”), maleimidopropanoyl (“MP”), valine-citrulline (“val-cit”), alanine-phenylalanine (“ala-phe”), p-aminobenzyloxycarbonyl (“PAB”), N-Succinimidyl 4-(2-pyridylthio)pentanoate (“SPP”), N-Succinimidyl 4-(N-maleimidomethyl)cyclohexane-1 carboxylate (“SMCC”), and N-Succinimidyl (4-iodo-acetyl)aminobenzoate (“SIAB”). Additional linker components are known in the art and some are described herein. See also “Monomethylvaline Compounds Capable of Conjugation to Ligands”, U.S. Ser. No. 10/983,340, filed Nov. 5, 2004, the contents of which are hereby incorporated by reference in its entirety.

In some embodiments, the linker may comprise amino acid residues. Exemplary amino acid linker components include a dipeptide, a tripeptide, a tetrapeptide or a pentapeptide. Exemplary dipeptides include: valine-citrulline (vc or val-cit), alanine-phenylalanine (af or ala-phe). Exemplary tripeptides include: glycine-valine-citrulline (gly-val-cit) and glycine-glycine-glycine (gly-gly-gly). Amino acid residues which comprise an amino acid linker component include those occurring naturally, as well as minor amino acids and non-naturally occurring amino acid analogs, such as citrulline. Amino acid linker components can be designed and optimized in their selectivity for enzymatic cleavage by a particular enzyme, for example, a tumor-associated protease, cathepsin B, C and D, or a plasmin protease.

Nucleophilic groups on antibodies include, but are not limited to: (i) N-terminal amine groups, (ii) side chain amine groups, e.g. lysine, (iii) side chain thiol groups, e.g. cysteine, and (iv) sugar hydroxyl or amino groups where the antibody is glycosylated. Amine, thiol, and hydroxyl groups are nucleophilic and capable of reacting to form covalent bonds with electrophilic groups on linker moieties and linker reagents including: (i) active esters such as NHS esters, HOBt esters, haloformates, and acid halides; (ii) alkyl and benzyl halides such as haloacetamides; (iii) aldehydes, ketones, carboxyl, and maleimide groups. Certain antibodies have reducible interchain disulfides, i.e. cysteine bridges. Antibodies may be made reactive for conjugation with linker reagents by treatment with a reducing agent such as DTT (dithiothreitol). Each cysteine bridge will thus form, theoretically, two reactive thiol nucleophiles. Additional nucleophilic groups can be introduced into antibodies through the reaction of lysines with 2-iminothiolane (Traut's reagent) resulting in conversion of an amine into a thiol. Reactive thiol groups may be introduced into the antibody (or fragment thereof) by introducing one, two, three, four, or more cysteine residues (e.g., preparing mutant antibodies comprising one or more non-native cysteine amino acid residues).

Antibody drug conjugates of the invention may also be produced by modification of the antibody to introduce electrophilic moieties, which can react with nucleophilic substituents on the linker reagent or drug. The sugars of glycosylated antibodies may be oxidized, e.g. with periodate oxidizing reagents, to form aldehyde or ketone groups which may react with the amine group of linker reagents or drug moieties. The resulting imine Schiff base groups may form a stable linkage, or may be reduced, e.g. by borohydride reagents to form stable amine linkages. In one embodiment, reaction of the carbohydrate portion of a glycosylated antibody with either galactose oxidase or sodium meta-periodate may yield carbonyl (aldehyde and ketone) groups in the protein that can react with appropriate groups on the drug (Hermanson, Bioconjugate Techniques). In another embodiment, proteins containing N-terminal serine or threonine residues can react with sodium meta-periodate, resulting in production of an aldehyde in place of the first amino acid (Geoghegan & Stroh, (1992) Bioconjugate Chem. 3:138-146; U.S. Pat. No. 5,362,852). Such aldehyde can be reacted with a drug moiety or linker nucleophile.

Likewise, nucleophilic groups on a drug moiety include, but are not limited to: amine, thiol, hydroxyl, hydrazide, oxime, hydrazine, thiosemicarbazone, hydrazine carboxylate, and arylhydrazide groups capable of reacting to form covalent bonds with electrophilic groups on linker moieties and linker reagents including: (i) active esters such as NHS esters, HOBt esters, haloformates, and acid halides; (ii) alkyl and benzyl halides such as haloacetamides; (iii) aldehydes, ketones, carboxyl, and maleimide groups.

Alternatively, a fusion protein comprising the antibody and cytotoxic agent may be made, e.g., by recombinant techniques or peptide synthesis. The length of DNA may comprise respective regions encoding the two portions of the conjugate either adjacent one another or separated by a region encoding a linker peptide which does not destroy the desired properties of the conjugate.

In yet another embodiment, the antibody may be conjugated to a “receptor” (such streptavidin) for utilization in tumor pre-targeting wherein the antibody-receptor conjugate is administered to the patient, followed by removal of unbound conjugate from the circulation using a clearing agent and then administration of a “ligand” (e.g., avidin) which is conjugated to a cytotoxic agent (e.g., a radionucleotide).

### RNAi

In certain embodiments, the cytotoxic agent is a gene modifier, e.g, RNAi molecule. Methods for making specific RNAi (RNA interference) nucleic acids are described in the art (see, e.g., U.S. Pat. No. 6,506,559; WO 01/75164; WO 99/32619; Elbashir et al., Nature 411:494-98 (2001); Zhang et al., Curr. Pharm. Biotech. 5:1-7 (2004); Paddison et al., Curr. Opin. Mol. Ther. 5:217-24 (2003).

RNA interference refers to the process of sequence-specific post transcriptional gene silencing in animals mediated by short interfering RNAs (siRNA) (Fire et al., 1998, Nature, 391, 806). The corresponding process in plants is commonly referred to as post transcriptional gene silencing or RNA silencing and is also referred to as quelling in fungi. The process of post transcriptional gene silencing is thought to be an evolutionarily conserved cellular defense mechanism used to prevent the expression of foreign genes which is commonly shared by diverse flora and phyla (Fire et al., 1999, Trends Genet., 15, 358). Such protection from foreign gene expression may have evolved in response to the production of double stranded RNAs (dsRNA) derived from viral infection or the random integration of transposon elements into a host genome via a cellular response that specifically destroys homologous single stranded RNA or viral genomic RNA. The presence of dsRNA in cells triggers the RNAi response though a mechanism that has yet to be fully characterized. This mechanism appears to be different from the interferon response that results from dsRNA mediated activation of protein kinase PKR and 2′,5′-oligoadenylate synthetase resulting in non-specific cleavage of mRNA by ribonuclease L.

The presence of long dsRNAs in cells stimulates the activity of a ribonuclease III enzyme referred to as dicer. Dicer is involved in the processing of the dsRNA into short pieces of dsRNA known as short interfering RNAs (siRNA) (Berstein et al., 2001, Nature, 409, 363). Short interfering RNAs derived from dicer activity are typically about 21-23 nucleotides in length and comprise about 19 base pair duplexes. Dicer has also been implicated in the excision of 21 and 22 nucleotide small temporal RNAs (stRNA) from precursor RNA of conserved structure that are implicated in translational control (Hutvagner et al., 2001, Science, 293, 834). The RNAi response also features an endonuclease complex containing a siRNA, commonly referred to as an RNA-induced silencing complex (RISC), which mediates cleavage of single stranded RNA having sequence complementary to the antisense strand of the siRNA duplex. Cleavage of the target RNA takes place in the middle of the region complementary to the antisense strand of the siRNA duplex (Elbashir et al., 2001, Genes Dev., 15, 188).

### Pharmaceutical Formulations

Therapeutic formulations comprising an antibody of the invention are prepared for storage by mixing the antibody having the desired degree of purity with optional physiologically acceptable carriers, excipients or stabilizers (Remington: The Science and Practice of Pharmacy 20th edition (2000)), in the form of aqueous solutions, lyophilized or other dried formulations. Acceptable carriers, excipients, or stabilizers are nontoxic to recipients at the dosages and concentrations employed, and include buffers such as phosphate, citrate, histidine and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

The formulation herein may also contain more than one active compound as necessary for the particular indication being treated, preferably those with complementary activities that do not adversely affect each other. Such molecules are suitably present in combination in amounts that are effective for the purpose intended.

The active ingredients may also be entrapped in microcapsule prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nano-particles and nanocapsules) or in macroemulsions. Such techniques are disclosed in Remington: The Science and Practice of Pharmacy 20th edition (2000).

The formulations to be used for in vivo administration must be sterile. This is readily accomplished by filtration through sterile filtration membranes.

### Therapeutic and Non-Therapeutic Uses

Any one or more of the anti-Her3 antibodies described herein can be used in a method for binding an antigen, preferably Her3 receptor protein in a subject suffering from a disorder associated with increased antigen expression and/or activity, comprising administering to the subject an antibody of the invention such that the antigen in the subject is bound. Preferably, the antigen is a human protein molecule and the subject is a human subject. Consequently, an antibody of the invention can be administered to a human subject for therapeutic purposes. As well, an antibody of the invention can be administered to a non-human mammal expressing an antigen with which the immunoglobulin cross-reacts (e.g., a primate, pig or mouse) for veterinary purposes or as an animal model of human disease. Regarding the latter, such animal models may be useful for evaluating the therapeutic efficacy of antibodies of the invention (e.g., testing of dosages and time courses of administration).

The present Her3 receptor antagonist and neutralizing antibodies are useful as therapeutic reagents for treating a Her3 receptor expressing cancer or alleviating one or more symptoms of the cancer in a mammal. The antibodies of the invention can also be used to treat other Her3-medaisted disorders such as inflammatory disorders etc. The antibody is able to bind to at least a portion of the cancer cells that express a Her3 receptor in the mammal and preferably destroy or kill Her3 receptor-expressing tumor cells or inhibit the growth of such tumor cells, in vitro, ex vivo or in vivo. Such an antibody include a naked anti-Her3 receptor antibody (not conjugated to any agent). Naked antibodies that have cytotoxic or cell growth inhibition properties can be further harnessed with a cytotoxic agent to render them even more potent in tumor cell destruction. Cytotoxic properties can be conferred to an anti-Her3 receptor antibody by, e.g., conjugating the antibody with a cytotoxic agent, to form an immunoconjugate as described herein. Alternative embodiments include Her3 specific agonist antibodies.

In one aspect, the invention provides methods for treating or preventing a tumor, a cancer, and/or a cell proliferative disorder associated with increased expression and/or activity of Her3, the methods comprising administering an effective amount of an anti-Her3 antibody to a subject in need of such treatment.

In one aspect, the invention provides methods for reducing, inhibiting, blocking, or preventing growth of a tumor or cancer, the methods comprising administering an effective amount of an anti-Her3 antibody to a subject in need of such treatment.

In one aspect, the invention provides methods for treating a tumor, a cancer, and/or a cell proliferative disorder comprising administering an effective amount of an anti-Her3 antibody to a subject in need of such treatment.

In one aspect, the invention provides methods for inhibiting cellular proliferative disorders including angiogenesis comprising administering an effective amount of an anti-Her3 antibody to a subject in need of such treatment.

In one aspect, the invention provides methods for treating a pathological condition associated with a cellular proliferative disorder comprising administering an effective amount of an anti-Her3 antibody to a subject in need of such treatment. In some embodiments, the pathological condition associated with said cellular proliferative disorder is a tumor and/or cancer.

The antibodies in accordance with the present invention may be used to deliver a variety of cytotoxic drugs including therapeutic drugs, a compound emitting radiation, molecules of plants, fungal, or bacterial origin, biological proteins, and mixtures thereof. The cytotoxic drugs can be intracellularly acting cytotoxic drugs, such as short-range radiation emitters, including, for example, short-range, high-energy .alpha.-emitters.

The antibodies of the invention can be used to treat, inhibit, delay progression of, prevent/delay recurrence of, ameliorate, or prevent diseases, disorders or conditions associated with expression and/or activity of one or more antigen molecules.

Exemplary disorders include those described supra. Also included are Her3-receptor mediated leukemias, disorders involving neovascularization, cardiovascular disease associated with Her3 polymorphism etc.

In certain embodiments, an immunoconjugate comprising an antibody conjugated with one or more cytotoxic agent(s) is administered to the patient. In some embodiments, the immunoconjugate and/or antigen to which it is bound is/are internalized by the cell, resulting in increased therapeutic efficacy of the immunoconjugate in killing the target cell to which it binds. In one embodiment, the cytotoxic agent targets or interferes with nucleic acid in the target cell. In one embodiment, the cytotoxic agent targets or interferes with microtubule polymerization. Examples of such cytotoxic agents include any of the chemotherapeutic agents noted herein (such as a maytansinoid, auristatin, dolastatin, or a calicheamicin), a radioactive isotope, or a ribonuclease or a RNAi or a DNA endonuclease.

The anti-Her3 antibodies or immunoconjugates are administered to a human patient, in accord with known methods, such as intravenous administration, e.g., as a bolus or by continuous infusion over a period of time, by intramuscular, intraperitoneal, intracerebrospinal, subcutaneous, intra-articular, intrasynovial, intrathecal, oral, topical, or inhalation routes. Intravenous or subcutaneous administration of the antibody is preferred.

Other therapeutic regimens may be combined with the administration of the anti-Her3 receptor antibody. The combined administration includes co-administration, using separate formulations or a single pharmaceutical formulation, and consecutive administration in either order, wherein preferably there is a time period while both (or all) active agents simultaneously exert their biological activities. Preferably such combined therapy results in a synergistic therapeutic effect.

It may also be desirable to combine administration of the anti-Her3 receptor antibody or antibodies, with administration of an antibody directed against another tumor antigen associated with the particular cancer. In another embodiment, the invention antibody is a bispecific construct targeting two distinct epitopes. The epitopes may be on the same antigen or on separate antigens, one of which is Her3 receptor protein.

Antibodies of the invention can be used either alone or in combination with other compositions in a therapy. For instance, an antibody of the invention may be co-administered with another antibody, chemotherapeutic agent(s) (including cocktails of chemotherapeutic agents), other cytotoxic agent(s), anti-angiogenic agent(s), cytokines, and/or growth inhibitory agent(s). Where an antibody of the invention inhibits tumor growth, it may be particularly desirable to combine it with one or more other therapeutic agent(s) which also inhibits tumor growth, e.g. anti-EGFR agents including antibodies to any one or more of the EGFR family of receptors. Alternatively, or additionally, the patient may receive combined radiation therapy (e.g. external beam irradiation or therapy with a radioactive labeled agent, such as an antibody). Such combined therapies noted above include combined administration (where the two or more agents are included in the same or separate formulations), and separate administration, in which case, administration of the antibody of the invention can occur prior to, and/or following, administration of the adjunct therapy or therapies.

It is to be further understood that a cocktail of different monoclonal antibodies, such as a mixture of the specific monoclonal antibodies described herein, or their binding fragments, may be administered, if necessary or desired, for cancer treatment. Indeed, using a mixture of monoclonal antibodies, or binding fragments thereof, in a cocktail to target several antigens, or different epitopes, on cancer cells, is an advantageous approach, particularly to prevent evasion of tumor cells and/or cancer cells due to down regulation of one of the antigens.

### Combination Therapies

As indicated above, the invention provides combined therapies in which an anti-Her3 antibody is administered with another therapy. For example, anti-Her3 antibodies are used in combinations with anti-cancer therapeutics or anti-neovascularization therapeutics to treat various neoplastic or non-neoplastic conditions. In one embodiment, the neoplastic or non-neoplastic condition is characterized by pathological disorder associated with aberrant or undesired angiogenesis. The anti-Her3 antibody can be administered serially or in combination with another agent that is effective for those purposes, either in the same composition or as separate compositions. Alternatively, or additionally, multiple inhibitors of Her3 can be administered.

The administration of the anti-Her3 antibody can be done simultaneously, e.g., as a single composition or as two or more distinct compositions using the same or different administration routes. Alternatively, or additionally, the administration can be done sequentially, in any order. In certain embodiments, intervals ranging from minutes to days, to weeks to months, can be present between the administrations of the two or more compositions. For example, the anti-cancer agent may be administered first, followed by the Her3 inhibitor. However, simultaneous administration or administration of the anti-Her3 antibody first is also contemplated.

The effective amounts of therapeutic agents administered in combination with an anti-Her3 antibody will be at the physician's or veterinarian's discretion. Dosage administration and adjustment is done to achieve maximal management of the conditions to be treated. The dose will additionally depend on such factors as the type of therapeutic agent to be used and the specific patient being treated. Suitable dosages for the anti-cancer agent are those presently used and can be lowered due to the combined action (synergy) of the anti-cancer agent and the anti-Her3 antibody. In certain embodiments, the combination of the inhibitors potentiates the efficacy of a single inhibitor. The term “potentiate” refers to an improvement in the efficacy of a therapeutic agent at its common or approved dose. See also the section entitled “Pharmaceutical Compositions” herein.

Typically, the anti-Her3 antibodies and anti-cancer agents are suitable for the same or similar diseases to block or reduce a pathological disorder such as tumor growth or growth of a cancer cell. In one embodiment the anti-cancer agent is an anti-angiogenesis agent.

“Hyper cellular proliferative” therapy in relationship to cancer is a cancer treatment strategy aimed at inhibiting the neoplastic growth of tumor cells expressing the Her3 receptor protein as well as preventing metastasis of tumors at the secondary sites, therefore allowing attack of the tumors by other therapeutics.

The anti-Her3 antibodies may also be used in addition to or in conjunction with anti-angiogenic agents that inhibit excessive tumor vascular development. Consequently, hyper-cellular proliferative therapy contemplated herein may be combined with an “antiangiogenic” therapy comprising anti-angiogenic compounds, of which many have been identified and are known in the arts, including those listed herein, e.g., listed under Definitions, and by, e.g., Carmeliet and Jain, Nature 407:249-257 (2000); Ferrara et al., Nature Reviews. Drug Discovery, 3:391-400 (2004); and Sato Int. J. Clin. Oncol., 8:200-206 (2003). In one embodiment, an anti-Her3 antibody is used in combination with an anti-Her2 antibody (or fragment) and/or another Her2 antagonist. Alternatively, or additionally, the anti-Her3 antibody may be used in combination with an anti-IGF-1r antibody or antagonist. See, U.S. Pat. No. 7,244,444. In addition or alternatively, two or more angiogenesis inhibitors may optionally be co-administered to the patient, an anti-her3 antibody and another antiangiogenic moiety. In certain embodiment, one or more additional therapeutic agents, e.g., anti-cancer agents, can be administered in combination with anti-Her3 antibody, the Her2 or IGF-1R antagonist, and an anti-angiogenesis agent.

In certain aspects of the invention, other therapeutic agents useful for combination tumor therapy with an anti-Her3 antibody include other cancer therapies, (e.g., surgery, radiological treatments (e.g., involving irradiation or administration of radioactive substances), chemotherapy, treatment with anti-cancer agents listed herein and known in the art, or combinations thereof). Alternatively, or additionally, two or more antibodies binding the same or two or more different antigens disclosed herein can be co-administered to the patient. Sometimes, it may be beneficial to also administer one or more cytokines to the patient.

Yet another embodiment provides a method for treating an Her3 mediated cancer comprising: a) obtaining a sample of diseased tissue from a patient in need of treatment of said cancer; b) determining the level of expression of Her3 levels in the tissue sample; c) scoring the samples for expression of Her3 levels; d) correlating the score to identify patients likely to benefit from treatment with an Her3 antagonist, wherein the step of correlating comprises comparing said scoring to that obtained from a control sample, e) treating the patient with a therapeutic regime known to improve the prognosis for the particular cancer. In certain embodiments, the method further proposes f) repeating steps “a” and “b”, and g) adjusting the therapeutic regime known to improve the prognosis for the cancer; h) repeating steps a-f as frequently as deemed appropriate. Exemplary and non-limiting methods of “detecting” Her3 expression for staging or scoring purposes is provided here below. Refer to, for example, section marked “Detection of Her3 antigen.”

Additional combination therapies include combining any one or more of the invention antibodies with glucocorticoids (US 20060003927) and gamma-secretase inhibitors (20080206753 including references cited therein e.g., Lanz, T. A., Hosley, J. D., Adams, W. J., and Merchant, K. M. 2004. Studies of Abeta pharmacodynamics in the brain, cerebrospinal fluid, and plasma in young (plaque-free) Tg2576 mice using the gamma-secretase inhibitor N2-[(2S)-2-(3,5-difluorophenyl)-2-hydroxyethanoyl]-N1-[(7S)-5-methyl-6-ox-o-6,7-dihydro-5H-dibenzo[b,d]azepin-7-yl]-L-alaninamide (LY-411575). J Pharmacol Exp Ther 309:49-55.)

### Chemotherapeutic Agents

In certain aspects, the invention provides a method of blocking or reducing tumor growth or growth of a cancer cell, by administering effective amounts of an antagonist of Her3 and/or an angiogenesis inhibitor(s) and one or more chemotherapeutic agents to a patient susceptible to, or diagnosed with, cancer. A variety of chemotherapeutic agents may be used in the combined treatment methods of the invention. An exemplary and non-limiting list of chemotherapeutic agents contemplated is provided herein under “Definitions.”

Likewise, in certain aspects, the invention provides for the use of “agonist” anti-Her3 antibodies.

As will be understood by those of ordinary skill in the art, the appropriate doses of chemotherapeutic agents will be generally around those already employed in clinical therapies wherein the chemotherapeutics are administered alone or in combination with other chemotherapeutics. Variation in dosage will likely occur depending on the condition being treated. The physician administering treatment will be able to determine the appropriate dose for the individual subject.

The invention also provides methods and compositions for inhibiting or preventing relapse tumor growth or relapse cancer cell growth. Relapse tumor growth or relapse cancer cell growth is used to describe a condition in which patients undergoing or treated with one or more currently available therapies (e.g., cancer therapies, such as chemotherapy, radiation therapy, surgery, hormonal therapy and/or biological therapy/immunotherapy, anti-VEGF antibody therapy, particularly a standard therapeutic regimen for the particular cancer) is not clinically adequate to treat the patients or the patients are no longer receiving any beneficial effect from the therapy such that these patients need additional effective therapy. As used herein, the phrase can also refer to a condition of the “non-responsive/refractory” patient, e.g., which describe patients who respond to therapy yet suffer from side effects, develop resistance, do not respond to the therapy, do not respond satisfactorily to the therapy, etc. In various embodiments, a cancer is relapse tumor growth or relapse cancer cell growth where the number of cancer cells has not been significantly reduced, or has increased, or tumor size has not been significantly reduced, or has increased, or fails any further reduction in size or in number of cancer cells. The determination of whether the cancer cells are relapse tumor growth or relapse cancer cell growth can be made either in vivo or in vitro by any method known in the art for assaying the effectiveness of treatment on cancer cells, using the art-accepted meanings of “relapse” or “refractory” or “non-responsive” in such a context. A tumor resistant to anti-EGFR treatment is an example of a relapse tumor growth.

The invention provides methods of blocking or reducing relapse tumor growth or relapse cancer cell growth in a subject by administering one or more anti-Her3 antibodies to block or reduce the relapse tumor growth or relapse cancer cell growth in subject. In certain embodiments, the Her3 antagonist or agonist antibodies in accordance with the invention can be administered subsequent to the cancer therapeutic. In certain embodiments, the anti-Her3 antibodies are administered simultaneously with cancer therapy. Alternatively, or additionally, the anti-Her3 antibody therapy alternates with another cancer therapy, which can be performed in any order. The invention also encompasses methods for administering one or more inhibitory antibodies to prevent the onset or recurrence of cancer in patients predisposed to having cancer. Generally, the subject was or is concurrently undergoing cancer therapy. In one embodiment, the cancer therapy is treatment with an anti-angiogenesis agent, e.g., an anti-her2 or anti-IGF-1R antagonist. In one embodiment, the anti-angiogenesis agent is an anti-IGF-1R antagonist antibody or an IGF-1R neutralizing antibody or fragment or an anti-Her2 antibody (e.g. HERCEPTIN™ (Genentech, South San Francisco, Calif.) or an EGFR antagonist (erlotinib). Additional agents can be administered in combination with an anti-her2 or anti-IGF-1R antagonist and an anti-Her3 antibody for blocking or reducing relapse tumor growth or relapse cancer cell growth, e.g., see section entitled Combination Therapies herein.

The antibody of the invention (and adjunct therapeutic agent) is/are administered by any suitable means, including parenteral, subcutaneous, intraperitoneal, intrapulmonary, and intranasal, and, if desired for local treatment, intralesional or intravitreal administration. Parenteral infusions include intramuscular, intravenous, intraarterial, intraperitoneal, or subcutaneous administration. In addition, the antibody is suitably administered by pulse infusion, particularly with declining doses of the antibody. Dosing can be by any suitable route, e.g. by injections, such as intravenous or subcutaneous injections, depending in part on whether the administration is brief or chronic.

The antibody composition of the invention will be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The antibody need not be, but is optionally formulated with one or more agents currently used to prevent or treat the disorder in question. The effective amount of such other agents depends on the amount of antibodies of the invention present in the formulation, the type of disorder or treatment, and other factors discussed above. These are generally used in the same dosages and with administration routes as used hereinbefore or about from 1 to 99% of the heretofore employed dosages.

For the prevention or treatment of disease, the appropriate dosage of an antibody of the invention (when used alone or in combination with other agents such as chemotherapeutic agents) will depend on the type of disease to be treated, the type of antibody, the severity and course of the disease, whether the antibody is administered for preventive or therapeutic purposes, previous therapy, the patient's clinical history and response to the antibody, and the discretion of the attending physician. The antibody is suitably administered to the patient at one time or over a series of treatments. Depending on the type and severity of the disease, about 1 μg/kg to 15 mg/kg (e.g. 0.1 mg/kg-10 mg/kg) of antibody is an initial candidate dosage for administration to the patient, whether, for example, by one or more separate administrations, or by continuous infusion. One typical daily dosage might range from about 1 μg/kg to 100 mg/kg or more, depending on the factors mentioned above. For repeated administrations over several days or longer, depending on the condition, the treatment is sustained until a desired suppression of disease symptoms occurs. One exemplary dosage of the antibody would be in the range from about 0.05 mg/kg to about 10 mg/kg. Thus, one or more doses of about 0.5 mg/kg, 2.0 mg/kg, 4.0 mg/kg or 10 mg/kg (or any combination thereof) may be administered to the patient. Such doses may be administered intermittently, e.g. every week or every three weeks (e.g. such that the patient receives from about two to about twenty, e.g. about six doses of the antibody). An initial higher loading dose, followed by one or more lower doses may be administered. An exemplary dosing regimen comprises administering an initial loading dose of about 4 mg/kg, followed by a weekly maintenance dose of about 2 mg/kg of the antibody. However, other dosage regimens may be useful. The progress of this therapy is easily monitored by conventional techniques and assays.

Detection of Her3 antigen

It is well accepted that cell surface growth receptor proteins, especially those whose expression correlates with an oncogenic disorder, e.g., Her3 are excellent targets for drug candidates or tumor (e.g., cancer) treatment. The state of the art now concludes that such proteins may also find use in non-therapeutic applications. The exquisite specificity of the anti-Her3 antibodies for the Her3 receptor protein can be exploited for various uses including diagnostic and prognostic reagents. The proposed uses exploit the observation that (i) the anti-Her3 antibodies of the invention including antigen binding fragments thereof specifically bind Her3 and (ii) the target receptor bound by the antibodies of the invention is highly expressed on cancerous cells. Expressly contemplated also are the use of the invention antibodies in detecting, monitoring, and quantifying Her3 expression (e.g. in an ELISA or a Western blot); purification or immunoprecipitation of Her3 from cells, to kill and eliminate Her3-expressing cells from a population of mixed cells as a step in the purification of other cells. Proposed methods of diagnosis can be performed in vitro using a cellular sample (e.g., lymph node biopsy or tissue) from a patient or be performed by in vivo imaging. Diagnostic and prognostic applications include scoring tumors as well as staging Her3-expressing cancers (e.g., in radioimaging). They may be used alone or in combination with other Her3 related cancer markers. The diagnostic uses of the antibodies according to the present invention embrace primary tumors and cancers, as well as metastases. Other cancers and tumors bearing the antigen are also amenable to these diagnostic and imaging procedures.

In one embodiment, the invention antibody or binding fragments thereof will be very useful in cancer diagnosis and prognosis by effectively allowing one skilled in the art to quantitate or quantify the expression levels of Her3 in whatever kind of “sample” it may occur. This can be achieved, for example, by immunofluorescence techniques employing a fluorescently labeled antibody, coupled with light microscopic, flow cytometric, or fluorometric detection. In addition, the antibodies, or binding fragments thereof, according to the present invention may additionally be employed histologically, as in immunofluorescence, immunoelectron microscopy, or non-immuno assays, for in situ detection of the cancer-specific antigen on cells, such as for use in monitoring, diagnosing, or detection assays. See, for example, Zola, Monoclonal Antibodies: A Manual of Techniques, pp. 147 158 (CRC Press, Inc. 1987).

In another aspect, the invention provides methods for detection of Her3, the methods comprising detecting Her3-anti-Her3 antibody complex in the sample. The term “detection” as used herein includes qualitative and/or quantitative detection (measuring levels) with or without reference to a control.

In another aspect, the invention provides methods for diagnosing a disorder associated with Her3 expression and/or activity, the methods comprising detecting Her3-anti-Her3 antibody complex in a biological sample from a patient having or suspected of having the disorder. In some embodiments, the Her3 expression is increased expression or abnormal (undesired) expression. In some embodiments, the disorder is a tumor, cancer, and/or a cell proliferative disorder.

In another aspect, the invention provides any of the anti-Her3 antibodies described herein, wherein the anti-Her3 antibody comprises a detectable label.

In another aspect, the invention provides a complex of any of the anti-Her3 antibodies described herein and Her3. In some embodiments, the complex is in vivo or in vitro. In some embodiments, the complex comprises a cancer cell. In some embodiments, the anti-Her3 antibody is detectably labeled.

Anti-Her3 antibodies can be used for the detection of Her3 in any one of a number of well known detection assay methods. For example, a biological sample may be assayed for Her3 by obtaining the sample from a desired source, admixing the sample with anti-Her3 antibody to allow the antibody to form antibody/Her3 complex with any Her3 present in the mixture, and detecting any antibody/Her3 complex present in the mixture. The biological sample may be prepared for assay by methods known in the art which are suitable for the particular sample. The methods of admixing the sample with antibodies and the methods of detecting antibody/Her3 complex are chosen according to the type of assay used. Such assays include immunohistochemistry, competitive and sandwich assays, and steric inhibition assays.

Analytical methods for Her3 all use one or more of the following reagents: labeled Her3 analogue, immobilized Her3 analogue, labeled anti-Her3 antibody, immobilized anti-Her3 antibody and steric conjugates. The labeled reagents also are known as “tracers.”

For diagnostic and imaging applications, the antibodies of the invention may be labeled. The label used is any detectable functionality that does not interfere with the binding of Her3 and anti-Her3 antibody and it can bind to the antibodies by means of physical binding, chemical binding or the like, thus allowing them to be detected. Numerous labels are known for use in immunoassay, examples including moieties that may be detected directly, such as fluorochrome, chemiluminescent, and radioactive labels, as well as moieties, such as enzymes, that must be reacted or derivatized to be detected. Specific examples of labeling substances include enzymes, fluorescent substances, chemiluminescent substances, biotin, avidin, radioactive isotopes and the like. When the fluorescently labeled antibody is exposed to light of the proper wavelength, its presence can then be detected due to fluorescence. The radioactive isotopes and fluorescent substances detailed herein independently produce detectable signals, but the enzymes, chemiluminescent substances, biotin and avidin do not independently produce detectable signals, but instead produce detectable signals when they react with at least one other substance. For example, in the case of an enzyme at least a substrate is required, and a variety of substrates are used depending on the method of measuring enzyme activity (colorimetry, fluorescence method, bioluminescence method or chemoluminescence method). In the case of biotin generally at least avidin or enzyme-modified avidin is reacted. A variety of colorants dependent on the substrate can also be used as necessary.

Examples of such labels include: Examples of such labels include the radioisotopes ³²P, ¹⁴C, ¹²⁵I, ³H, and ¹³¹I, fluorophores such as rare earth chelates or fluorescein and its derivatives, rhodamine and its derivatives, dansyl, umbelliferone, luciferases, e.g., firefly luciferase and bacterial luciferase (U.S. Pat. No. 4,737,456), luciferin, 2,3-dihydrophthalazinediones, horseradish peroxidase (HRP), alkaline phosphatase, .beta.-galactosidase, glucoamylase, lysozyme, saccharide oxidases, e.g., glucose oxidase; galactose oxidase, and glucose-6-phosphate dehydrogenase, heterocyclic oxidases such as uricase and xanthine oxidase, coupled with an enzyme that employs hydrogen peroxide to oxidize a dye precursor such as HRP, lactoperoxidase, or microperoxidase, biotin/avidin, spin labels, bacteriophage labels, stable free radicals, and the like. The label may be directly conjugated to the antibodies or fragments thereof or indirectly conjugated. Indeed, numerous ways to detectably label protein molecules are known and practiced in the art. Means of indirect conjugation of a protein to a label are also well known. Indirect conjugation of the label to the antibody may, for example, be achieved by conjugating antibody to a small hapten (e.g., digoxin) and one of the different types of labels mentioned herein is conjugated with an anti-hapten antibody mutant (e.g., anti-digoxin antibody). See, e.g., Wagner et al., J. Nucl. Med. 20: 428 (1979) and Saha et al., J. Nucl. Med. 6:542 (1976), hereby incorporated by reference.

Conventional methods are available to bind these labels covalently to proteins or polypeptides. For instance, coupling agents such as dialdehydes, carbodiimides, dimaleimides, bis-imidates, bis-diazotized benzidine, and the like may be used to tag the antibodies with the above-described fluorescent, chemiluminescent, and enzyme labels. See, for example, U.S. Pat. Nos. 3,940,475 (fluorimetry) and 3,645,090 (enzymes); Hunter et al., Nature, 144: 945 (1962); David et al., Biochemistry, 13: 1014-1021 (1974); Pain et al., J. Immunol. Methods, 40: 219-230 (1981); and Nygren, J. Histochem. and Cytochem., 30: 407-412 (1982). Preferred labels herein are enzymes such as horseradish peroxidase and alkaline phosphatase. The conjugation of such label, including the enzymes, to the antibody is a standard manipulative procedure for one of ordinary skill in immunoassay techniques. See, for example, O'Sullivan et al., “Methods for the Preparation of Enzyme-antibody Conjugates for Use in Enzyme Immunoassay,” in Methods in Enzymology, ed. J. J. Langone and H. Van Vunakis, Vol. 73 (Academic Press, New York, N.Y., 1981), pp. 147-166.

Another way to label the antibodies of the invention is by linking the antibody to an enzyme, e.g., for use in an enzyme immunoassay (EIA), (A. Voller et al., 1978, “The Enzyme Linked Immunosorbent Assay (ELISA)”, Diagnostic Horizons, 2:1 7; Microbiological Associates Quarterly Publication, Walkersville, Md.; A. Voller et al., 1978, J. Clin. Pathol., 31:507 520; J. E. Butler et al., 1981, Meths. Enzymol., 73:482 523; Enzyme Immunoassay, 1980, (Ed.) E. Maggio, CRC Press, Boca Raton, Fla.; Enzyme Immunoassay, 1981, (Eds.) E. Ishikawa et al., Kgaku Shoin, Tokyo, Japan). The enzyme that is bound to the antibody reacts with an appropriate substrate, preferably a chromogenic substrate, so as to produce a chemical moiety which can be detected, for example, by spectrophotometric, fluorometric, or by visual detection means. Nonlimiting examples of enzymes which can be used to detectably label the antibodies include malate dehydrogenase, staphylococcal nuclease, delta-5-steroid isomerase, yeast alcohol dehydrogenase, alpha-glycerophosphate dehydrogenase, triose phosphate isomerase, horseradish peroxidase, alkaline phosphatase, ribonuclease, urease, catalase, glucose-6-phosphate dehydrogenase, glucoamylase and acetylcholinesterase. The detection can be accomplished by calorimetric methods, which employ a chromogenic substrate for the enzyme, or by visual comparison of the extent of enzymatic reaction of a substrate compared with similarly prepared standards or controls. Numerous other enzyme-substrate combinations are available to those skilled in the art. For a general review of these, see U.S. Pat. Nos. 4,275,149 and 4,318,980.

Techniques for conjugating enzymes to antibodies are described in O'Sullivan et al., Methods for the Preparation of Enzyme-Antibody Conjugates for use in Enzyme Immunoassay, in Methods in Enzym. (ed J. Langone & H. Van Vunakis), Academic press, New York, 73:147-166 (1981).

Examples of enzyme-substrate combinations include, for example:

(i) Horseradish peroxidase (HRPO) with hydrogen peroxidase as a substrate, wherein the hydrogen peroxidase oxidizes a dye precursor (e.g., orthophenylene diamine (OPD) or 3,3′,5,5′-tetramethyl benzidine hydrochloride (TMB));

(ii) alkaline phosphatase (AP) with para-Nitrophenyl phosphate as chromogenic substrate; and

(iii) .β.-D-galactosidase (.beta.-D-Gal) with a chromogenic substrate (e.g., p-nitrophenyl-.beta.-D-galactosidase) or fluorogenic substrate 4-methylumbelliferyl,beta.-D-galactosidase.

Immobilization of reagents is required for certain assay methods. Immobilization entails separating the anti-Her3 antibody from any Her3 that remains free in solution. This conventionally is accomplished by either insolubilizing the anti-Her3 antibody or Her3 analogue before the assay procedure, as by adsorption to a water-insoluble matrix or surface (Bennich et al., U.S. Pat. No. 3,720,760), by covalent coupling (for example, using glutaraldehyde cross-linking), or by insolubilizing the anti-Her3 antibody or Her3 analogue afterward, e.g., by immunoprecipitation.

Suitable subjects include those who are suspected of being at risk of a pathological effect of any hyperproliferative oncogenic disorders, particularly carcinoma and sarcomas mediated by Her3, are suitable for the detection, diagnosis and prognosis paradigms of the invention. Those with a history of cancer are especially suitable. Suitable human subjects for the diagnostic and prognostic therapies may comprise two groups, which can be distinguished by clinical criteria. Patients with “advanced disease” or “high tumor burden” are those who bear a clinically measurable tumor. A clinically measurable tumor is one that can be detected on the basis of tumor mass (e.g., by palpation, CAT scan, or X-Ray; positive biochemical or histopathological markers on their own may be insufficient to identify this population).

A second group of suitable subjects is known in the art as the “adjuvant group”. These are individuals who have had a history of cancer, but have been responsive to another mode of therapy. The prior therapy may have included, but is not restricted to, surgical resection, radiotherapy, and traditional chemotherapy. As a result, these individuals have no clinically measurable tumor. However, they are suspected of being at risk for progression of the disease, either near the original tumor site, or by metastases.

This group can be further subdivided into high-risk and low-risk individuals. The subdivision is made on the basis of features observed before or after the initial treatment. These features are known in the clinical arts, and are suitably defined for each different cancer. Features typical of high risk subgroups are those in which the tumor has invaded neighboring tissues, or who show involvement of lymph nodes.

Another suitable group of subjects is those with a genetic predisposition to cancer but who have not yet evidenced clinical signs of cancer. For instance, women with a family history of breast cancer, but still of childbearing age, may avail themselves of having their breast tissue examined for expression levels of Her3 and those testing positive, e.g., having higher than normal expression level of Her3 may wish to be monitored for presenting with breast cancer or alternatively avail themselves of preventive treatment with a conventional Her3 specific monoclonal therapy.

A variety of other immunoassays are available for detecting Her3. For example, by labeling the antibodies, or binding fragments thereof, with a radioisotope, a radioimmunoassay (RIA) can be used to detect cancer-specific antigens (e.g., Current Protocols in Immunology, Volumes 1 and 2, Coligen et al., Ed. Wiley-Interscience, New York, N.Y., Pubs. (19910, Colcher et al., 1981, Cancer Research, 41, 1451 1459; Weintraub, “Principles of Radioimmunoassays”, Seventh Training Course on Radioligand Techniques, The Endocrine Society, March, 1986). The radioactive isotope label can be detected by using a gamma counter or a scintillation counter or by radiography. Representative radioisotopes include ³⁵S, ¹⁴C,¹²⁵I, ³H, and ¹³¹I. Procedures for labeling biological agents with the radioactive isotopes are generally known in the art. Tritium labeling procedures are described in U.S. Pat. No. 4,302,438, which is hereby incorporated by reference. Iodinating, tritium labeling, and ³⁵S labeling procedures especially adapted for murine monoclonal antibodies are well known. Other procedures for iodinating biological agents, such as antibodies, binding portions thereof, probes, or ligands, are described by Hunter and Greenwood, Nature 144:945 (1962), David et al., Biochemistry 13:1014-1021 (1974), and U.S. Pat. Nos. 3,867,517 and 4,376,110, which are hereby incorporated by reference. Procedures for iodinating biological agents are described by Greenwood, F. et al., Biochem. J. 89:114-123 (1963); Marchalonis, J., Biochem. J. 113:299-305 (1969); and Morrison, M. et al., Immunochemistry, 289-297 (1971), which are hereby incorporated by reference. Procedures for ⁹⁹mTc-labeling are described by Rhodes, B. et al. in Burchiel, S. et al. (eds.), Tumor Imaging: The Radioimmunochemical Detection of Cancer, New York: Masson 111-123 (1982) and the references cited therein, which are hereby incorporated by reference. Procedures suitable for ¹¹¹In-labeling biological agents are described by Hnatowich, D. J. et al., J. Immul. Methods, 65:147-157 (1983), Hnatowich, D. et al., J. Applied Radiation, 35:554-557 (1984), and Buckley, R. G. et al., F.E.B.S. 166:202-204 (1984), which are hereby incorporated by reference.

The presently universally-accepted method for the diagnosis of solid cancer is the histologic determination of abnormal cellular morphology in surgically biopsied or resected tissue. Once removed, the tissue is preserved in a fixative, embedded in paraffin wax, cut into 5 μm-thick sections, and stained with two dyes: hematoxylin for the nucleus and eosin for the cytoplasm (“H&E staining”). This approach is simple, fast, reliable, and inexpensive. Histopathology allows the diagnosis of a variety of tissue and cell types. By providing an estimation of tumor “Grade” (cellular differentiation/tissue architecture) and “Stage” (depth of organ penetration) it also makes prognosis possible.

Immunohistochemistry (“IHC”) techniques utilize an antibody to probe and visualize cellular antigens in situ, generally by chromogenic or fluorescent methods. For sample preparation, a tissue or cell sample from a mammal (typically a human patient) may be used. Examples of samples include, but are not limited to, cancer cells such as colon, breast, prostate, ovary, lung, stomach, pancreas, lymphoma, and leukemia cancer cells. The sample can be obtained by a variety of procedures known in the art including, but not limited to surgical excision, aspiration or biopsy. The tissue may be fresh or frozen. In one embodiment, the sample is fixed and embedded in paraffin or the like. The tissue sample may be fixed (i.e. preserved) by conventional methodology. One of ordinary skill in the art will appreciate that the choice of a fixative is determined by the purpose for which the sample is to be histologically stained or otherwise analyzed. One of ordinary skill in the art will also appreciate that the length of fixation depends upon the size of the tissue sample and the fixative used.

IHC may be performed in combination with additional techniques such as morphological staining and/or fluorescence in-situ hybridization. Two general methods of IHC are available; direct and indirect assays. According to the first assay, binding of antibody to the target antigen (e.g., Her3) is determined directly. This direct assay uses a labeled reagent, such as a fluorescent tag or an enzyme-labeled primary antibody, which can be visualized without further antibody interaction. In a typical indirect assay, unconjugated primary antibody binds to the antigen and then a labeled secondary antibody binds to the primary antibody. Where the secondary antibody is conjugated to an enzymatic label, a chromogenic or fluorogenic substrate is added to provide visualization of the antigen. Signal amplification occurs because several secondary antibodies may react with different epitopes on the primary antibody.

The primary and/or secondary antibody used for immunohistochemistry typically will be labeled with a detectable moiety. Numerous labels are available, some of which are detailed herein.

Aside from the sample preparation procedures discussed above, further treatment of the tissue section prior to, during or following IHC may be desired, for example, epitope retrieval methods, such as heating the tissue sample in citrate buffer may be carried out (see, e.g., Leong et al. Appl. Immunohistochem. 4(3):201 (1996)).

Following an optional blocking step, the tissue section is exposed to primary antibody for a sufficient period of time and under suitable conditions such that the primary antibody binds to the target protein antigen in the tissue sample. Appropriate conditions for achieving this can be determined by routine experimentation. The extent of binding of antibody to the sample is determined by using any one of the detectable labels discussed above. Preferably, the label is an enzymatic label (e.g. HRPO) which catalyzes a chemical alteration of the chromogenic substrate such as 3,3′-diaminobenzidine chromogen. Preferably the enzymatic label is conjugated to antibody which binds specifically to the primary antibody (e.g. the primary antibody is rabbit polyclonal antibody and secondary antibody is goat anti-rabbit antibody).

Specimens thus prepared may be mounted and coverslipped. Slide evaluation is then determined, e.g. using a microscope.

Alternatively, one may also utilize microscope-based cell imaging, which uses conventional light microscopy combined with monochromatic light filters and computer software programs. The wavelengths of the light filters are matched to the colors of the antibody stain and the cell counterstain. The filters allow the microscopist to identify, classify and then measure differences in the optical density of specific colors of light transmitted through immunostained portions of tissue sections. See U.S. Pat. Nos. 5,235,522 and 5,252,487, both of which are incorporated herein by reference, for applications of these methods to tumor protein measurement. Yet other cell imaging systems (image cytometers) permit automated recognition of features, and combine this with automated calculation of feature areas, automated calibration, and automatic calculation of average and integrated (ΣOD) optical density. (See, e.g., U.S. Pat. Nos. 5,548,661, 5,787,189, both of which are incorporated herein by reference, and references therein.)

Since immunohistochemical staining of tissue sections has been shown to be a reliable method of assessing or detecting presence of proteins in a sample. Consequently, use of the antibodies described herein to score staining and/or detection levels are also contemplated.

Protein expression may be determined using a validated scoring method (Dhanasekaran et al., 2001, Nature 412, 822-826; Rubin et al., 2002, supra; Varambally et al., 2002, Nature 419, 624-629) where staining was evaluated for intensity and the percentage of cells staining positive. In cases where benign tissue and cancer are present, only one or the other tissue type is evaluated for purposes of analysis. Any of the methods of the invention may score the analysis by using a scale of 0 to 4, where 0 is negative (no detectable Her3 or level of expression same as that of a control sample) and 4 is high intensity staining in the majority of cells. In certain embodiments, the scoring may be used for diagnostic or prognostic purposes. For example, a score of 1, while a positive score, may indicate better prognosis than, say, a score of 3 or 4.

The information gathered in accordance with the invention will aid the physician in determining a course of treatment for a patient presenting with a Her3-mediated oncogenic disorder. For example, in the case of tumor cells comprising Her3 receptor expressing, a low score might dictate that additional intervention, e.g., surgery is not warranted. Typically, a staining pattern score of about 3+ or higher in an IHC assay is diagnostic and/or prognostic. In some embodiments, a staining pattern score of about 1+ or higher is diagnostic and/or prognostic. In other embodiments, a staining pattern score of about 2+ or higher is diagnostic and/or prognostic. It is understood that when cells and/or tissue from a tumor are examined using IHC, staining is generally determined or assessed in tumor cell and/or tissue (as opposed to stromal or surrounding tissue that may be present in the sample).

Other assay methods, known as competitive or sandwich assays, are well established and widely used in the commercial diagnostics industry.

Competitive assays rely on the ability of a tracer Her3 analogue to compete with the test sample Her3 for a limited number of anti-Her3 antibody antigen-binding sites. The anti-Her3 antibody generally is insolubilized before or after the competition and then the tracer and Her3 bound to the anti-Her3 antibody are separated from the unbound tracer and Her3. This separation is accomplished by decanting (where the binding partner was preinsolubilized) or by centrifuging (where the binding partner was precipitated after the competitive reaction). The amount of test sample Her3 is inversely proportional to the amount of bound tracer as measured by the amount of marker substance. Dose-response curves with known amounts of Her3 are prepared and compared with the test results to quantitatively determine the amount of Her3 present in the test sample. These assays are called ELISA systems when enzymes are used as the detectable markers.

Another species of competitive assay, called a “homogeneous” assay, does not require a phase separation. Here, a conjugate of an enzyme with the Her3 is prepared and used such that when anti-Her3 antibody binds to the Her3 the presence of the anti-Her3 antibody modifies the enzyme activity. In this case, the Her3 or its immunologically active fragments are conjugated with a bifunctional organic bridge to an enzyme such as peroxidase. Conjugates are selected for use with anti-Her3 antibody so that binding of the anti-Her3 antibody inhibits or potentiates the enzyme activity of the label. This method per se is widely practiced under the name of EMIT.

Steric conjugates are used in steric hindrance methods for homogeneous assay. These conjugates are synthesized by covalently linking a low-molecular-weight hapten to a small Her3 fragment so that antibody to hapten is substantially unable to bind the conjugate at the same time as anti-Her3 antibody. Under this assay procedure the Her3 present in the test sample will bind anti-Her3 antibody, thereby allowing anti-hapten to bind the conjugate, resulting in a change in the character of the conjugate hapten, e.g., a change in fluorescence when the hapten is a fluorophore.

Sandwich assays particularly are useful for the determination of Her3 or anti-Her3 antibodies. In sequential sandwich assays an immobilized anti-Her3 antibody is used to adsorb test sample Her3, the test sample is removed as by washing, the bound Her3 is used to adsorb a second, labeled anti-Her3 antibody and bound material is then separated from residual tracer. The amount of bound tracer is directly proportional to test sample Her3. In “simultaneous” sandwich assays the test sample is not separated before adding the labeled anti-Her3. A sequential sandwich assay using an anti-Her3 monoclonal antibody as one antibody and a polyclonal anti-Her3 antibody as the other is useful in testing samples for Her3.

In another embodiment, the invention provides a method for determining the effect of a therapeutic regimen for alleviating a Her3 mediated disorder, wherein the regimen comprises the use of an Her3 antagonist or an agonist antibody, the method comprising the steps of a) obtaining a cell or tissue sample from an individual undergoing the therapeutic regimen b) measuring the levels of Her3 in the cell or tissue sample; c) scoring the sample for Her3 protein levels, and d) comparing the levels to that of a control sample to predict the responsiveness of the Her3 mediated disorder to the therapeutic regimen. Thus, a low score, e.g., 0 or a lowering score over time suggests that the treatment regimen comprising a Her3 antagonist, e.g., Her3 specific antibody, is effective in reducing tumor burden or Her3 expressing cells or level of Her3 expression.

In certain embodiments, the methods of the invention propose contacting the sample of interest with an antibody to Her3. In certain embodiments, the detecting is done on histological or tissue sections or cytological preparations by immunohistochemistry or immunocytochemistry. As well, detecting Her3 may be done by immunoblotting or by Fluorescence-Activated Cell Sorting (FACS).

The invention is also directed to a method for predicting disease-free survival and/or overall survival in a patient diagnosed with an oncogenic disorder associated with Her3 expression comprising: a) obtaining a sample of diseased or cancerous tissue from an individual presenting with an oncogenic disorder, b) detecting levels of Her3 expressing cells in the cancer cells or cancer tissue of the sample, c) scoring the samples for expression of Her3 levels; and d) comparing the scoring to that obtained from a control sample to determine likelihood of disease-free survival and overall survival associated with Her3. Preferably, the scoring comprises using a scale of 0 to 4, where 0 is negative (no detectable Her3 or level of Her3 comparable to a control level), and 4 is high intensity staining in the majority of cells and wherein a score of 1 to 4 (i.e. a positive score) indicates a poor prognosis for disease free and overall survival in patients with said disorder.

A method for screening for metastatic potential of solid tumors is also provided. The method comprises a) obtaining a sample of tumor tissue from an individual in need of screening for metastatic potential of a solid tumor; b) reacting an antibody to Her3 with tumor tissue from the patient; c) detecting the extent of binding of the antibody to the tissue and d) correlating the extent of binding of the antibody with its metastatic potential.

The present invention further encompasses in vivo imaging methods useful for visualizing the presence of Her3 expressing cells indicative of an oncogenic disorder. Such techniques allow for a diagnosis without the use of an unpleasant biopsy or other invasive diagnostic technique. The concentration of detectably labeled anti-Her3 antibody of the invention which is administered should be sufficient such that the binding to those cells having or expressing the Her3 antigen is detectable compared to the background. Further, it is desirable that the detectably labeled anti-Her3 antibody of the invention be rapidly cleared from the circulatory system in order to give the best target-to-background signal ratio.

Imaging analysis is well known in the medical art, and includes, without limitation, x-ray analysis, magnetic resonance imaging (MRI) or computed tomography (CE). As indicated supra, preferably, the Her3 antibodies used in the in vivo (and also in vitro) diagnostic methods are directly or indirectly labeled with a detectable substance/label that can be imaged in a patient. Suitable detectable substances include various enzymes, prosthetic groups, fluorescent materials, luminescent materials and radioactive materials. As a rule, the dosage of detectably labeled anti-Her3 antibody of the invention for in vivo diagnosis is somewhat patient-specific and depends on such factors as age, sex, and extent of disease. Dosages may also vary, for example, depending on number of injections given, tumor burden, and other factors known to those of skill in the art. For instance, tumors have been labeled in vivo using cyanine-conjugated Mabs. Ballou et al. (1995) Cancer Immunol. Immunother. 41:257 263.

In the case of a radiolabeled biological agent, the biological agent is administered to the patient and is localized to the tumor bearing the antigen (Her3 receptor protein) with which the biological agent reacts, and is detected or “imaged” in vivo using known techniques such as radionuclear scanning using e.g., a gamma camera or emission tomography. See e.g., A. R. Bradwell et al., “Developments in Antibody Imaging”, Monoclonal Antibodies for Cancer Detection and Therapy, R. W. Baldwin et al., (eds.), pp. 65-85 (Academic Press 1985), which is hereby incorporated by reference. Alternatively, a positron emission transaxial tomography scanner, such as designated Pet VI located at Brookhaven National Laboratory, can be used where the radiolabel emits positrons (e.g., ¹¹C, ¹⁸F, ¹⁵O, and ¹³N).

In one embodiment the invention provides for the use of the Her3 antibodies in the diagnosis of cancer, by specifically allowing one to detect and visualize tissues that express Her3 or contain Her3 expressing cells (e.g., cancer). The method comprises: (i) administering to a subject (and optionally a control subject) a diagnostically effective amount of detectably labeled anti-Her3 antibody of the invention or an antigen-binding fragment thereof or a pharmaceutical composition thereof comprising as an active component the antibodies of the invention or binding fragments thereof that specifically bind Her3, under conditions that allow interaction of the antibodies to Her3 to occur; and (ii) detecting the binding agent, for example, to locate Her3 expressing tissues or otherwise identify Her3 expressing cells. The term “diagnostically effective” means that the amount of detectably labeled anti-Her3 antibody of the invention is administered in sufficient quantity to enable detection of neoplasia.

In certain embodiments, the antibodies of the invention may be labeled with a contrast agent, such as barium, which can be used for x-ray analysis, or a magnetic contrast agent, such as a gadolinium chelate, which can be used for MRI or CE.

In another embodiment of the method, a biopsy is obtained from the patient to determine whether the tissue of interest expresses Her3 rather than subjecting the patient to imaging analysis.

A radiolabeled antibody or immunoconjugate may comprise a gamma.-emitting radioisotope or a positron-emitter useful for diagnostic imaging. The label used will depend on the imaging modality chosen. The use of antibodies for in vivo diagnosis is well known in the art. Sumerdon et al., (Nucl. Med. Biol 17:247-254 (1990)) have described an optimized antibody-chelator for the radioimmunoscintographic imaging of tumors using Indium-111 as the label. Griffin et al., (J Clin Onc 9:631-640 [1991]) have described the use of this agent in detecting tumors in patients suspected of having recurrent colorectal cancer.

The methods of the present invention may also use paramagnetic isotopes for purposes of in vivo detection. The use of similar agents with paramagnetic ions as labels for magnetic resonance imaging is also known in the art—Lauffer, Magnetic Resonance in Medicine 22:339-342 (1991).

Radioactive labels such as Indium-111, Technetium-99m, or Iodine-131 can be used for planar scans or single photon emission computed tomography (SPECT). Positron emitting labels such as Fluorine-19 can also be used for positron emission tomography (PET). For MRI, paramagnetic ions such as Gadolinium (III) or Manganese (II) can be used.

For in vivo diagnostic imaging, the type of detection instrument available is a major factor in selecting a given radioisotope. The radioisotope chosen must have a type of decay which is detectable for a given type of instrument. Still another important factor in selecting a radioisotope for in vivo diagnosis is that the half-life of the radioisotope be long enough so that it is still detectable at the time of maximum uptake by the target, but short enough so that deleterious radiation with respect to the individual is minimized. Ideally, a radioisotope used for in vivo imaging lacks a particle emission, but produces a large number of photons in the 140 250 keV range, to be readily detected by conventional gamma cameras.

Radioactive metals with half-lives ranging from 1 hour to 3.5 days are available for conjugation to antibodies, such as scandium-47 (3.5 days) gallium-67 (2.8 days), gallium-68 (68 minutes), technetiium-99m (6 hours), and indium-111 (3.2 days), of which gallium-67, technetium-99m, and indium-111 are preferable for gamma camera imaging, gallium-68 is preferable for positron emission tomography. Labels such as Indium-111, Technetium-99m, or Iodine-131 can be used for planar scans or single photon emission computed tomography (SPECT).

In the case of the radiometals conjugated to the specific antibody, it is likewise desirable to introduce as high a proportion of the radiolabel as possible into the antibody molecule without destroying its immunospecificity. A further improvement may be achieved by effecting radiolabeling in the presence of the specific cancer marker of the present invention, to insure that the antigen binding site on the antibody will be protected. The antigen is separated after labeling.

Suitable radioisotopes, particularly in the energy range of 60 to 4,000 keV, include, ⁵¹Cr, ⁵⁷Co, ⁵⁸Co, ⁵⁹Fe, ¹³¹I, ¹²¹I, ¹²⁴I, ⁸⁶Y, ⁶²Cu, ⁶⁴Cu, ¹¹¹In, ⁶⁷Ga, ⁶⁸Ga, ⁹⁹mTc, ⁹⁴mTc, ¹⁸F, ¹¹C, ¹³N, ¹⁵O, ⁷⁵Br, ⁷⁵Se, ⁹⁷Ru, 99 mTc, ¹¹¹In, ¹⁴mIn, ¹²³I, ¹²⁵I, ¹³¹I, ¹⁶⁹Yb, ¹⁹⁷Hg, and ²⁰¹Tl, and the like. See for example, U.S. patent application entitled “Labeling Targeting Agents with Gallium-68”—Inventors G. L. Griffiths and W. J. McBride, (U.S. Provisional Application No. 60/342,104), which discloses positron emitters, such as ¹⁸F, ⁶⁸Ga, ⁹⁴mTc, and the like, for imaging purposes and which is incorporated in its entirety by reference. Particularly useful diagnostic/detection radionuclides include, but are not limited to, ¹⁸F. ⁵²Fe, ⁶²Cu, ⁶⁴Cu, ⁶⁷Cu, ⁶⁷Ga, ⁶⁸Ga, ⁸⁶Y, ⁸⁹Zr, ⁹⁴mTc, ⁹⁴mTc, ⁹⁹mTc, ¹¹¹In, ¹²³I, ¹²⁴I, ¹²⁵I, ¹³¹I, ¹⁵⁴⁻¹⁵⁸Gd, ³²P, ⁹⁰Y, ¹⁸⁸Re, and ¹⁷⁵Lu.

Decay energies of useful gamma-ray emitting radionuclides are preferably 20 2000 keV, more preferably 60 600 keV, and most preferably 100 300 keV.

Radionuclides useful for positron emission tomography include, but are not limited to: ¹⁸F, ¹Mn, ²mMn, ⁵²Fe, ⁵⁵Co, 52Cu, ⁶⁴Cu, ⁶⁸Ga, ⁷²As, ⁷⁵Br, ⁷⁶Br, ⁸²mRb, ⁸³Sr, ⁸⁶Y, ⁸⁹Zr, ⁹⁴mTc, ¹¹⁰In, ¹²⁰I, and ¹²⁴I. Total decay energies of useful positron-emitting radionuclides are preferably <2,000 keV, more preferably under 1,000 keV, and most preferably <700 keV.

Also contemplated by the present invention is the use of non-radioactive agents as diagnostic reagents. A suitable non-radioactive diagnostic agent is a contrast agent suitable for magnetic resonance imaging, computed tomography or ultrasound. Magnetic imaging agents include, for example, non-radioactive metals, such as manganese, iron and gadolinium, complexed with metal-chelate combinations that include 2-benzyl-DTPA and its monomethyl and cyclohexyl analogs, when used along with the antibodies of the invention. See U.S. Ser. No. 09/921,290 filed on Oct. 10, 2001, which is incorporated in its entirety by reference.

Bispecific antibodies are also useful in targeting methods and provide a preferred way to deliver two diagnostic agents to a subject. U.S. Ser. Nos. 09/362,186 and 09/337,756 discloses a method of pretargeting using a bispecific antibody, in which the bispecific antibody is labeled with ²⁵¹I and delivered to a subject, followed by a divalent peptide labeled with ⁹⁹ mTc and are incorporated herein by reference in their entirety. Pretargeting methods are also described in U.S. Pat. No. 6,962,702 (Hansen et al.), U.S. Ser. Nos. 10/150,654 (Goldenberg et al.), and Ser. No. 10/768,707 (McBride et al.), which are all also incorporated herein by reference in their entirety. The delivery results in excellent tumor/normal tissue ratios for ¹²⁵I and ⁹⁹mTc, thus showing the utility of two diagnostic radioisotopes. Any combination of known diagnostic agents can be used to label the antibodies. The binding specificity of the antibody component of the MAb conjugate, the efficacy of the therapeutic agent or diagnostic agent and the effector activity of the Fc portion of the antibody can be determined by standard testing of the conjugates.

A diagnostic agent can be attached at the hinge region of a reduced antibody component via disulfide bond formation. As an alternative, such peptides can be attached to the antibody component using a heterobifunctional cross-linker, such as N-succinyl 3-(2-pyridyldithio)propionate (SPDP). Yu et al., Int. J. Cancer 56: 244 (1994). General techniques for such conjugation are well-known in the art. See, for example, Wong, CHEMISTRY OF PROTEIN CONJUGATION AND CROSS-LINKING (CRC Press 1991); Upeslacis et al., “Modification of Antibodies by Chemical Methods,” in MONOCLONAL ANTIBODIES: PRINCIPLES AND APPLICATIONS, Birch et al. (eds.), pages 187 230 (Wiley-Liss, Inc. 1995); Price, “Production and Characterization of Synthetic Peptide-Derived Antibodies,” in MONOCLONAL ANTIBODIES: PRODUCTION, ENGINEERING AND CLINICAL APPLICATION, Ritter et al. (eds.), pages 60 84 (Cambridge University Press 1995).

Methods for conjugating peptides to antibody components via an antibody carbohydrate moiety are also well-known to those of skill in the art. See, for example, Shih et al., Int. J. Cancer 41: 832 (1988); Shih et al., hit. J. Cancer 46: 1101 (1990); and Shih et al., U.S. Pat. No. 5,057,313, all of which are incorporated in their entirety by reference. The general method involves reacting an antibody component having an oxidized carbohydrate portion with a carrier polymer that has at least one free amine function and that is loaded with a plurality of peptide. This reaction results in an initial Schiff base (imine) linkage, which can be stabilized by reduction to a secondary amine to form the final conjugate.

The Fc region is absent if the antibody used as the antibody component of the immunoconjugate is an antibody fragment. However, it is possible to introduce a carbohydrate moiety into the light chain variable region of a full length antibody or antibody fragment. See, for example, Leung et al., J. Immunol. 154: 5919 (1995); Hansen et al., U.S. Pat. No. 5,443,953 (1995), Leung et al, U.S. Pat. No. 6,254,868, all of which are incorporated in their entirety by reference. The engineered carbohydrate moiety is used to attach the therapeutic or diagnostic agent.

In situ detection can be accomplished by removing a histological specimen from a patient, and providing the combination of labeled antibodies of the present invention to such a specimen. The antibody (or fragment) is preferably provided by applying or by overlaying the labeled antibody (or fragment) to a biological sample. Through the use of such a procedure, it is possible to determine not only the presence of Her3 but also the distribution of Her3 in the examined tissue. Using the present invention, those of ordinary skill will readily perceive that any of a wide variety of histological methods (such as staining procedures) can be modified in order to achieve such in situ detection.

Also provided by the invention is in vivo biophotonic imaging (Xenogen, Almeda, Calif.) which utilizes real-time luciferase. The luciferase gene is incorporated into cells, microorganisms, and animals (e.g., as a fusion protein with a marker of the present invention). When active, it leads to a reaction that emits light. A CCD camera and software is used to capture the image and analyze it.

In another embodiment, the anti-Her3 antibody is unlabeled and imaged by administering a second antibody or other molecule that is detectable and that can bind the anti-Her3 antibody. A specifically bound and labeled antibody can be detected in the patient using known methods, including, but not limited to, radionuclide imaging, positron emission tomography, computerized axial tomography, X-ray or magnetic resonance imaging method, fluorescence detection, and chemiluminescent detection.

In vivo imaging methods can also be used for developing a prognostic evaluation of the condition of a patient suspected of exhibiting an oncogenic disorder mediated by Her3.

### Purification

Still further, the anti-Her3 antibodies described herein may also be used as affinity purification agents. In this process, the antibodies are immobilized on a solid phase such a Sephadex resin or filter paper, using methods well known in the art. The immobilized antibody is contacted with a sample containing the Her3 protein (or fragment thereof) to be purified, and thereafter the support is washed with a suitable solvent that will remove substantially all the material in the sample except the Her3 protein, which is bound to the immobilized antibody. Finally, the support is washed with another suitable solvent, such as glycine buffer, pH 5.0, that will release the Her3 protein from the antibody.

### Articles of Manufacture

In another aspect of the invention, an article of manufacture containing materials useful for the treatment, prevention and/or diagnosis of the disorders described above is provided. The article of manufacture comprises a container and a label or package insert on or associated with the container. Suitable containers include, for example, bottles, vials, syringes, etc. The containers may be formed from a variety of materials such as glass or plastic. The container holds a composition which is by itself or when combined with another composition(s) effective for treating, preventing and/or diagnosing the condition and may have a sterile access port (for example the container may be an intravenous solution bag or a vial having a stopper pierceable by a hypodermic injection needle). At least one active agent in the composition is an antibody of the invention. The label or package insert indicates that the composition is used for treating the condition of choice, such as cancer. Moreover, the article of manufacture may comprise (a) a first container with a composition contained therein, wherein the composition comprises an antibody of the invention; and (b) a second container with a composition contained therein, wherein the composition comprises a further therapeutic agent, including, e.g. a chemotherapeutic agent or an anti-angiogenesis agent, including, e.g., an anti-VEGF antibody (e.g. bevacizumab). The article of manufacture in this embodiment of the invention may further comprise a package insert indicating that the first and second antibody compositions can be used to treat a particular condition, e.g. cancer. Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

Additionally, the article of manufacture may further comprise a second container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

Kits are also provided that are useful for various purposes, e.g., for Her3 receptor cell killing assays, for purification or immunoprecipitation of Her3 receptor from cells. For isolation and purification of Her3 receptor, the kit can contain an anti-Her3 receptor antibody coupled to beads (e.g., sepharose beads). Kits can be provided which contain the antibodies for detection and quantitation of Her3 receptor in vitro, e.g. in an ELISA or a Western blot. As with the article of manufacture, the kit comprises a container and a label or package insert on or associated with the container. The container holds a composition comprising at least one anti-Her3 receptor antibody of the invention. Additional containers may be included that contain, e.g., diluents and buffers, control antibodies. The label or package insert may provide a description of the composition as well as instructions for the intended in vitro or diagnostic use.

The foregoing are merely exemplary detection assays for Her3. Other methods now or hereafter developed that use anti-Her3 antibody for the determination of Her3 are included within the scope hereof, including the bioassays described herein.

The following are examples of the methods and compositions of the invention. It is understood that various other embodiments may be practiced, given the general description provided above.

### Example 1

### Mouse Hybridoma Generation

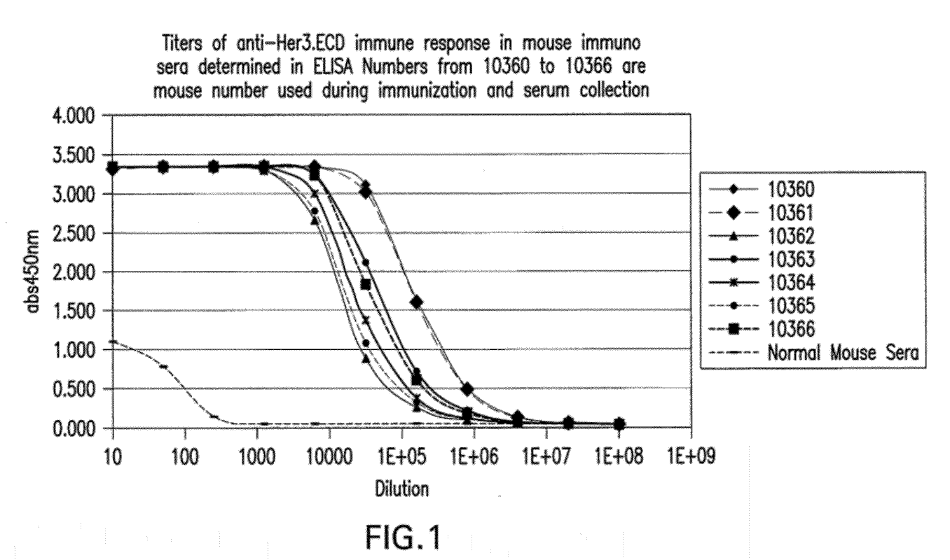

ErbB3 (Accn.# M34309) extracellular domain (ECD) (amino acids Met1-Thr643) protein was used to immunize mice. Two mouse strains, Balb/c and Swiss Webster, were used with 5 ug or 10 ug per mouse dosing. After initial immunization, two boost dosing with 2 weeks interval between dosing. Titer of anti-Her3 sera was determined by series of dilutions of serum in ELISA for binding of Her3. Typical titers reached 10⁵⁻⁶ (FIG. 1) before performing fusion.

Hybridoma cells were generated by fusion of mouse spleen cells with myeloma cells and were selected in a suitable culture medium which preferably contains one or more substances that inhibit the growth or survival of the unfused, parental myeloma cells. Primary hybridoma cells were screened for Her3 binding and positive hybrids were expanded from 96-well plate culture into 24-well plate culture. Culture medium has composition as following: complete medium of Iscove's Modified Dulbeccos Medium (IMDM) with L-Glutamine and 25 mM Hepes, 10% FBS, 0.6% 6 mM 2-Mercaptoethanol). 6 mM 2-Mercaptoethanol stock solution was prepared in basal medium and aseptically transferred to a 150 mL filter flask. Vacuum was applied to filter the stock solution and the filter reservoir was aseptically removed. The lid was screwed onto the flask and stock solution was stored at 4° C. until 3 months expiration. Hybridoma cultures were incubated at 37° C. with 5% CO₂. Once cells reached 100% confluency, they were subcultured with fresh media.

### Example 2

### Cell Line Construction

The full-length human Her3 coding region, along with their respective 5′- and 3′-flanking sequences, was purchased from GeneCopoeia™. The flanking sequences contain cloning site NspV in front of the ATG start codon at its 5′-end and stop codon followed by cloning site NotI at its 3′-end. The plasmids containing the NspV-NotI DNA fragments were received from GeneCopoeia™ and the NspV (Klenow DAN polymerase I to blunt the end DNA)-NotI digested fragments were isolated. The vector pcDNA5/FRT/T0 (Invitrogen) was used to express the full-length human Her3 open reading frame. The pcDNA5/FRT/T0 contains two features, together with matching cell lines, needed for the generation of human Her3 stable cell lines. For the first feature, the vector pcDNA5/FRT/T0 allows the expression of human HER3. The vector contains human cytomegalovirus (CMV) early promoter with two tetracycline operators to allow tight control of HER3 expression in cell lines where the tetracycline repressor is expressed. For the other feature, the vector pcDNA5/FRT/T0 contains the FRT sequence in front of promoter-less and ATG-less hygromycin gene, which allows the homologous recombination between the vector FRT sequence and the identical chromosome FRT sequence in the presence of the FLIP recombinase provided by co-transfection of pOG44 vector (Invitrogen). The matching cell line CHO TREX FLIP contains both tetracycline repressor allowing tight control of Her3 expression and FRT sequence permitting homologous recombination. The homologous recombination between the vector FRT sequence and host cell FRT sequence will allow the FLIP-IN of the entire plasmid sequence and hence the Her3 expression cassette. At the same time, the FRT sequence in the host cell chromosome was designed such that it can put the promoter-less and ATG-less hygromycin gene in frame with the start codon ATG to confer the transfected cells with expected homologous recombination hygromycin resistant. The vector pcDNA5/FRT/T0 was digested with EcorV (a blunt end)+NotI and the Her3 fragment digested with the matching set of enzymes was ligated with the linearized vector to form pcDNA5/FRT/T0-human-Her3. Ligation product was transformed into XL2 Blue competent cells and transformants were screened and positive clones sequencing confirmed.

Inducible expression stable cell lines were generated using Invitrogen FLPIn system by transfecting pcDNA5/FRT/T0-human-Her3 into CHO FLPIn TREX cells. The description and the rationale of the cell line are provided in the earlier section. To generate stable cell lines, pOG44, which provides constitutively expressed FLP recombinase (flp-F70L), and the pcDNA5/FRT based vector, pcDNA5/FRT/T0-human-HER3 were co-transfected into the CHO FLPIn/TREX host cell line. FLP recombinase mediates FRT site specific insertion of the construct by homologous recombination between FRT sites in the expression vector and those integrated into the CHO FLPIn TREX cell genome. After FRT insertion hygromycin resistance is used as a selection marker. pOG44 was co-transfected with pcDNA5/FRT/T0-human-Her3 in a 9:1 ratio (w/w) pOG44:expression plasmid using Fugene HD (Roche) lipid-based transfection reagent in a 4:1 (v/w) ratio. Cells are re-fed 48 hours post transfection with media containing hygromycin (500 ug/ml) selection agent. Colonies are expanded to generate a stable cell line to express human Her3.

EGFR, Her2, and Her4 cell lines were constructed in a similar way as described for human Her3 cell line and full length gene for EGFR, Her2, and Her4 were ordered from GeneCopoeia™.

### Example 3

### Her3 Binding on Cells: Her3-CHO Cells and Cancer Cell Lines

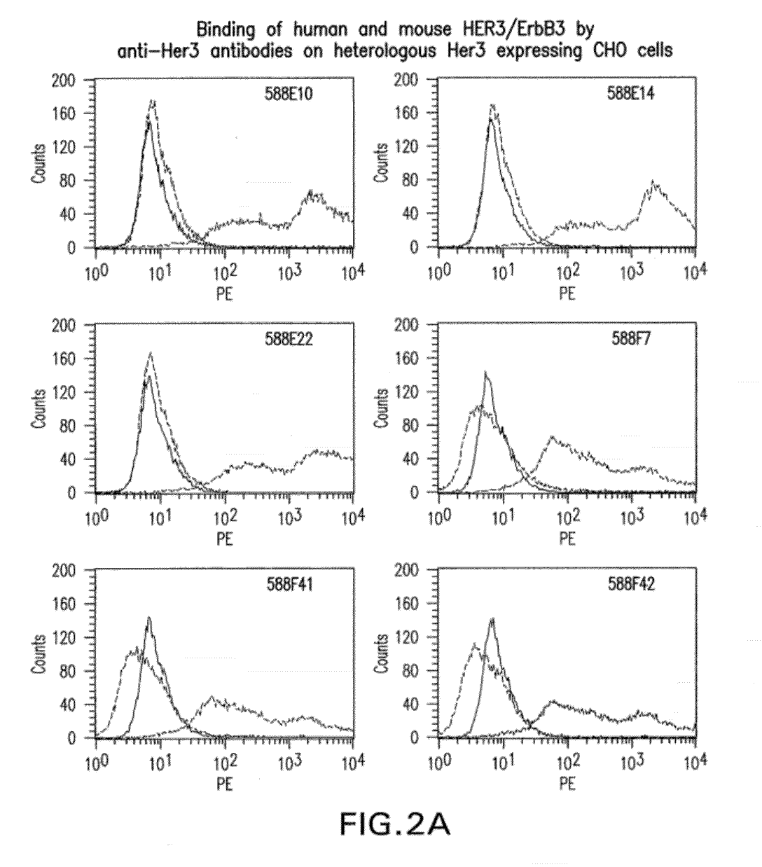

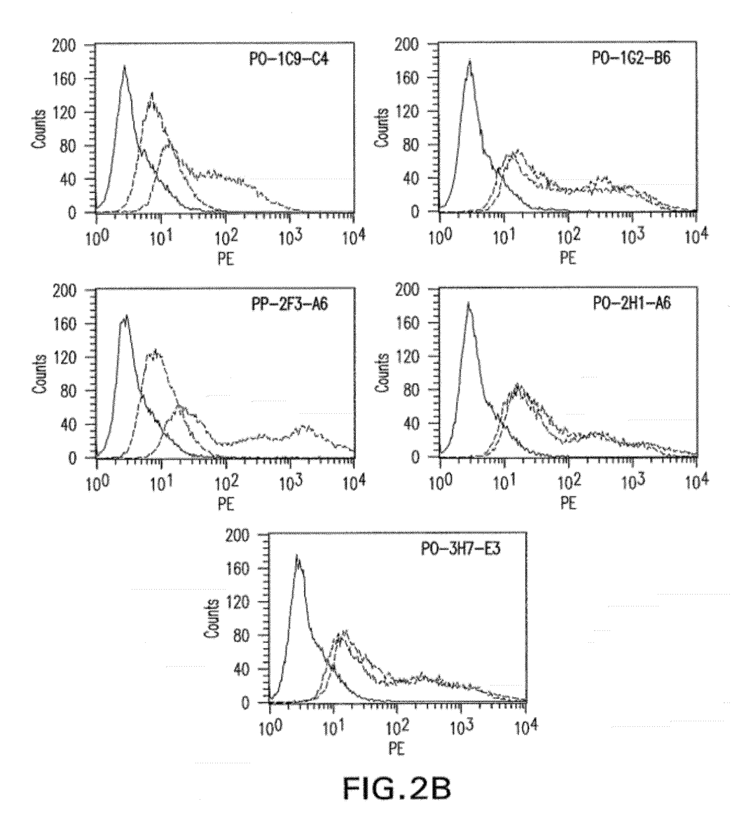

CHO cell lines expressing human or mouse Her3 receptors were used to study species-cross reactivity. Cells were harvested and resuspended in PBS, 2% FBS at a concentration of approximately 1×10⁶/ml. Cells were stained with purified IgGs at 4° C. for 40 min. Cells were washed with 4 mL PBS, 2% FBS, then centrifuged at 1200 rpm for 10 minutes. Supernatant was removed. Cells were stained with R-PE conjugated anti-mouse IgG at 4° C. for 30 min. Cells were washed with 4 mL PBS, 2% FBS, then centrifuged at 1200 rpm for 10 minutes. Supernatant was removed and cell pellets were fixed in 1% formaldehyde. Cells were analyzed on a FACS Calibur (BD Biosciences) by collecting 20,000 events from gate 1. Binding of selected Her3 hybridoma clones on mouse and human Her3 on cells is shown in FIG. 2.

### Example 4

Cross Reactivity with Mouse Her3 and Other Her Family Members in FACS

CHO-Her2, CHO-Her4, CHO-EGFR cells were harvested and resuspended in PBS, 2% FBS at a concentration of approximately 1×10⁶/ml. Cells were stained with purified IgGs at 4° C. for 40 min. Cells were washed with 4 mL PBS, 2% FBS, then centrifuged at 1200 rpm for 10 minutes. Supernatant was removed. Cells were stained with R-PE conjugated anti-mouse IgG at 4° C. for 30 min. Cells were washed with 4 mL PBS, 2% FBS, then centrifuged at 1200 rpm for 10 minutes. Supernatant was removed and cell pellets were fixed in 1% formaldehyde. Cells were analyzed on a FACS Calibur (BD Biosciences) by collecting 20,000 events from gate 1. Binding of selected Her3 hybridoma clones on different Her cell lines is summarized in Table 2.

### Example 5

### Her3 Binding Affinity in Biacore Analysis

All experiments were performed at 25° C. at a flow rate of 40 μl/min. To prepare a BIAcore assay, an anti-human IgG-Fc antibody (50 μg/ml each in acetate buffer, pH 5.0) was immobilized onto a carboxymethyl dextran sensorchip (CM5) using amine coupling procedures as described by the manufacturer. 10000 resonance units (RU) of anti-mouse IgG Fc antibodies were linked respectively onto Flowcells (FC) 1 and 2. Purified Mabs to be tested were diluted at a concentration of 5 μg/ml in 0.5% P20, HBS-EP buffer and injected on FC2 to reach 500 to 1000 RU. FC1 was used as the reference cell. Specific signals correspond to the difference of signals obtained on FC2 versus FC1. The analyte (Recombinant Human Her3, apparent molecular weight 97 kDa) was injected during 90 sec at 5 different concentrations (100, 50, 25, 12.5 and 6.25 nM) in 0.5% P20, HBS-EP buffer. These concentrations were prepared from stock solution in 0.5% P20, HBS-EP. The dissociation phase of the analyte was monitored over a 10 minutes period. Running buffer was also injected under the same conditions as a double reference. After each cycle (antibody+Her3 injection), both Flowcells were regenerated by injecting 20 to 45 μl of Glycine-HCl buffer pH 1.5. This regeneration is sufficient to eliminate all Mabs and Mabs/Her3 complexes captured on the sensorchip. Table 1 listed binding KD for Her3 binding monoclonal antibodies.

### Example 6

Internalization Study with Purified Mouse IgGs

T47D cells were harvested and resuspended in complete culture medium at a concentration of approximately 1×10⁷/ml. 1000 cells were incubated at 37° C. for 3 hr with 100 μl human IgG (Jackson ImmunoResearch, Cat#009-000-003), mouse hybridoma purified IgGs at final concentrations of 0.1 μg/ml. Cells were transferred into FACS tubes and washed with 4 mL PBS, 2% FBS, then centrifuged at 1200 rpm for 10 minutes. Supernatant was removed. Cells were stained with 0.5 μg mouse IgG1 (BD Pharmingen, Cat#555746), mouse hybridoma purified IgGs at 4° C. for 40 minutes. Cells were washed with 4 mL PBS, 2% FBS, then centrifuged at 1200 rpm for 10 minutes. Supernatant was removed. Cells were stained with 0.5 μg R-PE conjugated rat anti-mouse Ig (BD Pharmingen, Cat#559940) at 4° C. for 30 minutes, covered with foil. Cells were washed with 4 mL PBS, 2% FBS, then centrifuged at 1200 rpm for 10 minutes. Supernatant was removed and cell pellets were fixed in 340 μl PBS/10% Formaldehyde. Cells were analyzed on a FACS Calibur (BD Biosciences) by collecting 20,000 events from gate 1. Percentage of Her3 receptor internalization mediated by isolated mouse hybridoma monoclonal antibodies is listed in Table 3.

### Example 7

Inhibition of pHer3, IC₅₀ with Purified Mouse IgGs

MCF7 cells were seeded at cell density approximately 3×10e5 cells/ml in 96 well plate and on the following day, cells were serum deprived overnight. Cells were treated with Her3-targeting agents, in medium containing 0.1% BSA for 2 hr at 37° C. Cells were unstimulated and stimulated with 3.3 nM rhNRG1-β1 in medium containing 0.1% BSA for 20 min at 37° C. During lysate preparation, coated plate (overnight coating with 4 ug/ml mouse anti-human ErbB3 antibody) was blocked with PBS containing 1% BSA. Cells were rinsed 3×s with cold PBS and lysed with Cell Extraction Buffer (containing fresh 1 mM PMSF, protease inhibitor cocktail, and phosphatase inhibitor cocktail) for 30 min with gentle rocking at 4° C. After lysate were mixed, the plate was centrifuged at 3,000 rpm for 10 min. 100 ul supernatant was transferred to blocked assay plate and incubated for 2 hr at RT. Wells were washed with 300 ul PBST 5 times and detected with Anti-phospho tyrosine HRP Detection Antibody during 2 hr incubation at RT. Wash was repeated and HRP chemiluminescence substrate was added for 2 min with gentle rocking at RT. Plate was read on Victor plate reader and data was analyzed.

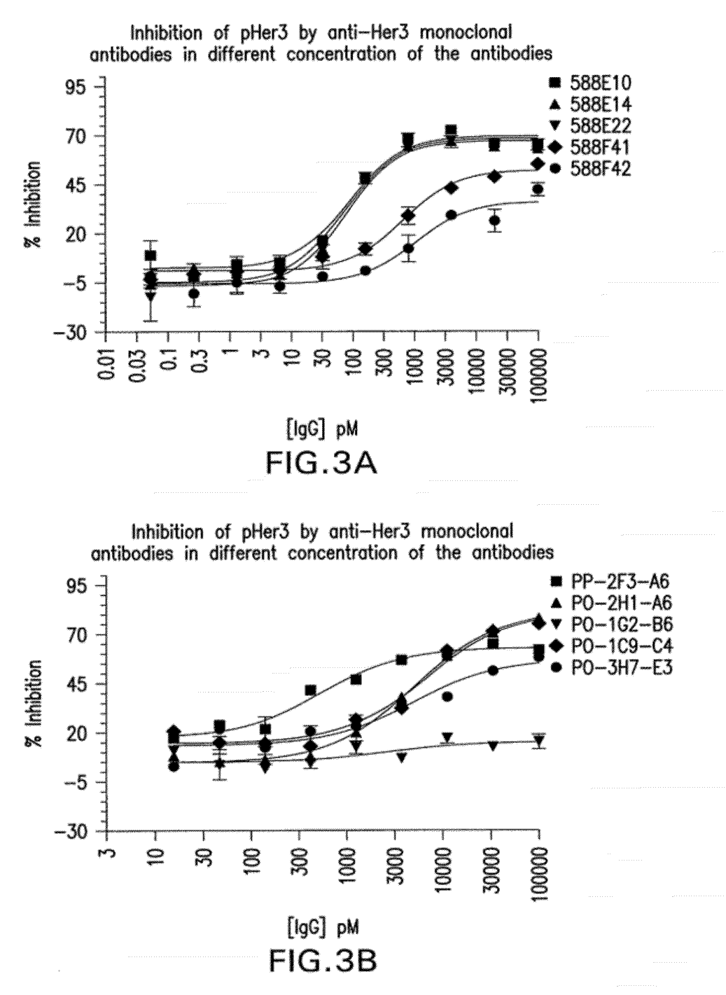

FIGS. 3a and 3b show titration of purified Her3 IgGs generated in two stages, respectively. Table 4 shows IC₅₀ determination of purified Her3 IgGs.

### Example 8

Inhibition of pAKT, IC₅₀ with Purified Mouse IgGs

MCF7 cells were seeded at cell density approximately 3×10e5 cells/ml in 96 well plate and on the following day, cells were serum deprived overnight. Cells were treated with Her3-targeting agents, in medium containing 0.1% BSA for 2 hr at 37° C. Cells were unstimulated and stimulated with 3.3 nM rhNRG1-β1 in medium containing 0.1% BSA for 20 min at 37° C. During lysate preparation, coated plate (overnight coating with 2 ug/ml mouse anti-human Akt (Pan) antibody) was blocked with PBS containing 1% BSA. Cells were rinsed 3×s with cold PBS and lysed with Cell Extraction Buffer (containing fresh 1 mM PMSF, protease inhibitor cocktail, and phosphatase inhibitor cocktail) for 30 min with gentle rocking at 4° C. After lysate were mixed, the plate was centrifuged at 3,000 rpm for 10 min. 100 ul supernatant was transferred to blocked assay plate and incubated for 2 hr at RT. Wells were washed with 300 ul PBST 5 times and detected with biotinylated rabbit anti-human phospho-Akt (Pan) (S473) during 2 hr incubation at RT. Wells were incubated with secondary antibody, Streptavidin HRP for 20 min at RT. Wash was repeated and HRP chemiluminescence substrate was added for 2 min with gentle rocking at RT. Plate was read on Victor plate reader (Perkin-Elmer) and data was analyzed.

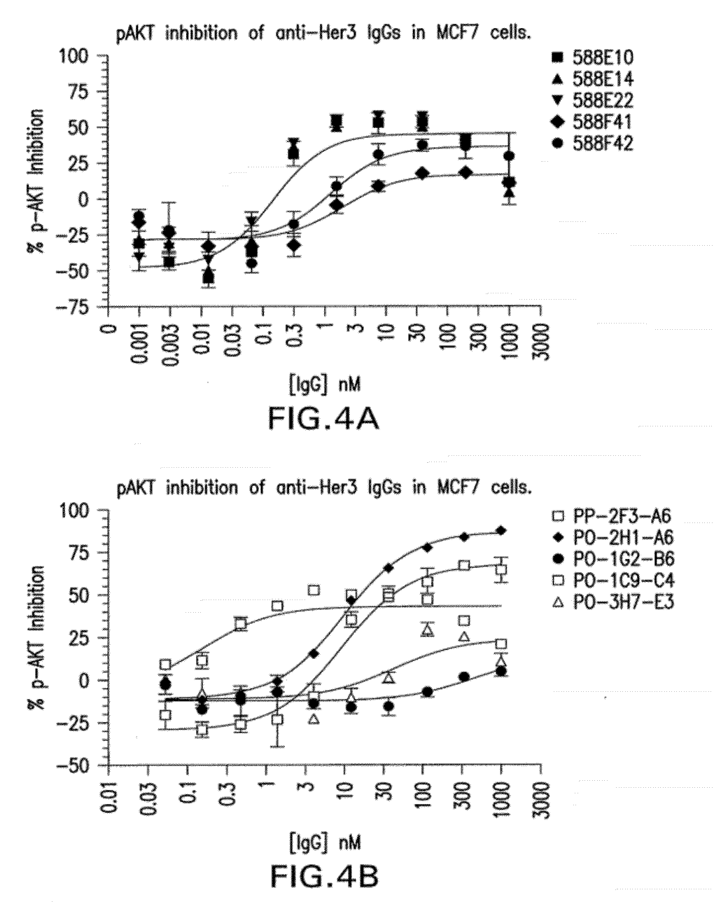

Mouse IgG was used as negative control. FIG. 4 shows pAKT inhibition of Her3 IgGs at a series of antibody concentrations in MCF7 cells. Inhibition IC₅₀ values were listed in Table 5 and were estimated using FIG. 4 titration curve fitting.

### Example 9

Inhibition of pERK, IC₅₀ with Purified Mouse IgGs

MCF7 cells were seeded at cell density approximately 3×10e5 cells/ml in 96 well plate and on the following day, cells were serum deprived overnight. Cells were treated with Her3-targeting agents, in medium containing 0.1% BSA for 2 hr at 37° C. Cells were unstimulated and stimulated with 3.3 nM rhNRG1-β1 in medium containing 0.1% BSA for 20 min at 37° C. During lysate preparation, blocked Meso Scale Discovery (MSD) pre-coated plate with TRIS containing 3% BSA with shaking. Cells were rinsed 3×s with cold PBS and lysed with Cell Extraction Buffer (containing fresh 1 mM PMSF, protease inhibitor cocktail, and phosphatase inhibitor cocktail) for 30 min with gentle rocking at 4° C. After lysate were mixed, the plate was centrifuged at 3,000 rpm for 10 min. 25 μl supernatant was transferred to blocked assay plate and incubated for 2 hr at RT with shaking. Wells were washed with 300 μl Tris 3 times and detected with MSD anti-phospho-ERK1/2 (T/Y:202/204; 185/187) labeled with an electrochemiluminescent compound, MSD SULFO-TAC™ label during 1 hr incubation with shaking. Wash was repeated and MSD Read Buffer T, with surfactant was added to plate. Plate was read on MSD SECTOR™ Imager2400 and data was analyzed.

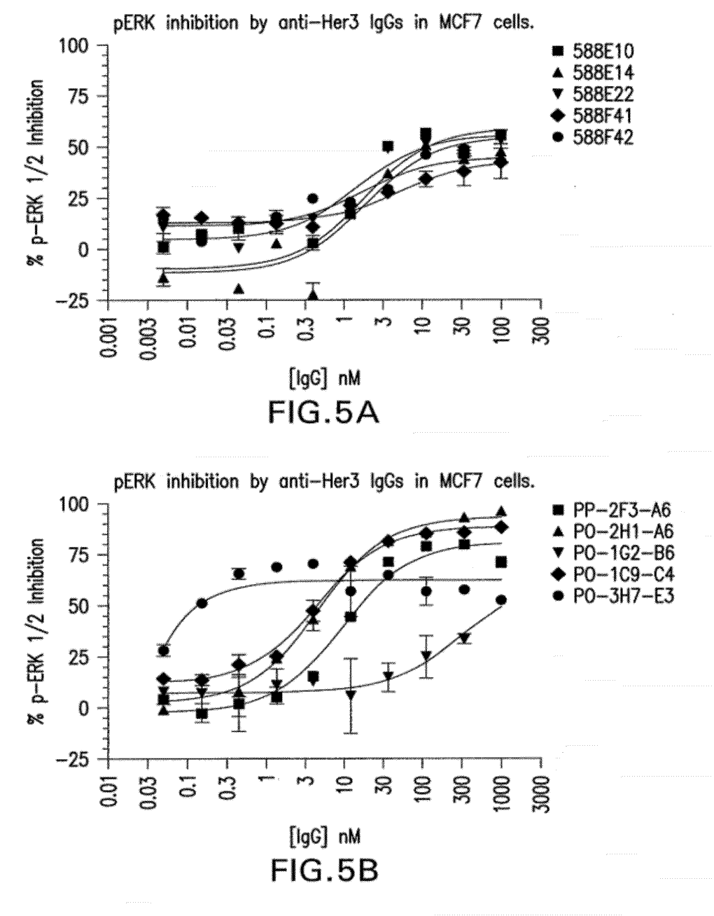

FIGS. 5(A&B) shows titration of two sets of purified anti-Her3 IgGs, produced by SDI and Genovac, respectively. Table 6 shows IC₅₀ of inhibition of pERK by purified Her3 IgGs.

### Example 10

Inhibition of Ligand Stimulated Cell Proliferation with Purified Mouse IgGs

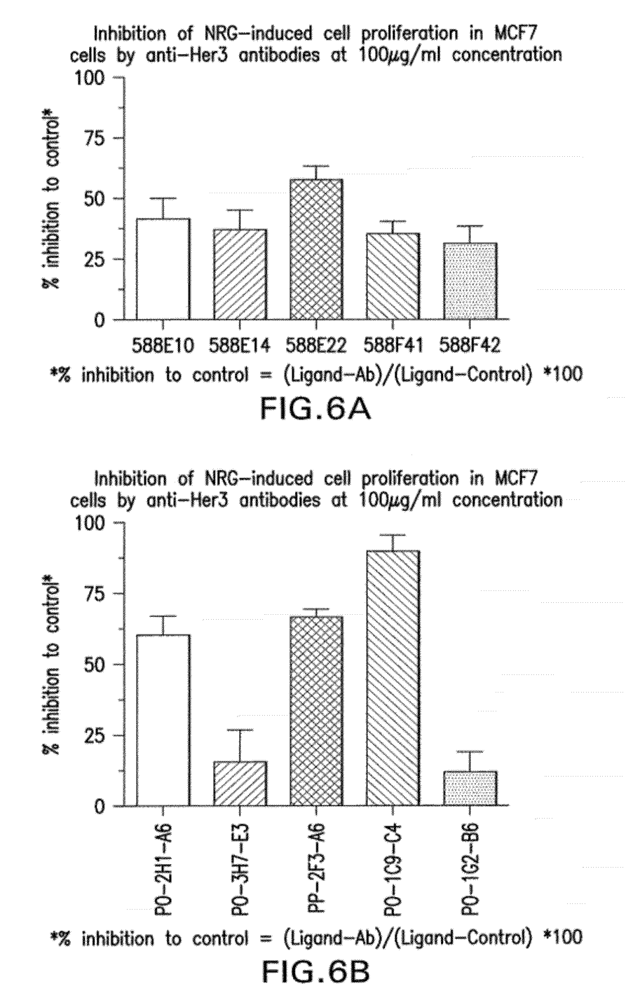

In vitro experiments were conducted to determine the ability of the antibodies to inhibit NRG-stimulated cell proliferation. 3000 MCF7 (human breast cancer) cells were seeded in 100 μl of 10% FBS medium on 96-well a 37° C. incubator overnight. The medium was changed with 0.5% FBS medium and antibodies were added to the cells for one hour at 37° C. Cells were stimulated with 10, 50 and 100 ng/ml β-NRG by adding the ligand directly to antibody medium and were then left to grow for 72 hours. 10 μl of AlamarBlue™ (BIOSOURCE) was added to the medium and the cells were incubated at 37° C. incubator. Fluorescence (535/590 nm) was measured very hour. The data were taken two hours after addition of AlamarBlue™. The results as indicated in FIGS. 6 (A&B) show that the antibodies inhibit NRG-induced cell growth in MCF7 cells and date are shown as percentage of reduction by the antibodies relative to no antibody treated.

### Example 11

Ligand Blocking Assay Using Alpha Screen Format, IC₅₀, with Purified Mouse IgGs

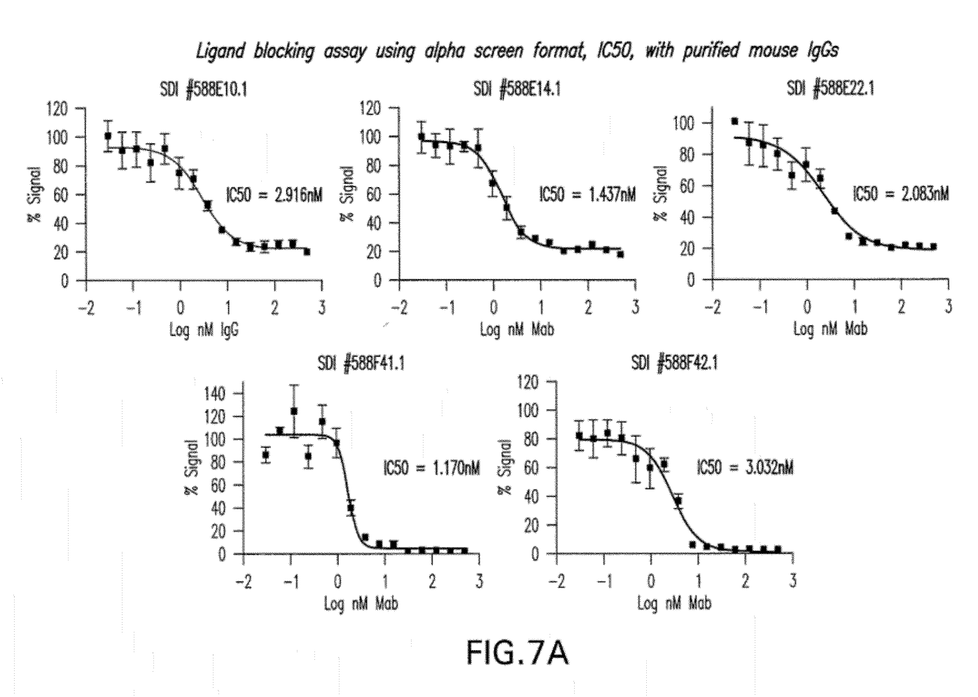

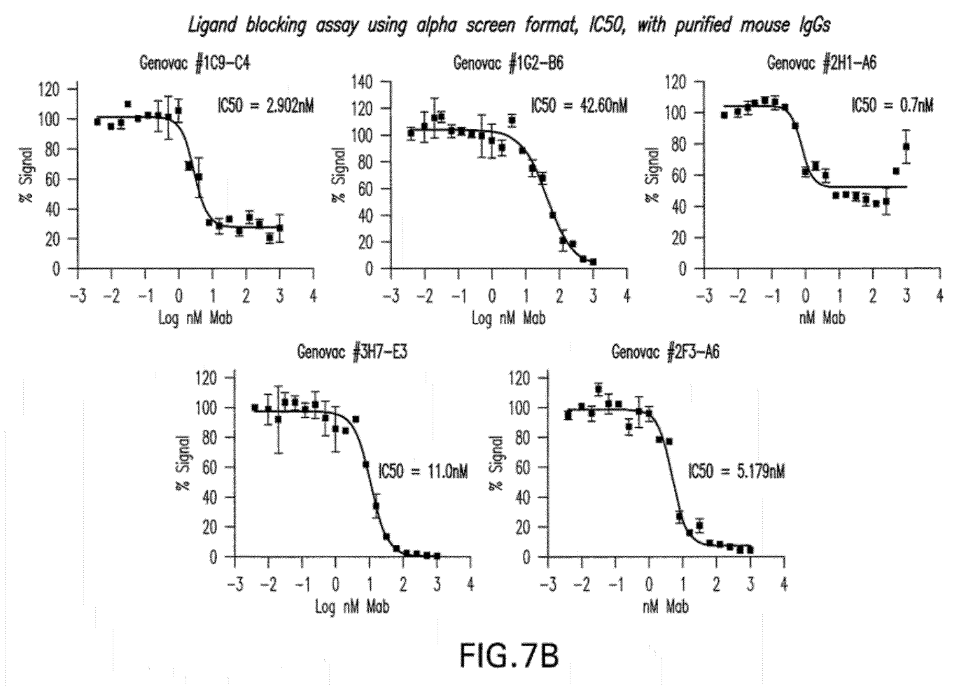

SDI or Genovac purified anti-hHer3 Mabs were serially diluted two-fold into an assay buffer made from 25 mM Hepes, 100 mM NaCl, 0.5% BSA and 0.05% Tween20 in an opaque/white, half-well area plate (Costar). Mouse IgG1 antibody was used as a negative control (Southern Biotech; cat #010201). Biotinylated NRG-1 beta ligand and Her3/Avi/His₁₀ receptor were added sequentially at 10 nM concentration to the assay plate containing serially diluted antibodies. This mixture of receptor, ligand and the antibodies was incubated at room temperature with gentle shaking for 90 minutes. AlphaScreen Streptavidin Donor beads and His-Nickel chelate Acceptor beads were added into the assay at a final concentration of 20 μg/ml each. The light sensitive beads were handled under chromagreen filtered light. The assay plates were covered with foil and incubated at room temperature with gentle shaking for 1 hour. The plates were read in PE EnVision plate reader equipped with an Alpha module (dedicated laser for excitation and designed to measure the signal above the well). Table 7 listed IC₅₀ values in ligand blocking that were estimated using four parameter curve fitting with concentration titration graphs (FIGS. 7 A&B).

### Example 12

cDNA sequence cloning from mouse hybridoma clones

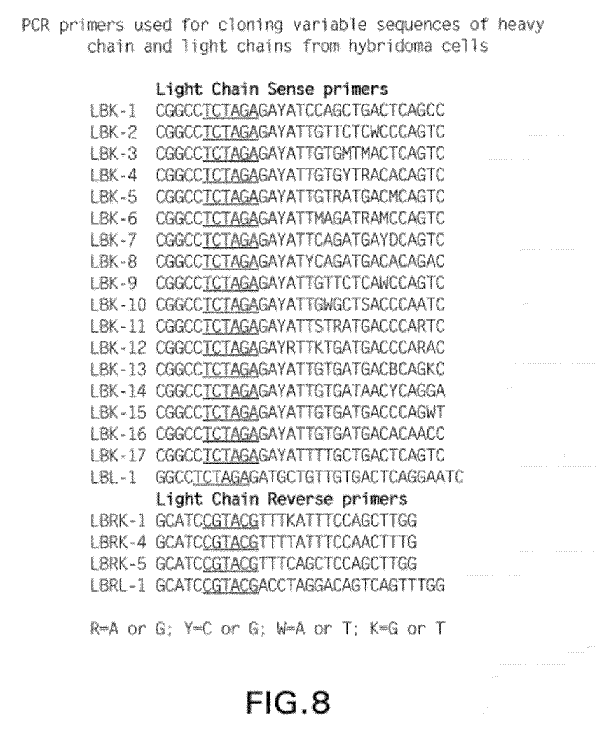

Total RNA was isolated from human Her3 mouse hybridomas (588E10, 588E14, 588E22, 588F41, 588F42, PO-2H1-A6, PO-3H7-E3, PP-2F3-A6, PO-1C9-C4 and PO-1G2-B6) with RNeasy protect cell mini kit (QIAGE). cDNA was synthesized with Superscript III (Invitrogen). IgG light chain variable region (LV) and heavy chain variable region (HV) were amplified with PCR using mixed primers (FIG. 8) and cDNA as template DNA. LV and HV were cloned into pCR4BluntTOPO vector (Invitrogen)). cDNA was sequenced by GENEWIZ™. Both light chain variable and heavy chain variable DNA and amino acid sequences are listed in appendix I & II. CDRs of light chain and heavy chain are listed in Table 8&9.

### SUMMARY OF TABLES

Table 1. Her3 antibody binding kinetic constants on human Her3/ErbB3 extracellular domain determined using BIAcore (SPR) analysis
Table 2. Anti-Her3 antibody cross reactivity with other epidermal growth factor receptor family members, EGFR, Her2, and Her4
Table 3. percentage of Her3 receptor internalization mediated by anti-Her3 antibodies
Table 4. Inhibition of pHer3, IC₅₀ estimated from dose dependence titration curves
Table 5. Inhibition of pAKT, IC₅₀ estimated from dose dependence titration curves
Table 6. Inhibition of pERK, IC₅₀ estimated from dose dependence titration curves
Table 7. Blocking ligand binding by anti-Her3 antibodies, IC₅₀ values estimated using concentration titration graphs
Table 8. Heavy chain CDR sequences for 12 Anti-Her3 clones
Table 9. Light chain CDR sequences for 12 Anti-Her3 binding clones

### APPENDIX

Appendix I. Variable DNA sequences of anti-Her3 antibodies
Appendix II. Variable amino acid sequences of anti-Her3 antibodies
Appendix III. Full length human Her3 amino acid sequences

TABLE 1
Her3 ECD binding affinity (K_{D}) measured using SPR method

| Anti-Her3 antibodies | K_{D} (nM) |
|---|---|
| PP-2F3-6A | 7.19 ± 0.36 |
| PO-2H1-A6 | 7.4 ± 0.31 |
| PO-1G2-B6 | 5.5 ± 0.66 |
| PO-1C9-C4 | 2.4 ± 0.13 |
| PO-3H7-E3 | 1.6 ± 0.20 |
| 588E10 | 3.31 ± 0.69 |
| 588E14 | 3.04 ± 0.51 |
| 588E22 | 2.78 ± 0.41 |
| 588F41 | 3.2 ± 0.17 |
| 588F42 | 3.8 ± 0.18 |

TABLE 2
Cross reactivity of the mouse anti-Her3 antibodies with
other HER family receptors

| Sample | | | |
|---|---|---|---|
| Name | Her2 | Her4 | EGFR |
| 588E10 | negative | negative | negative |
| 588E14 | negative | negative | negative |
| 588E22 | negative | negative | negative |
| 588F7 | negative | negative | negative |
| 588F41 | negative | negative | negative |
| 588F42 | negative | negative | negative |
| PO-1C9-C4 | negative | negative | negative |
| PO-1G2-B6 | negative | negative | negative |
| PO-2H1-A6 | negative | negative | negative |
| PO-3H7-E3 | negative | negative | negative |
| PP-2F3-A6 | negative | negative | negative |

TABLE 3
Percentage of Her3 receptor internalization mediated by
isolated mouse hybridoma monoclonal antibodies

| | % Receptor |
|---|---|
| Mouse anti-Her3 IgGs | Internalization |
| 588E10 | 51 ± 2.5 |
| 588E14 | 52 ± 2.8 |
| 588E22 | 53 ± 1.6 |
| 588F41 | 59 ± 2.0 |
| 588F42 | 57 ± 1.6 |
| PO-1C9-C4 | 41 ± 5.0 |
| PO-1G2-B6 | 13 ± 0.4 |
| PP-2F3-A6 | 65 ± 1.4 |
| PO-2H1-A6 | 49 ± 1.4 |
| PO-3H7-E3 | 14 ± 0.7 |

TABLE 4
Inhibition of pHer3, IC₅₀ for anti-Her3 mouse IgGs

| Antibody Name | IC₅₀, nM |
|---|---|
| 588E10 | 0.09 |
| 588E14 | 0.07 |
| 588E22 | 0.09 |
| 588F41 | 0.66 |
| 588F42 | 1.04 |
| PP-2F3-A6 | 0.55 |
| PO-2H1-A6 | 5.03 |
| PO-1G2-B6 | 2.91 |
| PO-1C9-C4 | 6.99 |
| PO-3H7-E3 | 4.98 |

TABLE 5
IC₅₀ of pAKT inhibition by anti-Her3 IgGs

| Anti-Her3 antibodies | nM |
|---|---|
| 588E10 | 0.14 |
| 588E22 | 0.09 |
| 588F41 | 2.05 |
| 588F42 | 1.36 |
| PP-2F3-A6 | 0.15 |
| PO-2H1-A6 | 10.10 |
| PO-1G2-B6 | N/A* |
| PO-1C9-C4 | 9.47 |
| PO-3H7-E3 | 45.17 |

*N/A, activity was too low and IC₅₀ was not determined.

TABLE 6
IC₅₀ of pERK inhibition by anti-Her3 IgGs

| Anti-Her3 IgGs | nM |
|---|---|
| 588E10 | 1.72 |
| 588E14 | 1.82 |
| 588E22 | 1.44 |
| 588F41 | 4.91 |
| 588F42 | 1.80 |
| PP-2F3-A6 | 9.88 |
| PO-2H1-A6 | 4.83 |
| PO-1G2-B6 | 289.80 |
| PO-1C9-C4 | 4.62 |
| PO-3H7-E3 | N/A* |

*N/A, activity was too low and IC₅₀ was not determined.

TABLE 7
IC₅₀ values of ligand blocking estimated using concentration
titration curves fitting, n = 3

| Anti-Her3 | |
|---|---|
| monoclonal antibodies | IC₅₀, nM |
| 588E10 | 4.9 ± 2.8 |
| 588E14 | 2.3 ± 1.2 |
| 588E22 | 3.6 ± 1.7 |
| 588F41 | 4.1 ± 3.4 |
| 588F42 | 4.6 ± 2.3 |
| PP-2F3-A6 | 4.4 ± 1.2 |
| PO-2H1-A6 | 1.0 ± 0.4 |
| PO-1G2-B6 | 72.8 ± 42.7 |
| PO-1C9-C4 | 2.7 ± 0.4 |
| PO-3H7-E3 | 12.7 ± 2.4 |

TABLE 8
Heavy chain CDR amino acid sequences of
anti-Her3 mouse hybridoma clones

| Clone | | | | |
|---|---|---|---|---|
| Name | Germlines | CDR1 | CDR2 | CDR3 |
| 588E10HC | IGHV1-67* | GYTFTDYG | ITIYDGH | ARGDYDLFWYLDV |
| | 01 | (SEQ ID | (SEQ ID | (SEQ ID |
| | | NO: 3) | NO: 4) | NO: 5) |
| 588F7-HC | IGHV1S130* | GYTFTNSW | IHPNSGN | ARYEGH |
| | 01 | (SEQ ID | (SEQ ID | (SEQ ID |
| | | NO: 6) | NO: 7) | NO: 8) |
| 588F40HC | IGHV1- | GYTFTSYV | INPYNDG | AREASYGNPFAY |
| | 14*02 | (SEQ ID | (SEQ ID | (SEQ ID |
| | | NO: 9) | NO: 10) | NO: 11) |
| PO-3H7- | IGHV1- | GYSFTGYY | INPYYGG | ARKGDLYYSMDY |
| E3HC | 19*01 | (SEQ ID | (SEQ ID | (SEQ ID |
| | | NO: 12) | NO: 13) | NO: 14) |
| PP-2F3- | IGHV3- | GYSITSDYA | INYSGS | ARDDYDGYYFDY |
| A6HC | 2*02 | (SEQ ID | (SEQ ID | (SEQ ID |
| | | NO: 15) | NO: 16) | NO: 17) |
| PO-1C9- | IGHV1S22* | GYTFTIFW | IYPGSGG | TRWGTGKDY |
| C4HC | 01 | (SEQ ID | (SEQ ID | (SEQ ID |
| | | NO: 18) | NO: 19) | NO: 20) |
| PO-2H1- | IGHV8-8* | GFSLSTYGMG | IWWNDD | VQIANPYWYFDV |
| A6HC | 01 | (SEQ ID | (SEQ ID | (SEQ ID |
| | | NO: 21) | NO: 22) | NO: 23) |
| PO-1G2- | IGHV1- | GYSFTGYH | INPYSGV | ARTDYYGPWFAY |
| B6HC | 31*01 | (SEQ ID | (SEQ ID | (SEQ ID |
| | | NO: 24) | NO: 25) | NO: 26) |

TABLE 9
Light chain CDR amino acid sequences of
anti-Her3 mouse hybridoma clones

| Clone | | | | |
|---|---|---|---|---|
| Name | Germlines | CDR1 | CDR2 | CDR3 |
| 588E10LC | IGKV1- | QNIKHSDGNTY | KV | FQGSHVPLT |
| | 117*01 | (SEQ ID | (SEQ ID | (SEQ ID |
| | | NO: 27) | NO: 28) | NO: 29) |
| 588F7LC | IGKV1- | QSLLDSDGKTY | LV | WQGTHFPQT |
| | 135*01 | (SEQ ID | (SEQ ID | (SEQ ID |
| | | NO: 30) | NO: 31) | NO: 32) |
| 588F40LC | IGKV12- | ENIYSY | NT | QHHYDTPWT |
| | 44*01 | (SEQ ID | (SEQ ID | (SEQ ID |
| | | NO: 33) | NO: 34) | NO: 35) |
| PO-3H7- | IGKV1- | QSIVHSNGNTY | KV | FQGSHVPPT |
| E3LC | 117*01 | (SEQ ID | (SEQ ID | (SEQ ID |
| | | NO: 36) | NO: 37) | NO: 38) |
| PP-2F3- | IGKV3- | KSVSTSGYSY | LV | QHIRELTR |
| A6LC | 12*01 | (SEQ ID | (SEQ ID | (SEQ ID |
| | | NO: 39) | NO: 40) | NO: 41) |
| PO-1C9- | IGKV4- | SSVSSSY | ST | QQYDSSPFT |
| C4LC | 78*01 | (SEQ ID | (SEQ ID | (SEQ ID |
| | | NO: 42) | NO: 43) | NO: 44) |
| PO-2H1- | IGKV3- | KSVSTSGYSY | LV | QHIRELTR |
| A6LC | 12*01 | (SEQ ID | (SEQ ID | (SEQ ID |
| | | NO: 45) | NO: 46) | NO: 47) |
| PO-1G2- | IGKV1- | PSLVHSNGNTY | TV | SQTTHVPWT |
| B6LC | 110*01 | (SEQ ID | (SEQ ID | (SEQ ID |
| | | NO: 48) | NO: 50) | NO: 49) |

### Appendix I

### List of DNA Sequences for Anti-Her3 Murine Antibody Clones

>588E10-HC
(SEQ ID NO: 51)
CTCGAGGAGGTGCAGCTTCAGGAGTCTGGGCCTAAACTGGTGGGGCCTGGGGAATCAGTGAAGA
TTTCCTGCAAGGGTTCCGGCTACACATTCACTGATTACGGTATGCACTGGGTGAAACAGAGTCA
TGCAAAGAGGCTCGAGTGGATTGGAGTTATCACTATTTATGATGGTCATACAAACTACAACCAG
AACTTTAAGGGCAAGGCCGCATTGACTGTAGACAAATCCTCCAACACAGCCTATCTGGAACTTG
CCAGAATGACTTCTGAGGATTCTGCCATCTATTACTGTGCAAGGGGAGATTACGACCTCTTCTG
GTATTTGGATGTCTGGGGCGCAGGGACCACTCTCACAGTCTCCTCGGCCTCCACCAAGGGCCC
>588E10-LC
(SEQ ID NO: 52)
TCTAGAGATGTTGTGATGACCCAGACTCCACTCTCCCTGACTGTCAGTCTTGGAGATCAAGCCT
CCATCTCTTGCAGATCTAGTCAGAACATTAAACATAGTGATGGAAACACCTATTTAGAATGGTA
CCTGCAGAAACCAGGCCAGTCTCCAAAGCTCCTGATCTACAAAGTTTCCAACCGATTTTCTGGG
GTCCCAGACAGGTTCAGTGGCAGTGGATCAGGGACAGATTTCACACTCAAGATCAGTAGAGTGG
AGGCTGAGGATCTGGGAGTTTATTTCTGCTTTCAAGGTTCACATGTTCCTCTCACGTTCGGTGC
TGGGACCAAGCTGGAGCTGAAACGTACG
>588E14-HC
(SEQ ID NO: 53)
CTCGAGGAGGTGCAGCTGCAGCAGTCTGGGCCTGAACTGGTGGGGCCTGGGGAATCAGTGAAGA
TTTCCTGCAAGGGTTCCGGCTACGCATTCACTGATTACGGTATGCACTGGGTGAAACAGAGTCA
TGCAAAGAGGCTCGAGTGGATTGGAGTTATCACTATTTATGATGGTCATACAAACTACAACCAG
AACTTTAAGGGCAAGGCCGCATTGACTGTAGACAAATCCTCCAACACAGCCTATCTGGAACTTG
CCAGAATGACTTCTGAGGATTCTGCCATCTATTACTGTGCAAGGGGAGATTACGACCTCTTCTG
GTATTTGGATGTCTGGGGCGCAGGAACCTCAGTCACCGTCTCCTCGGCCTCCACCAAGGGCCC
>588E14-LC
(SEQ ID NO: 54)
TCTAGAGACATTGTGATGACCCAAACTCCACTCTCCCTGACTGTCAGTCTTGGAGATCAAGCCT
CCATCTCTTGCAGATCTAGTCAGAACATTAAACATAGTGATGGAAACACCTATTTAGAATGGTA
CCTGCAGAAACCAGGCCAGTCTCCAAAGCTCCTGATCTACAAAGTTTCCAACCGATTTTCTGGG
GTCCCAGACAGGTTCAGTGGCAGTGGATCAGGGACAGATTTCACACTCAAGATCAGTAGAGTGG
AGGCTGAGGATCTGGGAGTTTATTTCTGCTTTCAAGGTTCACATGTTCCTCTCACGTTCGGTGC
TGGGACCAAGCTGGAGCTGAAACGTACG
>588E22-RC
(SEQ ID NO: 55)
CTCGAGGATGTGCAGCTTCAGGAGTCTGGGCCTGAACTGGTGGGGCCTGGGGAATCAGTGAAGA
TTTCCTGCAAGGGTTCCGGCTACACATTCACTGATTACGGTATGCACTGGGTGAAACAGAGTCA
TGCAAAGAGGCTCGAGTGGATTGGAGTTATCACTATTTATGATGGTCATACAAACTACAACCAG
AACTTTAAGGGCAAGGCCGCATTGACTGTAGACAAATCCTCCAACACAGCCTATCCGGAACTTG
CCAGAATGACTTCTGAGGATTCTGCCATCTATTACTGTGCAAGGGGAGATTACGACCTCTTCTG
GTATTTGGATGTCTGGGGCGCAGGGACCACGGTCACCGTTTCCTCGGCCTCCACCAAGGGCCC
>588E22-LC
(SEQ ID NO: 56)
TCTAGAGATGTTGTGATGACCCAAACTCCACTCTCCCTGACTGTCAGTCTTGGAGATCAAGCCT
CCATCTCTTGCAGATCTAGTCAGAACATTAAACATAGTGATGGAAACACCTATTTAGAATGGTA
CCTGCAGAAACCAGGCCAGTCTCCAAAGCTCCTGATCTACAAAGTTTCCAACCGATTTTCTGGG
GTCCCAGACAGGTTCAGTGGCAGTGGATCAGGGACAGATTTCACACTCAAGATCAGTAGAGTGG
AGGCTGAGGATCTGGGAGTTTATTTCTGCTTTCAAGGTTCACATGTTCCTCTCACGTTCGGTGC
TGGGACCAAGCTGGAGCTGAAACGTACG
>588F7-HC
(SEQ ID NO: 57)
CTCGAGCAGGTCCAACTACAGCAACCTGGGTCTGTGCTGGTGAGGCCTGGAGCTTCAGTGAAGC
TGTCCTGCAAGGCTTCTGGCTACACCTTCACCAACTCCTGGATACTCTGGGCGAAGCGGAGGCC
TGGACAAGGCCTTGAGTGGATTGGAGAGATTCATCCTAATAGTGGTAATACTAACTATAATGAG
AAGTTCAAGGGCCAGGCCACACTGACTGTAGACACATCCTCCACCACAGCCTACGTGGATCTCA
GCAGGCTGACATCTGAGGACTCTGCGGTCTATTACTGTGCAAGATATGAGGGACACTGGGGCCA
AGGGACTCTGGTCACTGTCTCTGCGGCCTCCACCAAAGGGCCC
>588F7-LC
(SEQ ID NO: 58)
TCTAGAGATGTTGTGATGACCCAGACTCCACTCACTTTGTCGGTTACCATTGGACAACCAGCCT
CCATCTCTTGTAAGTCAAGTCAGAGCCTCTTAGATAGTGATGGAAAGACATATTTGAATTGGTT
GTTACAGAGGTCAGGCCAGTCTCCAAAGCGCCTAATCTATCTGGTGTCTAAACTGGACTCTGGA
GTCCCTGACAGGTTCACTGGCAGTGGATCAGGGACAGATTTCACACTGAAAATCAGCAGAGTGG
AGGCTGAGGATTTGGGAGTTTATTATTGCTGGCAAGGTACACATTTTCCTCAGACGTTCGGTGG
AGGCACCAAGCTGGAAATCAAACGTACG
>588F40-HC
(SEQ ID NO: 59)
CTCGAGGAGGTGAAGCTGATGGAGTCTGGACCTGAACTGGTTAAGCCTGGGGCTTCAGTGAAGA
TGTCCTGCAAGGCTTCTGGATACACATTCACTAGCTATGTTATGCACTGGGTGAAGCAGAAGCC
TGGGCAGGGCCTTGAGTGGCTTGGATATATTAACCCTTACAATGATGGTACTAAGTACAATGAG
AGGTTCAAAGGCRAGGCCACACTGACTTCAGACAAATCCTCCAGCACAGCCTACATGGAGCTCA
TCAGCCTGACCTCTGAGGACTCTGCGGTCTATTACTGTGCAAGAGAGGCCTCCTATGGTAACCC
CTTTGCTTACTGGGGCCAAGGGACTCTGGTCACTGTCTCTGCGGCCTCCACCCCAAGGGCCC
>588F40-LC
(SEQ ID NO: 60)
TCTAGAGACATTCAGATGATGCAGTCTCCAGCCTCCCTATCTGCATCTGTGGGAGAAACTGTCA
CCATCACATGTCGAACAAGTGAGAATATTTACAGTTATTTAGCATGGTTTCAGCAGAAACAGGG
AAATTCTCCTCAGCTCCTGGTCTATAATACAAAAACCTTAGCGGAAGGTGTGCCATCAAGGTTC
AGTGGCAGTGGATCAGGCACACAGTTTTCTCTGAAGATCAACAGCCTGCAGCCTGAAGATTTTG
GGAATTATTACTGTCAACATCATTATGATACTCCGTGGACGTTCGGTGGAGGCACCAAGCTGGA
AATCAAACGTACG
>F41-HC
(SEQ ID NO: 61)
CTCGAGGAGGTTCAGCTTCAGCAGTCTGGACCTGAACTGGTTAAGCCTGGGGCTTCAGTGAAGA
TGTCCTGCAAGGCTTCTGGATACACATTCACTAGCTATGTTATGCACTGGGTGAAGCAGAAGCC
TGGGCAGGGCCTTGAGTGGCTTGGATATATTAACCCTTACAATGATGGTACTAAGTACAATGAG
AAGTTCAAAGGCATGGCCACACTGACTTCAGACAAATCCTCCAGCACAGCCTACATGGAGCTCA
TCAGCCTGACCTCTGAGGACTCTGCGGTCTATTACTGTGCAAGAGAGGCCTCCTATGGTAACCC
CTTTGCTTACTGGGGCCAAGGGACTCTGGTCACTGTCTCTGCGGCCTCCACCAAGGGCCC
>588F41-LC
(SEQ ID NO: 62)
TCTAGAGACATTGTGATGACCCAGTCTCCAGCCTCCCTATCTGCATCTGTGGGAGAAACTGTCA
CCATCACATGTCGAACAAGTGAGAATATTTACAGTTATTTAGCATGGTTTCAGCAGAAACAGGG
AAATTCTCCTCAGCTCCTGGTCTATAATACAAAAACCTTAGCGGAAGGTGTGCCATCAAGGTTC
AGTGGCAGTGGATCAGGCACACAGTTTTCTCTGAAGATCAACAGCCTGCAGCCTGAAGATTTTG
GGAATTATTACTGTCAACATCATTATGATACTCCGTGGACGTTCGGTGGAGGCACCAAGCTGGA
AATCAAACGTACG
>588F42-HC
(SEQ ID NO: 63)
CTCGAGGAGGTGCAGCTTCAGCAGTCTGGACCTGAACTGGTTAAGCCTGGGGCTTCAGTGAAGA
TGTCCTGCAAGGCTTCTGGATACACATTCACTAGCTATGTTATGCACTGGGTGAAGCAGAAGCC
TGGGCAGGGCCTTGAGTGGCTTGGATATATTAACCCTTACAATGATGGTACTAAGTACAATGAG
AAGTTCAAAGGCAAGGCCACACTGACTTCAGACAAATCCTCCAGCACAGCCTACATGGAGCTCA
TCAGCCTGACCTCTGAGGACTCTGCGGTCTATTACTGTGCAAGAGAGGCCTCCTATGGTAACCC
CTTTGCTTACTGGGGCCAAGGGACTCTGGTCACTGTCTCTGCGGCCTCCCCACCCAAGGGCCC
>588F42-LC
(SEQ ID NO: 64)
TCTAGAGACATTGTGATGACCCAGTCTCCAGCCTCCCTATCTGCATCTGTGGGAGAAACTGTCA
CCATCACATGTCGAACAAGTGAGAATATTTACAGTTATTTAGCATGGTTTCAGCAGAAACAGGG
AAATTCTCCTCAGCTCCTGGTCTATAATACAAAAACCTTAGCGGAAGGTGTGCCATCAAGGTTC
AGTGGCAGTGGATCAGGCACACAGTTTTCTCTGAAGATCAACAGCCTGCAGCCTGAAGATTTTG
GGAATTATTACTGTCAACATCATTATGATACTCCGTGGACGTTCGGTGGAGGCACCAAGCTGGA
AATAAAACGTACG
>PO-3H7-E3-HC
(SEQ ID NO: 65)
CTCGAGGAGGTGCAGCTGCAGCAGTCTGGAGCTGAGCTGGTGAAGCCTGGGGCTTCAGTGAAGA
TATCCTGCAAGGCTTCTGGTTACTCATTCACTGGCTACTACATGAACTGGGTGAAGCAGAGCCA
TGGAAAGAGCCTTGAGTGGATTGGAAATATTAATCCTTACTATGGCGGTAGTAACTACAATCAG
AAGTTCAAGGCCAGGGCCACATTGACTGTAGACAAATCTTCCAGCACAGCCTACATGCAGCTCA
ACAGCCTGACATCGGAGGACTCTGCAGTCTATTACTGTGCAAGAAAAGGTGACCTTTACTATTC
TATGGACTACTGGGGTCAAGGAACCTCAGTCACCGTCTCCTCGGCCCTCCCACCCAAGGGCCC
>PO-3H7-E3-LC
(SEQ ID NO: 66)
TCTAGAGACATTGTGATGACCCAAACTCCTCTCTCCCTGCCTGTCAGTCTTGGAGATCAGGCCT
CCATTTCTTGCAGATCTAGTCAGAGCATTGTACATAGTAATGGAAACACCTATTTAGAATGGTA
CCTACAGAAACCAGGCCAGTCCCCAAAGCTCCTCATCTACAAAGTTTCCAACCGATTTTCTGGG
GTCCCAGACAGGTTCAGTGGCAGTGGATCAGGGACAGATTTCACACTCAAGATCAGCAGAGTGG
AGGCTGAGGATCTGGGAATTTATTACTGCTTTCAAGGTTCACATGTTCCTCCGACGTTCGGTGG
AGGCACCAAGCTGGAGCTGAAACGTACG
>PP-2F3-A6-HC
(SEQ ID NO: 67)
CTCGAGGATGTGAAGCTTCAGGAGTCGGGACCTGGCCTGGTGAAACCTTCTCAGTCTCTGTCCC
TCACCTGCACTGTCACTGGCTACTCAATCACCAGTGATTATGCCTGGAACTGGATCCGGCAGTT
TCCAGGAAACAAACTGGAGTGGATGGGCTACATAAACTACAGTGGCAGCACTAACTATAACCCA
TCTCTCAAAAGTCGAATCTCTATCACTCGAGACACATCCAAGAACCAGTTCTTCCTGAAGCTGA
ATTCTGTGACCTCTGAGGACACAGCCACATATTACTGTGCAAGGGATGATTACGACGGGTACTA
CTTTGACTACTGGGGCCAAGGCACCACGGTCACCGTTTCCTCGGCCTCCACCAAGGGCCC
>PP-2F3-A6-LC
(SEQ ID NO: 68)
TCTAGAGACATTGTGTTAACACAGTCTCCTGCTTCCTTAGCTGTATCTCTGGGGCAGAGGGCCA
CCATCTCATACAGGGCCAGCAAAAGTGTCAGTACATCTGGCTATAGTTATATGCACTGGAACCA
ACAGAAACCAGGACAGCCACCCAGACTCCTCATCTATCTTGTATCCAACCTAGAATCTGGGGTC
CCTGCCAGGTTCAGTGGCAGTGGGTCTGGGACAGACTTCACCCTCAACATCCATCCTGTGGAGG
AGGAGGATGCTGCAACCCATTACTGTCAGCACATTAGGGAGCTTACACGTTCGGAGGGGGGACC
AAGCTGGAAAATCAAACGTACG
>PO-1C9-C4-HC
(SEQ ID NO: 69)
CTCGAGCAGGTCCAACTGCAGCAGCCTGGGTCTGAGCTGGTGAGGCCTGGAGCTTCAGTGAAGC
TGTCCTGCAAGGCTTCTGGCTACACATTCACCATCTTCTGGATCCACTGGGTGAAGCAGAGGCC
TGGACAAGGCCTTGAGTGGATTGGAAATATTTATCCTGGTAGTGGTGGAACTAACTACGATGAG
AAATTCAAGAGCAAGGCCACACTGACTGTAGACACATTTTCCAGCACAGCCTACATGCAGCTCA
GTAGCCTGACATCTGAGGACTCTGCGGTCTATTACTGTACAAGATGGGGGACTGGGAAGGACTA
CTGGGGCCAAGGCACCACTCTCACAGTCTCCTCGGCCTCCACCAAGGGCCC
>PO-1C9-C4-LC
(SEQ ID NO: 70)
TCTAGAGACATTGTTCTCACCCAGTCTCCAGCAATCATGTCTGCATCTCCAGGGGAGAAGGTCA
CCATGACCTGCAGGGCCAGGTCAAGTGTAAGTTCCAGTTACTTGCACTGGTACCAGCAGAAGCC
AGGATCTTCCCCCAAACTCTGGATTTATAGCACATCCAATCTGGCTTTAGGGGTCCCAGCTCGC
TTCAGTGGCAGTGGGTCTGGGACCTCTTACTCTCTCACAATCAGTAGTGTGGAGGCTGAGGATG
CTGCCACTTATTACTGCCAGCAGTATGATAGTTCCCCATTCACGTTCGGCACGGGGACAAAGTT
GGAAATAAAACGTACG
>PO-2H1-A6-HC
(SEQ ID NO: 71)
CTCGAGCAGGTTCAGCTGCANCAATCTGGCCCTGGGAAATTGCAGCCCTCCCAGACCCTCAGTC
TGACTTGTTCTTTTTCTGGGTTTTCACTGAGCACTTATGGTATGGGTGTAGGTTGGATTCGTCA
GCCTTTAGGGAAGGGTCTGGAGTGGCTGGCCAACATTTGGTGGAATGATGATAAGTACTATAAT
TCAGCCCTGAAGAGCCGGCTCACAATCTCCAAGGATACCTCCAACAACCAGGTTTTCCTCAAGA
TCTCCAGTGTGGACACTGCAGATGCTGCCACATACTACTGTGTTCAAATAGCTAACCCCTATTG
GTACTTCGATGTCTGGGGCGCAGGGACCTCAGTCACCGTCTCCTCGGCCTCCACCAAGGGCCC
>PO-2H1-A6-LC
(SEQ ID NO: 72)
TCTAGAGATATTCAGATGACTCAGTCTCCTGCTTCCTTAGCTGTATCTCTGGGGCAGAGGGCCA
CCATCTCATACAGGGCCAGCAAAAGTGTCAGTACATCTGGCTATAGTTATATGCACTGGAACCA
ACAGAAACCAGGACAGCCACCCAGACTCCTCATCTATCTTGTATCCAACCTAGAATCTGGGGTC
CCTGCCAGGTTCAGTGGCAGTGGGTCTGGGACAGACTTCACCCTCAACATCCATCCTGTGGAGG
AGGAGGATGCTGCAACCTATTACTGTCAGCACATTAGGGAGCTTACACGTTCGGAGGGGGGACC
AAGCTGGAAATCAAAACGTACG
>PO-1G2-B6-HC
(SEQ ID NO: 73)
CTCGAGGAGGTGCAGCTGCAGCAGTCTGGACCTGAGCTGGTGAAGCCTGGGGCTTCAGTGAAGA
TATCCTGCAAGGCTTCTGGTTACTCATTCACTGGCTACCACATACACTGGATGAAGCAAAGCCA
TGTAGAGAGCCTTGAGTGGATTGGACGTATTAATCCTTACAGTGGTGTTACTGACTACAACCAG
AATTTCAAGGACAAGGCCAACTTGACTGTAGATCAGTCCTCCAGCACAGCCTACATGGAACTCC
ACAGCCTGACATCTGAGGACTCTGCATTCTATTACTGTGCAAGGACGGATTACTACGGCCCCTG
GTTTGCTTACTGGGGCCAAGGGACTCTGGTCACTGTCTCTGCGGCCTCCACCAAGGGCCC
>PO-1G2-B6-LC
(SEQ ID NO: 74)
TCTAGAGATATTCAGATGACGCAGTCTCCACTCTCCCTGCCTGTCAGTCTTGGAGATCAAGCCT
CCATCTCTTGCAGATCTAGTCCGAGCCTTGTACACAGTAATGGAAACACCTATTTACATTGGTA
CCTGCAGAAGCCAGGCCAGTCTCCAAAGCTCCTGATCTACACAGTTTCCAACCGATTTTCTGGG
GTCCCAGACAGGTTCAGTGGCAGTGGATCAGGGACAGATTTCACACTCAAGATCAACAGAGTGG
AGGCTGAGGATCTGGGAGTTTATTTCTGCTCTCAAACTACACATGTTCCGTGGACGTTCGGTGG
AGGCACCAAGCTGGAAATAAAACGTACG

### Appendix II

### List of Amino Acid Sequences for Anti-Her3 Murine Antibody Clones

>588E10-HC
(SEQ ID NO: 75)
EVQLQQSGPKLVGPGESVKISCKGSGYTFTDYGMHWVKQSHAKRLEWIGVITIYDGHTNYNQNF
KGKAALTVDKSSNTAYLELARMTSEDSAIYYCARGDYDLFWYLDVWGAGTTLTVSS
>588E10-LC
(SEQ ID NO: 76)
DVVMTQTPLSLTVSLGDQASISCRSSQNIKHSDGNTYLEWYLQKPGQSPKLLIYKVSNRFSGVP
DRFSGSGSGTDFTLKISRVEAEDLGVYFCFQGSHVPLTFGAGTKLELKRT
>588E14-HC
(SEQ ID NO: 77)
EVQLQQSGPELVGPGESVKISCKGSGYAFTDYGMHWVKQSHAKRLEWIGVITIYDGHTNYNQNF
KGKAALTVDKSSNTAYLELARMTSEDSAIYYCARGDYDLEWYLDVWGAGTSVTVSS
>588E14-LC
(SEQ ID NO: 78)
DIVMTQTPLSLTVSLGDQASISCRSSQNIKHSDGNTYLEWYLQKPGQSPKLLIYKVSNRFSGVP
DRFSGSGSGTDFTLKISRVEAEDLGVYFCFQGSHVALTEGAGTKLELKRT
>588E22-HC
(SEQ ID NO: 79)
DVQLQESGPELVGPGESVKISCKGSGYTFTDYGMHWVKQSHAKRLEWIGVITIYDGHTNYNQNF
KGKAALTVDKSSNTAYPELARMTSEDSAIYYCARGDYDLFWYLDVWGAGTTVTVSS
>588E22-LC
(SEQ ID NO: 80)
DVVMTQTPLSLTVSLGDQASISCRSSQNIKHSDGNTYLEWYLQKPGQSPKLLIYKVSNRFSGVP
DRFSGSGSGTDFTLKISRVEAEDLGVYFCFQGSHVPLTFGAGTKLELKRT
>588F7-HC
(SEQ ID NO: 81)
QVQLQQPGSVLVRPGASVKLSCKASGYTFTNSWILWAKRRPGQGLEWIGEIHPNSGNTNYNEKF
KGQATLTVDTSSTTAYVDLSRLTSEDSAVYYCARYEGHWGQGTLVTVSA
>588F7-LC
(SEQ ID NO: 82)
DVVMTQTPLTLSVTIGQPASISCKSSQSLLDSDGKTYLNWLLQRSGQSPKRLIYLVSKLDSGVP
DRFTGSGSGTDFTLKISRVEAEDLGVYYCWQGTHFPQTFGGGTKLEIKRT
>588F40-HC
(SEQ ID NO: 83)
EVKLMESGPELVKPGASVKMSCKASGYIFTSYVMHWVKQKPGQGLEWLGYINPYNDGTKYNERF
KGKATLTSDKSSSTAYMELISLTSEDSAVYYCAREASYGNPFAYWGQGTLVTVSA
>588F40-LC
(SEQ ID NO: 84)
DIQMMQSPASLSASVGETVTITCRTSENIYSYLAWFQQKQGNSPQLLVYNTKTLAEGVPSRFSG
SGSGTQFSLKINSLQPEDFGNYYCQHHYDTPWTFGGGTKLEIKRT
>588F41-HC
(SEQ ID NO: 85)
EVQLQQSGPEINKPGASVKMSCKASGYTFTSYVMHWVKQKPGQGLEWLGYINPYNDGTKYNEKF
KGMATLTSDKSSSTAYMELISLTSEDSAVYYCAREASYGNPFAYWGQGTLVTVSA
>588F41-LC
(SEQ ID NO: 86)
DIVMTQSPASLSASVGETVTITCRTSENIYSYLAWFQQKQGNSPQLLVYNTKTLAEGVPSRFSG
SGSGTQFSLKINSLQPEDFGNYYCQHHYDTPWTFGGGTKLEIKRT
>588F42-HC
(SEQ ID NO: 87)
EVQLQQSGPELVKPGASVKMSCKASGYTFTSYVMHWVKQKPGQGLEWLGYINPYNDGTKYNEKF
KGKATLTSDKSSSTAYMELISLTSEDSAVYYCAREASYGNPFAYWGQGTLVTVSA
>588F42-LC
(SEQ ID NO: 88)
DIVMTQSPASLSASVGETVTITCRTSENIYSYLAWFQQKQGNSPQLLVYNTKTLAEGVPSRFSG
SGSGTQFSLKINSLQPEDFGNYYCQHHYDTPWTFGGGTKLEIK
>PO-3H7-E3-HC
(SEQ ID NO: 89)
EVQLQQSGAELVKPGASVKISCKASGYSFTGYYMNWVKQSHGKSLEWIGNINPYYGGSNYNQKF
KARATLTVDKSSSTAYMQLNSLTSEDSAVYYCARKGDLYYSMDYWGQGTSVTVSS
>PO-3H7-E3-LC
(SEQ ID NO: 90)
DIVMTQTPLSLPVSLGDQASISCRSSQSIVHSNGNTYLEWYLQKPGQSPKLLIYKVSNRFSGVP
DRFSGSGSGTDFTLKISRVEAEDLGIYYCFQGSHVPPTFGGGTKLELKRT
>PP-2F3-A6-HC
(SEQ ID NO: 91)
DVKLQESGPGLVKPSQSLSLTCTVTGYSITSDYAWNWIRQFPGNKLEWMGYINYSGSTNYNPSL
KSRISITRDTSKNQFFLKLNSVTSEDTATYYCARDDYDGYYFDYWGQGTTVTVSS
>PP-2F3-A6-LC
(SEQ ID NO: 92)
DIVLTQSPASLAVSLGQRATISYRASKSVSTSGYSYMHWNQQKPGQPPRLLIYLVSNLESGVPA
RFSGSGSGTDFTLNIHPVEEEDAATHYCQHIRELTRSEGGPSWKIKRT
>PO-1C9-C4-HC
(SEQ ID NO: 93)
QVQLQQPGSELVRPGASVKLSCKASGYTFTIFWIHWVKQRPGQGLEWIGNIYPGSGGTNYDEKF
KSKATLTVDTFSSTAYMQLSSLTSEDSAVYYCTRWGTGKDYWGQGTTLTVSS
>PO-1C9-C4-LC
(SEQ ID NO: 94)
DIVLTQSPAIMSASPGEKVTMTCRARSSVSSSYLHWYQQKPGSSPKLWIYSTSNLALGVPARFS
GSGSGTSYSLTISSVEAEDAATYYCQQYDSSPFTFGTGTKLEIKRT
>PO-2H1-A6-HC
(SEQ ID NO: 95)
QVQLXQSGPGKLQPSQTLSLTCSFSGFSLSTYGMGVGWIRQPLGKGLEWLANIWWNDDKYYNSA
LKSRLTISKDTSNNQVFLKISSVDTADAATYYCVQIANPYWYFDVWGAGTSVTVSS
>PO-2H1-A6-LC
(SEQ ID NO: 96)
DIQMTQSPASLAVSLGQRATISYRASKSVSTSGYSYMHWNQQKPGQPPRLLIYLVSNLESGVPA
RFSGSGSGTDFTLNIHPVEEEDAATYYCQHIRELTRSEGGPSWKSKRT
>PO-1G2-B6-HC
(SEQ ID NO: 97)
EVQLQQSGPELVKPGASVKISCKASGYSFTGYHIHWMKQSHVESLEWIGRINPYSGVTDYNQNF
KDKANLTVDQSSSTAYMELHSLTSEDSAFYYCARTDYYGPWFAYWGQGTLVTVSA
>PO-1G2-B6-LC
(SEQ ID NO: 98)
DIQMTQSPLSLPVSLGDQASISCRSSPSLVHSNGNTYLHWYLQKPGQSPKLLIYTVSNRFSGVP
DRFSGSGSGTDFTLKINRVEAEDLGVYFCSQTTHVPWTFGGGTKLEIKRT

### Appendix III

### Full Length Her3 DNA Sequences

>HER3/ERBB3
(SEQ ID NO: 1)
MRANDALQVLGLLFSLARGSEVGNSQAVCPGTLNGLSVTGDAENQYQTLYKLYERCEVVMGNLE
IVLTGHNADLSFLQWIREVTGYVLVAMNEFSTLPLPNLRVVRGTQVYDGKFAIFVMLNYNTNSS
HALRQLRLTQLTEILSGGVYIEKNDKLCHMDTIDWRDIVRDRDAEIVVKDNGRSCPPCHEVCKG
RCWGPGSEDCQTLTKTICAPQCNGHCFGPNPNQCCHDECAGGCSGPQDTDCFACRHFNDSGACV
PRCPQPLVYNKLTFQLEPNPHTKYQYGGVCVASCPHNFVVDQTSCVRACPPDKMEVDKNGLKMC
EPCGGLCPKACEGTGSGSRFQTVDSSNIDGFVNCTKILGNLDFLITGLNGDPWHKIPALDPEKL
NVFRTVREITGYLNIQSWPPHMHNFSVFSNLTTIGGRSLYNRGFSLLIMKNLNVTSLGFRSLKE
ISAGRIYISANRQLCYHHSLNWTKVLRGPTEERLDIKHNRPRRDCVAEGKVCDPLCSSGGCWGP
GPGQCLSCRNYSRGGVCVTHCNFLNGEPREFAHEAECFSCHPECQPMEGTATCNGSGSDTCAQC
AHFRDGPHCVSSCPHGVLGAKGPIYKYPDVQNECRPCHENCTQGCKGPELQDCLGQTLVLIGKT
HLTMALTVIAGLVVIFMMLGGTFLYWRGRRIQNKRAMRRYLERGESIEPLDPSEKANKVLARIF
KETELRKLKVLGSGVFGTVHKGVWIPEGESIKIPVCIKVIEDKSGRQSFQAVTDHMLAIGSLDH
AHIVRLLGLCPGSSLQLVTQYLPLGSLLDHVRQHRGALGPQLLLNWGVQIAKGMYYLEEHGMVH
RNLAARNVLLKSPSQVQVADFGVADLLPPDDKQLLYSEAKTPIKWMALESIHFGKYTHQSDVWS
YGVTVWELMTFGAEPYAGLRLAEVPDLLEKGERLAQPQICTIDVYMVMVKCWMIDENIRPTFKE
LANEFTRMARDPPRYLVIKRESGPGIAPGPEPHGLTNKKLEEVELEPELDLDLDLEAEEDNLAT
TTLGSALSLPVGTLNRPRGSQSLLSPSSGYMPMNQGNLGESCQESAVSGSSERCPRPVSLHPMP
RGCLASESSEGHVTGSEAELQEKVSMCRSRSRSRSPRPRGDSAYHSQRHSLLTPVTPLSPPGLE
EEDVNGYVMPDTHLKGTPSSREGTLSSVGLSSVLGTEEEDEDEEYEYMNRRRRHSPPHPPRPSS
LEELGYEYMDVGSDLSASLGSTQSCPLHPVPIMPTAGTTPDEDYEYMNRQRDGGGPGGDYAAMG
ACPASEQGYEEMRAFQGPGHQAPHVHYARLKTLRSLEATDSAFDNPDYWHSRLFPKANAQRT
>HER3
(SEQ ID NO: 2)
CTCTCACACACACACACCCCTCCCCTGCCATCCCTCCCCGGACTCCGGCTCCGGCTCCGATTGC
AATTTGCAACCTCCGCTGCCGTCGCCGCAGCAGCCACCAATTCGCCAGCGGTTCAGGTGGCTCT
TGCCTCGATGTCCTAGCCTAGGGGCCCCCGGGCCGGACTTGGCTGGGCTCCCTTCACCCTCTGC
GGAGTCATGAGGGCGAACGACGCTCTGCAGGTGCTGGGCTTGCTTTTCAGCCTGGCCCGGGGCT
CCGAGGTGGGCAACTCTCAGGCAGTGTGTCCTGGGACTCTGAATGGCCTGAGTGTGACCGGCGA
TGCTGAGAACCAATACCAGACACTGTACAAGCTCTACGAGAGGTGTGAGGTGGTGATGGGGAAC
CTTGAGATTGTGCTCACGGGACACAATGCCGACCTCTCCTTCCTGCAGTGGATTCGAGAAGTGA
CAGGCTATGTCCTCGTGGCCATGAATGAATTCTCTACTCTACCATTGCCCAACCTCCGCGTGGT
GCGAGGGACCCAGGTCTACGATGGGAAGTTTGCCATCTTCGTCATGTTGAACTATAACACCAAC
TCCAGCCACGCTCTGCGCCAGCTCCGCTTGACTCAGCTCACCGAGATTCTGTCAGGGGGTGTTT
ATATTGAGAAGAACGATAAGCTTTGTCACATGGACACAATTGACTGGAGGGACATCGTGAGGGA
CCGAGATGCTGAGATAGTGGTGAAGGACAATGGCAGAAGCTGTCCCCCCTGTCATGAGGTTTGC
AAGGGGCGATGCTGGGGTCCTGGATCAGAAGACTGCCAGACATTGACCAAGACCATCTGTGCTC
CTCAGTGTAATGGTCACTGCTTTGGGCCCAACCCCAACCAGTGCTGCCATGATGAGTGTGCCGG
GGGCTGCTCAGGCCCTCAGGACACAGACTGCTTTGCCTGCCGGCACTTCAATGACAGTGGAGCC
TGTGTACCTCGCTGTCCACAGCCTCTTGTCTACAACAAGCTAACTTTCCAGCTGGAACCCAATC
CCCACACCAAGTATCAGTATGGAGGAGTTTGTGTAGCCAGCTGTCCCCATAACTTTGTGGTGGA
TCAAACATCCTGTGTCAGGGCCTGTCCTCCTGACAAGATGGAAGTAGATAAAAATGGGCTCAAG
ATGTGTGAGCCTTGTGGGGGACTATGTCCCAAAGCCTGTGAGGGAACAGGCTCTGGGAGCCGCT
TCCAGACTGTGGACTCGAGCAACATTGATGGATTTGTGAACTGCACCAAGATCCTGGGCAACCT
GGACTTTCTGATCACCGGCCTCAATGGAGACCCCTGGCACAAGATCCCTGCCCTGGACCCAGAG
AAGCTCAATGTCTTCCGGACAGTACGGGAGATCACAGGTTACCTGAACATCCAGTCCTGGCCGC
CCCACATGCACAACTTCAGTGTTTTTTCCAATTTGACAACCATTGGAGGCAGAAGCCTCTACAA
CCGGGGCTTCTCATTGTTGATCATGAAGAACTTGAATGTCACATCTCTGGGCTTCCGATCCCTG
AAGGAAATTAGTGCTGGGCGTATCTATATAAGTGCCAATAGGCAGCTCTGCTACCACCACTCTT
TGAACTGGACCAAGGTGCTTCGGGGGCCTACGGAAGAGCGACTAGACATCAAGCATAATCGGCC
GCGCAGAGACTGCGTGGCAGAGGGCAAAGTGTGTGACCCACTGTGCTCCTCTGGGGGATGCTGG
GGCCCAGGCCCTGGTCAGTGCTTGTCCTGTCGAAATTATAGCCGAGGAGGTGTCTGTGTGACCC
ACTGCAACTTTCTGAATGGGGAGCCTCGAGAATTTGCCCATGAGGCCGAATGCTTCTCCTGCCA
CCCGGAATGCCAACCCATGGGGGGCACTGCCACATGCAATGGCTCGGGCTCTGATACTTGTGCT
CAATGTGCCCATTTTCGAGATGGGCCCCACTGTGTGAGCAGCTGCCCCCATGGAGTCCTAGGTG
CCAAGGGCCCAATCTACAAGTACCCAGATGTTCAGAATGAATGTCGGCCCTGCCATGAGAACTG
CACCCAGGGGTGTAAAGGACCAGAGCTTCAAGACTGTTTAGGACAAACACTGGTGCTGATCGGC
AAAACCCATCTGACAATGGCTTTGACAGTGATAGCAGGATTGGTAGTGATTTTCATGATGCTGG
GCGGCACTTTTCTCTACTGGCGTGGGCGCCGGATTCAGAATAAAAGGGCTATGAGGCGATACTT
GGAACGGGGTGAGAGCATAGAGCCTCTGGACCCCAGTGAGAAGGCTAACAAAGTCTTGGCCAGA
ATCTTCAAAGAGACAGAGCTAAGGAAGCTTAAAGTGCTTGGCTCGGGTGTCTTTGGAACTGTGC
ACAAAGGAGTGTGGATCCCTGAGGGTGAATCAATCAAGATTCCAGTCTGCATTAAAGTCATTGA
GGACAAGAGTGGACGGCAGAGTTTTCAAGCTGTGACAGATCATATGCTGGCCATTGGCAGCCTG
GACCATGCCCACATTGTAAGGCTGCTGGGACTATGCCCAGGGTCATCTCTGCAGCTTGTCACTC
AATATTTGCCTCTGGGTTCTCTGCTGGATCATGTGAGACAACACCGGGGGGCACTGGGGCCACA
GCTGCTGCTCAACTGGGGAGTACAAATTGCCAAGGGAATGTACTACCTTGAGGAACATGGTATG
GTGCATAGAAACCTGGCTGCCCGAAACGTGCTACTCAAGTCACCCAGTCAGGTTCAGGTGGCAG
ATTTTGGTGTGGCTGACCTGCTGCCTCCTGATGATAAGCAGCTGCTATACAGTGAGGCCAAGAC
TCCAATTAAGTGGATGGCCCTTGAGAGTATCCACTTTGGGAAATACACACACCAGAGTGATGTC
TGGAGCTATGGTGTGACAGTTTGGGAGTTGATGACCTTCGGGGCAGAGCCCTATGCAGGGCTAC
GATTGGCTGAAGTACCAGACCTGCTAGAGAAGGGGGAGCGGTTGGCACAGCCCCAGATCTGCAC
AATTGATGTCTACATGGTGATGGTCAAGTGTTGGATGATTGATGAGAACATTCGCCCAACCTTT
AAAGAACTAGCCAATGAGTTCACCAGGATGGCCCGAGACCCACCACGGTATCTGGTCATAAAGA
GAGAGAGTGGGCCTGGAATAGCCCCTGGGCCAGAGCCCCATGGTCTGACAAACAAGAAGCTAGA
GGAAGTAGAGCTGGAGCCAGAACTAGACCTAGACCTAGACTTGGAAGCAGAGGAGGACAACCTG
GCAACCACCACACTGGGCTCCGCCCTCAGCCTACCAGTTGGAACACTTAATCGGCCACGTGGGA
GCCAGAGCCTTTTAAGTCCATCATCTGGATACATGCCCATGAACCAGGGTAATCTTGGGGGGTC
TTGCCAGGAGTCTGCAGTTTCTGGGAGCAGTGAACGGTGCCCCCGTCCAGTCTCTCTACACCCA
ATGCCACGGGGATGCCTGGCATCAGAGTCATCAGAGGGGCATGTAACAGGCTCTGAGGCTGAGC
TCCAGGAGAAAGTGTCAATGTGTAGAAGCCGGAGCAGGAGCCGGAGCCCACGGCCACGCGGAGA
TAGCGCCTACCATTCCCAGCGCCACAGTCTGCTGACTCCTGTTACCCCACTCTCCCCACCCGGG
TTAGAGGAAGAGGATGTCAACGGTTATGTCATGCCAGATACACACCTCAAAGGTACTCCCTCCT
CCCGGGAAGGCACCCTTTCTTCAGTGGGTCTCAGTTCTGTCCTGGGTACTGAAGAAGAAGATGA
AGATGAGGAGTATGAATACATGAACCGGAGGAGAAGGCACAGTCCACCTCATCCCCCTAGGCCA
AGTTCCCTTGAGGAGCTGGGTTATGAGTACATGGATGTGGGGTCAGACCTCAGTGCCTCTCTGG
GCAGCACACAGAGTTGCCCACTCCACCCTGTACCCATCATGCCCACTGCAGGCACAACTCCAGA
TGAAGACTATGAATATATGAATCGGCAACGAGATGGAGGTGGTCCTGGGGGTGATTATGCAGCC
ATGGGGGCCTGCCCAGCATCTGAGCAAGGGTATGAAGAGATGAGAGCTTTTCAGGGGCCTGGAC
ATCAGGCCCCCCATGTCCATTATGCCCGCCTAAAAACTCTACGTAGCTTAGAGGCTACAGACTC
TGCCTTTGATAACCCTGATTACTGGCATAGCAGGCTTTTCCCCAAGGCTAATGCCCAGAGAACG
TAACTCCTGCTCCCTGTGGCACTCAGGGAGCATTTAATGGCAGCTAGTGCCTTTAGAGGGTACC
GTCTTCTCCCTATTCCCTCTCTCTCCCAGGTCCCAGCCCCTTTTCCCCAGTCCCAGACAATTCC
ATTCAATCTTTGGAGGCTTTTAAACATTTTGACACAAAATTCTTATGGTATGTAGCCAGCTGTG
CACTTTCTTCTCTTTCCCAACCCCAGGAAAGGTTTTCCTTATTTTGTGTGCTTTCCCAGTCCCA
TTCCTCAGCTTCTTCACAGGCACTCCTGGAGATATGAAGGATTACTCTCCATATCCCTTCCTCT
CAGGCTCTTGACTACTTGGAACTAGGCTCTTATGTGTGCCTTTGTTTCCCATCAGACTGTCAAG
AAGAGGAAAGGGAGGAAACCTAGCAGAGGAAAGTGTAATTTTGGTTTATGACTCTTAACCCCCT
AGAAAGACAGAAGCTTAAAATCTGTGAAGAAAGAGGTTAGGAGTAGATATTGATTACTATCATA
ATTCAGCACTTAACTATGAGCCAGGCATCATACTAAACTTCACCTACATTATCTCACTTAGTCC
TTTATCATCCTTAAAACAATTCTGTGACATACATATTATCTCATTTTACACAAAGGGAAGTCGG
GCATGGTGGCTCATGCCTGTAATCTCAGCACTTTGGGAGGCTGAGGCAGAAGGATTACCTGAGG
CAAGGAGTTTGAGACCAGCTTAGCCAACATAGTAAGACCCCCATCTC

1. An isolated or purified Her3-specific antibody molecule or an antigen binding portion thereof comprising at least one light chain sequence comprising a sequence of amino acids selected from the group listed in Appendix II and at least one heavy chain sequence comprising a sequence of amino acids selected from the group listed in Appendix II, or at least one light chain comprising an amino acid sequence having at least 80% identity with said at least one light chain listed in Appendix II and wherein said heavy chain comprises an amino acid sequence having at least 80% identity with at least one sequence listed in Appendix II.

2. The antibody according to claim 1, wherein said antibody is humanized.

3. The antigen-binding portion according to claim 1, wherein said portion is selected from the group consisting of: a Fab fragment, an F(ab′)2 fragment and an Fv fragment.

4. An isolated or purified Her3-specific antibody molecule comprising a heavy chain variable region comprising at least one amino acid sequence as set forth in Table II or a glycosylation variant, fusion molecule or a chemical derivative thereof or an antigen-binding region thereof that specifically binds Her3.

5. The antibody of claim 1 that comprises a mutant immunoglobulin chain, the mutant antibody having higher affinity for an antigen than a parent antibody that comprises a parent immunoglobulin chain, wherein the mutant immunoglobulin chain comprises an amino acid substitution that eliminates a variable region glycosylation site of the parent immunoglobulin chain, said elimination having the effect of increasing the affinity of the mutant antibody relative to the parent antibody.

6-8. (canceled)

9. A method for determining the prognosis of the course of a malignant disease associated with expression of Her3, comprising obtaining a sample from a subject suspected of containing tumor cells, contacting said sample with the antibody of claim 1 or an antigen-binding portion thereof, wherein binding of the antibody or the antigen-binding portion thereof with tumor cells in the sample is indicative of a tumor and gives a prognoses for the course of a malignant disease in said subject.

10. A method for selecting a therapy for a patient or a patient population with a tumor associated with or mediated by expression of Her3 comprising: (a) determining whether the patient's tumor is known to over express Her3 bearing cells relative to normal and (b) selecting an Her3 inhibitory agent as the therapy if the patient's tumor is known to over express said Her3.

11. (canceled)

12. A method for following progress of a therapeutic regime designed to alleviate an oncogenic disorder associated with or characterized by expression of Her3 comprising:
(a) assaying a biological sample from a subject to determine level of Her3 at a first time point by contacting said sample with the antibody according to claim 1;
(b) assaying level of Her3 at a second time point; and
(c) comparing said level at said second time point to the level determined in (a) as a determination of effect of said therapeutic regime.

13-14. (canceled)

15. A method for determining the prognosis for survival for a patient presenting with a cancer mediated by Her3, comprising: (a) measuring a level of Her3 receptor polypeptide in a cancer cell-containing sample from said patient, and (b) comparing the level of Her3 receptor polypeptide in said sample to a reference level of Her3 polypeptide from normal tissue, wherein a lower level of Her3 polypeptide relative to said reference level correlates with increased survival of said patient, wherein step (a) comprises measuring level of Her 3 using the antibody of claim 1.

16-22. (canceled)

23. A method for monitoring the efficacy of an antibody in correcting an abnormal, level of Her3 in a subject presenting with an oncogenic disorder associated with increased of Her3, comprising
i) administering an effective amount of a conventional Her3 antibody other than the antibody of claim 1 to said subject; and
ii) determining a level of Her3 in said subject following the administration of the conventional antibody, wherein a change in the level of Her3 towards a normal level is indicative of the efficacy of said antibody.

24-31. (canceled)

32. An in vivo method of imaging an oncogenic disorder associated with expression of Her3 comprising the steps of:
(a) administering to a subject an imaging-effective amount of the labeled monoclonal antibody according to claim 1 or fragment thereof and a pharmaceutically effective carrier; and
(b) detecting the binding of said labeled monoclonal antibody or fragment thereof to Her3 expressing cells associated with said disorder.

33-49. (canceled)

50. A method for determining a chemotherapeutic regimen comprising an Her 3 targeted agent, for treating a tumor in a patient comprising: (a) obtaining a tissue sample of the tumor; (b) detecting levels of Her3 levels in said sample, (c) scoring said sample for expression of Her3 levels, (d) repeating steps (b)-(c) in a matching non-malignant tissue sample to obtain a threshold level (e) determining a chemotherapeutic regimen by comparing the differential Her3 expression level of step (c) and the threshold level of step (d), wherein an increase in differential Her3 expression level in step (c) relative to step (d) dictate placing said patient in the chemotherapeutic regimen wherein step (b) comprises contacting said tissue sample with the antibody of claim 1.

51-53. (canceled)

54. A method for treating an Her3 mediated cancer comprising: a) obtaining a sample of diseased tissue from a patient in need of treatment of said cancer; b) determining the level of expression of Her3 levels in said tissue sample; c) scoring said samples for expression of Her3 levels; d) correlating said score to identify patients likely to benefit from treatment with an Her3 antagonist, wherein said step of correlating comprises comparing said scoring to that obtained from a control sample, e) treating said patient with a therapeutic regime known to improve the prognosis for said cancer; f) repeating steps “a” and “b”, and g) adjusting the therapeutic regime known to improve the prognosis for said cancer; h) repeating steps a-f as frequently as deemed appropriate, wherein step (b) comprises contacting said tissue sample with the antibody of claim 1.

55. (canceled)

56. A method of treating a Her3 mediated disorder comprising administrating to a patient in need thereof the antibody of claim 1 sufficient to treat said disorder, wherein said antibody is an antagonist antibody specific for Her3.

57. (canceled)

58. A method for stratifying a patient presenting with an oncogenic disorder mediated by Her3 for a clinical trial comprising: a) obtaining a tissue sample from said patient, b) detecting levels of Her3 protein in said sample, c) scoring said sample for Her3 protein levels; and d) stratifying said patient for said clinical trial based on the results of the scoring step, wherein step (b) comprises contacting said tissue sample with the antibody of claim 1.

59. (canceled)

60. A method of classifying or staging a breast tumor characterized by expression of Her3 comprising the steps of: i) providing a breast tumor sample, ii) detecting expression Her3 in the sample, iii) scoring the sample for Her3 expression level, and iv) classifying the tumor as belonging to a tumor subclass based on the results of the scoring step, wherein step ii) comprises contacting said tissue sample with the antibody of claim 1.

61. (canceled)

62. A method of treating a human tumor susceptible to an antibody induced cellular cytotoxicity in a mammal, wherein said human tumor expresses an antigen which specifically binds to the monoclonal antibody which has the cellular cytotoxicity inducing characteristics of the antibody of claim 1, comprising administering to said mammal said antibody or said antigen binding portion thereof in an amount effective to induce cellular cytotoxicity and thereby reduce said mammal's tumor burden.

63-66. (canceled)

67. The isolated antibody or antigen binding portion of claim 1 conjugated with a member selected from the group consisting of cytotoxic moieties, enzymes, radioactive compounds, and hematogenous cells.

68. The isolated antibody according to claim 1 wherein said antibody is a multivalent antibody.

69. The antibody according to claim 68, wherein said antibody is a bispecific, tetravalent antibody specific for Her3.

70. The isolated antibody of claim 1 which comprises an antigen binding region and a variant Fc region, wherein said variant Fc region; (A) differs from a wild-type Fe region by comprising an amino acid modification and (B) binds an FcyR with an increased affinity relative to a said wild-type Fc region.

71. The antibody according to claim 70 wherein said FcyR is FcyRIIIA.

72. The antibody according to claim 71, wherein said variant Fc region of said antibody has decreased affinity for FcyRIIB relative to said wild-type Fc region.

73. A variant antibody derived from the antibody of claim 1, wherein said antibody comprises an Fc region, said variant mediates antibody-dependent cell-mediated cytotoxicity (ADCC) in the presence of human effector cells more effectively, or binds an Fcγ receptor (FcyR) with better affinity, than the parent polypeptide and comprises at least one amino acid modification in the Fc region.

74. A method of treating a human tumor susceptible to antibody induced cellular cytotoxicity in a mammal, wherein said human tumor expresses a Her3 receptor which specifically binds to a monoclonal antibody which has the cellular cytotoxicity inducing characteristics of the antibody of claim 1, comprising administering to said mammal said monoclonal antibody or said antigen binding portion thereof in an amount effective to induce cellular cytotoxicity and thereby reduce said mammal's tumor burden.

75-78. (canceled)

79. The isolated antibody or antigen binding portion of claim 1 conjugated with a member selected from the group consisting of cytotoxic moieties, enzymes and radioactive compounds.


SEQUENCE LISTING (98 sequences)

SEQ ID NO: 1 - protein, 1342 aa, Artificial Sequence
  cDNA and proteins encoding a human protein
MRANDALQVLGLLFSLARGSEVGNSQAVCPGTLNGLSVTGDAENQYQTLYKLYERCEVVMGNLEIVLTGHNADLSFLQWIREVTGYVLVAMNEFSTLPLPNLRVVRGTQVYDGKFAIFVMLNYNTNSSHALRQLRLTQLTEILSGGVYIEKNDKLCHMDTIDWRDIVRDRDAEIVVKDNGRSCPPCHEVCKGRCWGPGSEDCQTLTKTICAPQCNGHCFGPNPNQCCHDECAGGCSGPQDTDCFACRHFNDSGACVPRCPQPLVYNKLTFQLEPNPHTKYQYGGVCVASCPHNFVVDQTSCVRACPPDKMEVDKNGLKMCEPCGGLCPKACEGTGSGSRFQTVDSSNIDGFVNCTKILGNLDFLITGLNGDPWHKIPALDPEKLNVFRTVREITGYLNIQSWPPHMHNFSVFSNLTTIGGRSLYNRGFSLLIMKNLNVTSLGFRSLKEISAGRIYISANRQLCYHHSLNWTKVLRGPTEERLDIKHNRPRRDCVAEGKVCDPLCSSGGCWGPGPGQCLSCRNYSRGGVCVTHCNFLNGEPREFAHEAECFSCHPECQPMEGTATCNGSGSDTCAQCAHFRDGPHCVSSCPHGVLGAKGPIYKYPDVQNECRPCHENCTQGCKGPELQDCLGQTLVLIGKTHLTMALTVIAGLVVIFMMLGGTFLYWRGRRIQNKRAMRRYLERGESIEPLDPSEKANKVLARIFKETELRKLKVLGSGVFGTVHKGVWIPEGESIKIPVCIKVIEDKSGRQSFQAVTDHMLAIGSLDHAHIVRLLGLCPGSSLQLVTQYLPLGSLLDHVRQHRGALGPQLLLNWGVQIAKGMYYLEEHGMVHRNLAARNVLLKSPSQVQVADFGVADLLPPDDKQLLYSEAKTPIKWMALESIHFGKYTHQSDVWSYGVTVWELMTFGAEPYAGLRLAEVPDLLEKGERLAQPQICTIDVYMVMVKCWMIDENIRPTFKELANEFTRMARDPPRYLVIKRESGPGIAPGPEPHGLTNKKLEEVELEPELDLDLDLEAEEDNLATTTLGSALSLPVGTLNRPRGSQSLLSPSSGYMPMNQGNLGESCQESAVSGSSERCPRPVSLHPMPRGCLASESSEGHVTGSEAELQEKVSMCRSRSRSRSPRPRGDSAYHSQRHSLLTPVTPLSPPGLEEEDVNGYVMPDTHLKGTPSSREGTLSSVGLSSVLGTEEEDEDEEYEYMNRRRRHSPPHPPRPSSLEELGYEYMDVGSDLSASLGSTQSCPLHPVPIMPTAGTTPDEDYEYMNRQRDGGGPGGDYAAMGACPASEQGYEEMRAFQGPGHQAPHVHYARLKTLRSLEATDSAFDNPDYWHSRLFPKANAQRT

SEQ ID NO: 2 - DNA, 4975 bp, Artificial Sequence
  cDNA and proteins encoding a human protein
ctctcacacacacacacccctcccctgccatccctccccggactccggctccggctccgattgcaatttgcaacctccgctgccgtcgccgcagcagccaccaattcgccagcggttcaggtggctcttgcctcgatgtcctagcctaggggcccccgggccggacttggctgggctcccttcaccctctgcggagtcatgagggcgaacgacgctctgcaggtgctgggcttgcttttcagcctggcccggggctccgaggtgggcaactctcaggcagtgtgtcctgggactctgaatggcctgagtgtgaccggcgatgctgagaaccaataccagacactgtacaagctctacgagaggtgtgaggtggtgatggggaaccttgagattgtgctcacgggacacaatgccgacctctccttcctgcagtggattcgagaagtgacaggctatgtcctcgtggccatgaatgaattctctactctaccattgcccaacctccgcgtggtgcgagggacccaggtctacgatgggaagtttgccatcttcgtcatgttgaactataacaccaactccagccacgctctgcgccagctccgcttgactcagctcaccgagattctgtcagggggtgtttatattgagaagaacgataagctttgtcacatggacacaattgactggagggacatcgtgagggaccgagatgctgagatagtggtgaaggacaatggcagaagctgtcccccctgtcatgaggtttgcaaggggcgatgctggggtcctggatcagaagactgccagacattgaccaagaccatctgtgctcctcagtgtaatggtcactgctttgggcccaaccccaaccagtgctgccatgatgagtgtgccgggggctgctcaggccctcaggacacagactgctttgcctgccggcacttcaatgacagtggagcctgtgtacctcgctgtccacagcctcttgtctacaacaagctaactttccagctggaacccaatccccacaccaagtatcagtatggaggagtttgtgtagccagctgtccccataactttgtggtggatcaaacatcctgtgtcagggcctgtcctcctgacaagatggaagtagataaaaatgggctcaagatgtgtgagccttgtgggggactatgtcccaaagcctgtgagggaacaggctctgggagccgcttccagactgtggactcgagcaacattgatggatttgtgaactgcaccaagatcctgggcaacctggactttctgatcaccggcctcaatggagacccctggcacaagatccctgccctggacccagagaagctcaatgtcttccggacagtacgggagatcacaggttacctgaacatccagtcctggccgccccacatgcacaacttcagtgttttttccaatttgacaaccattggaggcagaagcctctacaaccggggcttctcattgttgatcatgaagaacttgaatgtcacatctctgggcttccgatccctgaaggaaattagtgctgggcgtatctatataagtgccaataggcagctctgctaccaccactctttgaactggaccaaggtgcttcgggggcctacggaagagcgactagacatcaagcataatcggccgcgcagagactgcgtggcagagggcaaagtgtgtgacccactgtgctcctctgggggatgctggggcccaggccctggtcagtgcttgtcctgtcgaaattatagccgaggaggtgtctgtgtgacccactgcaactttctgaatggggagcctcgagaatttgcccatgaggccgaatgcttctcctgccacccggaatgccaacccatggggggcactgccacatgcaatggctcgggctctgatacttgtgctcaatgtgcccattttcgagatgggccccactgtgtgagcagctgcccccatggagtcctaggtgccaagggcccaatctacaagtacccagatgttcagaatgaatgtcggccctgccatgagaactgcacccaggggtgtaaaggaccagagcttcaagactgtttaggacaaacactggtgctgatcggcaaaacccatctgacaatggctttgacagtgatagcaggattggtagtgattttcatgatgctgggcggcacttttctctactggcgtgggcgccggattcagaataaaagggctatgaggcgatacttggaacggggtgagagcatagagcctctggaccccagtgagaaggctaacaaagtcttggccagaatcttcaaagagacagagctaaggaagcttaaagtgcttggctcgggtgtctttggaactgtgcacaaaggagtgtggatccctgagggtgaatcaatcaagattccagtctgcattaaagtcattgaggacaagagtggacggcagagttttcaagctgtgacagatcatatgctggccattggcagcctggaccatgcccacattgtaaggctgctgggactatgcccagggtcatctctgcagcttgtcactcaatatttgcctctgggttctctgctggatcatgtgagacaacaccggggggcactggggccacagctgctgctcaactggggagtacaaattgccaagggaatgtactaccttgaggaacatggtatggtgcatagaaacctggctgcccgaaacgtgctactcaagtcacccagtcaggttcaggtggcagattttggtgtggctgacctgctgcctcctgatgataagcagctgctatacagtgaggccaagactccaattaagtggatggcccttgagagtatccactttgggaaatacacacaccagagtgatgtctggagctatggtgtgacagtttgggagttgatgaccttcggggcagagccctatgcagggctacgattggctgaagtaccagacctgctagagaagggggagcggttggcacagccccagatctgcacaattgatgtctacatggtgatggtcaagtgttggatgattgatgagaacattcgcccaacctttaaagaactagccaatgagttcaccaggatggcccgagacccaccacggtatctggtcataaagagagagagtgggcctggaatagcccctgggccagagccccatggtctgacaaacaagaagctagaggaagtagagctggagccagaactagacctagacctagacttggaagcagaggaggacaacctggcaaccaccacactgggctccgccctcagcctaccagttggaacacttaatcggccacgtgggagccagagccttttaagtccatcatctggatacatgcccatgaaccagggtaatcttggggggtcttgccaggagtctgcagtttctgggagcagtgaacggtgcccccgtccagtctctctacacccaatgccacggggatgcctggcatcagagtcatcagaggggcatgtaacaggctctgaggctgagctccaggagaaagtgtcaatgtgtagaagccggagcaggagccggagcccacggccacgcggagatagcgcctaccattcccagcgccacagtctgctgactcctgttaccccactctccccacccgggttagaggaagaggatgtcaacggttatgtcatgccagatacacacctcaaaggtactccctcctcccgggaaggcaccctttcttcagtgggtctcagttctgtcctgggtactgaagaagaagatgaagatgaggagtatgaatacatgaaccggaggagaaggcacagtccacctcatccccctaggccaagttcccttgaggagctgggttatgagtacatggatgtggggtcagacctcagtgcctctctgggcagcacacagagttgcccactccaccctgtacccatcatgcccactgcaggcacaactccagatgaagactatgaatatatgaatcggcaacgagatggaggtggtcctgggggtgattatgcagccatgggggcctgcccagcatctgagcaagggtatgaagagatgagagcttttcaggggcctggacatcaggccccccatgtccattatgcccgcctaaaaactctacgtagcttagaggctacagactctgcctttgataaccctgattactggcatagcaggcttttccccaaggctaatgcccagagaacgtaactcctgctccctgtggcactcagggagcatttaatggcagctagtgcctttagagggtaccgtcttctccctattccctctctctcccaggtcccagccccttttccccagtcccagacaattccattcaatctttggaggcttttaaacattttgacacaaaattcttatggtatgtagccagctgtgcactttcttctctttcccaaccccaggaaaggttttccttattttgtgtgctttcccagtcccattcctcagcttcttcacaggcactcctggagatatgaaggattactctccatatcccttcctctcaggctcttgactacttggaactaggctcttatgtgtgcctttgtttcccatcagactgtcaagaagaggaaagggaggaaacctagcagaggaaagtgtaattttggtttatgactcttaaccccctagaaagacagaagcttaaaatctgtgaagaaagaggttaggagtagatattgattactatcataattcagcacttaactatgagccaggcatcatactaaacttcacctacattatctcacttagtcctttatcatccttaaaacaattctgtgacatacatattatctcattttacacaaagggaagtcgggcatggtggctcatgcctgtaatctcagcactttgggaggctgaggcagaaggattacctgaggcaaggagtttgagaccagcttagccaacatagtaagacccccatctc

SEQ ID NO: 3 - protein, 8 aa, Mus Musculus
GYTFTDYG

SEQ ID NO: 4 - protein, 7 aa, Mus Musculus
ITIYDGH

SEQ ID NO: 5 - protein, 13 aa, Mus Musculus
ARGDYDLFWYLDV

SEQ ID NO: 6 - protein, 8 aa, Mus Musculus
GYTFTNSW

SEQ ID NO: 7 - protein, 7 aa, Mus Musculus
IHPNSGN

SEQ ID NO: 8 - protein, 6 aa, Mus Musculus
ARYEGH

SEQ ID NO: 9 - protein, 8 aa, Mus Musculus
GYTFTSYV

SEQ ID NO: 10 - protein, 7 aa, Mus Musculus
INPYNDG

SEQ ID NO: 11 - protein, 12 aa, Mus Musculus
AREASYGNPFAY

SEQ ID NO: 12 - protein, 8 aa, Mus Musculus
GYSFTGYY

SEQ ID NO: 13 - protein, 7 aa, Mus Musculus
INPYYGG

SEQ ID NO: 14 - protein, 12 aa, Mus Musculus
ARKGDLYYSMDY

SEQ ID NO: 15 - protein, 9 aa, Mus Musculus
GYSITSDYA

SEQ ID NO: 16 - protein, 6 aa, Mus Musculus
INYSGS

SEQ ID NO: 17 - protein, 12 aa, Mus Musculus
ARDDYDGYYFDY

SEQ ID NO: 18 - protein, 8 aa, Mus Musculus
GYTFTIFW

SEQ ID NO: 19 - protein, 7 aa, Mus Musculus
IYPGSGG

SEQ ID NO: 20 - protein, 9 aa, Mus Musculus
TRWGTGKDY

SEQ ID NO: 21 - protein, 10 aa, Mus Musculus
GFSLSTYGMG

SEQ ID NO: 22 - protein, 6 aa, Mus Musculus
IWWNDD

SEQ ID NO: 23 - protein, 12 aa, Mus Musculus
VQIANPYWYFDV

SEQ ID NO: 24 - protein, 8 aa, Mus Musculus
GYSFTGYH

SEQ ID NO: 25 - protein, 7 aa, Mus Musculus
INPYSGV

SEQ ID NO: 26 - protein, 12 aa, Mus Musculus
ARTDYYGPWFAY

SEQ ID NO: 27 - protein, 11 aa, Mus Musculus
QNIKHSDGNTY

SEQ ID NO: 28 - protein, 2 aa, Mus Musculus
KV

SEQ ID NO: 29 - protein, 9 aa, Mus Musculus
FQGSHVPLT

SEQ ID NO: 30 - protein, 11 aa, Mus Musculus
QSLLDSDGKTY

SEQ ID NO: 31 - protein, 2 aa, Mus Musculus
LV

SEQ ID NO: 32 - protein, 9 aa, Mus Musculus
WQGTHFPQT

SEQ ID NO: 33 - protein, 6 aa, Mus Musculus
ENIYSY

SEQ ID NO: 34 - protein, 2 aa, Mus Musculus
NT

SEQ ID NO: 35 - protein, 9 aa, Mus Musculus
QHHYDTPWT

SEQ ID NO: 36 - protein, 11 aa, Mus Musculus
QSIVHSNGNTY

SEQ ID NO: 37 - protein, 2 aa, Mus Musculus
KV

SEQ ID NO: 38 - protein, 9 aa, Mus Musculus
FQGSHVPPT

SEQ ID NO: 39 - protein, 10 aa, Mus Musculus
KSVSTSGYSY

SEQ ID NO: 40 - protein, 2 aa, Mus Musculus
LV

SEQ ID NO: 41 - protein, 8 aa, Mus Musculus
QHIRELTR

SEQ ID NO: 42 - protein, 7 aa, Mus Musculus
SSVSSSY

SEQ ID NO: 43 - protein, 2 aa, Mus Musculus
ST

SEQ ID NO: 44 - protein, 9 aa, Mus Musculus
QQYDSSPFT

SEQ ID NO: 45 - protein, 10 aa, Mus Musculus
KSVSTSGYSY

SEQ ID NO: 46 - protein, 2 aa, Mus Musculus
LV

SEQ ID NO: 47 - protein, 8 aa, Mus Musculus
QHIRELTR

SEQ ID NO: 48 - protein, 11 aa, Mus Musculus
PSLVHSNGNTY

SEQ ID NO: 49 - protein, 2 aa, Mus Musculus
TV

SEQ ID NO: 50 - protein, 9 aa, Mus Musculus
SQTTHVPWT

SEQ ID NO: 51 - DNA, 383 bp, Mus Musculus
ctcgaggaggtgcagcttcagcagtctgggcctaaactggtggggcctggggaatcagtgaagatttcctgcaagggttccggctacacattcactgattacggtatgcactgggtgaaacagagtcatgcaaagaggctcgagtggattggagttatcactatttatgatggtcatacaaactacaaccagaactttaagggcaaggccgcattgactgtagacaaatcctccaacacagcctatctggaacttgccagaatgacttctgaggattctgccatctattactgtgcaaggggagattacgacctcttctggtatttggatgtctggggcgcagggaccactctcacagtctcctcggcctccaccaagggccc

SEQ ID NO: 52 - DNA, 348 bp, Mus Musculus
tctagagatgttgtgatgacccagactccactctccctgactgtcagtcttggagatcaagcctccatctcttgcagatctagtcagaacattaaacatagtgatggaaacacctatttagaatggtacctgcagaaaccaggccagtctccaaagctcctgatctacaaagtttccaaccgattttctggggtcccagacaggttcagtggcagtggatcagggacagatttcacactcaagatcagtagagtggaggctgaggatctgggagtttatttctgctttcaaggttcacatgttcctctcacgttcggtgctgggaccaagctggagctgaaacgtacg

SEQ ID NO: 53 - DNA, 383 bp, Mus Musculus
ctcgaggaggtgcagctgcagcagtctgggcctgaactggtggggcctggggaatcagtgaagatttcctgcaagggttccggctacgcattcactgattacggtatgcactgggtgaaacagagtcatgcaaagaggctcgagtggattggagttatcactatttatgatggtcatacaaactacaaccagaactttaagggcaaggccgcattgactgtagacaaatcctccaacacagcctatctggaacttgccagaatgacttctgaggattctgccatctattactgtgcaaggggagattacgacctcttctggtatttggatgtctggggcgcaggaacctcagtcaccgtctcctcggcctccaccaagggccc

SEQ ID NO: 54 - DNA, 348 bp, Mus Musculus
tctagagacattgtgatgacccaaactccactctccctgactgtcagtcttggagatcaagcctccatctcttgcagatctagtcagaacattaaacatagtgatggaaacacctatttagaatggtacctgcagaaaccaggccagtctccaaagctcctgatctacaaagtttccaaccgattttctggggtcccagacaggttcagtggcagtggatcagggacagatttcacactcaagatcagtagagtggaggctgaggatctgggagtttatttctgctttcaaggttcacatgttcctctcacgttcggtgctgggaccaagctggagctgaaacgtacg

SEQ ID NO: 55 - DNA, 383 bp, Mus Musculus
ctcgaggatgtgcagcttcaggagtctgggcctgaactggtggggcctggggaatcagtgaagatttcctgcaagggttccggctacacattcactgattacggtatgcactgggtgaaacagagtcatgcaaagaggctcgagtggattggagttatcactatttatgatggtcatacaaactacaaccagaactttaagggcaaggccgcattgactgtagacaaatcctccaacacagcctatccggaacttgccagaatgacttctgaggattctgccatctattactgtgcaaggggagattacgacctcttctggtatttggatgtctggggcgcagggaccacggtcaccgtttcctcggcctccaccaagggccc

SEQ ID NO: 56 - DNA, 348 bp, Mus Musculus
tctagagatgttgtgatgacccaaactccactctccctgactgtcagtcttggagatcaagcctccatctcttgcagatctagtcagaacattaaacatagtgatggaaacacctatttagaatggtacctgcagaaaccaggccagtctccaaagctcctgatctacaaagtttccaaccgattttctggggtcccagacaggttcagtggcagtggatcagggacagatttcacactcaagatcagtagagtggaggctgaggatctgggagtttatttctgctttcaaggttcacatgttcctctcacgttcggtgctgggaccaagctggagctgaaacgtacg

SEQ ID NO: 57 - DNA, 363 bp, Mus Musculus
ctcgagcaggtccaactacagcaacctgggtctgtgctggtgaggcctggagcttcagtgaagctgtcctgcaaggcttctggctacaccttcaccaactcctggatactctgggcgaagcggaggcctggacaaggccttgagtggattggagagattcatcctaatagtggtaatactaactataatgagaagttcaagggccaggccacactgactgtagacacatcctccaccacagcctacgtggatctcagcaggctgacatctgaggactctgcggtctattactgtgcaagatatgagggacactggggccaagggactctggtcactgtctctgcggcctccaccaaagggccc

SEQ ID NO: 58 - DNA, 348 bp, Mus Musculus
tctagagatgttgtgatgacccagactccactcactttgtcggttaccattggacaaccagcctccatctcttgtaagtcaagtcagagcctcttagatagtgatggaaagacatatttgaattggttgttacagaggtcaggccagtctccaaagcgcctaatctatctggtgtctaaactggactctggagtccctgacaggttcactggcagtggatcagggacagatttcacactgaaaatcagcagagtggaggctgaggatttgggagtttattattgctggcaaggtacacattttcctcagacgttcggtggaggcaccaagctggaaatcaaacgtacg

SEQ ID NO: 59 - DNA, 382 bp, Mus Musculus
ctcgaggaggtgaagctgatggagtctggacctgaactggttaagcctggggcttcagtgaagatgtcctgcaaggcttctggatacacattcactagctatgttatgcactgggtgaagcagaagcctgggcagggccttgagtggcttggatatattaacccttacaatgatggtactaagtacaatgagaggttcaaaggcaaggccacactgacttcagacaaatcctccagcacagcctacatggagctcatcagcctgacctctgaggactctgcggtctattactgtgcaagagaggcctcctatggtaacccctttgcttactggggccaagggactctggtcactgtctctgcggcctccaccccaagggccc

SEQ ID NO: 60 - DNA, 333 bp, Mus Musculus
tctagagacattcagatgatgcagtctccagcctccctatctgcatctgtgggagaaactgtcaccatcacatgtcgaacaagtgagaatatttacagttatttagcatggtttcagcagaaacagggaaattctcctcagctcctggtctataatacaaaaaccttagcggaaggtgtgccatcaaggttcagtggcagtggatcaggcacacagttttctctgaagatcaacagcctgcagcctgaagattttgggaattattactgtcaacatcattatgatactccgtggacgttcggtggaggcaccaagctggaaatcaaacgtacg

SEQ ID NO: 61 - DNA, 380 bp, Mus Musculus
ctcgaggaggttcagcttcagcagtctggacctgaactggttaagcctggggcttcagtgaagatgtcctgcaaggcttctggatacacattcactagctatgttatgcactgggtgaagcagaagcctgggcagggccttgagtggcttggatatattaacccttacaatgatggtactaagtacaatgagaagttcaaaggcatggccacactgacttcagacaaatcctccagcacagcctacatggagctcatcagcctgacctctgaggactctgcggtctattactgtgcaagagaggcctcctatggtaacccctttgcttactggggccaagggactctggtcactgtctctgcggcctccaccaagggccc

SEQ ID NO: 62 - DNA, 333 bp, Mus Musculus
tctagagacattgtgatgacccagtctccagcctccctatctgcatctgtgggagaaactgtcaccatcacatgtcgaacaagtgagaatatttacagttatttagcatggtttcagcagaaacagggaaattctcctcagctcctggtctataatacaaaaaccttagcggaaggtgtgccatcaaggttcagtggcagtggatcaggcacacagttttctctgaagatcaacagcctgcagcctgaagattttgggaattattactgtcaacatcattatgatactccgtggacgttcggtggaggcaccaagctggaaatcaaacgtacg

SEQ ID NO: 63 - DNA, 383 bp, Mus Musculus
ctcgaggaggtgcagcttcagcagtctggacctgaactggttaagcctggggcttcagtgaagatgtcctgcaaggcttctggatacacattcactagctatgttatgcactgggtgaagcagaagcctgggcagggccttgagtggcttggatatattaacccttacaatgatggtactaagtacaatgagaagttcaaaggcaaggccacactgacttcagacaaatcctccagcacagcctacatggagctcatcagcctgacctctgaggactctgcggtctattactgtgcaagagaggcctcctatggtaacccctttgcttactggggccaagggactctggtcactgtctctgcggcctccccacccaagggccc

SEQ ID NO: 64 - DNA, 333 bp, Mus Musculus
tctagagacattgtgatgacccagtctccagcctccctatctgcatctgtgggagaaactgtcaccatcacatgtcgaacaagtgagaatatttacagttatttagcatggtttcagcagaaacagggaaattctcctcagctcctggtctataatacaaaaaccttagcggaaggtgtgccatcaaggttcagtggcagtggatcaggcacacagttttctctgaagatcaacagcctgcagcctgaagattttgggaattattactgtcaacatcattatgatactccgtggacgttcggtggaggcaccaagctggaaataaaacgtacg

SEQ ID NO: 65 - DNA, 383 bp, Mus Musculus
ctcgaggaggtgcagctgcagcagtctggagctgagctggtgaagcctggggcttcagtgaagatatcctgcaaggcttctggttactcattcactggctactacatgaactgggtgaagcagagccatggaaagagccttgagtggattggaaatattaatccttactatggcggtagtaactacaatcagaagttcaaggccagggccacattgactgtagacaaatcttccagcacagcctacatgcagctcaacagcctgacatcggaggactctgcagtctattactgtgcaagaaaaggtgacctttactattctatggactactggggtcaaggaacctcagtcaccgtctcctcggccctcccacccaagggccc

SEQ ID NO: 66 - DNA, 348 bp, Mus Musculus
tctagagacattgtgatgacccaaactcctctctccctgcctgtcagtcttggagatcaggcctccatttcttgcagatctagtcagagcattgtacatagtaatggaaacacctatttagaatggtacctacagaaaccaggccagtccccaaagctcctcatctacaaagtttccaaccgattttctggggtcccagacaggttcagtggcagtggatcagggacagatttcacactcaagatcagcagagtggaggctgaggatctgggaatttattactgctttcaaggttcacatgttcctccgacgttcggtggaggcaccaagctggagctgaaacgtacg

SEQ ID NO: 67 - DNA, 380 bp, Mus Musculus
ctcgaggatgtgaagcttcaggagtcgggacctggcctggtgaaaccttctcagtctctgtccctcacctgcactgtcactggctactcaatcaccagtgattatgcctggaactggatccggcagtttccaggaaacaaactggagtggatgggctacataaactacagtggcagcactaactataacccatctctcaaaagtcgaatctctatcactcgagacacatccaagaaccagttcttcctgaagctgaattctgtgacctctgaggacacagccacatattactgtgcaagggatgattacgacgggtactactttgactactggggccaaggcaccacggtcaccgtttcctcggcctccaccaagggccc

SEQ ID NO: 68 - DNA, 342 bp, Mus Musculus
tctagagacattgtgttaacacagtctcctgcttccttagctgtatctctggggcagagggccaccatctcatacagggccagcaaaagtgtcagtacatctggctatagttatatgcactggaaccaacagaaaccaggacagccacccagactcctcatctatcttgtatccaacctagaatctggggtccctgccaggttcagtggcagtgggtctgggacagacttcaccctcaacatccatcctgtggaggaggaggatgctgcaacccattactgtcagcacattagggagcttacacgttcggaggggggaccaagctggaaaatcaaacgtacg

SEQ ID NO: 69 - DNA, 371 bp, Mus Musculus
ctcgagcaggtccaactgcagcagcctgggtctgagctggtgaggcctggagcttcagtgaagctgtcctgcaaggcttctggctacacattcaccatcttctggatccactgggtgaagcagaggcctggacaaggccttgagtggattggaaatatttatcctggtagtggtggaactaactacgatgagaaattcaagagcaaggccacactgactgtagacacattttccagcacagcctacatgcagctcagtagcctgacatctgaggactctgcggtctattactgtacaagatgggggactgggaaggactactggggccaaggcaccactctcacagtctcctcggcctccaccaagggccc

SEQ ID NO: 70 - DNA, 336 bp, Mus Musculus
tctagagacattgttctcacccagtctccagcaatcatgtctgcatctccaggggagaaggtcaccatgacctgcagggccaggtcaagtgtaagttccagttacttgcactggtaccagcagaagccaggatcttcccccaaactctggatttatagcacatccaatctggctttaggggtcccagctcgcttcagtggcagtgggtctgggacctcttactctctcacaatcagtagtgtggaggctgaggatgctgccacttattactgccagcagtatgatagttccccattcacgttcggcacggggacaaagttggaaataaaacgtacg

SEQ ID NO: 71 - DNA, 383 bp, Mus Musculus
  misc_feature 21: n = A,T,C or G
ctcgagcaggttcagctgcancaatctggccctgggaaattgcagccctcccagaccctcagtctgacttgttctttttctgggttttcactgagcacttatggtatgggtgtaggttggattcgtcagcctttagggaagggtctggagtggctggccaacatttggtggaatgatgataagtactataattcagccctgaagagccggctcacaatctccaaggatacctccaacaaccaggttttcctcaagatctccagtgtggacactgcagatgctgccacatactactgtgttcaaatagctaacccctattggtacttcgatgtctggggcgcagggacctcagtcaccgtctcctcggcctccaccaagggccc

SEQ ID NO: 72 - DNA, 342 bp, Mus Musculus
tctagagatattcagatgactcagtctcctgcttccttagctgtatctctggggcagagggccaccatctcatacagggccagcaaaagtgtcagtacatctggctatagttatatgcactggaaccaacagaaaccaggacagccacccagactcctcatctatcttgtatccaacctagaatctggggtccctgccaggttcagtggcagtgggtctgggacagacttcaccctcaacatccatcctgtggaggaggaggatgctgcaacctattactgtcagcacattagggagcttacacgttcggaggggggaccaagctggaaatcaaaacgtacg

SEQ ID NO: 73 - DNA, 380 bp, Mus Musculus
ctcgaggaggtgcagctgcagcagtctggacctgagctggtgaagcctggggcttcagtgaagatatcctgcaaggcttctggttactcattcactggctaccacatacactggatgaagcaaagccatgtagagagccttgagtggattggacgtattaatccttacagtggtgttactgactacaaccagaatttcaaggacaaggccaacttgactgtagatcagtcctccagcacagcctacatggaactccacagcctgacatctgaggactctgcattctattactgtgcaaggacggattactacggcccctggtttgcttactggggccaagggactctggtcactgtctctgcggcctccaccaagggccc

SEQ ID NO: 74 - DNA, 348 bp, Mus Musculus
tctagagatattcagatgacgcagtctccactctccctgcctgtcagtcttggagatcaagcctccatctcttgcagatctagtccgagccttgtacacagtaatggaaacacctatttacattggtacctgcagaagccaggccagtctccaaagctcctgatctacacagtttccaaccgattttctggggtcccagacaggttcagtggcagtggatcagggacagatttcacactcaagatcaacagagtggaggctgaggatctgggagtttatttctgctctcaaactacacatgttccgtggacgttcggtggaggcaccaagctggaaataaaacgtacg

SEQ ID NO: 75 - protein, 120 aa, Mus Musculus
EVQLQQSGPKLVGPGESVKISCKGSGYTFTDYGMHWVKQSHAKRLEWIGVITIYDGHTNYNQNFKGKAALTVDKSSNTAYLELARMTSEDSAIYYCARGDYDLFWYLDVWGAGTTLTVSS

SEQ ID NO: 76 - protein, 114 aa, Mus Musculus
DVVMTQTPLSLTVSLGDQASISCRSSQNIKHSDGNTYLEWYLQKPGQSPKLLIYKVSNRFSGVPDRFSGSGSGTDFTLKISRVEAEDLGVYFCFQGSHVPLTFGAGTKLELKRT

SEQ ID NO: 77 - protein, 120 aa, Mus Musculus
EVQLQQSGPELVGPGESVKISCKGSGYAFTDYGMHWVKQSHAKRLEWIGVITIYDGHTNYNQNFKGKAALTVDKSSNTAYLELARMTSEDSAIYYCARGDYDLFWYLDVWGAGTSVTVSS

SEQ ID NO: 78 - protein, 114 aa, Mus Musculus
DIVMTQTPLSLTVSLGDQASISCRSSQNIKHSDGNTYLEWYLQKPGQSPKLLIYKVSNRFSGVPDRFSGSGSGTDFTLKISRVEAEDLGVYFCFQGSHVPLTFGAGTKLELKRT

SEQ ID NO: 79 - protein, 120 aa, Mus Musculus
DVQLQESGPELVGPGESVKISCKGSGYTFTDYGMHWVKQSHAKRLEWIGVITIYDGHTNYNQNFKGKAALTVDKSSNTAYPELARMTSEDSAIYYCARGDYDLFWYLDVWGAGTTVTVSS

SEQ ID NO: 80 - protein, 114 aa, Mus Musculus; identical to SEQ ID NO: 76

SEQ ID NO: 81 - protein, 113 aa, Mus Musculus
QVQLQQPGSVLVRPGASVKLSCKASGYTFTNSWILWAKRRPGQGLEWIGEIHPNSGNTNYNEKFKGQATLTVDTSSTTAYVDLSRLTSEDSAVYYCARYEGHWGQGTLVTVSA

SEQ ID NO: 82 - protein, 114 aa, Mus Musculus
DVVMTQTPLTLSVTIGQPASISCKSSQSLLDSDGKTYLNWLLQRSGQSPKRLIYLVSKLDSGVPDRFTGSGSGTDFTLKISRVEAEDLGVYYCWQGTHFPQTFGGGTKLEIKRT

SEQ ID NO: 83 - protein, 119 aa, Mus Musculus
EVKLMESGPELVKPGASVKMSCKASGYTFTSYVMHWVKQKPGQGLEWLGYINPYNDGTKYNERFKGKATLTSDKSSSTAYMELISLTSEDSAVYYCAREASYGNPFAYWGQGTLVTVSA

SEQ ID NO: 84 - protein, 109 aa, Mus Musculus
DIQMMQSPASLSASVGETVTITCRTSENIYSYLAWFQQKQGNSPQLLVYNTKTLAEGVPSRFSGSGSGTQFSLKINSLQPEDFGNYYCQHHYDTPWTFGGGTKLEIKRT

SEQ ID NO: 85 - protein, 119 aa, Mus Musculus
EVQLQQSGPELVKPGASVKMSCKASGYTFTSYVMHWVKQKPGQGLEWLGYINPYNDGTKYNEKFKGMATLTSDKSSSTAYMELISLTSEDSAVYYCAREASYGNPFAYWGQGTLVTVSA

SEQ ID NO: 86 - protein, 109 aa, Mus Musculus
DIVMTQSPASLSASVGETVTITCRTSENIYSYLAWFQQKQGNSPQLLVYNTKTLAEGVPSRFSGSGSGTQFSLKINSLQPEDFGNYYCQHHYDTPWTFGGGTKLEIKRT

SEQ ID NO: 87 - protein, 119 aa, Mus Musculus
EVQLQQSGPELVKPGASVKMSCKASGYTFTSYVMHWVKQKPGQGLEWLGYINPYNDGTKYNEKFKGKATLTSDKSSSTAYMELISLTSEDSAVYYCAREASYGNPFAYWGQGTLVTVSA

SEQ ID NO: 88 - protein, 107 aa, Mus Musculus
DIVMTQSPASLSASVGETVTITCRTSENIYSYLAWFQQKQGNSPQLLVYNTKTLAEGVPSRFSGSGSGTQFSLKINSLQPEDFGNYYCQHHYDTPWTFGGGTKLEIK

SEQ ID NO: 89 - protein, 119 aa, Mus Musculus
EVQLQQSGAELVKPGASVKISCKASGYSFTGYYMNWVKQSHGKSLEWIGNINPYYGGSNYNQKFKARATLTVDKSSSTAYMQLNSLTSEDSAVYYCARKGDLYYSMDYWGQGTSVTVSS

SEQ ID NO: 90 - protein, 114 aa, Mus Musculus
DIVMTQTPLSLPVSLGDQASISCRSSQSIVHSNGNTYLEWYLQKPGQSPKLLIYKVSNRFSGVPDRFSGSGSGTDFTLKISRVEAEDLGIYYCFQGSHVPPTFGGGTKLELKRT

SEQ ID NO: 91 - protein, 119 aa, Mus Musculus
DVKLQESGPGLVKPSQSLSLTCTVTGYSITSDYAWNWIRQFPGNKLEWMGYINYSGSTNYNPSLKSRISITRDTSKNQFFLKLNSVTSEDTATYYCARDDYDGYYFDYWGQGTTVTVSS

SEQ ID NO: 92 - protein, 112 aa, Mus Musculus
DIVLTQSPASLAVSLGQRATISYRASKSVSTSGYSYMHWNQQKPGQPPRLLIYLVSNLESGVPARFSGSGSGTDFTLNIHPVEEEDAATHYCQHIRELTRSEGGPSWKIKRT

SEQ ID NO: 93 - protein, 116 aa, Mus Musculus
QVQLQQPGSELVRPGASVKLSCKASGYTFTIFWIHWVKQRPGQGLEWIGNIYPGSGGTNYDEKFKSKATLTVDTFSSTAYMQLSSLTSEDSAVYYCTRWGTGKDYWGQGTTLTVSS

SEQ ID NO: 94 - protein, 110 aa, Mus Musculus
DIVLTQSPAIMSASPGEKVTMTCRARSSVSSSYLHWYQQKPGSSPKLWIYSTSNLALGVPARFSGSGSGTSYSLTISSVEAEDAATYYCQQYDSSPFTFGTGTKLEIKRT

SEQ ID NO: 95 - protein, 120 aa, Mus Musculus
  VARIANT 5: Xaa = Any Amino Acid
QVQLXQSGPGKLQPSQTLSLTCSFSGFSLSTYGMGVGWIRQPLGKGLEWLANIWWNDDKYYNSALKSRLTISKDTSNNQVFLKISSVDTADAATYYCVQIANPYWYFDVWGAGTSVTVSS

SEQ ID NO: 96 - protein, 112 aa, Mus Musculus
DIQMTQSPASLAVSLGQRATISYRASKSVSTSGYSYMHWNQQKPGQPPRLLIYLVSNLESGVPARFSGSGSGTDFTLNIHPVEEEDAATYYCQHIRELTRSEGGPSWKSKRT

SEQ ID NO: 97 - protein, 119 aa, Mus Musculus
EVQLQQSGPELVKPGASVKISCKASGYSFTGYHIHWMKQSHVESLEWIGRINPYSGVTDYNQNFKDKANLTVDQSSSTAYMELHSLTSEDSAFYYCARTDYYGPWFAYWGQGTLVTVSA

SEQ ID NO: 98 - protein, 114 aa, Mus Musculus
DIQMTQSPLSLPVSLGDQASISCRSSPSLVHSNGNTYLHWYLQKPGQSPKLLIYTVSNRFSGVPDRFSGSGSGTDFTLKINRVEAEDLGVYFCSQTTHVPWTFGGGTKLEIKRT